(19) United States
(12) Patent Application Publication
(10) Pub. No.: US 2016/0000799 A1
(43) Pub. Date: Jan. 7, 2016

(54) PHARMACEUTICAL COMPOSITIONS OF CETP INHIBITORS

(71) Applicant: DR. REDDY'S LABORATORIES LTD., Hyderabad, Andhra Pradesh (IN)

(72) Inventors: Vijay Kumar Nekkanti, Andhra Pradesh (IN); Shantanu Yeshwant Damle, Maharastra (IN); Bijay Kumar Padhi, Orissa (IN); Rajesh Gandhi, Madhya Pradesh (IN); Rajeev Singh Raghuvanshi, Haryana (IN)

(21) Appl. No.: 14/769,017

(22) PCT Filed: Feb. 21, 2014
(86) PCT No.: PCT/IB2014/000715

§ 371 Date: Aug. 19, 2015

(30) Foreign Application Priority Data
Feb. 21, 2013 (IN) 760/CHEC/2013

(51) Int. Cl. A61K 31/5377; A61K 31/53; A61K 31/496; A61K 31/506; A61K 31/4709; A61K 31/47

(52) U.S. Cl. CPC A61K 31/5377; A61K 31/4709; A61K 31/47; A61K 31/496; A61K 31/506; A61K 31/53

(57) ABSTRACT

The present application discloses a pharmaceutical composition comprising a class of cholesteryl ester transfer protein (CETP) inhibitors with improved oral bioavailability. The application further discloses compositions comprising a class of CETP inhibitor and at least one surfactant in the form of a solution, suspension, emulsion or a pre-concentrate.

### FIELD OF THE APPLICATION

This invention is directed to pharmaceutical compositions containing cholesteryl ester transfer protein (CETP) inhibitors. This invention is further directed to the use of such compositions to elevate certain plasma lipid levels, including high density lipoprotein (HDL)-cholesterol and to lower certain other plasma lipid levels, such as low density lipoprotein (LDL)-cholesterol and triglycerides. Thus, this invention is also directed to treat diseases which are affected by low levels of HDL cholesterol and/or high levels of LDL-cholesterol and triglycerides, such as atherosclerosis and cardiovascular diseases.

### BACKGROUND

Hyperlipidemia or an elevation in serum lipids is associated with an increase incidence of cardiovascular disease and atherosclerosis. Primary hyperlipidemia is a term used to describe a defect in lipoprotein metabolism. The lipoproteins commonly affected are low density lipoprotein (LDL) cholesterol, which transports mainly cholesterol, and very low density lipoprotein-cholesterol (VLDL-cholesterol), which transports mainly triglycerides (TG). Most subjects with hyperlipidemia have a defect in LDL metabolism, characterized by raised cholesterol, LDL-C levels, with or without raised triglyceride levels; such subjects are termed hypercholesterolemic (Fredrickson Type II). Familial hypercholesterolemia (FH) is caused by any one of a number of genetically-determined defects in the LDL receptor, which is important for the entry of cholesterol into cells. The condition is characterized by a reduced number of functional LDL receptors, and is therefore associated with raised serum LDL-C levels due to an increase in LDL.

It is reasonably known in the art that the likelihood of cardiovascular disease can be decreased, if the serum lipids, and in particular LDL-C, can be reduced. It is further known that the progression of atherosclerosis can be retarded or the regression of atherosclerosis can be induced if serum lipids can be lowered. In such cases, individuals diagnosed with hyperlipidemia or hypercholesteremia should consider lipid-lowering therapy to retard the progression or induce the regression of atherosclerosis for purposes of reducing their risk of cardiovascular disease, and in particular coronary artery disease.

Cholesteryl ester-transfer protein (CETP) is an important player in metabolism of lipoproteins, such as, for example, a high density lipoprotein (HDL). CETP is a 70 kDa plasma glycoprotein that is physically associated with HDL particles. It facilitates the transport of cholesteryl ester from HDL to apolipoprotein B-containing lipoproteins. This transfer is accompanied by transfer of triglycerides in the opposite direction. Thus, a decrease in CETP activity can result in an increase in the level of HDL cholesterol and a decrease in the level of very low density lipoprotein (VLDL) and low density lipoprotein (LDL). CETP can therefore simultaneously affect the concentrations of pro-atherogenic (for example, LDL) and anti-atherogenic (for example, HDL) lipoproteins.

Several CETP inhibitors are currently in various clinical phases of development for treating various aforementioned disorders. In spite of having various advantages, CETP inhibitors are proven to be difficult to formulate for oral administration. CETP inhibitors are of a highly lipophilic nature and have extremely low solubility in water. Due to their poor solubility, bioavailability of conventional oral compositions is very poor. The lipophilic nature of CETP inhibitors not only leads to low solubility but also tends to poor wettability, further reducing their tendency to be absorbed from the gastrointestinal tract. In addition to the low solubility, CETP inhibitors also tend to have significant, “food effect”, where a significant difference in rate and amount of drug absorption is observed when the drug is administered with or without a meal. This “food effect”, often complicates the dosing regimen and may require high dosing to achieve the desired therapeutic effect, resulting in potentially unwanted side effects.

Several attempts have been made to improve the solubility of CETP inhibitors, but have generally ended up with limited success. At the outset, most methods aimed at enhancing aqueous concentration and bioavailability of low-solubility drugs only offer moderate improvements. References describing improving the dissolution of poorly soluble drugs include: U.S. Pat. Nos. 5,456,923, 5,993,858, 6,057,289, 6,096,338, 6,267,985, 6,280,770, 6,436,430, 6,451,339, 6,531,139, 6,555,558, 6,638,522, 6,962,931 and 7,374,779.

In view of the foregoing, there remains a long felt need for developing compositions containing CETP inhibitors with improved bioavailability and minimal or less food effect.

### SUMMARY

In one aspect, the present application relates to a pharmaceutical composition comprising:
a) a cholesteryl ester transfer protein (CETP) inhibitor having formula (I) or (Ia′) or (II) or (III),
b) at least one surfactant,
c) optionally a carrier material, and
d) optionally one or more pharmaceutically acceptable excipients.

In another aspect, the present application relates to a pharmaceutical composition comprising:
a) a cholesteryl ester transfer protein (CETP) inhibitor having formula (I) or (Ia′) or (II) or (III),
b) at least one hydrophilic surfactant,
c) optionally a carrier material, and
d) optionally one or more pharmaceutically acceptable excipients.

In another aspect, the present application provides a composition in which the CETP inhibitor of formula (I), (Ia′), (II) or (III) is combined with at least one surfactant in a sufficient amount so that the composition provides maximum drug availability for absorption.

In yet another aspect, the present application provides a composition comprising a CETP inhibitor of formula (I), (Ia′), (II) or (III) and at least one surfactant in the form of a solution, suspension, emulsion or a pre-concentrate.

In one embodiment, the present application provides a composition comprising a CETP inhibitor of formula (I), (Ia′), (II) or (III) and at least one surfactant, wherein said composition
releases not less than 20% at a period of 15 minutes or
releases not less than 40% at a period of 30 minutes or
releases not less than 75% at a period of 60 minutes.

in 900 ml of 0.01N HCl, when tested in a USP Type 2 apparatus at 50 rpm and 37° C.

In one embodiment, the present application provides a composition comprising a CETP inhibitor of formula (I), (Ia′), (II) or (III) and at least one surfactant, wherein said composition
releases not less than 25% at a period of 15 minutes or
releases not less than 50% at a period of 30 minutes or
releases not less than 90% at a period of 60 minutes in 900 ml of 0.01N HCl, when tested in a USP Type 2 apparatus at 50 rpm and 37° C.

In another aspect, the present application provides a method of administering to a patient a pharmaceutical composition as described herein.

### DETAILED DESCRIPTION

The present application will be described in more detail below.

While the specification concludes with the claims particularly pointing and distinctly claiming the invention, it is believed that the present invention will be better understood from the following description. The present invention can comprise (open ended) or consist essentially of the components of the present invention as well as other ingredients or elements described herein. As used herein, “comprising” means the elements recited, or their equivalent in structure or function, plus any other element or elements which are not recited. The terms “having,” “including,” and “comprised of” are also to be construed as open ended unless the context suggests otherwise. As used herein, “consisting essentially of” means that the invention may include ingredients in addition to those recited in the claim, but only if the additional ingredients do not materially alter the basic and novel characteristics of the claimed invention. Generally, such additives may not be present at all or only in trace amounts. However, it may be possible to include up to about 10% by weight of materials that could materially alter the basic and novel characteristics of the invention as long as the utility of the compounds (as opposed to the degree of utility) is maintained. All ranges recited herein include the endpoints, including those that recite a range “between” two values. Terms such as “about,” “generally,” “substantially,” and the like are to be construed as modifying a term or value such that it is not an absolute. Such terms will be defined by the circumstances and the terms that they modify as those terms are understood by those of skill in the art. This includes, at very least, the degree of expected experimental error, technique error and instrument error for a given technique used to measure a value.

The definitions of the groups and other variables mentioned in formula (I) and (Ia′) are as defined in US2006/0178514 and are described in detail below.

Definitions of the groups and other variables mentioned in formula (I) have the meaning as defined below:

The terms “halogen” or “halo” includes fluorine, chlorine, bromine, or iodine.

The term “alkyl” group is used to refer to both linear and branched alkyl groups. Exemplary alkyl groups include, but are not limited to, methyl, ethyl, propyl, butyl, pentyl, hexyl, heptyl, octyl, nonyl, or decyl, and the like. Unless otherwise specified, an alkyl group has from 1 to 12 carbon atoms. Also unless otherwise specified, all structural isomers of a given structure, for example, all enantiomers and all diasteriomers, are included within this definition. For example, unless otherwise specified, the term propyl is meant to include n-propyl and iso-propyl, while the term butyl is meant to include n-butyl, iso-butyl, t-butyl, sec-butyl, and so forth.

The term “aryl” refers to an optionally substituted monocyclic or polycyclic aromatic ring system of 6 to 14 carbon atoms. Exemplary groups include phenyl, naphthyl, 1,2,3,4-tetrahydronaphthalene, indane, fluorene, and the like. Unless otherwise specified, an aryl group typically has from 6 to 14 carbon atoms.

“Aralkyl” refers to an aryl substituted alkyl group, wherein the aryl group and the alkyl group are defined herein. Typically, the aryl group can have from 6 to 14 carbon atoms, and the alkyl group can have up to 10 carbon atoms. Exemplary aralkyl groups include, but are not limited to, benzyl, phenylethyl, phenylpropyl, phenylbutyl, propyl-2-phenylethyl and the like.

The term “haloalkyl” refers to a group containing at least one halogen and an alkyl portion as define above, that is, a haloalkyl is a substituted alkyl group that is substituted with one or more halogens. Unless otherwise specified, all structural isomers of a given structure, for example, all enantiomers and all diasteriomers, are included within this definition. Exemplary haloalkyl groups include fluoromethyl, chloromethyl, fluoroethyl, chloroethyl, trifluoromethyl, and the like. Unless otherwise specified, a haloalkyl group has from 1 to 12 carbon atoms.

A “cycloalkyl” group refers to a cyclic alkyl group which can be mono or polycyclic. Exemplary cycloalkyl groups include cyclopropyl, cyclobutyl, cyclopentyl, cyclohexyl, cycloheptyl, cyclooctyl, cyclononyl, and cyclodecyl. Unless otherwise specified, a cycloalkyl group has from 3 to 12 carbon atoms.

An “alkoxy” group refers to an —O(alkyl) group, where alkyl is as defined herein. Therefore, unless otherwise specified, all isomers of a given structure are included within a definition. Exemplary alkyl groups include methoxy, ethoxy, n-propoxy, iso-propoxy, n-butoxy, iso-butoxy, t-butoxy, and the like. Unless otherwise specified, an alkoxy group has from 1 to 12 carbon atoms. Unless otherwise specified, all structural isomers of a given structure, for example, all enantiomers and all diasteriomers, are included within this definition. For example, unless otherwise specified, the term propoxy is meant to include n-propoxy and iso-propoxy.

An “aryloxy” group refers to an —O(aryl) group, where aryl is as defined herein. Thus, the aryl portion of an aryloxy group can be substituted or unsubstituted. Exemplary aryloxy groups include, but are not limited to, phenoxy, naphthyl, and the like. Unless otherwise specified, an aryloxy group typically has from 6 to 14 carbon atoms.

“Haloalkoxy” refers to an alkoxy group with a halo substituent, where alkoxy and halo groups are as defined above. Exemplary haloalkoxy groups include fluoromethoxy, chloromethoxy, trifluoromethoxy, trichloroethoxy, fluoroethoxy, chloroethoxy, trifluoroethoxy, perfluoroethoxy (—OCF₂CF₃), trifluoro-t-butoxy, hexafluoro-t-butoxy, perfluoro-t-butoxy (—OC(CF₃)₃), and the like. Unless otherwise specified, an haloalkoxy group typically has from 1 to 12 carbon atoms.

“Alkylthio” refers to an —S(alkyl) group, where alkyl group is as defined above. Exemplary alkyl groups include methylthio, ethylthio, propylthio, butylthio, iso-propylthio, iso-butylthio, and the like. Unless otherwise specified, an alkylthio group typically has from 1 to 12 carbon atoms.

“Heteroaryl” is an aromatic monocyclic or polycyclic ring system of 4 to 10 carbon atoms, having at least one heteroatom or heterogroup selected from —O—, >N—, —S—, >NH or NR, and the like, wherein R is a substituted or unsubstituted alkyl, aryl, or acyl, as defined herein. In this aspect, >NH or NR are considered to be included when the heteroatom or heterogroup can be >N—. Exemplary heteroaryl groups include as pyrazinyl, isothiazolyl, oxazolyl, pyrazolyl, pyrrolyl, triazolyl, tetrazolyl, oxatriazolyl, oxadiazolyl, pyridazinyl, thienopyrimidyl, furanyl, indolyl, isoindolyl, benzo[1,3]dioxolyl, 1,3-benzoxathiole, quinazolinyl, isoquinolinyl, quinolinyl, pyridyl, 1,2,3,4-tetrahydro-isoquinolinyl, 1,2,3,4-tetrahydro-quinolinyl pyridyl, thiophenyl, and the like. Unless otherwise specified, a heteroaryl group typically has from 4 to 10 carbon atoms. Moreover, the heteroaryl group can be bonded to the heterocyclic core structure at a ring carbon atom, or, if applicable for a N-substituted heteroaryl such as pyrrole, can be bonded to the heterocyclic core structure through the heteroatom that is formally deprotonated to form a direct heteroatom-pyrimdine ring bond.

“Heterocyclyl” is a non-aromatic, saturated or unsaturated, monocyclic or polycyclic ring system of 3 to 10 member having at least one heteroatom or heterogroup selected from —O—, >N—, —S—, >NR, >SO₂, >CO₃ and the like, wherein R is hydrogen or a substituted or an unsubstituted alkyl, aryl, or acyl, as defined herein. Exemplary heterocyclyl groups include aziridinyl, imidazolidinyl, 2,5-dihydro-[1,2,4]oxadiazolenyl, oxazolidinyl, isooxazolidinyl, pyrrolidinyl, piperdinyl, piperazinyl, morpholinyl, thiomorpholinyl, thiazolidinyl, 1,3-dioxolanyl, 1,4-dioxanyl, 2,5-dihydro-1H-imidazolyl, and the like. Unless otherwise specified, a heterocyclyl group typically has from 2 to 10 carbon atoms. A heterocyclyl group can be bonded through a heteroatom that is formally deprotonated or a heterocyclyl group can be bonded through a carbon atom of the heterocyclyl group.

“Heterocycloalkyl” refers to the saturated subset of a heterocyclyl, that is, a non-aromatic, saturated monocyclic or polycyclic ring system of 3 to 10 members having at least one heteroatom or heterogroup selected from —O—, >N—, —S—, >NR, >SO₂, >CO₃ and the like, wherein R is hydrogen or a substituted or an unsubstituted alkyl, aryl, or acyl, as defined herein. Exemplary heterocycloalkyl groups include aziridinyl, piperdinyl, piperazinyl, morpholinyl, thiazolidinyl, 1,3-dioxolanyl, 1,4-dioxanyl, and the like. Unless otherwise specified, a heterocycloalkyl group typically has from 2 to 10 carbon atoms, or in another aspect, from 2 to 6 carbon atoms. A heterocycloalkyl group can be bonded through a heteroatom that is formally deprotonated or a heterocycloalkyl group can be bonded through a carbon atom of the heterocycloalkyl group.

A “heteroaryloxy” group refers to an aryloxy-type analog of a heteroaryl group. Thus, a heteroaryloxy group is intended to describe a heteroaryl group as defined herein, that is bonded to an oxygen atom, to form a formal [O-heteroaryl] moiety. Unless otherwise specified, a heteroaryloxy group typically comprises from 4 to 10 carbon atoms.

A “cyclic” moiety, including a monocyclic moiety or a bicyclic moiety, unless otherwise specified, is intended to be inclusive of all the cyclic groups disclosed herein, for example, a heteroaryl group, a heterocyclyl group, a heterocycloalkyl group, and/or a heteroaryloxy group.

An “alkoxycarbonyl” group refers to a —C(O)O(alkyl) group, wherein the alkyl portion of the alkoxycarbonyl group is defined as herein. Examples of alkoxycarbonyl groups include, but are not limited to, methoxycarbonyl, ethoxycarbonyl, t-butoxycarbonyl and the like.

An “alkenyl” group is an aliphatic hydrocarbon group comprising an alkene functionality, regardless of the regiochemistry of the alkene functionality within the aliphatic hydrocarbon group. Unless otherwise specified, an alkenyl group typically has from 2 to 12 carbon atoms, and in another aspect, is a C₂-C₁₀ alkenyl group. Exemplary alkenyl groups include ethenyl, propenyl, butenyl, and the like, including all regiochemistries, thus, “butenyl” includes 1-butenyl, 2-butenyl, and 3-butenyl.

An “alkynyl” group is an aliphatic hydrocarbon group comprising an alkyne functionality, regardless of the regiochemistry of the alkyne functionality within the aliphatic hydrocarbon group. Unless otherwise specified, an alkynyl group typically has from 2 to 12 carbon atoms, and in another aspect, is a C₂-C₁₀ alkynyl group. Exemplary alkynyl groups include ethynyl, propynyl, butynyl, and the like, including all regiochemistries. Thus, “butynyl” includes 1-butynyl, 2-butynyl, and 3-butynyl.

An “alkoxyalkyl” group is an alkoxy-substituted alkyl group, wherein an alkoxy group and an alkyl group are defined herein. Unless otherwise specified, an alkoxyalkyl group typically has from 2 to 20 carbon atoms. In one aspect, an alkoxyalkyl group can be a (C₁-C₁₀) alkoxy group bonded to a (C₁-C₁₀) alkyl group, where alkoxy and alkyl groups are as defined here, including all stereochemistries and all regiochemistries. Exemplary alkoxyalkyl groups include methoxymethyl, methoxyethyl, methoxypropyl, ethoxymethyl, ethoxyethyl, methoxyisopropyl, ethoxyisobutyl, and the like.

An “aminoalkyl” group, as used herein, refers to an amino-substituted alkyl group, wherein an alkyl is defined herein. Unless otherwise specified, an aminoalkyl group can typically have from 1 to 12 carbon atoms, therefore, a typical aminoalkyl group can be an amino (C₁-C₁₂) alkyl, including all regiochemistries. Exemplary aminoalkyl groups include, but are not limited to, aminomethyl, aminoethyl, aminopropyl, and the like.

A “cycloalkyl-substituted alkyl” group, also termed a “cycloalkylalkyl” group, refers to an alkyl group that is substituted with a cycloalkyl substituent, wherein alkyl and cycloalkyl are defined herein. Thus, the cycloalkyl group portion can be a mono or polycyclic alkyl group. Unless otherwise specified, a cycloalkylalkyl group can have up to 20 carbon atoms, regardless of how the carbon atoms are distributed between the alkyl portion and the cycloalkyl portion of the group, and including all possible stereochemistries and all regiochemistries. For example, in one aspect, a cycloalkyl-substituted alkyl can comprise a (C₃-C₁₀) cycloalkyl bonded to a C₁-C₁₀ alkyl group, wherein the cycloalkyl portion can be mono or polycyclic. Exemplary cycloalkylalkyl groups include, but are not limited to, cyclopropylmethyl, cyclopropylethyl, cyclopropylpropyl, cyclobutylmethyl, cyclobutylethyl, cyclobutylpropyl, cyclopentylmethyl, cyclopentylethyl, cyclopentylpropyl, cyclohexylmethyl, cyclohexylethyl, cyclohexylpropyl, cycloheptylmethyl, cycloheptylethyl, cyclooctylmethyl, cyclooctylethyl, cyclooctylpropyl, and the like.

A “cycloalkoxy” group, also referred to as a “cycloalkyloxy” group, refers herein to an —O(cycloalkyl) substituent, that is, an alkoxide-type moiety comprising a cycloalkyl group, wherein a cycloalkyl is defined herein. Thus, the cycloalkyl group portion can be a mono or polycyclic alkyl group, and unless otherwise specified, a cycloalkylalkyl group can have up to 20 carbon atoms. In one aspect, a cycloalkoxy group can be a (C₃-C₁₀) cycloalkyl-O— group. Exemplary cycloalkoxy groups include cyclopropoxy, cyclobutoxy, cyclopentoxy, cyclohexoxy, and the like.

An “acyl” group refers to a (C₁-C₁₀) alkyl-CO— group, wherein the (C₁-C₁₀) alkyl group is used in this structure to refer to the alkyl-linker moiety bonded both to the CO group, and to another chemical group. Examples of acyl groups include, but are not limited to, methylcarbonyl, ethylcarbonyl, propylcarbonyl, isopropylcarbonyl, and the like.

An “alkenylene” group refers to a (C₂-C₁₀) hydrocarbon linker comprising at least one C═C double bond within the C₂-C₁₀ chain. Examples of alkenylene groups include, but are not limited to, —CH═CH—, —CH₂—CH═CH, —CH₂—CH═CH—CH₂—, —CH₂—CH═CH—CH═CH—, and the like. Thus, unless otherwise specified, an alkenylene group has from 2 to 10 carbon atoms.

A “haloalkoxyalkyl” group refers to a haloalkyl-O—(C₁-C₁₀)alkyl group, that is, a haloalkoxy-substituted alkyl group, wherein haloalkoxy and alkyl are defined herein. Unless otherwise specified, a cycloalkylalkyl group can have up to 20 carbon atoms, regardless of how the carbon atoms are distributed between the haloalkoxy portion and the alkyl portion of the group, and including all possible sterochemistries and all regiochemistries. In one aspect, for example, a haloalkoxyalkyl is haloalkyl-O—(C₁-C₁₀)alkyl, where group can be (C₁-C₁₀) haloalkyl group bonded to a (C₁-C₁₀) alkyl moiety. Exemplary haloalkoxyalkyl groups include trifluoromethoxymethyl, chloromethoxyethyl, flouroethoxyethyl, chloroethoxyethyl, trilfluoromethoxypropyl, hexafluoroethoxyethyl and the like.

A “monoalkylamino” group refers to an amino group that is substituted with a single alkyl group, that is, a mono(C₁-C₂₀)alkylamino group. Unless otherwise specified, a monoalkylamino group can have up to 20 carbon atoms. In one aspect, a monoalkylamino group can be a (C₁-C₁₀)alkyl-substituted amino group. Exemplary monoalkylamino groups include methylamino, ethylamino, propylamino, isopropylamino, and the like.

A “dialkylamino” group refers to an amino group that is substituted with two, independently-selected, alkyl groups, that is, a di (C₁-C₁₀) alkylamino group. Unless otherwise specified, a dialkylamino group can have up to 20 carbon atoms. Exemplary dialkylamino groups include dimethylamino, diethylamino, and the like.

Definitions of the groups and other variables mentioned in formula (II) and (III) have the meaning as defined below:

As used herein, the expression ‘alkyl’ group refers to linear or branched alkyl group with 1 to 10 carbon atoms. Exemplary alkyl group includes, but is not limited to, methyl, ethyl, n-propyl, iso-propyl, n-butyl, iso-butyl, t-butyl, n-pentyl, iso-pentyl, hexyl, heptyl, octyl and the like.

As used herein, the expression ‘alkoxy’ group refers to an —O (alkyl) group, wherein alkyl group is as defined above. Exemplary alkoxy groups include methoxy, ethoxy, n-propoxy, iso-propoxy, n-butoxy, iso-butoxy, t-butoxy, and the like. Unless otherwise specified, an alkoxy group has from 1 to 10 carbon atoms.

As used herein, the expression ‘alkoxyalkyl’ means at least one alkoxy group is substituted on an alkyl group. Both alkoxy and alkyl have the meaning as defined above. Representative examples of alkoxyalkyl groups include, but are not limited to, ethoxymethyl, methoxyethyl, isopropoxyethyl, 2-methoxybut-1-yl, 3,3-dimethoxyprop-1-yl, and the like. Unless otherwise specified, an alkoxyalkyl group typically has from 1 to 10 carbon atoms.

As used herein, the expression ‘acyl’ group refers to alkyl-CO— group, wherein alkyl group is as defined above. Acyl group refers to an alkyl-linker moiety bonded both to the CO group, and to another chemical group. Examples of acyl groups include, but are not limited to, acetyl, propionyl and the like. Acyl group includes formyl group too.

As used herein, the expression ‘aryl’ means substituted or unsubstituted phenyl or naphthyl. Specific examples of substituted phenyl or naphthyl include o-, p-, m-tolyl, 1,2-, 1,3-, 1,4-xylyl, 1-methylnaphthyl, 2-methylnaphthyl, etc. “Substituted phenyl” or “substituted naphthyl” also include any of the possible substituents as further defined herein or one known in the art. Derived expression, “arylsulfonyl,” is to be construed accordingly.

As used herein, the expression ‘Cycloalkyl’ group refers to a cyclic alkyl group which may be mono, bicyclic, polycyclic, or a fused/bridged ring system. Exemplary cycloalkyl groups include, but are not limited to, cyclopropyl, cyclobutyl, cyclopentyl, cyclohexyl, cycloheptyl, cyclooctyl, and the like. Unless otherwise specified, a cycloalkyl group typically has from 3 to about 10 carbon atoms. Typical bridged cycloalkyls include, but are not limited to adamantyl, noradamantyl, bicyclo[1.1.0]butanyl, norbornyl(bicyclo[2.2.1]heptanyl), norbornenyl(bicyclo[2.2.1]heptanyl), norbornadienyl(bicyclo[2.2.1]heptadienyl), bicyclo[2.2.1]heptanyl, bicyclo[3.2.1]octanyl, bicyclo[3.2.1]octadienyl, bicyclo[2.2.2]octanyl, bicyclo[2.2.2]octenyl, bicyclo[2.2.2]octadienyl, bicyclo[5.2.0]nonanyl, bicyclo[4.3.2]undecanyl, tricyclo[5.3.1.1]dodecanyl and the like.

As used herein, the expression ‘halogen or halo’ represents fluorine, chlorine, bromine, or iodine.

As used herein, the expression ‘haloalkyl’ means at least one halogen atom is substituted on an alkyl group. Both halogen and alkyl have the meaning as defined above. Representative examples of haloalkyl groups include, but are not limited to, fluoromethyl, chloromethyl, fluoroethyl, chloroethyl, difluoromethyl, trifluoromethyl, dichloroethyl, trichloroethyl and the like. Unless otherwise specified, a haloalkyl group typically has from 1 to 10 carbon atoms.

As used herein, the expression ‘haloalkoxy’ means at least one halogen atom is substituted on an alkoxy group, wherein alkoxy and halogen groups are as defined above. Exemplary haloalkoxy groups include, but not limited to, fluoromethoxy, chloromethoxy, trifluoromethoxy, trichloroethoxy, fluoroethoxy, chloroethoxy, trifluoroethoxy, perfluoroethoxy (—OCF₂CF₃), trifluoro-t-butoxy, hexafluoro-t-butoxy, perfluoro-t-butoxy (—OC(CF₃)₃), and the like. Unless otherwise specified, a haloalkoxy group typically has from 1 to 10 carbon atoms.

As used herein, the expression ‘heterocycle’ or ‘heterocyclyl’ or ‘heterocyclic’ is a saturated monocyclic or polycyclic ring system of 3 to 10 members having at least one heteroatom or heterogroup selected from —O—, —N—, —S—, —SO₂, or —CO. Exemplary heterocyclyl groups include, but not limited to, azetidinyl, oxazolidinyl, oxazolidinonyl, isoxazolidinyl, imidazolidin-2-onyl, pyrrolidinyl, pyrrolidin-2-onyl, piperidinyl, piperazinyl, morpholinyl, thiomorpholinyl, thiomorpholine-1,1-dioxide, thiazolidinyl, 1,3-dioxolanyl, 1,4-dioxanyl, and the like. Unless otherwise specified, a heterocyclyl group typically has from 3 to about 10 carbon atoms.

As used herein, the expression ‘heteroaryl’ is an unsaturated, aromatic or non-aromatic, monocyclic or polycyclic ring system of 3 to 10 members having at least one heteroatom or heterogroup selected from —O—, —N—, —S—, —SO₂, or —CO. Exemplary heteroaryl groups include, but not limited to, oxazolyl, isoxazolyl, thiazolyl, pyridinyl, pyrrolyl, pyrimidinyl, thiazinyl, pyrazinyl, pyrazolyl, tetrazolyl, imidazothiazolyl, indolizidinyl, indolyl, quinolinyl, quinoxalinyl, benzoxazolyl, benzoisoxazolyl, benzothiazolyl, benzodioxolyl, benzotriazolyl, indazolyl, quinoxalinyl, imidazolyl, pyrazolopyridinyl, and the like. Unless otherwise specified, a heteroaryl group typically has from 3 to about 10 carbon atoms.

As used herein, the expression ‘5-7 membered heterocyclic or heteroaryl group’ represents a heterocyclic or heteroaryl group as defined above having 5-7 ring atoms. Exemplary 5-7 membered heterocyclic or heteroaryl groups include, but not limited to, pyrazolyl, imidazolyl, isoxazolyl, oxazolyl, tetrazolyl, morpholinyl, oxazolidinonyl, and the like.

As used herein, the expression ‘OH’ represents a hydroxy group.

As used herein, the expression ‘CN’ represents a cyano group.

### Cholesterol Ester Transfer Protein (CETP) Inhibitor:

The CETP inhibitors that are essentially aqueous insoluble, highly hydrophobic, and are characterized by a set of physical properties. Several characteristic properties of this class of compounds are
(i) hydrophobic CETP inhibitors have extremely low aqueous solubility. Extremely low aqueous solubility is meant that the minimum aqueous solubility at physiologically relevant pH (pH of 1 to 8) is less than about 10 μg/ml, less than about 2 μg/ml, or less than about 1 μg/ml.
(ii) essentially insoluble, hydrophobic CETP inhibitors are that they are extremely hydrophobic. Extremely hydrophobic is meant that the C log P value of the drug, has a value of at least 4.0, a value of at least 5.0, or a value of at least 6.0.
(iii) a very high dose-to-solubility ratio. By “very high dose-to-solubility ratio” is meant that the dose-to-solubility ratio has a value of at least 1000 ml, preferably value of at least 5,000 ml, at least 8,000 ml or a value of at least 10,000 ml.
(iv) have very low absolute bioavailability. The absolute bioavailability of drugs in this subclass when dosed orally in their undispersed state is less than about 10% and more often less than about 5%.

Wherever CETP inhibitors are not limited by a particular structural class, the present application is not limited by any particular structure or group of CETP inhibitors. Rather, the application has general applicability to CETP inhibitors as a class, the class tending to be composed of compounds having low solubility.

In one aspect, the present application relates to a pharmaceutical composition comprising:
a) a cholesteryl ester transfer protein (CETP) inhibitor having formula (I) or (Ia′) or (II) or (III),
b) at least one surfactant,
c) optionally a carrier material, and
d) optionally one or more pharmaceutically acceptable excipients.

In one embodiment of the above aspect, the present application relates to a pharmaceutical composition comprising:
a) a cholesteryl ester transfer protein (CETP) inhibitor having formula (I),
b) at least one surfactant,
c) optionally a carrier material, and
d) optionally one or more pharmaceutically acceptable excipients; wherein formula (I) is defined as follows,

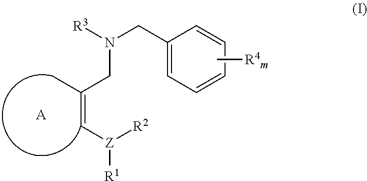

or a stereoisomer thereof or a pharmaceutically acceptable salt thereof, wherein:
A is a substituted or an unsubstituted quinoline moiety having the formula:

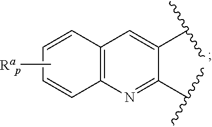

wherein R^{a}, in each occurrence, is selected independently from: 1) a halogen; a hydroxyl, or a cyano; 2) an alkyl or an alkoxy, any of which having up to 12 carbon atoms; or 3) CO₂R⁶; and p is an integer from 0 to 3, inclusive;
R¹ and R² are selected independently from: 1) hydrogen; 2) a substituted or an unsubstituted alkyl, cycloalkyl, haloalkyl, aryl, heterocyclyl, heteroaryl, any of which having up to 12 carbon atoms, wherein any heterocyclyl or heteroaryl comprises at least one heteroatom or heterogroup selected independently from O, N, S, NR¹⁰, SO₂, or CO; 3) CO₂R⁶, COR⁸, SO₂R⁸, SO₂NR⁶R⁷, or CONR⁶R⁷; or 4) (CHR^{x})ₙR⁵ or (CH₂)ₙR^{d}CO₂R^{e}, wherein n, in each occurrence, is 1, 2, or 3; R^{x}, in each occurrence, is selected independently from an alkyl or an alkoxy, either of which having up to 12 carbon atoms, or hydrogen; R^{d}, in each occurrence, is selected independently from an alkyl, a cycloalkyl, an aryl, a heterocyclyl, or a heteroaryl, any of which having up to 12 carbon atoms, wherein any heterocyclyl or heteroaryl comprises at least one heteroatom or heterogroup selected independently from O, N, S, NR¹⁰, SO₂, or CO; and R^{e}, in each occurrence, is selected independently from an alkyl or a cycloalkyl, either of which having up to 12 carbon atoms, or hydrogen;
or R¹ and R² together with the diradical Z to which they are attached form a substituted or an unsubstituted monocyclic or bicyclic moiety comprising up to 12 carbon atoms, and optionally comprising 1, 2, or 3 heteroatoms or heterogroups in addition to Z, selected independently from O, N, S, NR¹⁰, SO₂, or CO;
R³ is selected from: 1) hydrogen or cyano; 2) a substituted alkyl having up to 12 carbon atoms; 3) a substituted or an unsubstituted aryl, or a substituted or an unsubstituted 5-, 6-, or 7-membered heterocyclyl or heteroaryl, any of which having up to 12 carbon atoms, comprising 1, 2, or 3 heteroatoms or heterogroups selected independently from O, N, S, NR¹⁰, SO₂, or CO; or 4) CO₂R⁶, COR⁸, SO₂R⁸, SO₂NR⁶R⁷, CONR⁶R⁷, C(S)NR⁶R⁷, C(S)NC(O)OR⁸, or C(S)SR⁸; or 5) a substituted or an unsubstituted group selected from 4,5-dihydro-oxazolyl, tetrazolyl, isoxazolyl, pyridyl, pyrimidinyl, oxadiazolyl, thiazolyl, or oxazolyl; wherein any optional substituent is selected independently from: a) an alkyl or haloalkyl, any of which having up to 12 carbon atoms; or b) CO₂R⁹, wherein R⁹ is an alkyl having up to 12 carbon atoms;
wherein when R³ is an aryl, a heterocyclyl, or a heteroaryl, R³ is optionally substituted with up to three substituents selected independently from a halogen, a hydroxyl, a cyano, an alkoxy having up to 12 carbon atoms, or R¹¹;
R⁴, in each occurrence, is selected independently from: 1) halogen, cyano, or hydroxy; 2) an alkyl, a cycloalkyl, a cycloalkoxy, an alkoxy, a haloalkyl, or a haloalkoxy, any of which having up to 12 carbon atoms; 3) a substituted or an unsubstituted aryl, aralkyl, aryloxy, heteroaryl, or heteroaryloxy, any of which having up to 12 carbon atoms, wherein any heteroaryl or heteroaryloxy comprises at least one heteroatom or heterogroup selected independently from O, N, S, or NR¹⁰; or 4) CO₂R⁶, COR⁸, SO₂R⁸, SO₂NR⁶R⁷, CONR⁶R⁷, or (CH₂)_{q}NR⁶R⁷, wherein q is an integer from 0 to 5, inclusive;
m is an integer from 0 to 3, inclusive;
or R⁴ₘ is a fused cyclic moiety comprising from 3 to 5 additional ring carbon atoms, inclusive, and optionally comprising at least one heteroatom or heterogroup selected independently from O, N, S, NR¹⁰, SO₂, or CO;
R⁵, in each occurrence, is selected independently from: 1) an alkoxy, a haloalkoxy, or a cycloalkyl, any of which having up to 12 carbon atoms; 2) a substituted or an unsubstituted aryl, heterocyclyl, or heteroaryl, any of which having up to 12 carbon atoms, wherein any heterocyclyl or heteroaryl comprises at least one heteroatom or heterogroup selected independently from O, N, S, NR¹⁰, SO₂, or CO; 3) hydroxyl, NR⁶R⁷, CO₂R⁶, COR^{E}, or SO₂R⁸; or 4) a substituted or an unsubstituted heterocycloalkyl comprising from 3 to 7 ring carbon atoms, and from 1 to 3 heteroatoms or heterogroups, inclusive, selected independently from O, N, S, NR¹⁰, SO₂, or CO;
R⁶ and R⁷, in each occurrence, are selected independently from: 1) hydrogen; 2) an alkyl, a cycloalkyl, or a haloalkyl, any of which having up to 12 carbon atoms; or 3) a substituted or an unsubstituted aryl, aralkyl, heterocyclyl, or heteroaryl, any of which having up to 12 carbon atoms, wherein any heterocyclyl or heteroaryl comprises at least one heteroatom or heterogroup selected independently from O, N, S, NR¹⁰, SO₂, or CO;
or R⁶ and R⁷ together with the nitrogen atom to which they are attached form a substituted or an unsubstituted cyclic moiety having from 3 to 7 ring carbon atoms, and optionally comprising 1, 2, or 3 heteroatoms in addition to the nitrogen atom to which R⁶ and R⁷ are bonded, selected independently from O, N, S, or NR¹⁰;
R⁸, in each occurrence, is selected independently from: 1) an alkyl, a cycloalkyl, or a haloalkyl, any of which having up to 12 carbon atoms; or 2) a substituted or an unsubstituted aryl, heterocyclyl, or heteroaryl, any of which having up to 12 carbon atoms, wherein any heterocyclyl or heteroaryl comprises at least one heteroatom or heterogroup selected independently from O, N, S, NR¹⁰, SO₂, or CO;
R¹⁰, in each occurrence, is selected independently from: 1) hydrogen; or 2) an alkyl, a cycloalkyl, a haloalkyl, an aryl, or an aralkyl, any of which having up to 12 carbon atoms;
Z is N or CH; or the ZR¹ moiety is S, CO, or SO₂; or the ZR¹R² moiety is —C≡CR²;
R¹¹ is selected independently from:
1) an alkyl, a haloalkyl, a cycloalkyl, or an alkoxycarbonyl, any of which having up to 12 carbon atoms;
2) a substituted or an unsubstituted heteroaryl or heterocyclyl, any of which having up to 12 carbon atoms, comprises at least one heteroatom or heterogroup selected independently from O, N, S, NR¹⁰, SO₂, or CO, wherein any substituted heteroaryl or heterocyclyl is substituted with up to three substituents selected independently from an alkyl having up to 12 carbon atoms or a hydroxyl; or
3) —CO—Z²—R¹³, —CO—R¹², —CO—Z²—(CH₂)ᵣ—CO—Z²—R¹³, —NR¹⁵R¹⁶, Z²—CO—(CH₂)ᵣ—Z²—R¹³, —Z²—CO—(CH₂)ᵣ—CO—Z²—R¹³, —O—(CH₂)ᵣ—CO—Z²—R¹³, —O—(CH₂)ᵣ—R¹⁴, —O—R¹²—(CH₂)ᵣ—R¹³, —O—R¹⁴—CO—O—R¹³, —O—(CH₂)ᵣ—R¹², —O—(CH₂)ᵣ—NR′R″, —O—(CH₂)ᵣ—CO₂—(CH₂)ᵣ—R¹³, —O—(CH₂)ᵣ—SR⁸, —O—(CH₂)ᵣ—CO₂—R¹³, —O—(CH₂)ᵣ—CONR′R″, —O—(CH₂)ᵣ—CONH—(CH₂)ᵣ—OR¹³, —O—(CH₂)ᵣ—SO₂R⁸, —O—(CH₂)ᵣ—R¹³, —O—(CH₂)ᵣ—OR¹³, —O—(CH₂)ᵣ—O—(CH₂)ᵣ—OR¹³, —S—(CH₂)ᵣ—CONR′R″, —SO₂—(CH₂)ᵣ—OR¹³, —SO₂—(CH₂)ᵣ—CONR′R″, —(CH₂)ᵣ—O—CO—R⁸, —(CH₂)ᵣ—R¹², —(CH₂)ᵣ—R¹³, —(CH₂)ᵣ—CO—Z²—R¹³, —(CH₂)ᵣ—Z²—R¹³, or -alkenylene-CO₂—(CH₂)ᵣ—R¹³;
r, in each occurrence, is independently 1, 2, or 3;
R¹², in each occurrence, is independently selected from a substituted or an unsubstituted heterocyclyl having up to 12 carbon atoms, comprising at least one heteroatom or heterogroup selected independently from O, N, S, NR¹⁰, SO₂, or CO, wherein any substituted heterocyclyl is substituted with up to three substituents selected independently from an acyl, an alkyl, or an alkoxycarbonyl, any of which having up to 12 carbon atoms, or —COOH;
R¹³, in each occurrence, is independently selected from: 1) hydrogen; or 2) a cycloalkyl, an aryl, a haloalkyl, a heterocyclyl, or an alkyl group optionally substituted with at least one hydroxyl, any of which having up to 12 carbon atoms, wherein any heterocyclyl comprises at least one heteroatom or heterogroup selected independently from O, N, S, NR¹⁰, SO₂, or CO;
R¹⁴, in each occurrence, is independently selected from a heterocyclyl, a cycloalkyl, or an aryl, any of which having up to 12 carbon atoms, wherein any heterocyclyl comprises at least one heteroatom or heterogroup selected independently from O, N, S, NR¹⁰, SO₂, or CO;
Z², in each occurrence, is selected independently from NR¹⁰ or O;
R′ and R″, in each occurrence, are independently selected from hydrogen or an alkyl having up to 12 carbon atoms; and
R¹⁵ and R¹⁶, in each occurrence, are independently selected from: 1) hydrogen; 2) an alkyl having up to 12 carbon atoms; or 3) —(CH₂)ᵣ—O—R¹³, —(CH₂)ᵣ—R¹⁴, —COR¹³, —(CH₂)ᵣ—CO—Z²—R¹³, —CO₂R¹³, —CO₂—(CH₂)ᵣ—R¹³, —CO₂—(CH₂)ᵣ—R¹², —CO₂—(CH₂)ᵣ—CO—Z²—R¹³, —CO₂—(CH₂)ᵣ—OR¹³, —CO—(CH₂)ᵣ—O—(CH₂)ᵣ—O—(CH₂)ᵣ—R¹³, —CO—(CH₂)ᵣ—O(CH₂)ᵣ—OR¹³, or —CO—NH—(CH₂)ᵣ—OR¹³;
or R¹⁵ and R¹⁶ together with the nitrogen atom to which they are attached form a substituted or an unsubstituted cyclic moiety comprising up to 12 carbon atoms, optionally comprising at least one additional heteroatom or heterogroup selected independently from O, N, S, NR¹⁰, SO₂, or CO; wherein any substituted cyclic moiety is substituted with up to three substituents selected independently from: 1) hydroxyl; 2) an alkyl or a heteroaryl, any of which having up to 12 carbon atoms, wherein any heteroaryl comprises at least one heteroatom or heterogroup selected independently from O, N, S, or NR¹⁰; or 3) COOR¹³, —Z²—(CH₂)ᵣ—R¹³, —COR¹³, —CO₂—(CH₂)ᵣ—R¹³, —CO(CH₂)ᵣ—O—R¹³, —(CH₂)ᵣ—CO₂—R¹³, —SO₂R⁸, —SO₂NR′R″, or —NR′R″;
wherein the —(CH₂)ᵣ— linking moiety, in any occurrence, is optionally substituted with at least one group selected independently from hydroxyl, amino, or an alkyl having up to 3 carbon atoms;
when R¹ and R² do not form a monocyclic or bicyclic moiety, R¹ and R² are optionally substituted with 1 or 2 substituents, and when substituted, the substituents are selected independently from: 1) an alkyl, a cycloalkyl, a haloalkyl, an alkoxy, an aryl, a heteroaryl, or a heterocyclyl, any of which having up to 12 carbon atoms, wherein any heteroaryl or heterocyclyl comprises at least one heteroatom or heterogroup selected independently from O, N, S, NR¹⁰, SO₂, or CO; or 2) halogen, cyano, or hydroxyl;
when R¹ and R² together with the diradical Z to which they are attached form a monocyclic or a bicyclic moiety, the cyclic moiety is optionally substituted with at least one substituent selected independently from: 1) halogen, cyano, or hydroxyl; 2) an alkyl, a haloalkyl, a cycloalkyl, an alkoxy, a cycloalkyl-substituted alkyl, an alkoxyalkyl, a cycloalkoxy, a haloalkoxy, an aryl, an aryloxy, an aralkyl, a heteroaryl or a heteroaryloxy, any of which having up to 12 carbon atoms, wherein any heteroaryl or heteroaryloxy comprises at least one heteroatom or heterogroup selected independently from O, N, S, or NR¹⁰; or 3) CO₂R⁶, COR⁸, SO₂R⁸, SO₂NR⁶R², or CONR⁶R²;
R⁴, R⁶, R⁷, and R⁸ are optionally substituted with at least one substituent, and when substituted, the substituents are selected independently from: 1) halogen, hydroxy, cyano, or NR⁶R²; or 2) an alkyl or an alkoxy, any of which having up to 12 carbon atoms;

and R⁵ is optionally substituted with at least one substituent, and when substituted, the substituents are selected independently from: 1) halogen, hydroxy, cyano, or NR₆R₇; or 2) an alkyl having up to 12 carbon atoms.

In one embodiment of the above aspect, the present application relates to a pharmaceutical composition comprising:
a) a cholesteryl ester transfer protein (CETP) inhibitor having formula (Ia′),
b) at least one surfactant,
c) optionally a carrier material, and
d) optionally one or more pharmaceutically acceptable excipients; wherein formula (Ia′) is defined as follows,

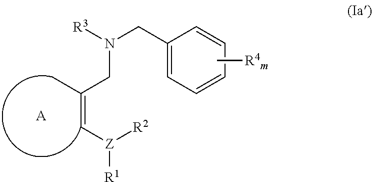

or a stereoisomer thereof or a pharmaceutically acceptable salt thereof, wherein A-ZR¹R² is:

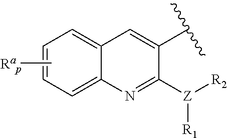

wherein R^{a}, in each occurrence, is selected independently from:
1) a hydrogen, a halogen, a cyano, or a hydroxyl; 2) an alkyl, a haloalkyl, a cycloalkyl, a (cycloalkyl)alkyl, an alkoxy, a cycloalkoxy, a haloalkoxy, an aryl, an aralkyl, a heteroaryl or a heterocyclyl, any of which having up to 12 carbon atoms, wherein any heteroaryl or heterocyclyl, comprises at least one heteroatom or heterogroup selected independently from O, N, S, NR¹⁰, SO₂, or CO; 3) CO₂R⁶, COR⁸, NR⁶R⁷ or SO₂R⁸;
p is an integer from 0 to 3, inclusive;
Z is N or CH; or the ZR¹ moiety is S, SO, CO, or SO₂; or the ZR¹R² moiety is C≡CR² or —C(O)Z³R^{f}, wherein R^{f} is an alkyl, a cycloalkyl, or a (cycloalkyl)alkyl, any of which having up to 12 carbon atoms, or hydrogen; and Z³ is O or NR^{k}, wherein R^{k} is an alkyl, a cycloalkyl, or a (cycloalkyl)alkyl, any of which having up to 12 carbon atoms, or hydrogen;
R¹ and R² are selected independently from: 1) hydrogen; 2) an alkyl having up to 6 carbon atoms; 3) a cycloalkyl having up to 6 carbon atoms; 4) COR⁸; or 5) (CH₂)ₙR⁵ or (CH₂)ₙR^{d}CO₂R^{e}; wherein n, in each occurrence, is 1 or 2; R^{d}, in each occurrence, is selected independently from an alkyl, a cycloalkyl, an aryl, a heterocyclyl, or a heteroaryl, any of which having up to 12 carbon atoms, wherein any heterocyclyl or heteroaryl comprises at least one heteroatom or heterogroup selected independently from O, N, S, NR¹⁰, SO₂, or CO; and R^{e}, in each occurrence, is selected independently from an alkyl or a cycloalkyl, either of which having up to 12 carbon atoms, or hydrogen;
or R¹ and R² together form a substituted or an unsubstituted monocyclic or bicyclic moiety comprising up to 12 carbon atoms, and optionally comprising 1 or 2 heteroatoms or heterogroups selected independently from O, N, or NR¹⁰; wherein any optional substituent on the cyclic moiety selected from: 1) a cycloalkyl having up to 6 carbon atoms; or 2) an alkyl having up to 2 carbon atoms;
R³ is selected from: 1) cyano; 2) a substituted or an unsubstituted alkyl having up to 12 carbon atoms; 3) a substituted or an unsubstituted aryl, or a substituted or an unsubstituted 5-, 6-, or 7-membered heterocyclyl or heteroaryl, comprising 1, 2, or 3 heteroatoms or heterogroups selected independently from O, N, S, NR¹⁰, SO₂, or CO; any of which having up to 12 carbon atoms; or 4) CO₂R⁶, COR⁸, SO₂R⁸, SO₂NR⁶R⁷, CONR⁶R⁷, C(S)NR⁶R⁷, C(═NH)OR⁸, C(S)NHC(O)OR⁸, or C(S)SR⁸; wherein when R³ is an alkyl, an aryl, a heterocyclyl, or a heteroaryl, R³ is optionally substituted with up to three substituents selected independently from R¹¹;
R⁴, in each occurrence, is selected independently from: 1) halogen, hydroxy or cyano; or 2) an alkyl, an alkoxy, a haloalkyl, or a haloalkoxy any of which having up to 4 carbon atoms; and m is an integer from 1-3, inclusive;
R⁵, in each occurrence, is selected independently from: 1) a substituted or an unsubstituted cycloalkyl, heterocyclyl, or heteroaryl, any of which having up to 12 carbon atoms, wherein any heterocyclyl or heteroaryl comprises at least one heteroatom or heterogroup selected independently from O, N, S, NR¹⁰, SO₂, or CO;
R⁶ and R⁷, in each occurrence, are selected independently from: 1) hydrogen; 2) an alkyl, a cycloalkyl, or a haloalkyl, any of which having up to 12 carbon atoms; or 3) a substituted or an unsubstituted aryl, aralkyl, heterocyclyl, or heteroaryl, any of which having up to 12 carbon atoms, wherein any heterocyclyl or heteroaryl comprises at least one heteroatom or heterogroup selected independently from O, N, S, NR¹⁰, SO₂, or CO;
R⁸, in each occurrence, is selected independently from: 1) an alkyl, a cycloalkyl, or a haloalkyl, any of which having up to 12 carbon atoms; or 2) a substituted or an unsubstituted aryl, heterocyclyl, or heteroaryl, any of which having up to 12 carbon atoms, wherein any heterocyclyl or heteroaryl comprises at least one heteroatom or heterogroup selected independently from O, N, S, NR¹⁰, SO₂, or CO;
R¹⁰, in each occurrence, is selected independently from: 1) hydrogen; or 2) an alkyl, a cycloalkyl, a haloalkyl, an aryl, or an aralkyl, any of which having up to 12 carbon atoms;
R¹¹ is selected independently from:
1) a halogen, a hydroxyl or a cyano;
2) an alkyl, a haloalkyl, an alkoxy, a cycloalkyl, or an alkoxycarbonyl, any of which having up to 12 carbon atoms;
3) a substituted or an unsubstituted heteroaryl or heterocyclyl, any of which having up to 12 carbon atoms, comprises at least one heteroatom or heterogroup selected independently from O, N, S, NR¹⁰, SO₂, or CO, wherein any substituted heteroaryl or heterocyclyl is substituted with up to three substituents selected independently from an alkyl having up to 12 carbon atoms or a hydroxyl; or
4) —CO—Z²—R¹³, —CO—R¹², —CO—Z²—(CH₂)ᵣ—CO—Z²—R¹³, —NR¹⁵R¹⁶,
   —Z²—CO—(CH₂)ᵣ—Z²—R¹³, —Z²—CO—(CH₂)ᵣ—CO—Z²—R¹³, —O—(CH₂)ᵣ—CO—Z²—R¹³,
   —O—(CH₂)ᵣ—R¹⁴, —O—R¹²—(CH₂)ᵣ—R¹³, —O—R¹⁴—CO—O—R¹³, —O—(CH₂)ᵣ—R¹²,
   —O—(CH₂)ᵣ—NR′R″, —O—(CH₂)ᵣ—CO₂—(CH₂)ᵣ—R¹³, —O—(CH₂)ᵣ—SR⁸, —O—(CH₂)ᵣ—CO₂—R¹³,
   —O—(CH₂)ᵣ—O—(CH₂)ᵣ—OR¹³,
   —O—(CH₂)ᵣ—CONR′R″, —O—(CH₂)ᵣ—CONH—(CH₂)ᵣ—OR¹³, —O—(CH₂)ᵣ—SO₂R⁸,
   —O—(CH₂)ᵣ—R¹³, —O—(CH₂)ᵣ—OR¹³, —S—(CH₂)ᵣ—CONR′R″, —SO₂—(CH₂)ᵣ—OR¹³, —SO₂—(CH₂)ᵣ—CONR′R″,
   —(CH₂)ᵣ—O—CO—R⁸, —(CH₂)ᵣ—R¹², —(CH₂)ᵣ—R¹³, —(CH₂)ᵣ—NH—(CH₂)ᵣ—OR¹³,
   —(CH₂)ᵣ—CO—Z²—R¹³, —(CH₂)ᵣ—Z²—R¹³, —(CH₂)ᵣ—NH—CO—Z²—R¹³, or -alkenylene-CO₂—(CH₂)ᵣ—R¹³;
r, in each occurrence, is independently 1, 2, or 3;
R¹², in each occurrence, is independently selected from a substituted or an unsubstituted heterocyclyl having up to 12 carbon atoms, comprising at least one heteroatom or heterogroup selected independently from O, N, S, NR¹⁰, SO₂, or CO, wherein any substituted heterocyclyl is substituted with up to three substituents selected independently from an acyl, an alkyl, or an alkoxycarbonyl, any of which having up to 12 carbon atoms, or —COOH;
R¹³, in each occurrence, is independently selected from: 1) hydrogen; or 2) a cycloalkyl, an aryl, a haloalkyl, a heterocyclyl, or an alkyl group optionally substituted with at least one hydroxyl, any of which having up to 12 carbon atoms, wherein any heterocyclyl comprises at least one heteroatom or heterogroup selected independently from O, N, S, NR¹⁰, SO₂, or CO;
R¹⁴, in each occurrence, is independently selected from a heterocyclyl, a cycloalkyl, or an aryl, any of which having up to 12 carbon atoms, wherein any heterocyclyl comprises at least one heteroatom or heterogroup selected independently from O, N, S, NR¹⁰, SO₂, or CO;
Z², in each occurrence, is selected independently from NR¹⁰ or O;
R′ and R″, in each occurrence, are independently selected from hydrogen or an alkyl having up to 12 carbon atoms; and
R¹⁵ and R¹⁶, in each occurrence, are independently selected from: 1) hydrogen; 2) an alkyl having up to 12 carbon atoms; or 3) —(CH₂)ᵣ—O—R¹³, —(CH₂)ᵣ—R¹⁴, —COR¹³, —(CH₂)ᵣ—CO—Z²—R¹³, —CO₂R¹³, —CO₂—(CH₂)ᵣ—R¹³, —CO₂—(CH₂)ᵣ—R¹², —CO₂—(CH₂)ᵣ—CO—Z²—R¹³, —CO₂—(CH₂)ᵣ—OR¹³, —CO—(CH₂)ᵣ—O—(CH₂)ᵣ—O—(CH₂)ᵣ—R¹³, —CO—(CH₂)ᵣ—O(CH₂)ᵣ—OR¹³, or —CO—NH—(CH₂)ᵣ—OR¹³;
or R¹⁵ and R¹⁶ together form a substituted or an unsubstituted cyclic moiety comprising up to 12 carbon atoms, optionally comprising at least one additional heteroatom or heterogroup selected independently from O, N, S, NR¹⁰, SO₂, or CO; wherein any substituted cyclic moiety is substituted with up to three substituents selected independently from: 1) hydroxyl; 2) an alkyl or a heteroaryl, any of which having up to 12 carbon atoms, wherein any heteroaryl comprises at least one heteroatom or heterogroup selected independently from O, N, S, or NR¹⁰; or 3) COOR¹³, —Z²—(CH₂)ᵣ—R¹³, —COR¹³, —CO₂—(CH₂)ᵣ—R¹³, —CO(CH₂)ᵣ—O—R¹³, —(CH₂)ᵣ—CO₂—R¹³, —SO₂R⁸, —SO₂NR′R″, or —NR′R″; and
wherein the —(CH₂)ᵣ— linking moiety, in any occurrence, is optionally substituted with at least one group selected independently from hydroxyl, amino, or an alkyl having up to 3 carbon atoms;
wherein when R¹ and R² do not form a monocyclic or bicyclic moiety, R¹ and R² are optionally substituted with 1 or 2 substituents, and when substituted, the substituents are selected independently from: 1) an alkyl, a cycloalkyl, a haloalkyl, an alkoxy, an aryl, a heteroaryl, or a heterocyclyl, any of which having up to 12 carbon atoms, wherein any heteroaryl or heterocyclyl comprises at least one heteroatom or heterogroup selected independently from O, N, S, NR¹⁰, SO₂, or CO; or 2) halogen, cyano, or hydroxyl;
wherein when R¹ and R² together form a monocyclic or a bicyclic moiety, the monocyclic or bicyclic moiety is optionally substituted with at least one substituent selected independently from: 1) halogen, cyano, or hydroxyl; 2) an alkyl, a haloalkyl, a cycloalkyl, an alkoxy, a cycloalkyl-substituted alkyl, an alkoxyalkyl, a cycloalkoxy, a haloalkoxy, an aryl, an aryloxy, an aralkyl, a heteroaryl or a heteroaryloxy, any of which having up to 12 carbon atoms, wherein any heteroaryl or heteroaryloxy comprises at least one heteroatom or heterogroup selected independently from O, N, S, or NR¹⁰; or 3) CO₂R⁶, (CH₂)_{q}COR⁸, SO₂R⁸, SO₂NR⁶R⁷, or CONR⁶R⁷; or 4) (CH₂)_{q}CO₂(CH₂)_{q}CH₃, wherein q is selected independently from an integer from 0 to 3, inclusive; and
R⁴, R⁶, R⁷, and R⁸ are optionally substituted with at least one substituent, and when substituted, the substituents are selected independently from: 1) halogen, hydroxy, cyano, or NR⁶R⁷; or 2) an alkyl or an alkoxy, any of which having up to 12 carbon atoms; and

R⁵ is optionally substituted with at least one substituent selected independently from: 1) halogen, hydroxy, cyano, or NR⁶R⁷; or 2) an alkyl or an alkoxy, any of which having up to 12 carbon atoms; or 3) (CH₂)ₜOR^{j} or (CH₂)ₜCOOR^{j} wherein t is an integer from 1 to 3, inclusive, and R^{j} is hydrogen or alkyl having up to 12 carbon atoms.

In another aspect, the present application relates to a pharmaceutical composition comprising
a) a cholesteryl ester transfer protein (CETP) inhibitor having formula (II),
b) at least one surfactant,
c) optionally a carrier material, and
d) optionally one or more pharmaceutically acceptable excipients; wherein formula (II) is defined as follows

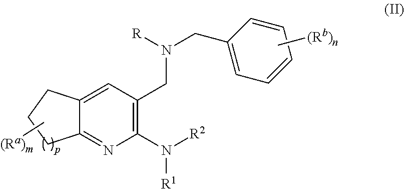

or a stereoisomer thereof or a pharmaceutically acceptable salt thereof; wherein,
R represents

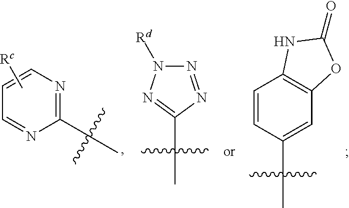

R¹ and R² are independently selected from hydrogen, acyl, haloalkyl, —(CHR^{e})_{q}R³, an optionally substituted group selected from alkyl or cycloalkyl, wherein optional substituent, in each occurrence, is independently selected from halogen, cyano, hydroxyl, an alkyl, a haloalkyl or an alkoxy;
R³ is a group selected from alkoxy, haloalkoxy, cycloalkyl, aryl, heterocyclyl or heteroaryl, wherein R³ is optionally substituted with a group selected from halogen, cyano, hydroxyl, alkyl, haloalkyl or alkoxy;
R^{a}, in each occurrence, is independently selected from halogen, cyano, hydroxy, alkyl, haloalkyl or alkoxy;
R^{b}, in each occurrence, is independently selected from halogen, alkyl, haloalkyl, hydroxy, alkoxy or haloalkoxy;
R^{c} is independently selected from hydrogen, cyano, halogen, —C(═O)—R^{t}, —CONR^{g}R^{h}, —C(═O)—C≡CH—NRⁱR^{j}, an optionally substituted group selected from cycloalkyl, aryl, heteroaryl or heterocyclyl ring, wherein the optional substituent, in each occurrence, is selected independently from hydrogen, halogen, cyano, hydroxyl, alkyl, haloalkyl, alkoxy, alkoxyalkyl or haloalkoxy;
R^{d} is hydrogen or alkyl;
R^{e}, in each occurrence, is independently selected from hydrogen, alkyl or alkoxy;
R^{f} is hydrogen or alkyl;
R^{g} and R^{h} independently represent hydrogen or alkyl;
Rⁱ and Rⱼ independently represent hydrogen or alkyl;
m is 0, 1 or 2;
n is 0, 1, 2 or 3;
p is 1 or 2; and
q is 0, 1, 2, 3, 4 or 5.

In yet another aspect, the present application relates to a pharmaceutical composition comprising:
a) a cholesteryl ester transfer protein (CETP) inhibitor having formula (III),
b) at least one surfactant,
c) optionally a carrier material, and
d) optionally one or more pharmaceutically acceptable excipients; wherein formula (III) is defined as follows,

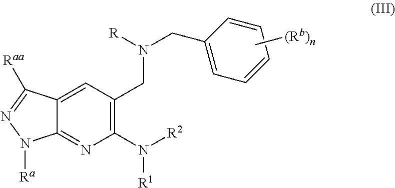

or a stereoisomer thereof or a pharmaceutically acceptable salt thereof, wherein,
R represents hydrogen or

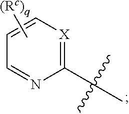

X represents —CH or —N;
R¹ and R² are independently of each other selected from hydrogen, acyl, alkyl or —(CH₂)ₚ-cycloalkyl;
R^{a} and R^{aa} are independently of each other selected from hydrogen or alkyl;
R^{b}, in each occurrence, is independently selected from halogen, alkyl, haloalkyl, hydroxy, alkoxy or haloalkoxy;
R^{c}, in each occurrence, is independently selected from hydrogen, cyano, halogen, alkyl, alkoxy, haloalkoxy, —COOR^{d}, —C(═O)—R^{e}, —CONR^{g}R^{h}, —C(═O)—CH═CH—NRⁱR^{j}, —NHCOR^{t}, an optionally substituted group selected from cycloalkyl, aryl, heteroaryl or heterocycle ring, wherein the optional substituent, in each occurrence, is selected independently from hydrogen, halogen, cyano, hydroxyl, alkyl, haloalkyl, alkoxy, alkoxyalkyl or haloalkoxy;
R^{d}, R^{e}, R^{g}, R^{h}, Rⁱ and R^{j}, in each occurrence, independently of each other represents hydrogen or alkyl;
R^{t} is selected from hydrogen, alkyl or cycloalkyl;
n is 0, 1, 2 or 3;
p is 0, 1, or 2; and
q is 1 or 2.

In another embodiment, the application provides pharmaceutical compositions comprising one or more specific compounds of formulae (I), (Ia′), (II) or (III) and is enumerated as follows:

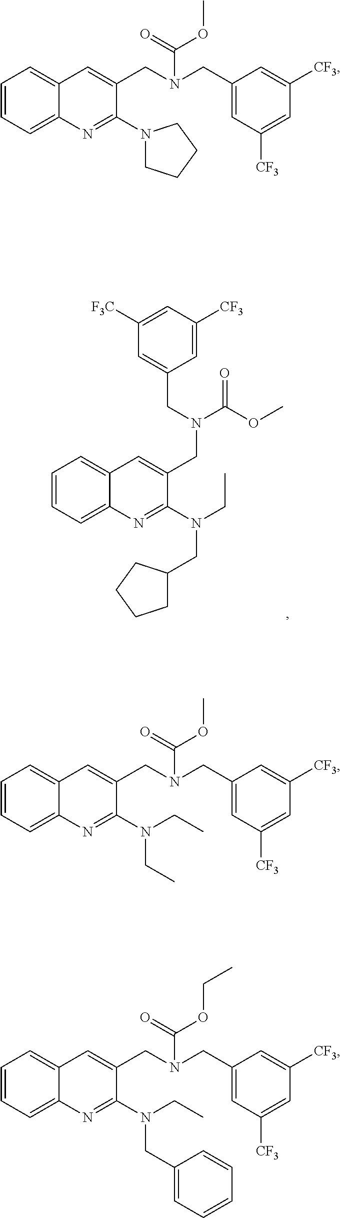 [SMILES: COC(=O)N(Cc1cc(C(F)(F)F)cc(C(F)(F)F)c1)Cc1cc2ccccc2nc1N1CCCC1 ; CCN(CC1CCCC1)c1nc2ccccc2cc1CN(Cc1cc(C(F)(F)F)cc(C(F)(F)F)c1)C(=O)OC ; CCN(CC)c1nc2ccccc2cc1CN(Cc1cc(C(F)(F)F)cc(C(F)(F)F)c1)C(=O)OC ; CCOC(=O)N(Cc1cc(C(F)(F)F)cc(C(F)(F)F)c1)Cc1cc2ccccc2nc1N(CC)Cc1ccccc1]

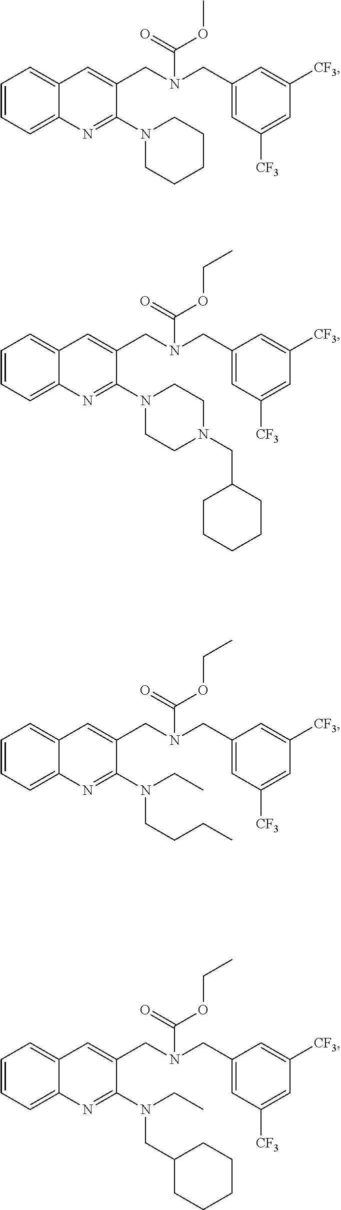 [SMILES: COC(=O)N(Cc1cc(C(F)(F)F)cc(C(F)(F)F)c1)Cc1cc2ccccc2nc1N1CCCCC1 ; CCOC(=O)N(Cc1cc(C(F)(F)F)cc(C(F)(F)F)c1)Cc1cc2ccccc2nc1N1CCN(CC2CCCCC2)CC1 ; CCCCN(CC)c1nc2ccccc2cc1CN(Cc1cc(C(F)(F)F)cc(C(F)(F)F)c1)C(=O)OCC ; CCOC(=O)N(Cc1cc(C(F)(F)F)cc(C(F)(F)F)c1)Cc1cc2ccccc2nc1N(CC)CC1CCCCC1]

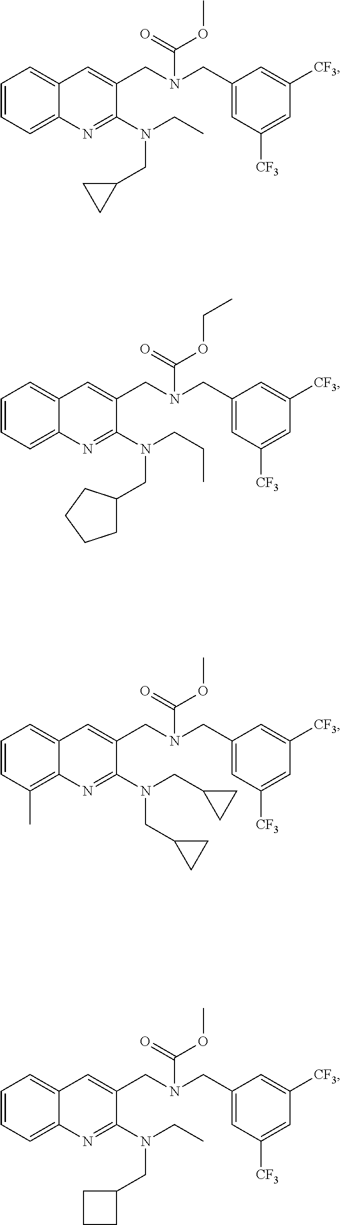 [SMILES: CCN(CC1CC1)c1nc2ccccc2cc1CN(Cc1cc(C(F)(F)F)cc(C(F)(F)F)c1)C(=O)OC ; CCCN(CC1CCCC1)c1nc2ccccc2cc1CN(Cc1cc(C(F)(F)F)cc(C(F)(F)F)c1)C(=O)OCC ; COC(=O)N(Cc1cc(C(F)(F)F)cc(C(F)(F)F)c1)Cc1cc2cccc(C)c2nc1N(CC1CC1)CC1CC1 ; CCN(CC1CCC1)c1nc2ccccc2cc1CN(Cc1cc(C(F)(F)F)cc(C(F)(F)F)c1)C(=O)OC]

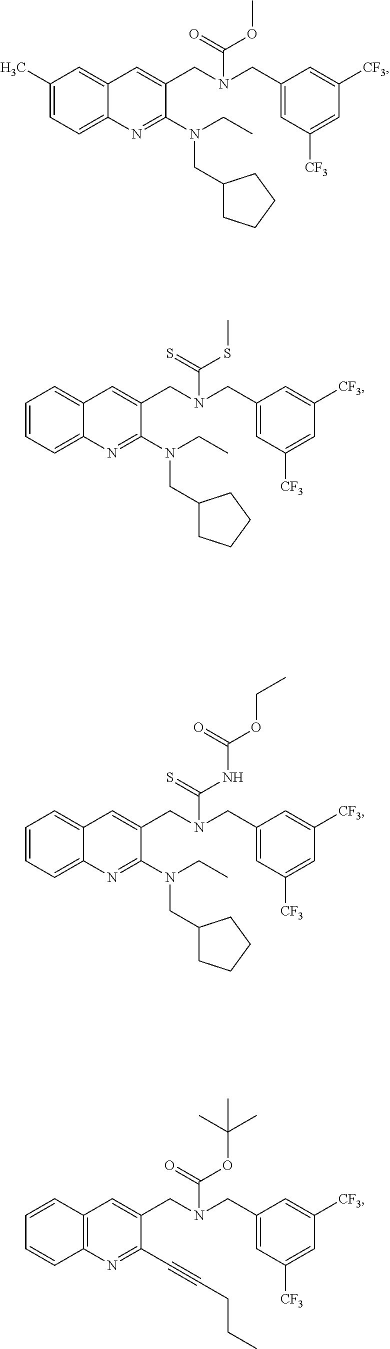 [SMILES: CCN(CC1CCCC1)c1nc2ccc(C)cc2cc1CN(Cc1cc(C(F)(F)F)cc(C(F)(F)F)c1)C(=O)OC ; CCN(CC1CCCC1)c1nc2ccccc2cc1CN(Cc1cc(C(F)(F)F)cc(C(F)(F)F)c1)C(=S)SC ; CCOC(=O)NC(=S)N(Cc1cc(C(F)(F)F)cc(C(F)(F)F)c1)Cc1cc2ccccc2nc1N(CC)CC1CCCC1 ; CCCC#Cc1nc2ccccc2cc1CN(Cc1cc(C(F)(F)F)cc(C(F)(F)F)c1)C(=O)OC(C)(C)C]

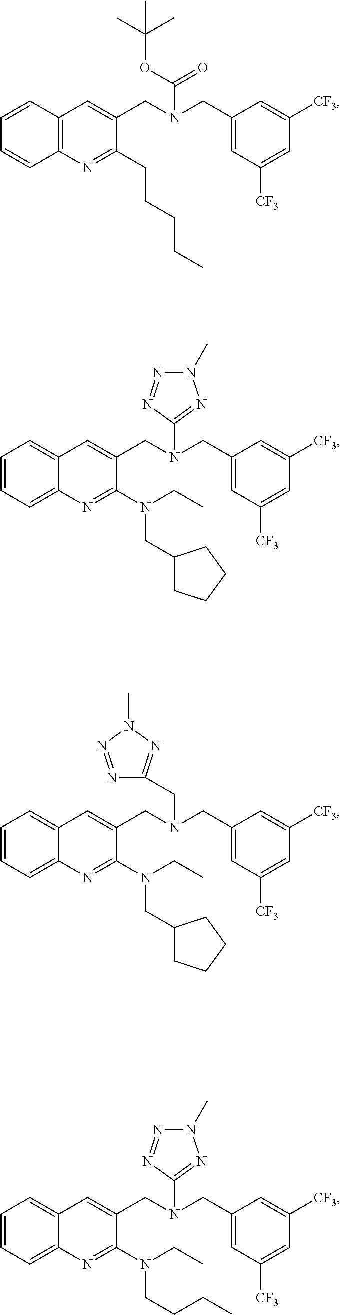 [SMILES: CCCCCc1nc2ccccc2cc1CN(Cc1cc(C(F)(F)F)cc(C(F)(F)F)c1)C(=O)OC(C)(C)C ; CCN(CC1CCCC1)c1nc2ccccc2cc1CN(Cc1cc(C(F)(F)F)cc(C(F)(F)F)c1)c1nnn(C)n1 ; CCN(CC1CCCC1)c1nc2ccccc2cc1CN(Cc1cc(C(F)(F)F)cc(C(F)(F)F)c1)Cc1nnn(C)n1 ; CCCCN(CC)c1nc2ccccc2cc1CN(Cc1cc(C(F)(F)F)cc(C(F)(F)F)c1)c1nnn(C)n1]

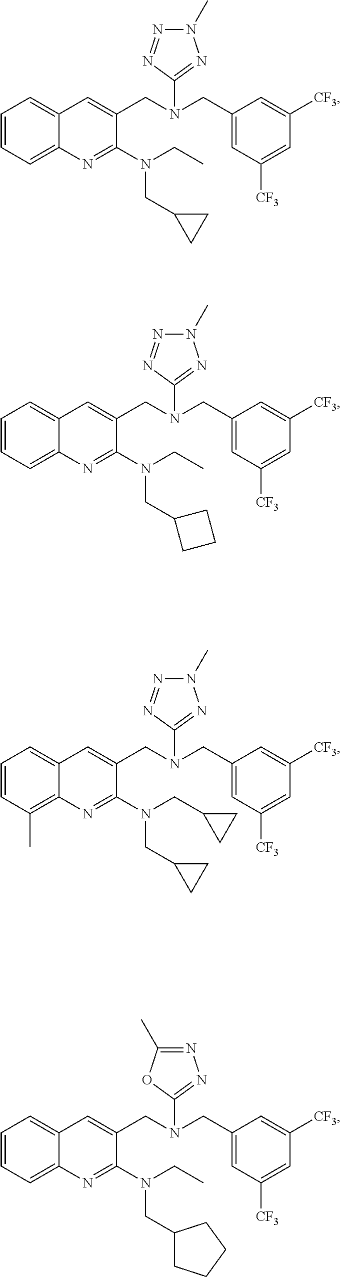 [SMILES: CCN(CC1CC1)c1nc2ccccc2cc1CN(Cc1cc(C(F)(F)F)cc(C(F)(F)F)c1)c1nnn(C)n1 ; CCN(CC1CCC1)c1nc2ccccc2cc1CN(Cc1cc(C(F)(F)F)cc(C(F)(F)F)c1)c1nnn(C)n1 ; Cc1cccc2cc(CN(Cc3cc(C(F)(F)F)cc(C(F)(F)F)c3)c3nnn(C)n3)c(N(CC3CC3)CC3CC3)nc12 ; CCN(CC1CCCC1)c1nc2ccccc2cc1CN(Cc1cc(C(F)(F)F)cc(C(F)(F)F)c1)c1nnc(C)o1]

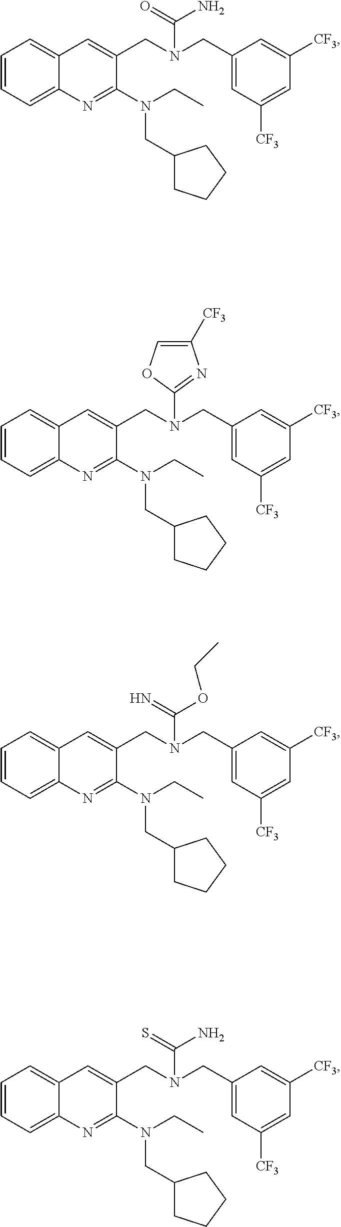 [SMILES: CCN(CC1CCCC1)c1nc2ccccc2cc1CN(Cc1cc(C(F)(F)F)cc(C(F)(F)F)c1)C(N)=O ; CCN(CC1CCCC1)c1nc2ccccc2cc1CN(Cc1cc(C(F)(F)F)cc(C(F)(F)F)c1)c1nc(C(F)(F)F)co1 ; CCOC(=N)N(Cc1cc(C(F)(F)F)cc(C(F)(F)F)c1)Cc1cc2ccccc2nc1N(CC)CC1CCCC1 ; CCN(CC1CCCC1)c1nc2ccccc2cc1CN(Cc1cc(C(F)(F)F)cc(C(F)(F)F)c1)C(N)=S]

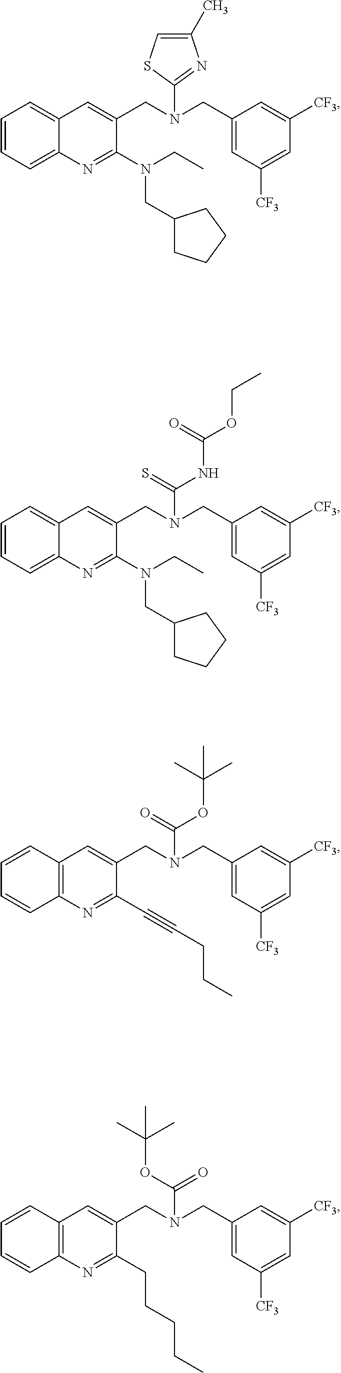 [SMILES: CCN(CC1CCCC1)c1nc2ccccc2cc1CN(Cc1cc(C(F)(F)F)cc(C(F)(F)F)c1)c1nc(C)cs1 ; CCOC(=O)NC(=S)N(Cc1cc(C(F)(F)F)cc(C(F)(F)F)c1)Cc1cc2ccccc2nc1N(CC)CC1CCCC1 ; CCCC#Cc1nc2ccccc2cc1CN(Cc1cc(C(F)(F)F)cc(C(F)(F)F)c1)C(=O)OC(C)(C)C ; CCCCCc1nc2ccccc2cc1CN(Cc1cc(C(F)(F)F)cc(C(F)(F)F)c1)C(=O)OC(C)(C)C]

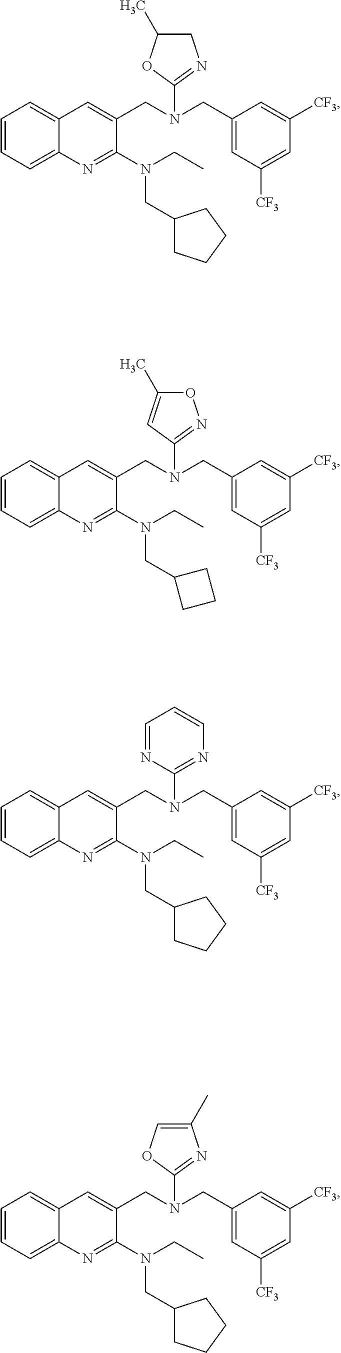 [SMILES: CCN(CC1CCCC1)c1nc2ccccc2cc1CN(Cc1cc(C(F)(F)F)cc(C(F)(F)F)c1)C1=NCC(C)O1 ; CCN(CC1CCC1)c1nc2ccccc2cc1CN(Cc1cc(C(F)(F)F)cc(C(F)(F)F)c1)c1cc(C)on1 ; CCN(CC1CCCC1)c1nc2ccccc2cc1CN(Cc1cc(C(F)(F)F)cc(C(F)(F)F)c1)c1ncccn1 ; CCN(CC1CCCC1)c1nc2ccccc2cc1CN(Cc1cc(C(F)(F)F)cc(C(F)(F)F)c1)c1nc(C)co1]

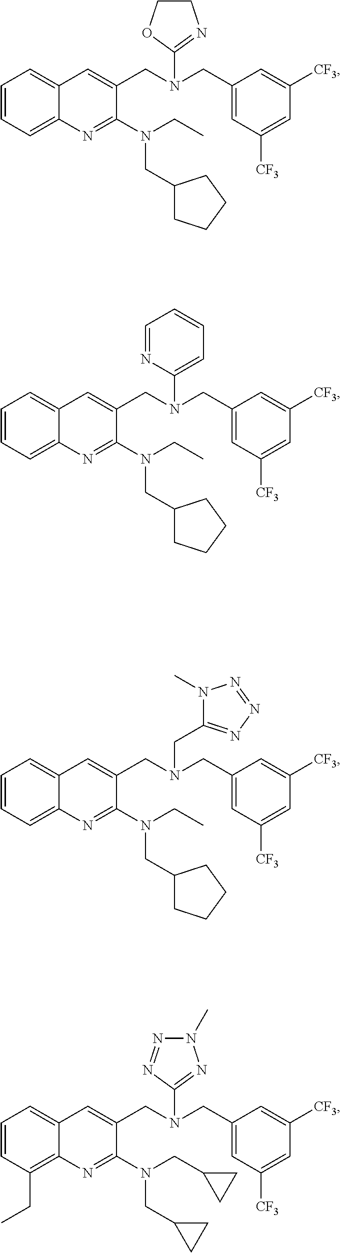 [SMILES: CCN(CC1CCCC1)c1nc2ccccc2cc1CN(Cc1cc(C(F)(F)F)cc(C(F)(F)F)c1)C1=NCCO1 ; CCN(CC1CCCC1)c1nc2ccccc2cc1CN(Cc1cc(C(F)(F)F)cc(C(F)(F)F)c1)c1ccccn1 ; CCN(CC1CCCC1)c1nc2ccccc2cc1CN(Cc1cc(C(F)(F)F)cc(C(F)(F)F)c1)Cc1nnnn1C ; CCc1cccc2cc(CN(Cc3cc(C(F)(F)F)cc(C(F)(F)F)c3)c3nnn(C)n3)c(N(CC3CC3)CC3CC3)nc12]

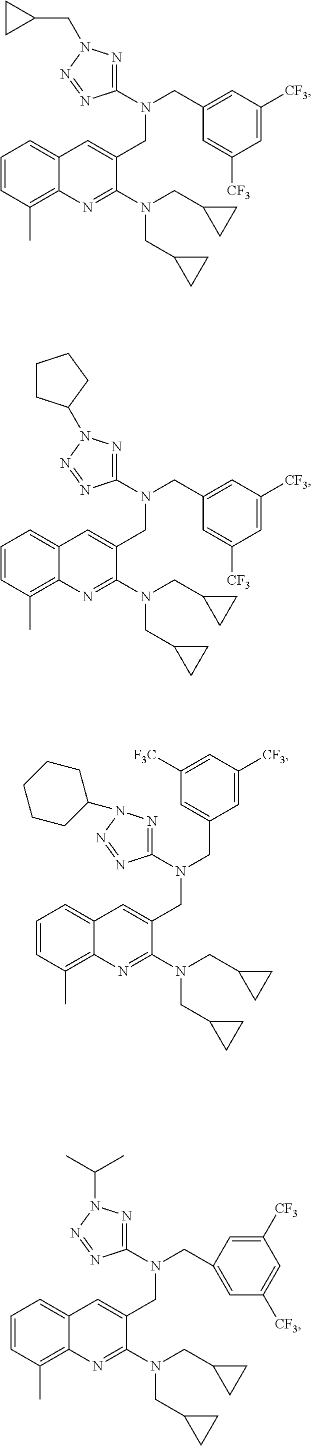 [SMILES: Cc1cccc2cc(CN(Cc3cc(C(F)(F)F)cc(C(F)(F)F)c3)c3nnn(CC4CC4)n3)c(N(CC3CC3)CC3CC3)nc12 ; Cc1cccc2cc(CN(Cc3cc(C(F)(F)F)cc(C(F)(F)F)c3)c3nnn(C4CCCC4)n3)c(N(CC3CC3)CC3CC3)nc12 ; Cc1cccc2cc(CN(Cc3cc(C(F)(F)F)cc(C(F)(F)F)c3)c3nnn(C4CCCCC4)n3)c(N(CC3CC3)CC3CC3)nc12 ; Cc1cccc2cc(CN(Cc3cc(C(F)(F)F)cc(C(F)(F)F)c3)c3nnn(C(C)C)n3)c(N(CC3CC3)CC3CC3)nc12]

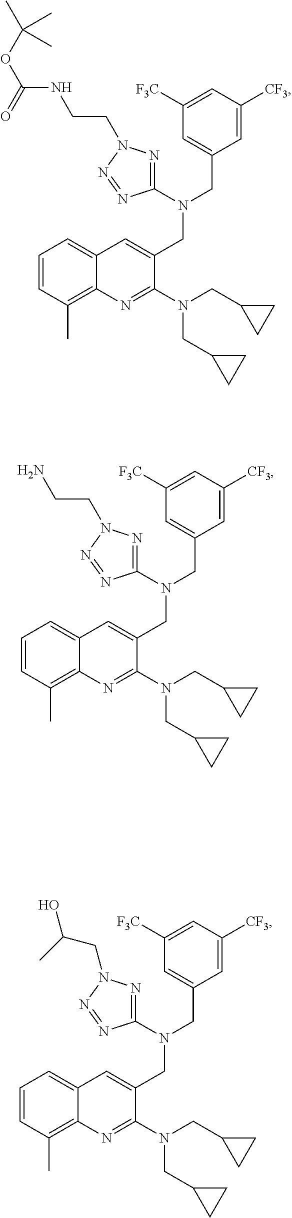 [SMILES: Cc1cccc2cc(CN(Cc3cc(C(F)(F)F)cc(C(F)(F)F)c3)c3nnn(CCNC(=O)OC(C)(C)C)n3)c(N(CC3CC3)CC3CC3)nc12 ; Cc1cccc2cc(CN(Cc3cc(C(F)(F)F)cc(C(F)(F)F)c3)c3nnn(CCN)n3)c(N(CC3CC3)CC3CC3)nc12 ; Cc1cccc2cc(CN(Cc3cc(C(F)(F)F)cc(C(F)(F)F)c3)c3nnn(CC(C)O)n3)c(N(CC3CC3)CC3CC3)nc12]

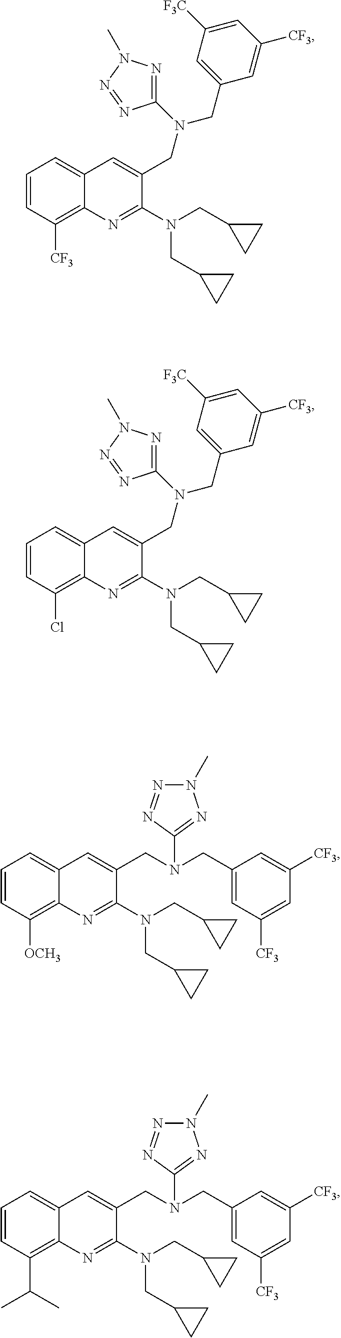 [SMILES: Cn1nnc(N(Cc2cc(C(F)(F)F)cc(C(F)(F)F)c2)Cc2cc3cccc(C(F)(F)F)c3nc2N(CC2CC2)CC2CC2)n1 ; Cn1nnc(N(Cc2cc(C(F)(F)F)cc(C(F)(F)F)c2)Cc2cc3cccc(Cl)c3nc2N(CC2CC2)CC2CC2)n1 ; COc1cccc2cc(CN(Cc3cc(C(F)(F)F)cc(C(F)(F)F)c3)c3nnn(C)n3)c(N(CC3CC3)CC3CC3)nc12 ; CC(C)c1cccc2cc(CN(Cc3cc(C(F)(F)F)cc(C(F)(F)F)c3)c3nnn(C)n3)c(N(CC3CC3)CC3CC3)nc12]

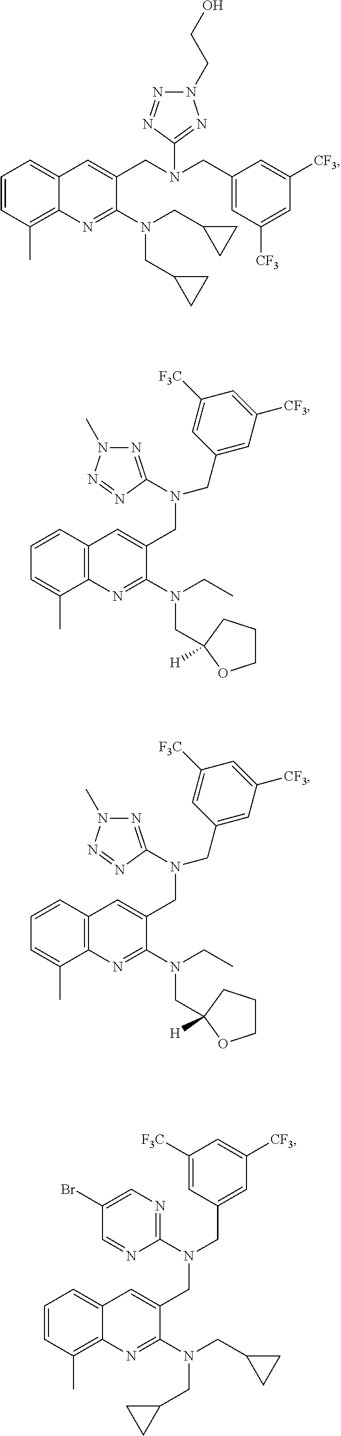 [SMILES: Cc1cccc2cc(CN(Cc3cc(C(F)(F)F)cc(C(F)(F)F)c3)c3nnn(CCO)n3)c(N(CC3CC3)CC3CC3)nc12 ; CCN(C[C@H]1CCCO1)c1nc2c(C)cccc2cc1CN(Cc1cc(C(F)(F)F)cc(C(F)(F)F)c1)c1nnn(C)n1 ; CCN(C[C@@H]1CCCO1)c1nc2c(C)cccc2cc1CN(Cc1cc(C(F)(F)F)cc(C(F)(F)F)c1)c1nnn(C)n1 ; Cc1cccc2cc(CN(Cc3cc(C(F)(F)F)cc(C(F)(F)F)c3)c3ncc(Br)cn3)c(N(CC3CC3)CC3CC3)nc12]

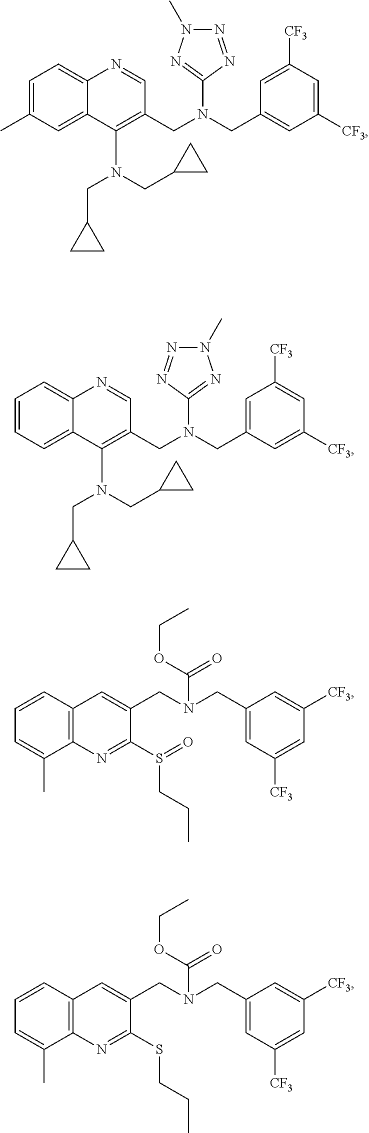 [SMILES: Cc1ccc2ncc(CN(Cc3cc(C(F)(F)F)cc(C(F)(F)F)c3)c3nnn(C)n3)c(N(CC3CC3)CC3CC3)c2c1 ; Cn1nnc(N(Cc2cc(C(F)(F)F)cc(C(F)(F)F)c2)Cc2cnc3ccccc3c2N(CC2CC2)CC2CC2)n1 ; CCCS(=O)c1nc2c(C)cccc2cc1CN(Cc1cc(C(F)(F)F)cc(C(F)(F)F)c1)C(=O)OCC ; CCCSc1nc2c(C)cccc2cc1CN(Cc1cc(C(F)(F)F)cc(C(F)(F)F)c1)C(=O)OCC]

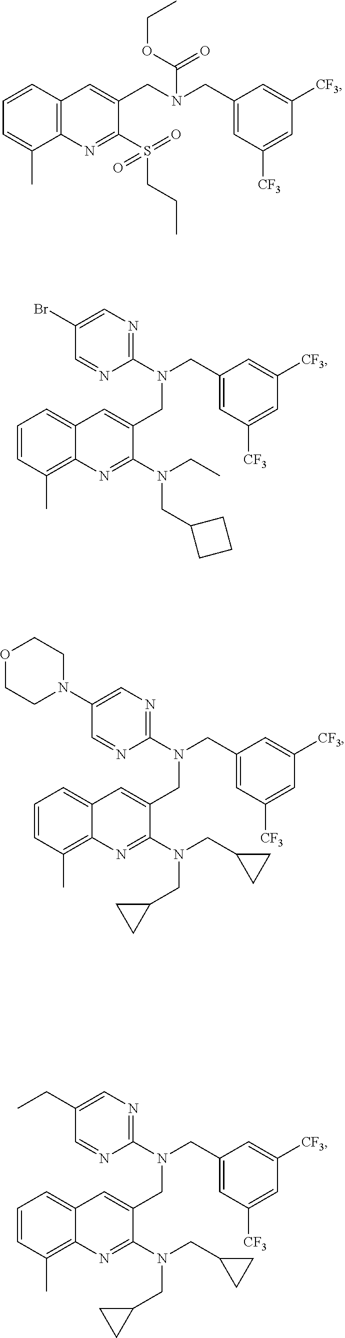 [SMILES: CCCS(=O)(=O)c1nc2c(C)cccc2cc1CN(Cc1cc(C(F)(F)F)cc(C(F)(F)F)c1)C(=O)OCC ; CCN(CC1CCC1)c1nc2c(C)cccc2cc1CN(Cc1cc(C(F)(F)F)cc(C(F)(F)F)c1)c1ncc(Br)cn1 ; Cc1cccc2cc(CN(Cc3cc(C(F)(F)F)cc(C(F)(F)F)c3)c3ncc(N4CCOCC4)cn3)c(N(CC3CC3)CC3CC3)nc12 ; CCc1cnc(N(Cc2cc(C(F)(F)F)cc(C(F)(F)F)c2)Cc2cc3cccc(C)c3nc2N(CC2CC2)CC2CC2)nc1]

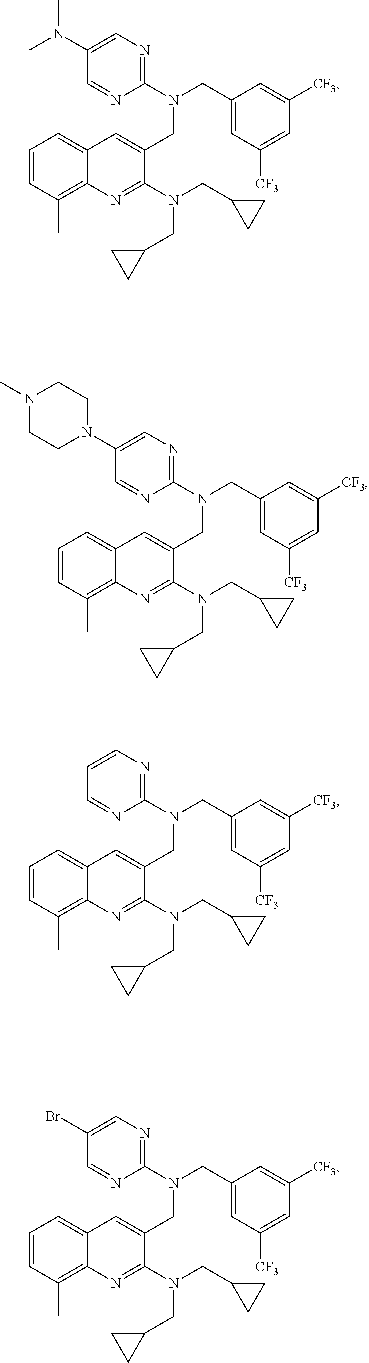 [SMILES: Cc1cccc2cc(CN(Cc3cc(C(F)(F)F)cc(C(F)(F)F)c3)c3ncc(N(C)C)cn3)c(N(CC3CC3)CC3CC3)nc12 ; Cc1cccc2cc(CN(Cc3cc(C(F)(F)F)cc(C(F)(F)F)c3)c3ncc(N4CCN(C)CC4)cn3)c(N(CC3CC3)CC3CC3)nc12 ; Cc1cccc2cc(CN(Cc3cc(C(F)(F)F)cc(C(F)(F)F)c3)c3ncccn3)c(N(CC3CC3)CC3CC3)nc12 ; Cc1cccc2cc(CN(Cc3cc(C(F)(F)F)cc(C(F)(F)F)c3)c3ncc(Br)cn3)c(N(CC3CC3)CC3CC3)nc12]

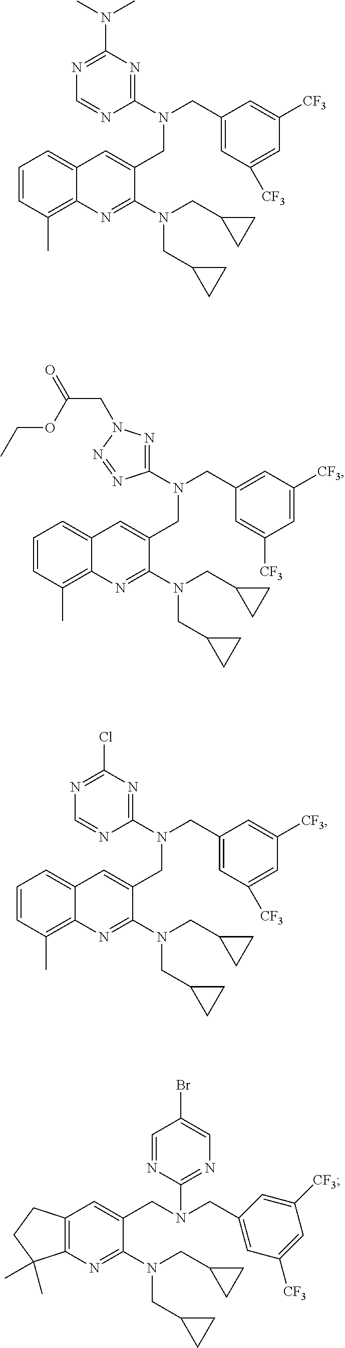 [SMILES: Cc1cccc2cc(CN(Cc3cc(C(F)(F)F)cc(C(F)(F)F)c3)c3ncnc(N(C)C)n3)c(N(CC3CC3)CC3CC3)nc12 ; CCOC(=O)Cn1nnc(N(Cc2cc(C(F)(F)F)cc(C(F)(F)F)c2)Cc2cc3cccc(C)c3nc2N(CC2CC2)CC2CC2)n1 ; Cc1cccc2cc(CN(Cc3cc(C(F)(F)F)cc(C(F)(F)F)c3)c3ncnc(Cl)n3)c(N(CC3CC3)CC3CC3)nc12 ; CC1(C)CCc2cc(CN(Cc3cc(C(F)(F)F)cc(C(F)(F)F)c3)c3ncc(Br)cn3)c(N(CC3CC3)CC3CC3)nc21]

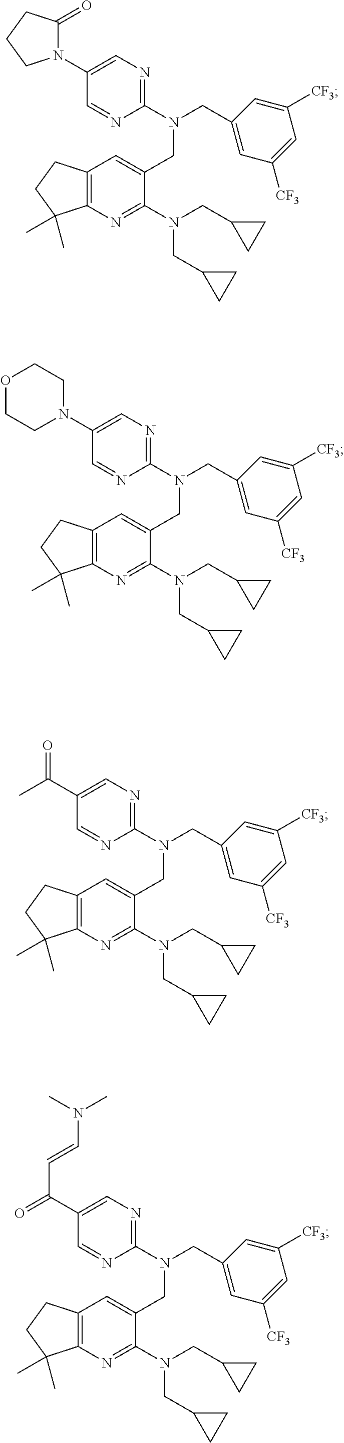 [SMILES: CC1(C)CCc2cc(CN(Cc3cc(C(F)(F)F)cc(C(F)(F)F)c3)c3ncc(N4CCCC4=O)cn3)c(N(CC3CC3)CC3CC3)nc21 ; CC1(C)CCc2cc(CN(Cc3cc(C(F)(F)F)cc(C(F)(F)F)c3)c3ncc(N4CCOCC4)cn3)c(N(CC3CC3)CC3CC3)nc21 ; CC(=O)c1cnc(N(Cc2cc(C(F)(F)F)cc(C(F)(F)F)c2)Cc2cc3c(nc2N(CC2CC2)CC2CC2)C(C)(C)CC3)nc1 ; CN(C)/C=C/C(=O)c1cnc(N(Cc2cc(C(F)(F)F)cc(C(F)(F)F)c2)Cc2cc3c(nc2N(CC2CC2)CC2CC2)C(C)(C)CC3)nc1]

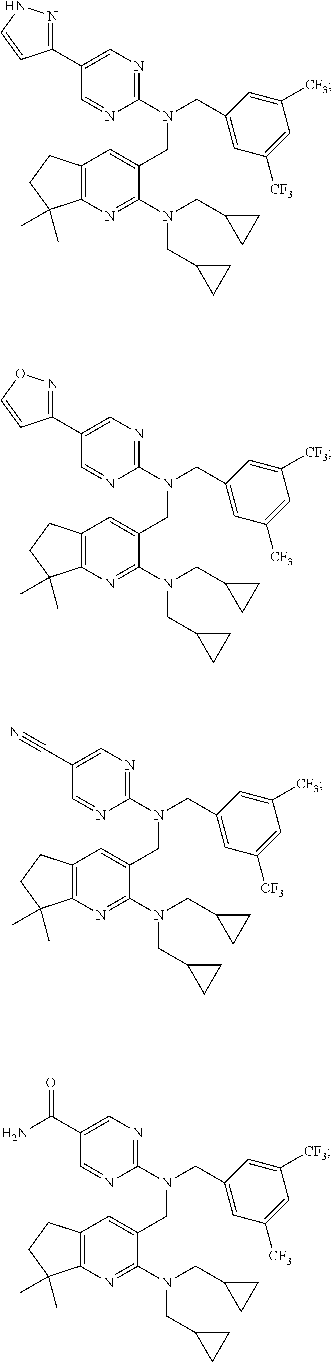 [SMILES: CC1(C)CCc2cc(CN(Cc3cc(C(F)(F)F)cc(C(F)(F)F)c3)c3ncc(-c4cc[nH]n4)cn3)c(N(CC3CC3)CC3CC3)nc21 ; CC1(C)CCc2cc(CN(Cc3cc(C(F)(F)F)cc(C(F)(F)F)c3)c3ncc(-c4ccon4)cn3)c(N(CC3CC3)CC3CC3)nc21 ; CC1(C)CCc2cc(CN(Cc3cc(C(F)(F)F)cc(C(F)(F)F)c3)c3ncc(C#N)cn3)c(N(CC3CC3)CC3CC3)nc21 ; CC1(C)CCc2cc(CN(Cc3cc(C(F)(F)F)cc(C(F)(F)F)c3)c3ncc(C(N)=O)cn3)c(N(CC3CC3)CC3CC3)nc21]

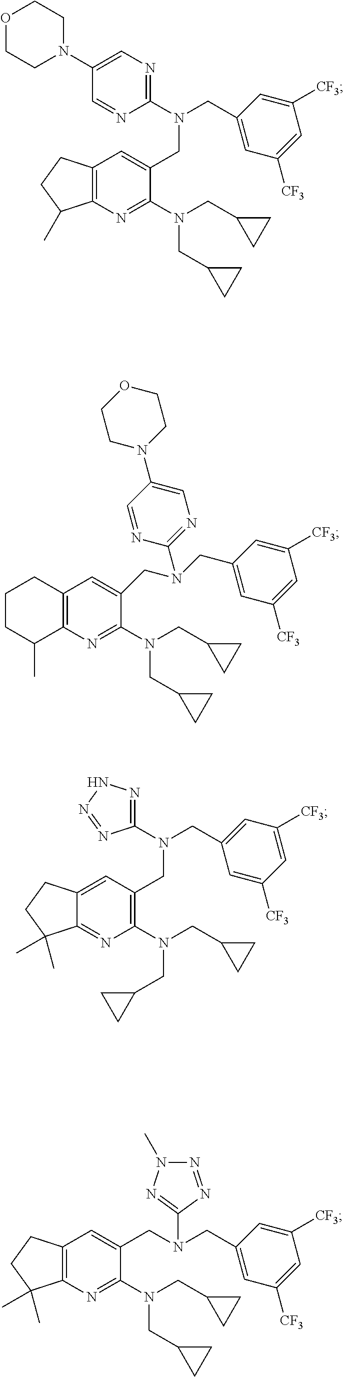 [SMILES: CC1CCc2cc(CN(Cc3cc(C(F)(F)F)cc(C(F)(F)F)c3)c3ncc(N4CCOCC4)cn3)c(N(CC3CC3)CC3CC3)nc21 ; CC1CCCc2cc(CN(Cc3cc(C(F)(F)F)cc(C(F)(F)F)c3)c3ncc(N4CCOCC4)cn3)c(N(CC3CC3)CC3CC3)nc21 ; CC1(C)CCc2cc(CN(Cc3cc(C(F)(F)F)cc(C(F)(F)F)c3)c3nn[nH]n3)c(N(CC3CC3)CC3CC3)nc21 ; Cn1nnc(N(Cc2cc(C(F)(F)F)cc(C(F)(F)F)c2)Cc2cc3c(nc2N(CC2CC2)CC2CC2)C(C)(C)CC3)n1]

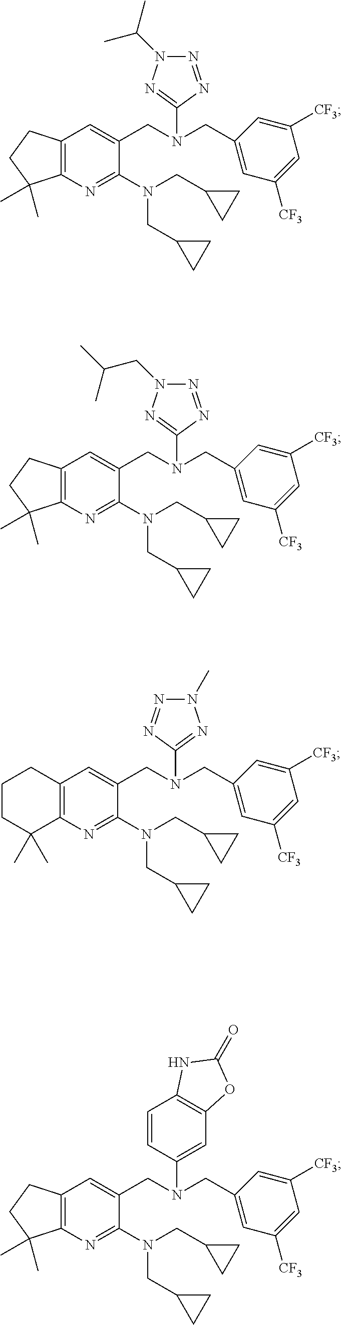 [SMILES: CC(C)n1nnc(N(Cc2cc(C(F)(F)F)cc(C(F)(F)F)c2)Cc2cc3c(nc2N(CC2CC2)CC2CC2)C(C)(C)CC3)n1 ; CC(C)Cn1nnc(N(Cc2cc(C(F)(F)F)cc(C(F)(F)F)c2)Cc2cc3c(nc2N(CC2CC2)CC2CC2)C(C)(C)CC3)n1 ; Cn1nnc(N(Cc2cc(C(F)(F)F)cc(C(F)(F)F)c2)Cc2cc3c(nc2N(CC2CC2)CC2CC2)C(C)(C)CCC3)n1 ; CC1(C)CCc2cc(CN(Cc3cc(C(F)(F)F)cc(C(F)(F)F)c3)c3ccc4[nH]c(=O)oc4c3)c(N(CC3CC3)CC3CC3)nc21]

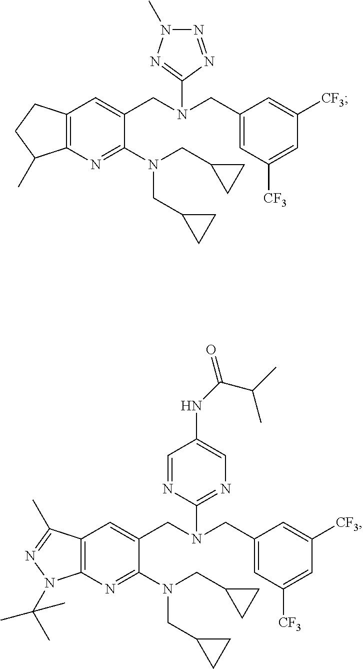 [SMILES: CC1CCc2cc(CN(Cc3cc(C(F)(F)F)cc(C(F)(F)F)c3)c3nnn(C)n3)c(N(CC3CC3)CC3CC3)nc21 ; Cc1nn(C(C)(C)C)c2nc(N(CC3CC3)CC3CC3)c(CN(Cc3cc(C(F)(F)F)cc(C(F)(F)F)c3)c3ncc(NC(=O)C(C)C)cn3)cc12]

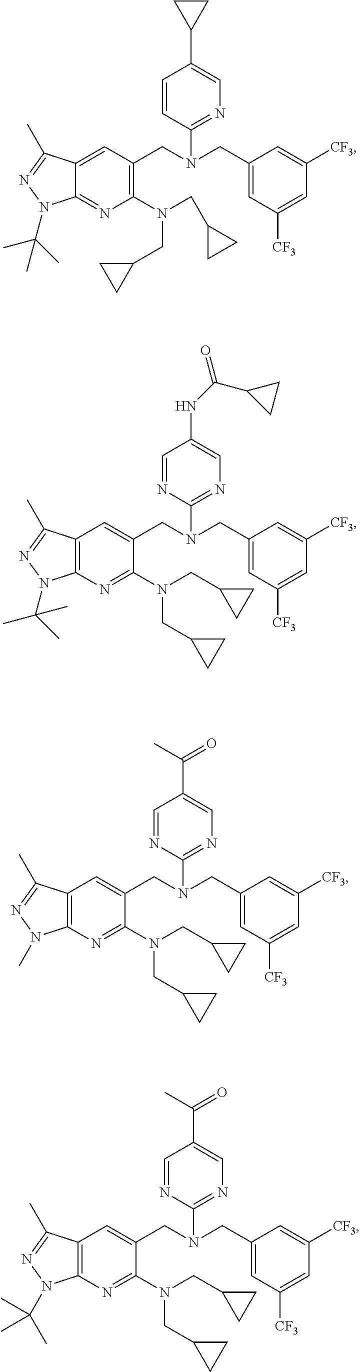 [SMILES: Cc1nn(C(C)(C)C)c2nc(N(CC3CC3)CC3CC3)c(CN(Cc3cc(C(F)(F)F)cc(C(F)(F)F)c3)c3ccc(C4CC4)cn3)cc12 ; Cc1nn(C(C)(C)C)c2nc(N(CC3CC3)CC3CC3)c(CN(Cc3cc(C(F)(F)F)cc(C(F)(F)F)c3)c3ncc(NC(=O)C4CC4)cn3)cc12 ; CC(=O)c1cnc(N(Cc2cc(C(F)(F)F)cc(C(F)(F)F)c2)Cc2cc3c(C)nn(C)c3nc2N(CC2CC2)CC2CC2)nc1 ; CC(=O)c1cnc(N(Cc2cc(C(F)(F)F)cc(C(F)(F)F)c2)Cc2cc3c(C)nn(C(C)(C)C)c3nc2N(CC2CC2)CC2CC2)nc1]

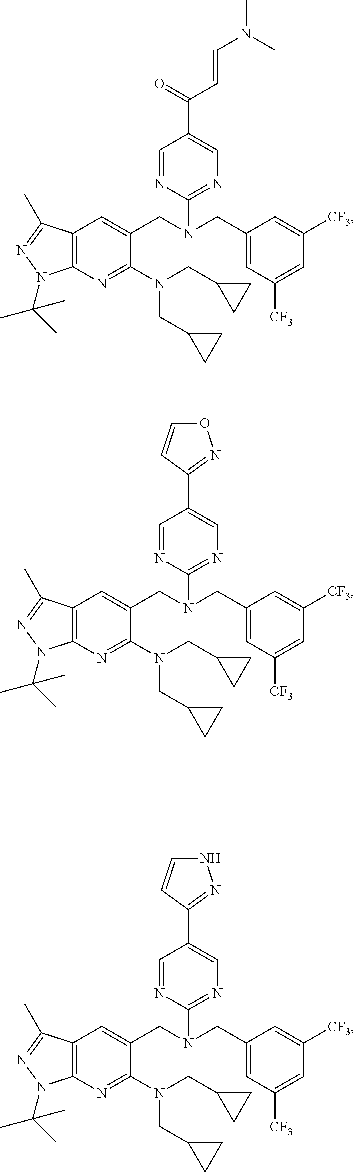 [SMILES: Cc1nn(C(C)(C)C)c2nc(N(CC3CC3)CC3CC3)c(CN(Cc3cc(C(F)(F)F)cc(C(F)(F)F)c3)c3ncc(C(=O)/C=C/N(C)C)cn3)cc12 ; Cc1nn(C(C)(C)C)c2nc(N(CC3CC3)CC3CC3)c(CN(Cc3cc(C(F)(F)F)cc(C(F)(F)F)c3)c3ncc(-c4ccon4)cn3)cc12 ; Cc1nn(C(C)(C)C)c2nc(N(CC3CC3)CC3CC3)c(CN(Cc3cc(C(F)(F)F)cc(C(F)(F)F)c3)c3ncc(-c4cc[nH]n4)cn3)cc12]

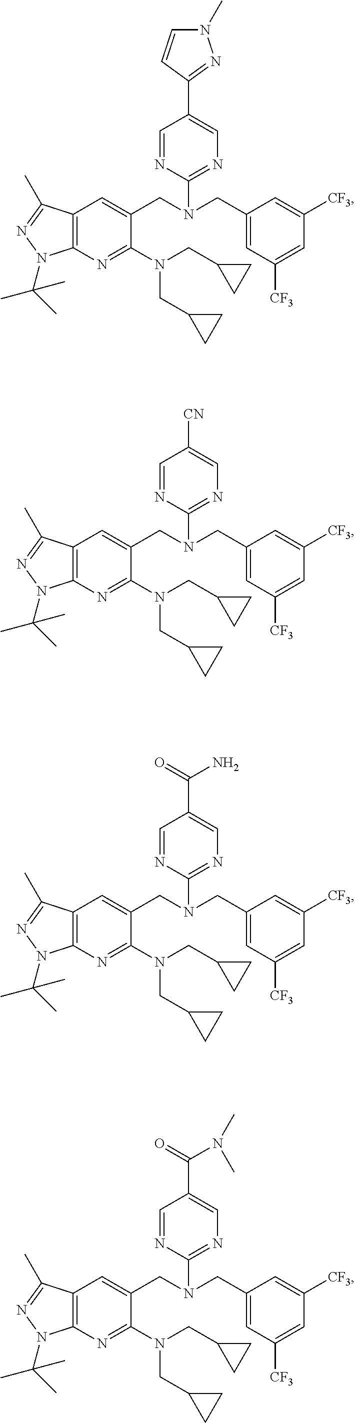 [SMILES: Cc1nn(C(C)(C)C)c2nc(N(CC3CC3)CC3CC3)c(CN(Cc3cc(C(F)(F)F)cc(C(F)(F)F)c3)c3ncc(-c4ccn(C)n4)cn3)cc12 ; Cc1nn(C(C)(C)C)c2nc(N(CC3CC3)CC3CC3)c(CN(Cc3cc(C(F)(F)F)cc(C(F)(F)F)c3)c3ncc(C#N)cn3)cc12 ; Cc1nn(C(C)(C)C)c2nc(N(CC3CC3)CC3CC3)c(CN(Cc3cc(C(F)(F)F)cc(C(F)(F)F)c3)c3ncc(C(N)=O)cn3)cc12 ; Cc1nn(C(C)(C)C)c2nc(N(CC3CC3)CC3CC3)c(CN(Cc3cc(C(F)(F)F)cc(C(F)(F)F)c3)c3ncc(C(=O)N(C)C)cn3)cc12]

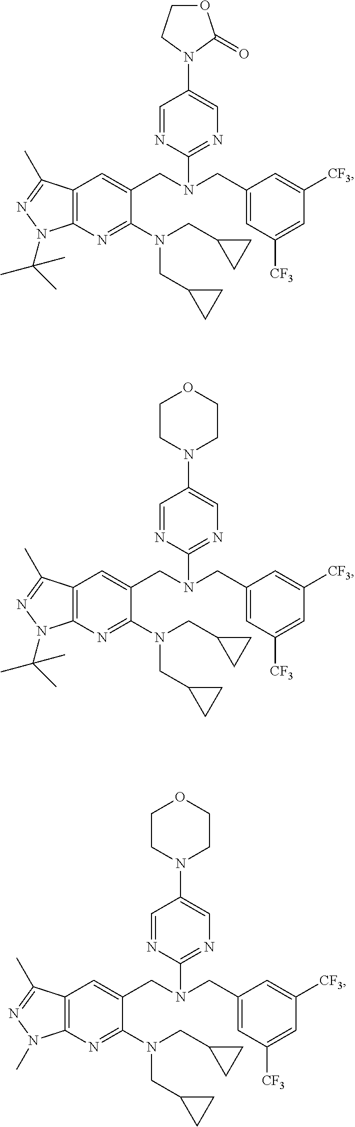 [SMILES: Cc1nn(C(C)(C)C)c2nc(N(CC3CC3)CC3CC3)c(CN(Cc3cc(C(F)(F)F)cc(C(F)(F)F)c3)c3ncc(N4CCOC4=O)cn3)cc12 ; Cc1nn(C(C)(C)C)c2nc(N(CC3CC3)CC3CC3)c(CN(Cc3cc(C(F)(F)F)cc(C(F)(F)F)c3)c3ncc(N4CCOCC4)cn3)cc12 ; Cc1nn(C)c2nc(N(CC3CC3)CC3CC3)c(CN(Cc3cc(C(F)(F)F)cc(C(F)(F)F)c3)c3ncc(N4CCOCC4)cn3)cc12]

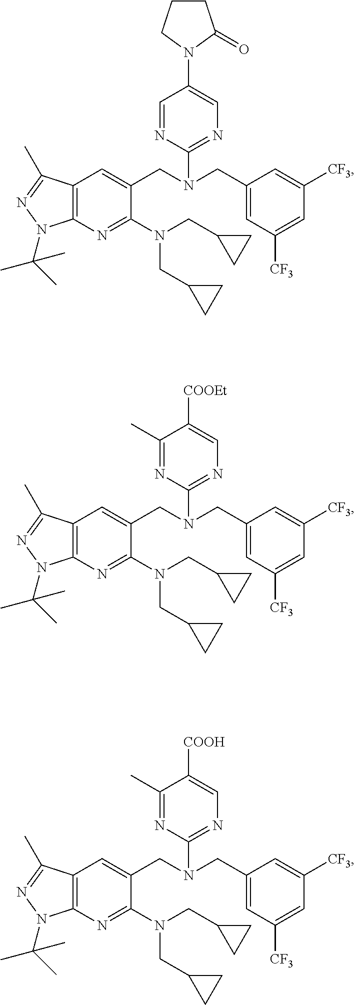 [SMILES: Cc1nn(C(C)(C)C)c2nc(N(CC3CC3)CC3CC3)c(CN(Cc3cc(C(F)(F)F)cc(C(F)(F)F)c3)c3ncc(N4CCCC4=O)cn3)cc12 ; CCOC(=O)c1cnc(N(Cc2cc(C(F)(F)F)cc(C(F)(F)F)c2)Cc2cc3c(C)nn(C(C)(C)C)c3nc2N(CC2CC2)CC2CC2)nc1C ; Cc1nc(N(Cc2cc(C(F)(F)F)cc(C(F)(F)F)c2)Cc2cc3c(C)nn(C(C)(C)C)c3nc2N(CC2CC2)CC2CC2)ncc1C(=O)O]

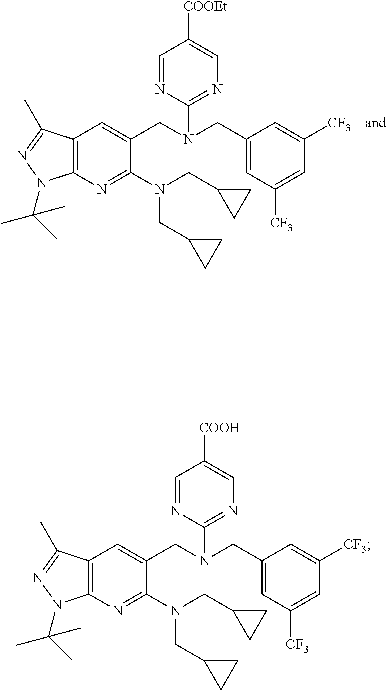 [SMILES: CCOC(=O)c1cnc(N(Cc2cc(C(F)(F)F)cc(C(F)(F)F)c2)Cc2cc3c(C)nn(C(C)(C)C)c3nc2N(CC2CC2)CC2CC2)nc1 ; Cc1nn(C(C)(C)C)c2nc(N(CC3CC3)CC3CC3)c(CN(Cc3cc(C(F)(F)F)cc(C(F)(F)F)c3)c3ncc(C(=O)O)cn3)cc12]

or a stereoisomer thereof or a pharmaceutically acceptable salt thereof.

In one embodiment, the application provides pharmaceutical compositions comprising one or more specific compounds of formula (I) and are enumerated as follows:

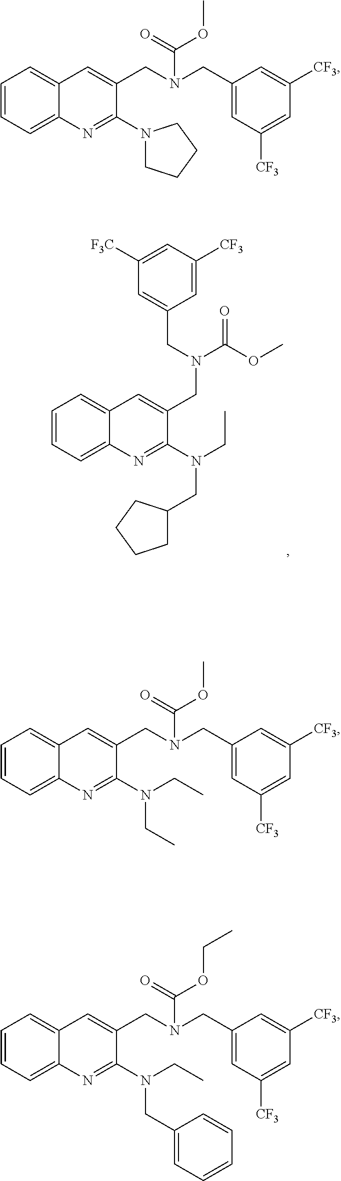 [SMILES: COC(=O)N(Cc1cc(C(F)(F)F)cc(C(F)(F)F)c1)Cc1cc2ccccc2nc1N1CCCC1 ; CCN(CC1CCCC1)c1nc2ccccc2cc1CN(Cc1cc(C(F)(F)F)cc(C(F)(F)F)c1)C(=O)OC ; CCN(CC)c1nc2ccccc2cc1CN(Cc1cc(C(F)(F)F)cc(C(F)(F)F)c1)C(=O)OC ; CCOC(=O)N(Cc1cc(C(F)(F)F)cc(C(F)(F)F)c1)Cc1cc2ccccc2nc1N(CC)Cc1ccccc1]

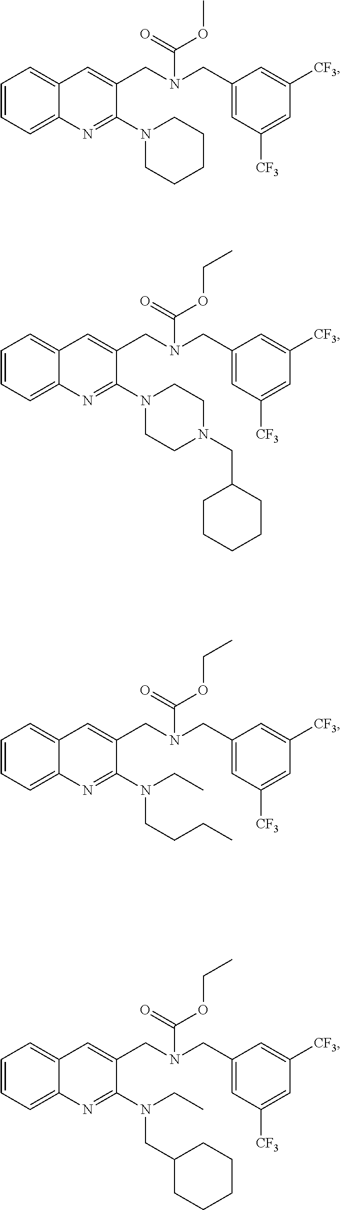 [SMILES: COC(=O)N(Cc1cc(C(F)(F)F)cc(C(F)(F)F)c1)Cc1cc2ccccc2nc1N1CCCCC1 ; CCOC(=O)N(Cc1cc(C(F)(F)F)cc(C(F)(F)F)c1)Cc1cc2ccccc2nc1N1CCN(CC2CCCCC2)CC1 ; CCCCN(CC)c1nc2ccccc2cc1CN(Cc1cc(C(F)(F)F)cc(C(F)(F)F)c1)C(=O)OCC ; CCOC(=O)N(Cc1cc(C(F)(F)F)cc(C(F)(F)F)c1)Cc1cc2ccccc2nc1N(CC)CC1CCCCC1]

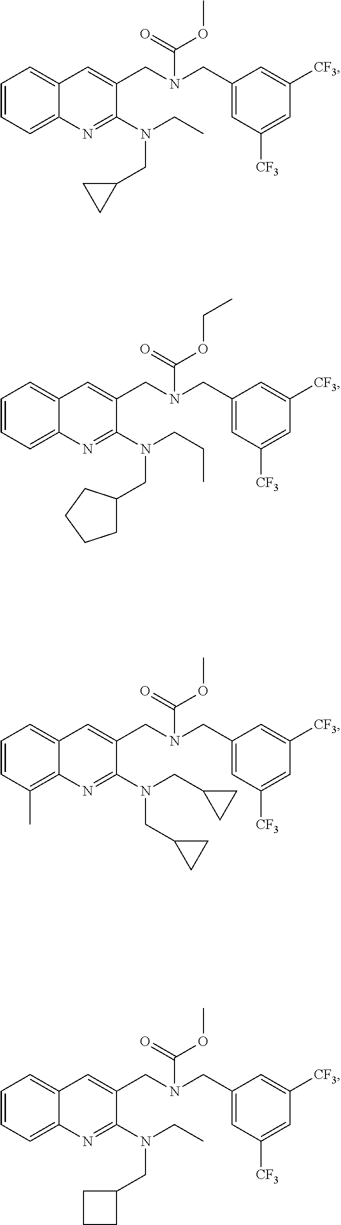 [SMILES: CCN(CC1CC1)c1nc2ccccc2cc1CN(Cc1cc(C(F)(F)F)cc(C(F)(F)F)c1)C(=O)OC ; CCCN(CC1CCCC1)c1nc2ccccc2cc1CN(Cc1cc(C(F)(F)F)cc(C(F)(F)F)c1)C(=O)OCC ; COC(=O)N(Cc1cc(C(F)(F)F)cc(C(F)(F)F)c1)Cc1cc2cccc(C)c2nc1N(CC1CC1)CC1CC1 ; CCN(CC1CCC1)c1nc2ccccc2cc1CN(Cc1cc(C(F)(F)F)cc(C(F)(F)F)c1)C(=O)OC]

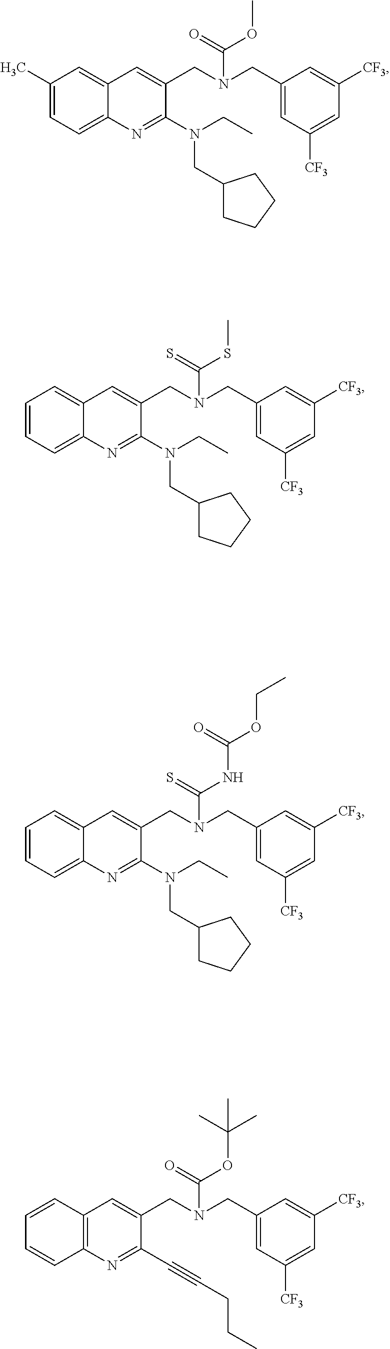 [SMILES: CCN(CC1CCCC1)c1nc2ccc(C)cc2cc1CN(Cc1cc(C(F)(F)F)cc(C(F)(F)F)c1)C(=O)OC ; CCN(CC1CCCC1)c1nc2ccccc2cc1CN(Cc1cc(C(F)(F)F)cc(C(F)(F)F)c1)C(=S)SC ; CCOC(=O)NC(=S)N(Cc1cc(C(F)(F)F)cc(C(F)(F)F)c1)Cc1cc2ccccc2nc1N(CC)CC1CCCC1 ; CCCC#Cc1nc2ccccc2cc1CN(Cc1cc(C(F)(F)F)cc(C(F)(F)F)c1)C(=O)OC(C)(C)C]

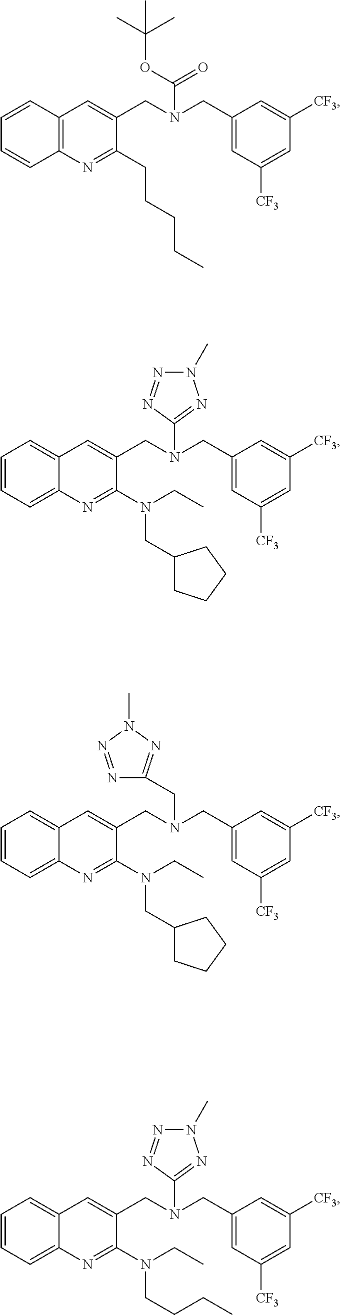 [SMILES: CCCCCc1nc2ccccc2cc1CN(Cc1cc(C(F)(F)F)cc(C(F)(F)F)c1)C(=O)OC(C)(C)C ; CCN(CC1CCCC1)c1nc2ccccc2cc1CN(Cc1cc(C(F)(F)F)cc(C(F)(F)F)c1)c1nnn(C)n1 ; CCN(CC1CCCC1)c1nc2ccccc2cc1CN(Cc1cc(C(F)(F)F)cc(C(F)(F)F)c1)Cc1nnn(C)n1 ; CCCCN(CC)c1nc2ccccc2cc1CN(Cc1cc(C(F)(F)F)cc(C(F)(F)F)c1)c1nnn(C)n1]

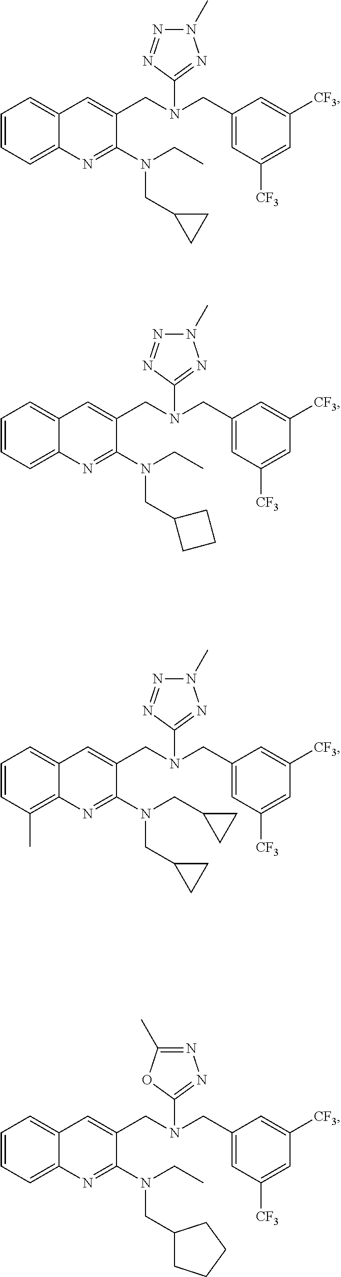 [SMILES: CCN(CC1CC1)c1nc2ccccc2cc1CN(Cc1cc(C(F)(F)F)cc(C(F)(F)F)c1)c1nnn(C)n1 ; CCN(CC1CCC1)c1nc2ccccc2cc1CN(Cc1cc(C(F)(F)F)cc(C(F)(F)F)c1)c1nnn(C)n1 ; Cc1cccc2cc(CN(Cc3cc(C(F)(F)F)cc(C(F)(F)F)c3)c3nnn(C)n3)c(N(CC3CC3)CC3CC3)nc12 ; CCN(CC1CCCC1)c1nc2ccccc2cc1CN(Cc1cc(C(F)(F)F)cc(C(F)(F)F)c1)c1nnc(C)o1]

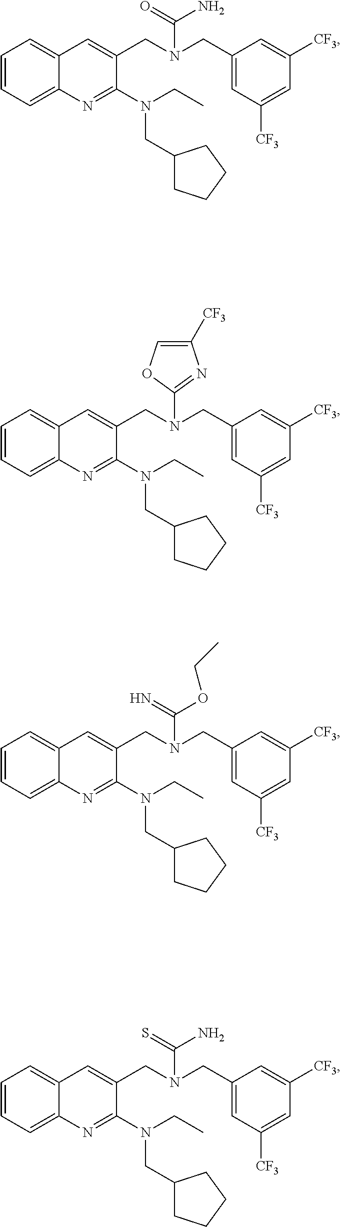 [SMILES: CCN(CC1CCCC1)c1nc2ccccc2cc1CN(Cc1cc(C(F)(F)F)cc(C(F)(F)F)c1)C(N)=O ; CCN(CC1CCCC1)c1nc2ccccc2cc1CN(Cc1cc(C(F)(F)F)cc(C(F)(F)F)c1)c1nc(C(F)(F)F)co1 ; CCOC(=N)N(Cc1cc(C(F)(F)F)cc(C(F)(F)F)c1)Cc1cc2ccccc2nc1N(CC)CC1CCCC1 ; CCN(CC1CCCC1)c1nc2ccccc2cc1CN(Cc1cc(C(F)(F)F)cc(C(F)(F)F)c1)C(N)=S]

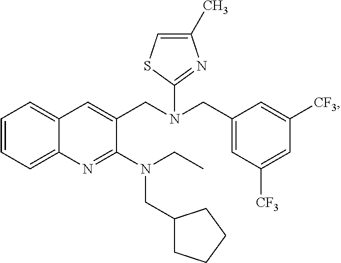 [SMILES: CCN(CC1CCCC1)c1nc2ccccc2cc1CN(Cc1cc(C(F)(F)F)cc(C(F)(F)F)c1)c1nc(C)cs1]

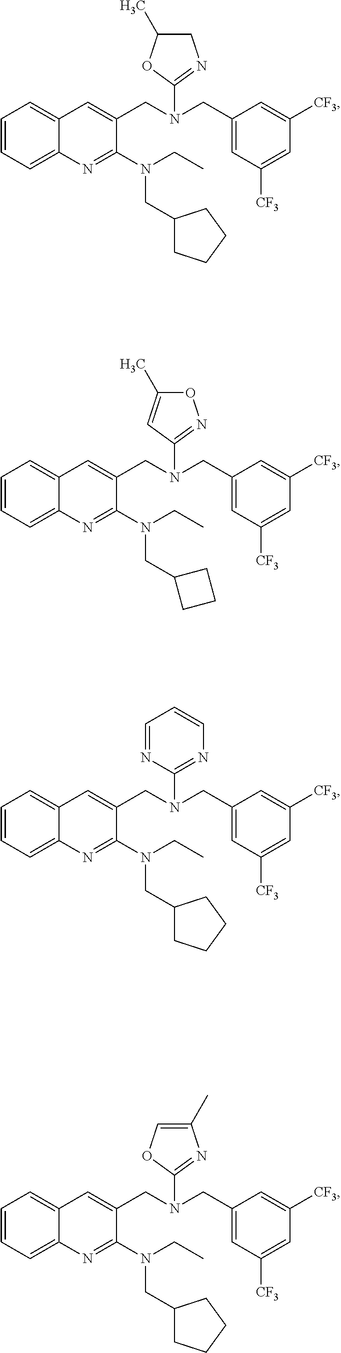 [SMILES: CCN(CC1CCCC1)c1nc2ccccc2cc1CN(Cc1cc(C(F)(F)F)cc(C(F)(F)F)c1)C1=NCC(C)O1 ; CCN(CC1CCC1)c1nc2ccccc2cc1CN(Cc1cc(C(F)(F)F)cc(C(F)(F)F)c1)c1cc(C)on1 ; CCN(CC1CCCC1)c1nc2ccccc2cc1CN(Cc1cc(C(F)(F)F)cc(C(F)(F)F)c1)c1ncccn1 ; CCN(CC1CCCC1)c1nc2ccccc2cc1CN(Cc1cc(C(F)(F)F)cc(C(F)(F)F)c1)c1nc(C)co1]

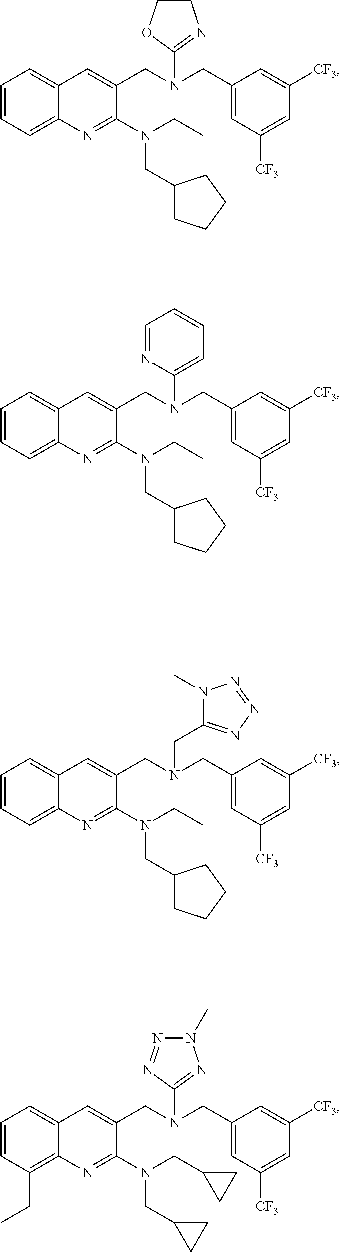 [SMILES: CCN(CC1CCCC1)c1nc2ccccc2cc1CN(Cc1cc(C(F)(F)F)cc(C(F)(F)F)c1)C1=NCCO1 ; CCN(CC1CCCC1)c1nc2ccccc2cc1CN(Cc1cc(C(F)(F)F)cc(C(F)(F)F)c1)c1ccccn1 ; CCN(CC1CCCC1)c1nc2ccccc2cc1CN(Cc1cc(C(F)(F)F)cc(C(F)(F)F)c1)Cc1nnnn1C ; CCc1cccc2cc(CN(Cc3cc(C(F)(F)F)cc(C(F)(F)F)c3)c3nnn(C)n3)c(N(CC3CC3)CC3CC3)nc12]

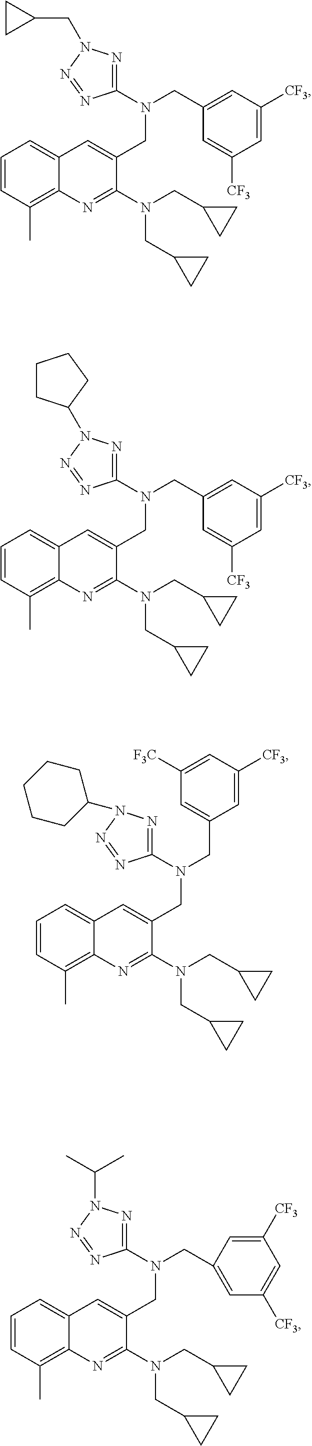 [SMILES: Cc1cccc2cc(CN(Cc3cc(C(F)(F)F)cc(C(F)(F)F)c3)c3nnn(CC4CC4)n3)c(N(CC3CC3)CC3CC3)nc12 ; Cc1cccc2cc(CN(Cc3cc(C(F)(F)F)cc(C(F)(F)F)c3)c3nnn(C4CCCC4)n3)c(N(CC3CC3)CC3CC3)nc12 ; Cc1cccc2cc(CN(Cc3cc(C(F)(F)F)cc(C(F)(F)F)c3)c3nnn(C4CCCCC4)n3)c(N(CC3CC3)CC3CC3)nc12 ; Cc1cccc2cc(CN(Cc3cc(C(F)(F)F)cc(C(F)(F)F)c3)c3nnn(C(C)C)n3)c(N(CC3CC3)CC3CC3)nc12]

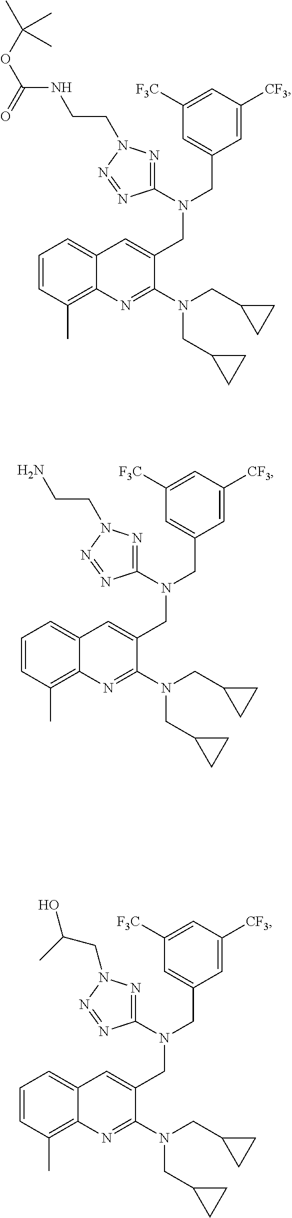 [SMILES: Cc1cccc2cc(CN(Cc3cc(C(F)(F)F)cc(C(F)(F)F)c3)c3nnn(CCNC(=O)OC(C)(C)C)n3)c(N(CC3CC3)CC3CC3)nc12 ; Cc1cccc2cc(CN(Cc3cc(C(F)(F)F)cc(C(F)(F)F)c3)c3nnn(CCN)n3)c(N(CC3CC3)CC3CC3)nc12 ; Cc1cccc2cc(CN(Cc3cc(C(F)(F)F)cc(C(F)(F)F)c3)c3nnn(CC(C)O)n3)c(N(CC3CC3)CC3CC3)nc12]

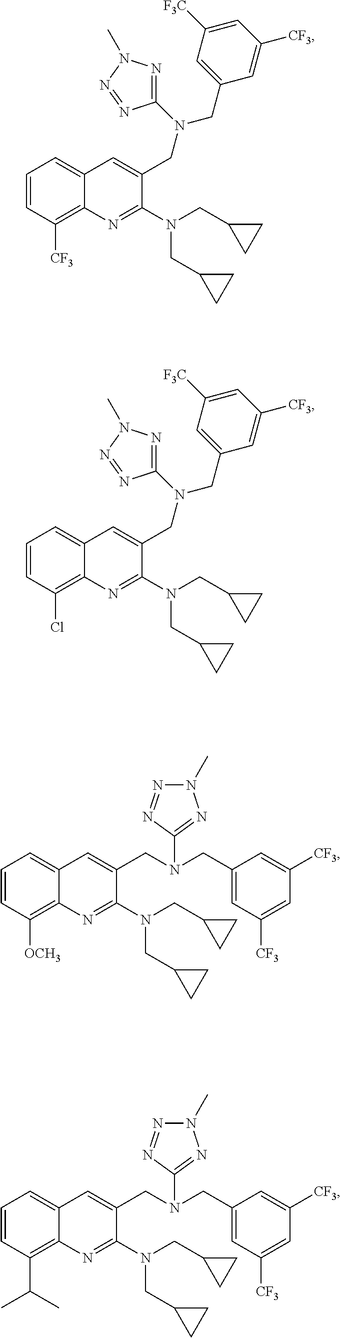 [SMILES: Cn1nnc(N(Cc2cc(C(F)(F)F)cc(C(F)(F)F)c2)Cc2cc3cccc(C(F)(F)F)c3nc2N(CC2CC2)CC2CC2)n1 ; Cn1nnc(N(Cc2cc(C(F)(F)F)cc(C(F)(F)F)c2)Cc2cc3cccc(Cl)c3nc2N(CC2CC2)CC2CC2)n1 ; COc1cccc2cc(CN(Cc3cc(C(F)(F)F)cc(C(F)(F)F)c3)c3nnn(C)n3)c(N(CC3CC3)CC3CC3)nc12 ; CC(C)c1cccc2cc(CN(Cc3cc(C(F)(F)F)cc(C(F)(F)F)c3)c3nnn(C)n3)c(N(CC3CC3)CC3CC3)nc12]

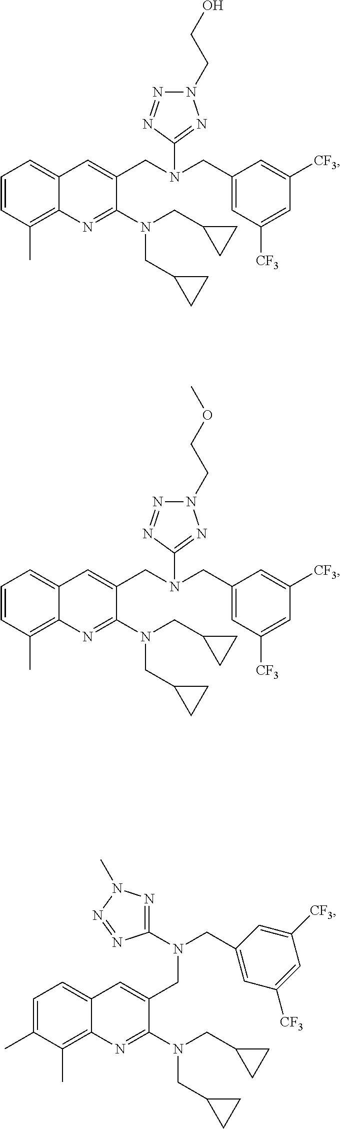 [SMILES: Cc1cccc2cc(CN(Cc3cc(C(F)(F)F)cc(C(F)(F)F)c3)c3nnn(CCO)n3)c(N(CC3CC3)CC3CC3)nc12 ; COCCn1nnc(N(Cc2cc(C(F)(F)F)cc(C(F)(F)F)c2)Cc2cc3cccc(C)c3nc2N(CC2CC2)CC2CC2)n1 ; Cc1ccc2cc(CN(Cc3cc(C(F)(F)F)cc(C(F)(F)F)c3)c3nnn(C)n3)c(N(CC3CC3)CC3CC3)nc2c1C]

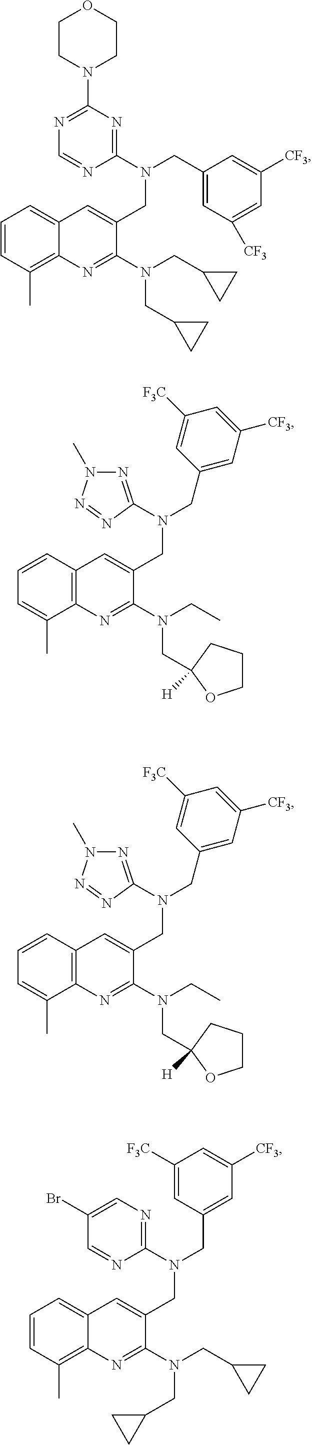 [SMILES: Cc1cccc2cc(CN(Cc3cc(C(F)(F)F)cc(C(F)(F)F)c3)c3ncnc(N4CCOCC4)n3)c(N(CC3CC3)CC3CC3)nc12 ; CCN(C[C@H]1CCCO1)c1nc2c(C)cccc2cc1CN(Cc1cc(C(F)(F)F)cc(C(F)(F)F)c1)c1nnn(C)n1 ; CCN(C[C@@H]1CCCO1)c1nc2c(C)cccc2cc1CN(Cc1cc(C(F)(F)F)cc(C(F)(F)F)c1)c1nnn(C)n1 ; Cc1cccc2cc(CN(Cc3cc(C(F)(F)F)cc(C(F)(F)F)c3)c3ncc(Br)cn3)c(N(CC3CC3)CC3CC3)nc12]

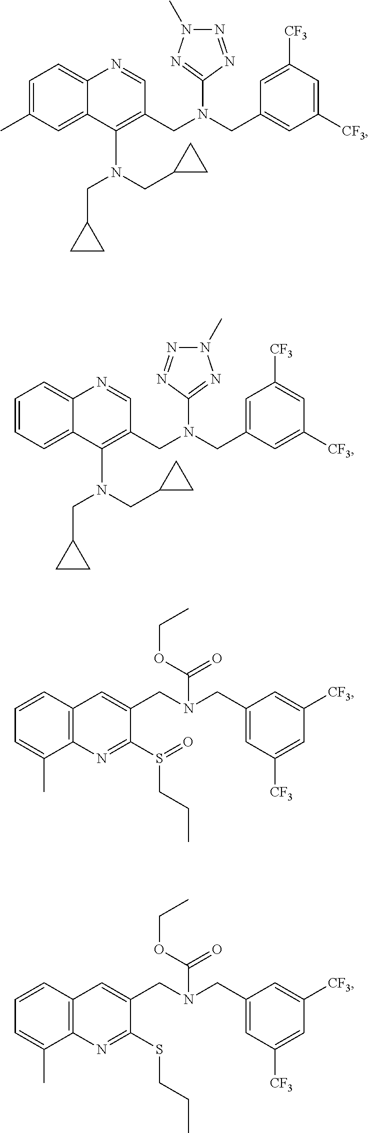 [SMILES: Cc1ccc2ncc(CN(Cc3cc(C(F)(F)F)cc(C(F)(F)F)c3)c3nnn(C)n3)c(N(CC3CC3)CC3CC3)c2c1 ; Cn1nnc(N(Cc2cc(C(F)(F)F)cc(C(F)(F)F)c2)Cc2cnc3ccccc3c2N(CC2CC2)CC2CC2)n1 ; CCCS(=O)c1nc2c(C)cccc2cc1CN(Cc1cc(C(F)(F)F)cc(C(F)(F)F)c1)C(=O)OCC ; CCCSc1nc2c(C)cccc2cc1CN(Cc1cc(C(F)(F)F)cc(C(F)(F)F)c1)C(=O)OCC]

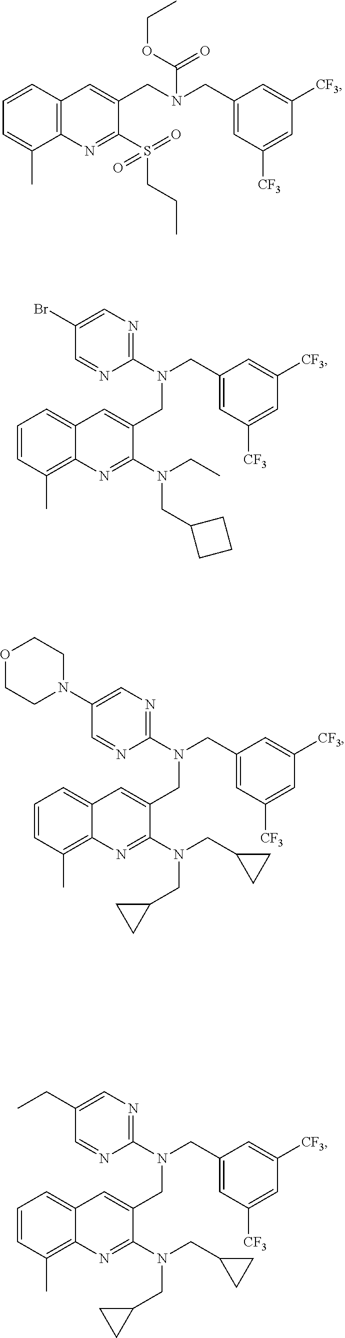 [SMILES: CCCS(=O)(=O)c1nc2c(C)cccc2cc1CN(Cc1cc(C(F)(F)F)cc(C(F)(F)F)c1)C(=O)OCC ; CCN(CC1CCC1)c1nc2c(C)cccc2cc1CN(Cc1cc(C(F)(F)F)cc(C(F)(F)F)c1)c1ncc(Br)cn1 ; Cc1cccc2cc(CN(Cc3cc(C(F)(F)F)cc(C(F)(F)F)c3)c3ncc(N4CCOCC4)cn3)c(N(CC3CC3)CC3CC3)nc12 ; CCc1cnc(N(Cc2cc(C(F)(F)F)cc(C(F)(F)F)c2)Cc2cc3cccc(C)c3nc2N(CC2CC2)CC2CC2)nc1]

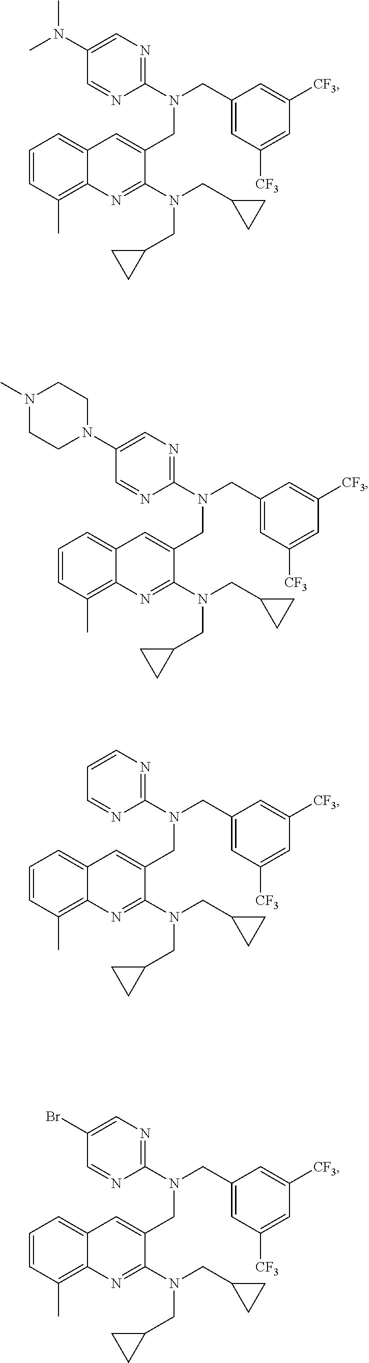 [SMILES: Cc1cccc2cc(CN(Cc3cc(C(F)(F)F)cc(C(F)(F)F)c3)c3ncc(N(C)C)cn3)c(N(CC3CC3)CC3CC3)nc12 ; Cc1cccc2cc(CN(Cc3cc(C(F)(F)F)cc(C(F)(F)F)c3)c3ncc(N4CCN(C)CC4)cn3)c(N(CC3CC3)CC3CC3)nc12 ; Cc1cccc2cc(CN(Cc3cc(C(F)(F)F)cc(C(F)(F)F)c3)c3ncccn3)c(N(CC3CC3)CC3CC3)nc12 ; Cc1cccc2cc(CN(Cc3cc(C(F)(F)F)cc(C(F)(F)F)c3)c3ncc(Br)cn3)c(N(CC3CC3)CC3CC3)nc12]

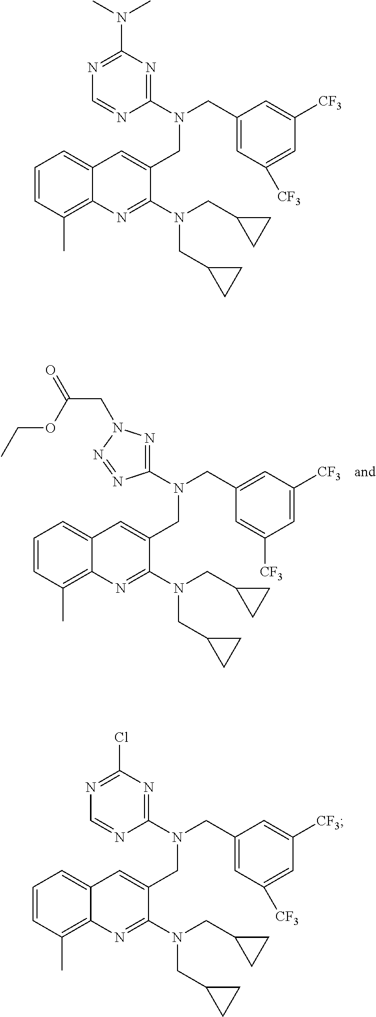 [SMILES: Cc1cccc2cc(CN(Cc3cc(C(F)(F)F)cc(C(F)(F)F)c3)c3ncnc(N(C)C)n3)c(N(CC3CC3)CC3CC3)nc12 ; CCOC(=O)Cn1nnc(N(Cc2cc(C(F)(F)F)cc(C(F)(F)F)c2)Cc2cc3cccc(C)c3nc2N(CC2CC2)CC2CC2)n1 ; Cc1cccc2cc(CN(Cc3cc(C(F)(F)F)cc(C(F)(F)F)c3)c3ncnc(Cl)n3)c(N(CC3CC3)CC3CC3)nc12]

or a stereoisomer thereof or a pharmaceutically acceptable salt thereof

In one embodiment, the application provides pharmaceutical compositions comprising one or more specific compounds of formula (II) and is enumerated as follows:

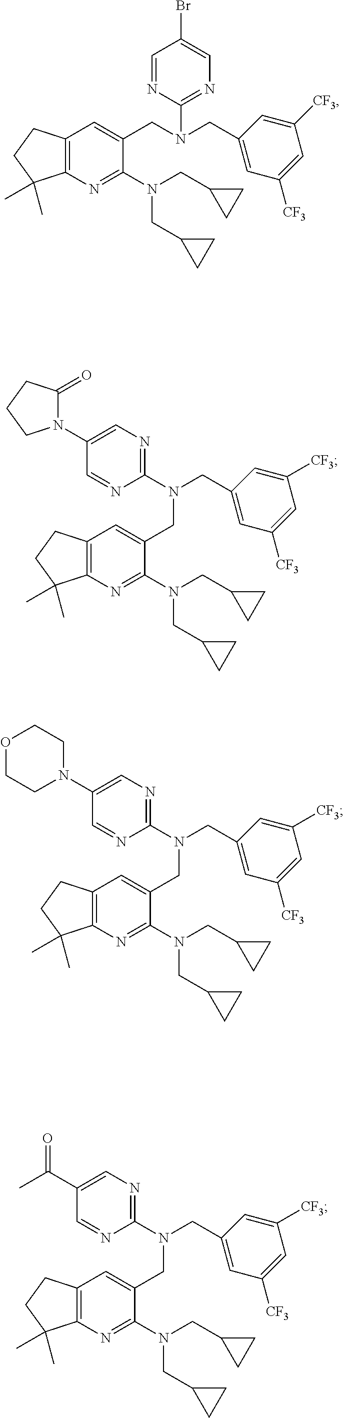 [SMILES: CC1(C)CCc2cc(CN(Cc3cc(C(F)(F)F)cc(C(F)(F)F)c3)c3ncc(Br)cn3)c(N(CC3CC3)CC3CC3)nc21 ; CC1(C)CCc2cc(CN(Cc3cc(C(F)(F)F)cc(C(F)(F)F)c3)c3ncc(N4CCCC4=O)cn3)c(N(CC3CC3)CC3CC3)nc21 ; CC1(C)CCc2cc(CN(Cc3cc(C(F)(F)F)cc(C(F)(F)F)c3)c3ncc(N4CCOCC4)cn3)c(N(CC3CC3)CC3CC3)nc21 ; CC(=O)c1cnc(N(Cc2cc(C(F)(F)F)cc(C(F)(F)F)c2)Cc2cc3c(nc2N(CC2CC2)CC2CC2)C(C)(C)CC3)nc1]

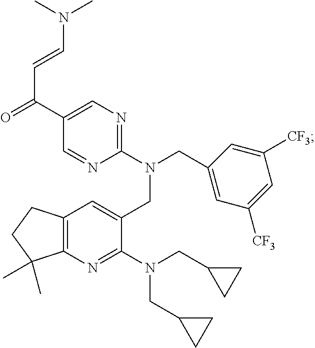 [SMILES: CN(C)/C=C/C(=O)c1cnc(N(Cc2cc(C(F)(F)F)cc(C(F)(F)F)c2)Cc2cc3c(nc2N(CC2CC2)CC2CC2)C(C)(C)CC3)nc1]

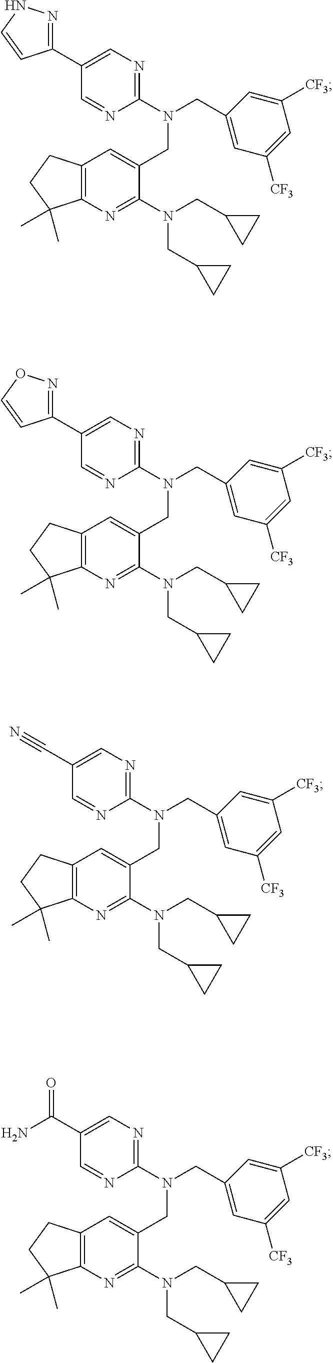 [SMILES: CC1(C)CCc2cc(CN(Cc3cc(C(F)(F)F)cc(C(F)(F)F)c3)c3ncc(-c4cc[nH]n4)cn3)c(N(CC3CC3)CC3CC3)nc21 ; CC1(C)CCc2cc(CN(Cc3cc(C(F)(F)F)cc(C(F)(F)F)c3)c3ncc(-c4ccon4)cn3)c(N(CC3CC3)CC3CC3)nc21 ; CC1(C)CCc2cc(CN(Cc3cc(C(F)(F)F)cc(C(F)(F)F)c3)c3ncc(C#N)cn3)c(N(CC3CC3)CC3CC3)nc21 ; CC1(C)CCc2cc(CN(Cc3cc(C(F)(F)F)cc(C(F)(F)F)c3)c3ncc(C(N)=O)cn3)c(N(CC3CC3)CC3CC3)nc21]

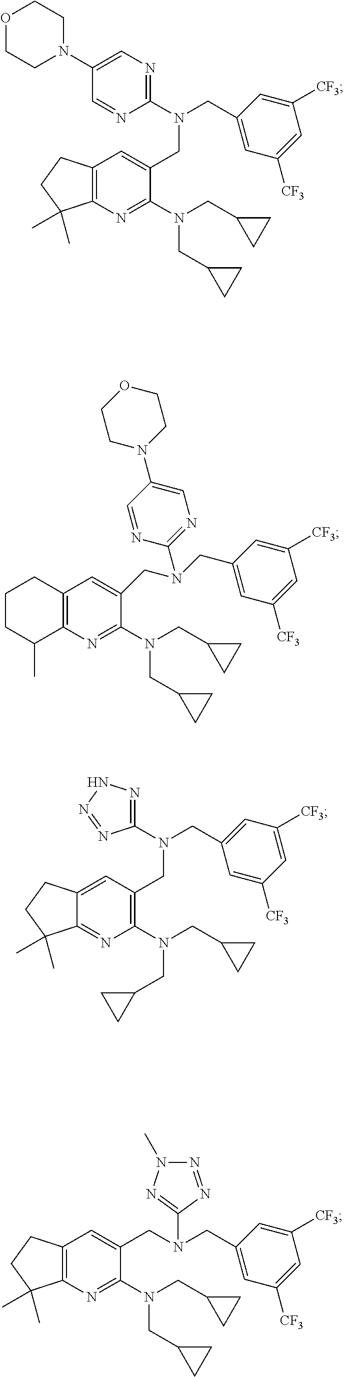 [SMILES: CC1(C)CCc2cc(CN(Cc3cc(C(F)(F)F)cc(C(F)(F)F)c3)c3ncc(N4CCOCC4)cn3)c(N(CC3CC3)CC3CC3)nc21 ; CC1CCCc2cc(CN(Cc3cc(C(F)(F)F)cc(C(F)(F)F)c3)c3ncc(N4CCOCC4)cn3)c(N(CC3CC3)CC3CC3)nc21 ; CC1(C)CCc2cc(CN(Cc3cc(C(F)(F)F)cc(C(F)(F)F)c3)c3nn[nH]n3)c(N(CC3CC3)CC3CC3)nc21 ; Cn1nnc(N(Cc2cc(C(F)(F)F)cc(C(F)(F)F)c2)Cc2cc3c(nc2N(CC2CC2)CC2CC2)C(C)(C)CC3)n1]

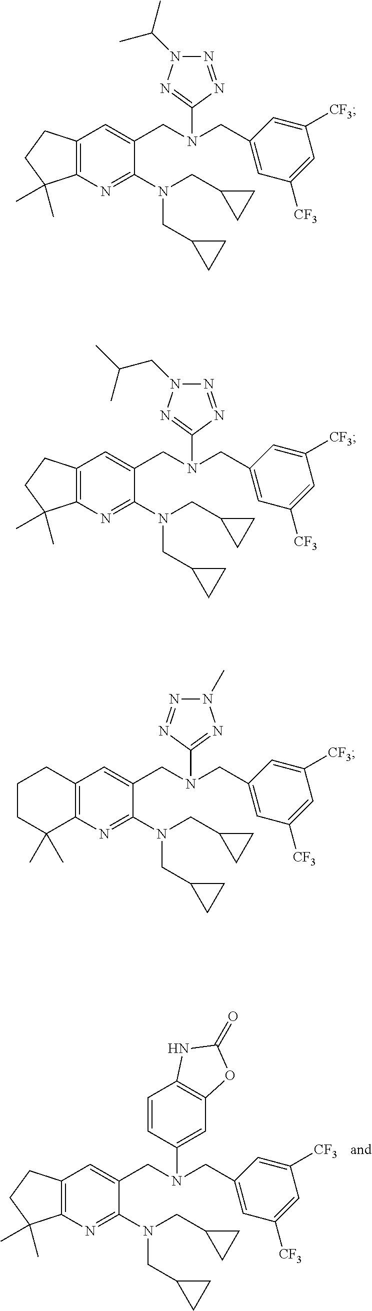 [SMILES: CC(C)n1nnc(N(Cc2cc(C(F)(F)F)cc(C(F)(F)F)c2)Cc2cc3c(nc2N(CC2CC2)CC2CC2)C(C)(C)CC3)n1 ; CC(C)Cn1nnc(N(Cc2cc(C(F)(F)F)cc(C(F)(F)F)c2)Cc2cc3c(nc2N(CC2CC2)CC2CC2)C(C)(C)CC3)n1 ; Cn1nnc(N(Cc2cc(C(F)(F)F)cc(C(F)(F)F)c2)Cc2cc3c(nc2N(CC2CC2)CC2CC2)C(C)(C)CCC3)n1 ; CC1(C)CCc2cc(CN(Cc3cc(C(F)(F)F)cc(C(F)(F)F)c3)c3ccc4[nH]c(=O)oc4c3)c(N(CC3CC3)CC3CC3)nc21]

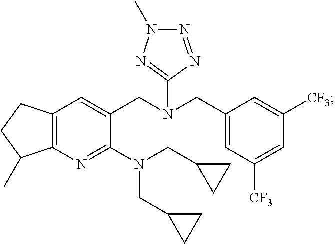 [SMILES: CC1CCc2cc(CN(Cc3cc(C(F)(F)F)cc(C(F)(F)F)c3)c3nnn(C)n3)c(N(CC3CC3)CC3CC3)nc21]

or a stereoisomer thereof or a pharmaceutically acceptable salt thereof

In one embodiment, the application provides pharmaceutical compositions comprising one or more specific compounds of formula (III) and is enumerated as follows:

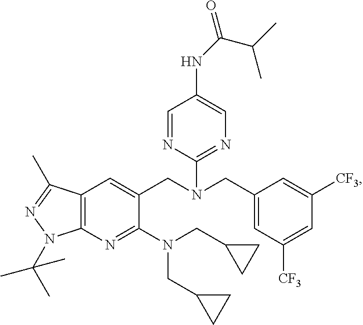 [SMILES: Cc1nn(C(C)(C)C)c2nc(N(CC3CC3)CC3CC3)c(CN(Cc3cc(C(F)(F)F)cc(C(F)(F)F)c3)c3ncc(NC(=O)C(C)C)cn3)cc12]

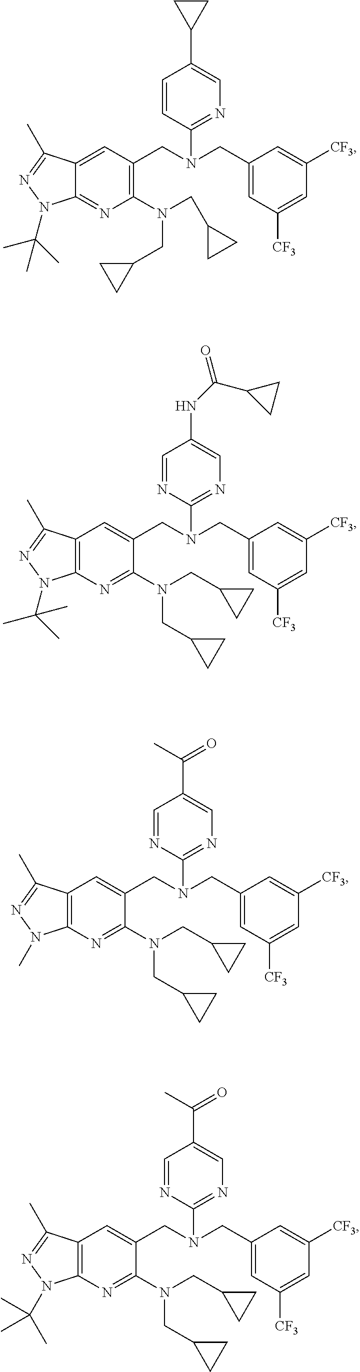 [SMILES: Cc1nn(C(C)(C)C)c2nc(N(CC3CC3)CC3CC3)c(CN(Cc3cc(C(F)(F)F)cc(C(F)(F)F)c3)c3ccc(C4CC4)cn3)cc12 ; Cc1nn(C(C)(C)C)c2nc(N(CC3CC3)CC3CC3)c(CN(Cc3cc(C(F)(F)F)cc(C(F)(F)F)c3)c3ncc(NC(=O)C4CC4)cn3)cc12 ; CC(=O)c1cnc(N(Cc2cc(C(F)(F)F)cc(C(F)(F)F)c2)Cc2cc3c(C)nn(C)c3nc2N(CC2CC2)CC2CC2)nc1 ; CC(=O)c1cnc(N(Cc2cc(C(F)(F)F)cc(C(F)(F)F)c2)Cc2cc3c(C)nn(C(C)(C)C)c3nc2N(CC2CC2)CC2CC2)nc1]

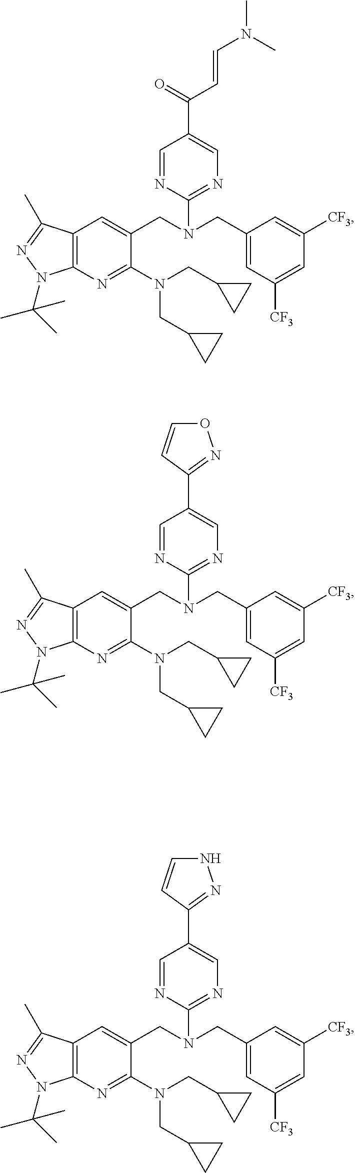 [SMILES: Cc1nn(C(C)(C)C)c2nc(N(CC3CC3)CC3CC3)c(CN(Cc3cc(C(F)(F)F)cc(C(F)(F)F)c3)c3ncc(C(=O)/C=C/N(C)C)cn3)cc12 ; Cc1nn(C(C)(C)C)c2nc(N(CC3CC3)CC3CC3)c(CN(Cc3cc(C(F)(F)F)cc(C(F)(F)F)c3)c3ncc(-c4ccon4)cn3)cc12 ; Cc1nn(C(C)(C)C)c2nc(N(CC3CC3)CC3CC3)c(CN(Cc3cc(C(F)(F)F)cc(C(F)(F)F)c3)c3ncc(-c4cc[nH]n4)cn3)cc12]

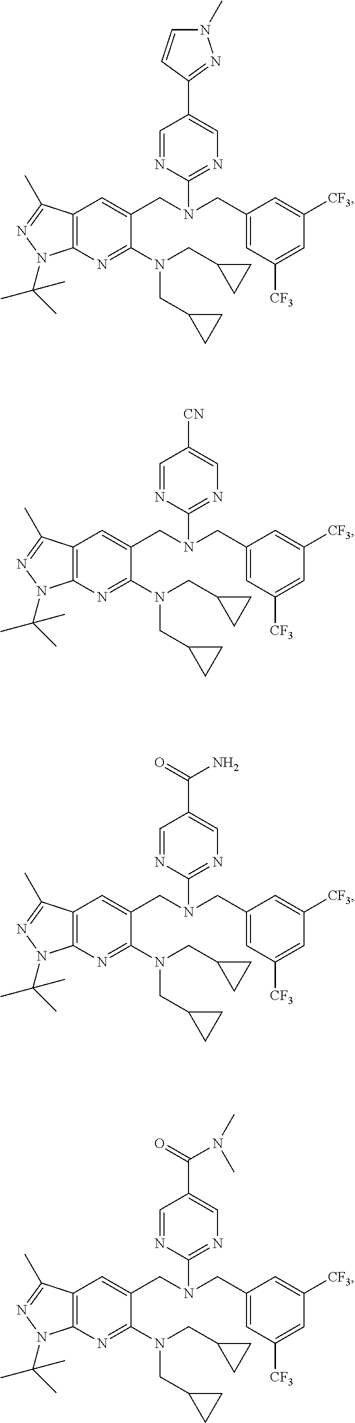 [SMILES: Cc1nn(C(C)(C)C)c2nc(N(CC3CC3)CC3CC3)c(CN(Cc3cc(C(F)(F)F)cc(C(F)(F)F)c3)c3ncc(-c4ccn(C)n4)cn3)cc12 ; Cc1nn(C(C)(C)C)c2nc(N(CC3CC3)CC3CC3)c(CN(Cc3cc(C(F)(F)F)cc(C(F)(F)F)c3)c3ncc(C#N)cn3)cc12 ; Cc1nn(C(C)(C)C)c2nc(N(CC3CC3)CC3CC3)c(CN(Cc3cc(C(F)(F)F)cc(C(F)(F)F)c3)c3ncc(C(N)=O)cn3)cc12 ; Cc1nn(C(C)(C)C)c2nc(N(CC3CC3)CC3CC3)c(CN(Cc3cc(C(F)(F)F)cc(C(F)(F)F)c3)c3ncc(C(=O)N(C)C)cn3)cc12]

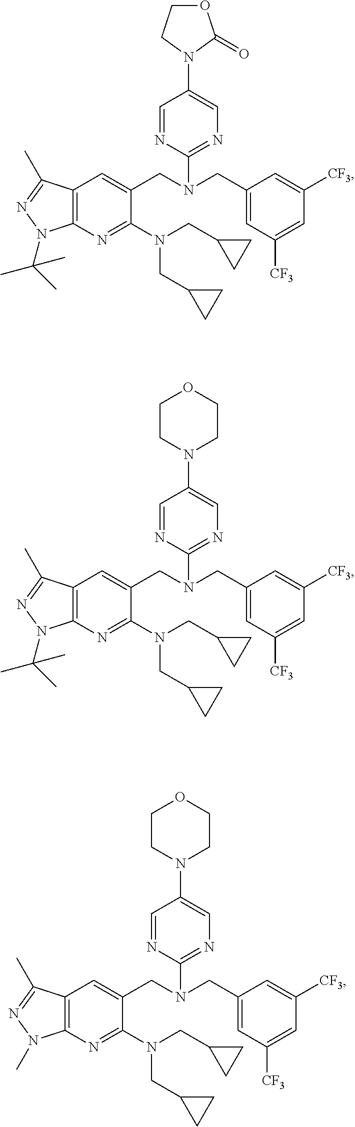 [SMILES: Cc1nn(C(C)(C)C)c2nc(N(CC3CC3)CC3CC3)c(CN(Cc3cc(C(F)(F)F)cc(C(F)(F)F)c3)c3ncc(N4CCOC4=O)cn3)cc12 ; Cc1nn(C(C)(C)C)c2nc(N(CC3CC3)CC3CC3)c(CN(Cc3cc(C(F)(F)F)cc(C(F)(F)F)c3)c3ncc(N4CCOCC4)cn3)cc12 ; Cc1nn(C)c2nc(N(CC3CC3)CC3CC3)c(CN(Cc3cc(C(F)(F)F)cc(C(F)(F)F)c3)c3ncc(N4CCOCC4)cn3)cc12]

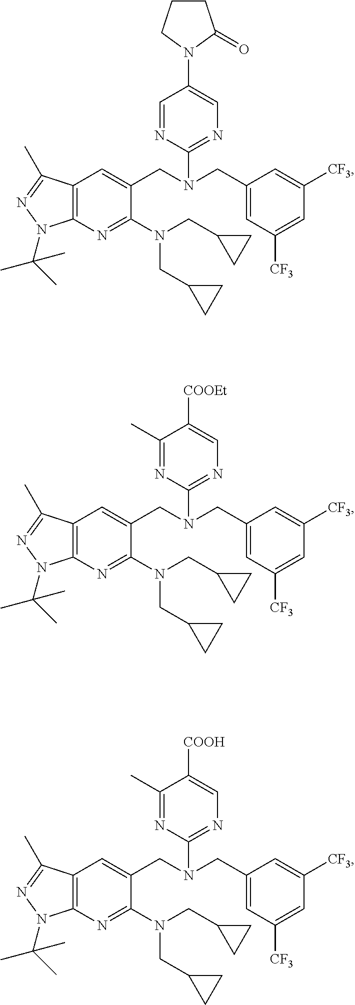 [SMILES: Cc1nn(C(C)(C)C)c2nc(N(CC3CC3)CC3CC3)c(CN(Cc3cc(C(F)(F)F)cc(C(F)(F)F)c3)c3ncc(N4CCCC4=O)cn3)cc12 ; CCOC(=O)c1cnc(N(Cc2cc(C(F)(F)F)cc(C(F)(F)F)c2)Cc2cc3c(C)nn(C(C)(C)C)c3nc2N(CC2CC2)CC2CC2)nc1C ; Cc1nc(N(Cc2cc(C(F)(F)F)cc(C(F)(F)F)c2)Cc2cc3c(C)nn(C(C)(C)C)c3nc2N(CC2CC2)CC2CC2)ncc1C(=O)O]

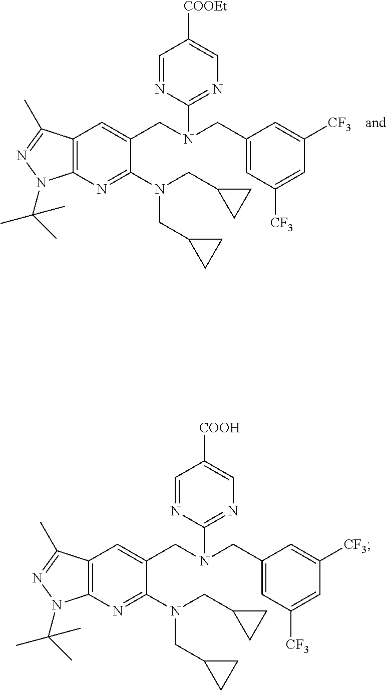 [SMILES: CCOC(=O)c1cnc(N(Cc2cc(C(F)(F)F)cc(C(F)(F)F)c2)Cc2cc3c(C)nn(C(C)(C)C)c3nc2N(CC2CC2)CC2CC2)nc1 ; Cc1nn(C(C)(C)C)c2nc(N(CC3CC3)CC3CC3)c(CN(Cc3cc(C(F)(F)F)cc(C(F)(F)F)c3)c3ncc(C(=O)O)cn3)cc12]

or a stereoisomer thereof or a pharmaceutically acceptable salt thereof.

In another embodiment, the application provides pharmaceutical compositions comprising one or more specific compounds of formulae (I), (Ia′), (II) or (III) and is enumerated as follows:

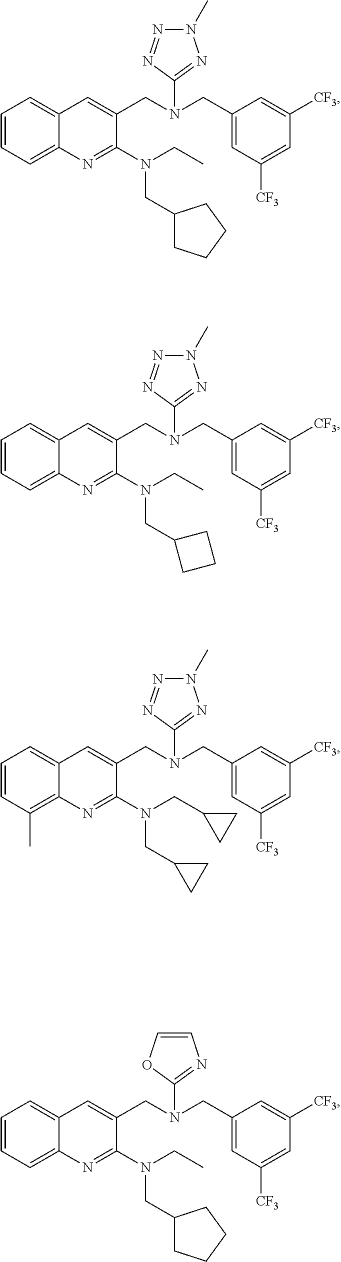 [SMILES: CCN(CC1CCCC1)c1nc2ccccc2cc1CN(Cc1cc(C(F)(F)F)cc(C(F)(F)F)c1)c1nnn(C)n1 ; CCN(CC1CCC1)c1nc2ccccc2cc1CN(Cc1cc(C(F)(F)F)cc(C(F)(F)F)c1)c1nnn(C)n1 ; Cc1cccc2cc(CN(Cc3cc(C(F)(F)F)cc(C(F)(F)F)c3)c3nnn(C)n3)c(N(CC3CC3)CC3CC3)nc12 ; CCN(CC1CCCC1)c1nc2ccccc2cc1CN(Cc1cc(C(F)(F)F)cc(C(F)(F)F)c1)c1ncco1]

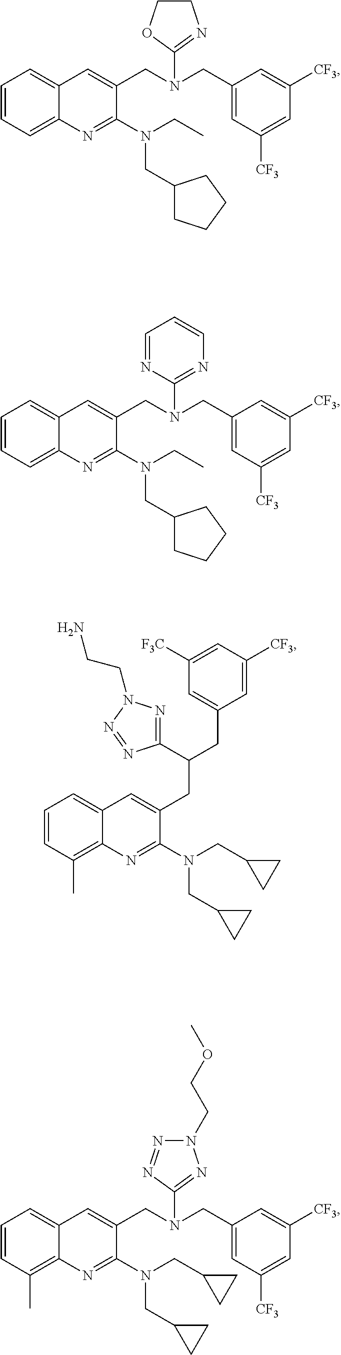 [SMILES: CCN(CC1CCCC1)c1nc2ccccc2cc1CN(Cc1cc(C(F)(F)F)cc(C(F)(F)F)c1)C1=NCCO1 ; CCN(CC1CCCC1)c1nc2ccccc2cc1CN(Cc1cc(C(F)(F)F)cc(C(F)(F)F)c1)c1ncccn1 ; Cc1cccc2cc(CC(Cc3cc(C(F)(F)F)cc(C(F)(F)F)c3)c3nnn(CCN)n3)c(N(CC3CC3)CC3CC3)nc12 ; COCCn1nnc(N(Cc2cc(C(F)(F)F)cc(C(F)(F)F)c2)Cc2cc3cccc(C)c3nc2N(CC2CC2)CC2CC2)n1]

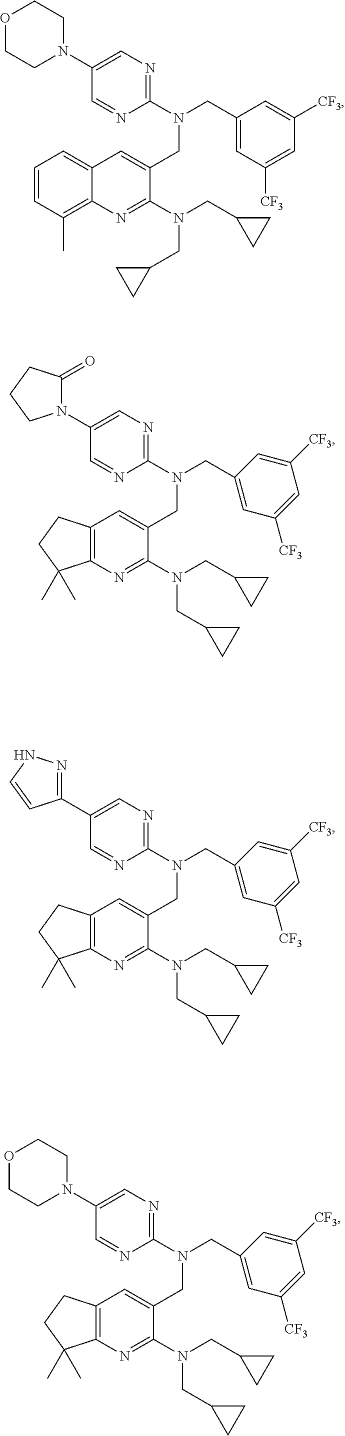 [SMILES: Cc1cccc2cc(CN(Cc3cc(C(F)(F)F)cc(C(F)(F)F)c3)c3ncc(N4CCOCC4)cn3)c(N(CC3CC3)CC3CC3)nc12 ; CC1(C)CCc2cc(CN(Cc3cc(C(F)(F)F)cc(C(F)(F)F)c3)c3ncc(N4CCCC4=O)cn3)c(N(CC3CC3)CC3CC3)nc21 ; CC1(C)CCc2cc(CN(Cc3cc(C(F)(F)F)cc(C(F)(F)F)c3)c3ncc(-c4cc[nH]n4)cn3)c(N(CC3CC3)CC3CC3)nc21 ; CC1(C)CCc2cc(CN(Cc3cc(C(F)(F)F)cc(C(F)(F)F)c3)c3ncc(N4CCOCC4)cn3)c(N(CC3CC3)CC3CC3)nc21]

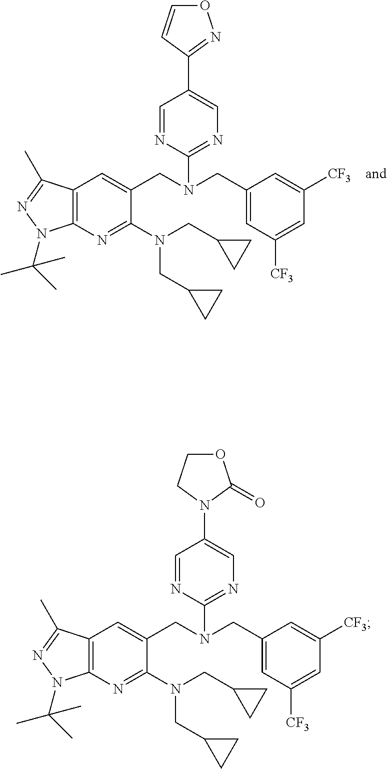 [SMILES: Cc1nn(C(C)(C)C)c2nc(N(CC3CC3)CC3CC3)c(CN(Cc3cc(C(F)(F)F)cc(C(F)(F)F)c3)c3ncc(-c4ccon4)cn3)cc12 ; Cc1nn(C(C)(C)C)c2nc(N(CC3CC3)CC3CC3)c(CN(Cc3cc(C(F)(F)F)cc(C(F)(F)F)c3)c3ncc(N4CCOC4=O)cn3)cc12]

or a stereoisomer thereof or a pharmaceutically acceptable salt thereof

In another aspect, the present application provides a composition in which the CETP inhibitor of formula (I), (Ia′), (II) or (III) is combined with at least one surfactant in a sufficient amount so that the composition provides maximum drug availability for absorption.

In one embodiment, the CETP inhibitor of formula (I), (Ia′), (II) or (III) is combined with at least one surfactant in the form of a solution, suspension, emulsion or a pre-concentrate.

In one embodiment, the CETP inhibitor of the present application is sparingly or poorly soluble in water. Generally CETP inhibitors exhibit an aqueous solubility (e.g. in water) of less than about 10 μg/mL measured at about 22° C. and at a physiologically relevant pH of from 1 to 8.

In another embodiment, solubility of a CETP inhibitor in an aqueous solution in the absence of said surfactant of less than 10 μg/ml at any pH of from 1 to 8.

In another embodiment, solubility of a CETP inhibitor in an aqueous solution in the absence of said surfactant of less than 2 μg/ml at any pH of from 1 to 8.

In another embodiment, solubility of a CETP inhibitor in an aqueous solution in the absence of said surfactant of less than 1 μg/ml at any pH of from 1 to 8.

In one embodiment, CETP inhibitor is selected from a compound of formula (I), which is as defined above.

In one embodiment, CETP inhibitor is selected from a compound of formula (Ia′), which is as defined above.

In one embodiment, CETP inhibitor is selected from a compound of formula (II), which is as defined above.

In one embodiment, CETP inhibitor is selected from a compound of formula (III), which is as defined above.

In another aspect, the present application relates to a pharmaceutical composition comprising:

a) a CETP inhibitor having formula (I) or (Ia′) or (II) or (III),

b) at least one surfactant,

c) optionally a carrier material, and

d) optionally one or more pharmaceutically acceptable excipients.

In yet another aspect, the present application provides a method of administering a pharmaceutical composition to a patient in need, wherein said composition comprising:

a) a CETP inhibitor having formula (I) or (Ia′) or (II) or (III),

b) at least one hydrophilic surfactant,

c) optionally a carrier material, and

d) optionally one or more pharmaceutically acceptable excipients.

In another embodiment, the compositions of the present application are useful in treating or preventing diseases that can be treated or prevented with CETP inhibitors, including atherosclerosis, peripheral vascular disease, dyslipidemia, hyperbetalipoproteinemia, hypoalphalipoproteinemia, hypercholesterolemia, hypertriglyceridemia, familial hypercholesterolemia, cardiovascular disorders, angina, ischemia, cardiac ischemia, stroke, myocardial infarction, reperfusion injury, angioplastic restenosis, hypertension, vascular complications of diabetes, obesity and endotoxemia. The compositions may also be useful in preventing or delaying the recurrence of certain diseases or adverse events, such as myocardial infarction, ischemia, cardiac ischemia, and stroke.

In another aspect, there is provided a process for preparing a pharmaceutical composition comprising:

a) a CETP inhibitor having formula (I) or (Ia′) or (II) or (III),

b) at least one surfactant,

c) optionally a carrier material, and

d) optionally one or more pharmaceutically acceptable excipients.

In one embodiment of the above aspect, wherein a CETP inhibitor is selected from compound of formula (I), which is defined as earlier.

In one embodiment of the above aspect, wherein a CETP inhibitor is selected from compound of formula (Ia′), which is defined as earlier.

In one embodiment of the above aspect, wherein a CETP inhibitor is selected from compound of formula (II), which is defined as earlier.

In one embodiment of the above aspect, wherein a CETP inhibitor is selected from compound of formula (III), which is defined as earlier.

In another aspect, a pharmaceutical composition comprising a CETP inhibitor and a surfactant, provides a maximum concentration of the CETP inhibitor in an use environment, that is at least about 10-fold the maximum concentration provided by a control composition comprising an equivalent amount of the CETP inhibitor and free from any surfactant. As used herein, “use environment” can be either the in vivo environment of the GI tract of an animal, such as a mammal, including a human, or the in vitro environment of a test solution, such as phosphate buffered saline (PBS) or fasted simulated gastric fluid or fasted simulated intestinal fluid.

In one aspect, the present application provides a composition comprising a CETP inhibitor of formula (I), (Ia′), (II) or (III) and at least one surfactant, wherein said composition releases not less than 20% at a period of 15 minutes in 900 ml of 0.01N HCl, when tested in a USP Type 2 apparatus at 50 rpm and 37° C.

In another aspect, the present application provides a composition comprising a CETP inhibitor of formula (I), (Ia′), (II) or (III) and at least one surfactant, wherein said composition releases not less than 40% at a period of 30 minutes in 900 ml of 0.01N HCl, when tested in a USP Type 2 apparatus at 50 rpm and 37° C.

In yet another aspect, the present application provides a composition comprising a CETP inhibitor of formula (I), (Ia′), (II) or (III) and at least one surfactant, wherein said composition releases not less than 75% at a period of 60 minutes in 900 ml of 0.01N HCl, when tested in a USP Type 2 apparatus at 50 rpm and 37° C.

In one embodiment, the composition of the present application may be in the form of pre-concentrate. The term “pre-concentrate” refer to its function of forming a stable emulsion when gently mixed with water or other aqueous medium, usually in the use environment. The pre-concentrate can be self-emulsifying or self-microemulsifying.

The term “self-emulsifying” refers to a composition which, when diluted by a factor of at least 100 by water or other aqueous medium and gently mixed, yields an opaque, stable oil/water emulsion with a mean droplet diameter less than about 5 microns, but greater than 100 nm, or greater than 50 nm or greater than 10 nm. Such emulsions yield no visibly detectable phase separation and that there is no visibly detectable crystallization of CETP inhibitor.

The term “self-microemulsifying” refers to a pre-concentrate which, upon at least 100× dilutions with an aqueous medium and gentle mixing, yields a non-opaque, stable oil/water microemulsion with an average droplet size of about 1 micron or less. Until the composition comes in contact with an aqueous medium, it does not form a microemulsion, instead, when the various components are mixed, it forms what in the art is known as a pre-concentrate of an emulsion, i.e., microemulsion pre-concentrate, a system capable of forming microemulsion respectively, on contact with water or aqueous medium. The microemulsion is thermodynamically stable and without any indication of crystallization of CETP inhibitor.

“Gentle mixing” as used above is understood in the art to refer to the formation of an emulsion by gentle hand (or machine) mixing, such as by repeated inversions on a standard laboratory mixing machine. High shear mixing is not required to form the emulsion. Such pre-concentrates generally emulsify nearly spontaneously when introduced into the use environment.

It has now been found surprisingly that the compositions of the present application exhibit dramatic enhancements in aqueous concentration and bioavailability when formulated using the compounds as described herein.

The compositions of present application include at least one surfactant. The term “surfactant” refers to a compound that necessarily includes polar or charged hydrophilic moieties as well as non-polar or hydrophobic (lipophilic) moieties; i.e., a surfactant compound must be amphiphilic. The surfactant generally lowers the surface tension of a liquid, allows easier spreading, and lowers the interfacial tension between two liquids there by facilitating dispersion process.

An empirical parameter commonly used to characterize the relative hydrophilicity and hydrophobicity of non-ionic amphiphilic compounds is the hydrophilic-lipophilic balance (“HLB” value). The HLB, an acronym for “hydrophobic-lipophilic balance”, is a rating scale which can range from 1-20 for non-ionic surfactants. Surfactants with lower HLB values are more hydrophobic, and have greater solubility in oils, while surfactants with higher HLB values are more hydrophilic, and have greater solubility in aqueous solutions. Using HLB values as a rough guide, hydrophilic surfactants are generally considered to be those compounds having an HLB value greater than about 10, as well as anionic, cationic, or zwitterionic compounds for which the HLB scale is not generally applicable. Similarly, hydrophobic surfactants are compounds having an HLB value less than about 10. The surfactant used in the compositions may be hydrophilic or hydrophobic in nature.

In one embodiment, the pharmaceutical compositions of present application comprise at least one surfactant, wherein said surfactant is hydrophilic, hydrophobic or mixtures thereof.

The hydrophilic surfactant can be any hydrophilic surfactant suitable for use in pharmaceutical compositions. Such surfactants can be anionic, cationic, zwitterionic or non-ionic. Mixtures of hydrophilic surfactants are also within the scope of the application. Similarly, the hydrophobic surfactant can be any hydrophobic surfactant suitable for use in pharmaceutical compositions. Mixtures of hydrophobic surfactants are also within the scope of the application. In addition to the above, combinations of both hydrophilic and hydrophobic surfactants are also within the scope of the application.

In another embodiment, the surfactant may typically comprise from about 1% to about 90%, from about 5% to about 85%, from about 10% to 80% weight of the composition.

Ionic hydrophilic surfactants may be selected from a group of: fatty acid salts, bile salts, phospholipids, phosphoric acid esters, carboxylates, acyl lactylates, alginate salts, sulfates and sulfonates, cationic surfactants and combinations comprising one or more of the foregoing material.

Suitable examples of fatty acid salts include, but are not limited to, sodium caproate, sodium caprylate, sodium caprate, sodium laurate, sodium myristate, sodium myristoleate, sodium palmitate, sodium palmitoleate, sodium oleate, sodium ricinoleate, sodium linoleate, sodium linolenate, sodium stearate, sodium lauryl sulfate, sodium tetradecyl sulfate, sodium lauryl sarcosinate and sodium dioctyl sulfosuccinate.

Suitable examples of bile salts include, but are not limited to, sodium cholate, sodium taurocholate, sodium glycocholate, sodium deoxycholate, sodium taurodeoxycholate, sodium glycodeoxycholate, sodium ursodeoxycholate, sodium chenodeoxycholate, sodium taurochenodeoxycholate, sodium glyco cheno deoxycholate, sodium cholylsarcosinate, sodium N-methyl taurocholate and sodium lithocholate.

Suitable examples of phospholipids include, but are not limited to, egg/soy lecithin, lyso egg/soy lecithin, hydroxylated lecithin, lysophosphatidylcholine, cardiolipin, sphingomyelin, phosphatidylcholine, phosphatidyl ethanolamine, phosphatidic acid, phosphatidyl glycerol and phosphatidyl serine.

Suitable examples of phosphoric acid esters include, but are not limited to, diethanolammonium polyoxyethylene-10 oleyl ether phosphate, esterification products of fatty alcohols or fatty alcohol ethoxylates with phosphoric acid or anhydride.

Suitable examples of carboxylates include, but are not limited to, ether carboxylates (by oxidation of terminal OH group of fatty alcohol ethoxylates), succinylated monoglycerides, sodium stearyl fumarate, stearoyl propylene glycol hydrogen succinate, mono/diacetylated tartaric acid esters of mono- and diglycerides, citric acid esters of mono-diglycerides and glyceryl-lacto esters of fatty acids.

Suitable examples of acyl lactylates include, but are not limited to, lactylic esters of fatty acids, calcium/sodium stearoyl-2-lactylate and calcium/sodium stearoyl lactylate.

Example for alginate salts include, but are not limited to, propylene glycol alginate.

Suitable examples of sulfates and sulfonates include, but are not limited to, ethoxylated alkyl sulfates, alkyl benzene sulfones, α-olefin sulfonates, acyl isethionate, acyl taurates, alkyl glyceryl ether sulfonates, octyl sulfosuccinate disodium and disodium undecylenamideo-MEA-sulfosuccinate.

Suitable examples of cationic surfactants include, but are not limited to, lauroyl carnitine, palmitoyl carnitine, myristoyl carnitine, hexadecyl triammonium bromide, decyl trimethyl ammonium bromide, cetyl trimethyl ammonium bromide, dodecyl ammonium chloride, alkyl benzyldimethylammonium salts, diisobutyl phenoxyethoxydimethyl benzylammonium salts, alkylpyridinium salts, betaines (trialkylglycine): lauryl betaine (N-lauryl, N,N-dimethylglycine) and ethoxylated amines: polyoxyethylene-15 coconut amine.

Non-ionic hydrophilic surfactants may be selected from a group of: polyethoxylated fatty Acids, PEG fatty acid diesters, PEG-fatty acid mono- and di-ester mixtures, polyethylene glycol glycerol fatty acid esters, alcohol-oil transesterification products, polyglycerized fatty acids, sterol and sterol derivatives, polyethylene glycol sorbitan fatty acid esters, polyethylene glycol alkyl ethers, sugar ethers, polyethylene glycol alkyl phenols, polyoxyethylene polyoxypropylene block copolymers and combinations comprising one or more of the foregoing material.

Suitable examples of polyethoxylated fatty acids include, but are not limited to, PEG 4-100 monolaurate, PEG 4-100 monooleate, PEG 4-100 monostearate, PEG 400 distearate, PEG 100, 200, 300 mono-laurate, PEG 100, 200, 300 mono-oleate, PEG 400 dioleate, PEG 400-1000 mono-stearate, PEG-7 oleate, PEG-6 laurate, PEG-7 laurate, PEG-8 laurate, PEG-8 oleate, PEG-8 stearate, PEG-9 oleate, PEG-9 stearate, PEG-10 laurate, PEG-10 oleate, PEG-10 stearate, PEG-12 laurate, PEG-12 oleate, PEG-12 ricinoleate, PEG-12 stearate, PEG-15 stearate, PEG-15 oleate, PEG-20 laurate, PEG-20 oleate, PEG-20 stearate, PEG-25 stearate, PEG-32 laurate, PEG-32 oleate, PEG-32 stearate, PEG-30 stearate, PEG-40 laurate, PEG-40 oleate, PEG-40 stearate, PEG-45 stearate, PEG-50 stearate, PEG-55 stearate, PEG-100 oleate, PEG-100 stearate, 19 PEG-200 oleate, PEG-400 oleate and PEG-600 oleate.

Suitable examples of PEG fatty acid diesters include, but are not limited to, PEG-8 dilaurate, PEG-8 distearate, PEG-10 dipalmitate, PEG-12 dilaurate, PEG-12 distearate, PEG-12 dioleate, PEG-20 dilaurate, PEG-20 dioleate, PEG-20 distearate, PEG-32 dilaurate, PEG-32 dioleate, PEG-32 distearate, PEG-400 dioleate and PEG-400 distearate.

Suitable examples of PEG-fatty acid mono- and di-ester mixtures include, but are not limited to, PEG 4-150 mono, dilaurate, PEG 4-150 mono, dioleate and PEG 4-150 mono, distearate.

Suitable examples of polyethylene glycol glycerol fatty acid esters include, but are not limited to, PEG-20 glyceryl laurate, PEG-30 glyceryl laurate, PEG-15 glyceryl laurate, PEG-40 glyceryl laurate, PEG-20 glyceryl stearate, PEG-20 glyceryl oleate and PEG-30 glyceryl oleate.

Suitable examples of alcohol-oil transesterification products include, but are not limited to, PEG-20 castor oil, PEG-23 castor oil, PEG-30 castor oil, PEG-35 castor oil (CREMOPHOR® EL and EL-P EMULPHOR® EL, INCROCAS™-35), PEG-38 castor oil, PEG-40 castor oil, PEG-50 castor oil, PEG-56 castor oil, PEG-60 castor oil, PEG-100 castor oil, PEG-200 castor oil, PEG-20 hydrogenated castor oil, PEG-25 hydrogenated castor oil, PEG-30 hydrogenated castor oil, PEG-40 hydrogenated castor oil (CREMOPHOR® RH 40), PEG-45 hydrogenated castor oil, PEG-50 hydrogenated castor oil, PEG-60 hydrogenated castor oil, PEG-80 hydrogenated castor oil, PEG-100 hydrogenated castor oil, PEG-25 trioleate, PEG-40 palm kernel oil, PEG-60 corn glycerides, PEG-60 almond glycerides, PEG-8 caprylic/capric glycerides (LABRASOL®, LABRAFAC® CM 10), PEG-6 caprylic/capric glycerides, Lauroyl macrogol-32 glyceride and stearoyl macrogol glyceride.

Suitable examples of polyglycerized fatty acids include, but are not limited to, polyglyceryl-10 laurate, polyglyceryl-10 oleate, polyglyceryl-10 stearate, polyglyceryl-10 linoleate and polyglyceryl-10 mono dioleate.

Suitable examples of sterol and sterol derivatives include, but are not limited to, PEG-24 cholesterol ether, PEG-30 cholestanol, PEG-25 phyto sterol and PEG-30 soya sterol.

Suitable examples of polyethylene glycol sorbitan fatty acid esters include, but are not limited to, PEG-10 sorbitan laurateb (LIPOSORB® L-10), PEG-20 sorbitan mono laurate (Tween-20), PEG-4 sorbitan mono laurate (Tween-21), PEG-80 sorbitan mono laurate, PEG-6 sorbitan mono laurate, PEG-20 sorbitan mono palmitate (Tween-40), PEG-20 sorbitan mono stearate (Tween-60), PEG-8 sorbitan mono stearate, PEG-80 sorbitan mono stearate, PEG-20 sorbitan tristearate (Tween-65), PEG-60 sorbitan tetra stearate, PEG-5 sorbitan monooleate (Tween-81), PEG-6 sorbitan monooleate, PEG-20 sorbitan mono oleate (Tween-80), PEG-40 sorbitan oleate, PEG-20 sorbitan trioleate (Tween-85), PEG-30 sorbitan tetraoleate, PEG-40 sorbitan tetraoleate, PEG-20 sorbitan mono-isostearate (Tween-120) and PEG sorbitol hexaoleate.

Suitable examples of polyethylene glycol alkyl ethers include, but are not limited to, PEG-10 oleyl ether oleth-10 (Volpo 10, Brij 96/97), PEG-20 oleyl ether oleth-20 (Volpo 20, Brij 98/99), PEG-9 lauryl ether, PEG-23 lauryl ether laureth-23, PEG-10 cetyl ether, PEG-20 cetyl ether, PEG-10 stearyl ether, PEG-20 stearyl ether and PEG-100 stearyl ether.

Suitable examples of sugar ethers include, but are not limited to, sucrose distearate/mono stearate (Sucrose ester, CRODESTA® F-10), sucrose monostearate, sucrose monopalmitate and sucrose monolaurate.

Suitable examples of polyethylene glycol alkyl phenols include, but are not limited to, PEG-10-100 nonyl phenol and PEG-15-100 octyl phenol.

Suitable examples of polyoxyethylene-polyoxypropylene block copolymers include, but are not limited to, Poloxamer 108, Poloxamer 188, Poloxamer 212, Poloxamer 215, Poloxamer 217, Poloxamer 231, Poloxamer 234, Poloxamer 235, Poloxamer 237, Poloxamer 238, Poloxamer 282, Poloxamer 284, Poloxamer 288, Poloxamer 333, Poloxamer 334, Poloxamer 335, Poloxamer 338, Poloxamer 401, Poloxamer 402, Poloxamer 403 and Poloxamer 407.

Hydrophobic surfactants can be reaction mixtures of polyols and fatty acids, glycerides, vegetable oils, hydrogenated vegetable oils and sterols. Hydrophobic surfactants may be selected from a group of: polyethoxylated fatty acids, PEG-fatty acid diesters, alcohol-oil transesterification products, polyglycerized fatty acids, propylene glycol fatty acid esters, mixtures of propylene glycol esters-glycerol esters, mono- and diglycerides, sterol and sterol derivatives, polyethylene glycol sorbitan fatty acid esters, polyethylene glycol alkyl ethers, sugar ethers, polyoxyethylene-polyoxypropylene block copolymers, sorbitan fatty acid esters, lower alcohol fatty acid esters surfactants and combinations comprising one or more of the foregoing material.

Suitable examples of polyethoxylated fatty acids include, but are not limited to, PEG-1 stearate, PEG-2 stearate, PEG-2 oleate, PEG-4 laurate, PEG 200, PEG-4 oleate, PEG-4 stearate, PEG-5 stearate, PEG-5 oleate, PEG-6 oleate and PEG-6 stearate.

Suitable examples of PEG-fatty acid diesters include, but are not limited to, PEG-4 dilaurate, PEG-4 dioleate, PEG-4 distearate, PEG-6 dilaurate, PEG-6 dioleat, PEG-6 distearate and PEG-8 dioleate.

Suitable examples of alcohol-oil transesterification products include, but are not limited to, PEG-3 castor oil, PEG-5, 9, and 16 castor oil, PEG-5 hydrogenated castor oil, PEG-7 hydrogenated castor oil, PEG-10 hydrogenated castor oil, PEG-6 corn oil (LABRAFIL®M 2125 CS), PEG-6 almond oil (LABRAFIL®M 1966 CS), PEG-6 apricot kernel oil (LABRAFIL®M 1944 CS), PEG-6 olive oil (LABRAFIL®M 1980 CS), PEG-6 peanut oil (LABRAFIL®M 1969 CS), PEG-6 hydrogenated palm (LABRAFIL®M 2130 BS), PEG-6 palm kernel oil (LABRAFIL®M 2130 CS), PEG-6 triolein (LABRAFIL®M 2735 CS), PEG-8 corn oil (LABRAFIL® WL 2609 BS), PEG-20 corn glycerides (CROVOL M40), PEG-20 almond glycerides (CROVOL A40), PEG-4 caprylic/capric triglyceride (LABRAFAC® Hydro), mono, di, tri, tetra esters of vegetable oils and sorbitol, pentaerythrityl tetraisostearate, pentaerythrityl distearate, pentaerythrityl tetraoleate, pentaerythrityl tetrastearate, pentaerythrityl tetracaprylate/tetracaprate and pentaerythrityl tetraoctanoate.

Suitable examples of polyglycerized fatty acids include, but are not limited to, polyglyceryl-2 stearate (Nikkol DGMS), polyglyceryl-2 oleate (Nikkol DGMO), polyglyceryl-2 isostearate (CAPROL®3GO), polyglyceryl-3 oleate, polyglyceryl-4 oleate, polyglyceryl-6 oleate, polyglyceryl-6 ricinoleate, polyglyceryl-6 pentaoleate, polyglyceryl-3 dioleate (CREMOPHORE® GO32), polyglyceryl-3 distearate (CREMOPHORE® GS32), polyglyceryl-4 pentaoleate, polyglyceryl-6 dioleate, polyglyceryl-2 dioleate, polyglyceryl-10 trioleate, polyglyceryl-10 pentaoleate, polyglyceryl-10 septaoleate, polyglyceryl-10 tetraoleate, polyglyceryl-10 decaisostearate, polyglyceryl-101 decaoleate and polyglyceryl polyricinoleate.

Suitable examples of propylene glycol fatty acid esters include, but are not limited to, propylene glycol mono caprylate (CAPRYOL® 90), propylene glycol mono-laurate (LAUROGLYCOL™ 90), propylene glycol oleate, propylene glycol myristate, propylene glycol mono stearate, propylene glycol hydroxy stearate, propylene glycol ricinoleate, propylene glycol isostearate, propylene glycol mono oleate (MYVEROL™ P-O6), propylene glycol dicaprylate/dicaprate (CAPTEX®200, MIGLYOL®840, NEOBEE®M-20), propylene glycol dioctanoate (CAPTEX®800), propylene glycol caprylate/caprate (LABRAFAC® PG), propylene glycol dilaurate, propylene glycol distearate, propylene glycol dicaprylate and propylene glycol dicaprate.

Suitable examples of mixtures of propylene glycol esters-glycerol esters include oleic (ATMOS 300, ARLACEL 186 (ICI)) and stearic (ATMOS 150).

Suitable examples of mono- and diglycerides include, but are not limited to, monopalmitolein (C16:1), monoelaidin (C18:1), monocaproin (C6), monocaprylin, monocaprin, monolaurin, glyceryl monomyristate (C14), glyceryl monooleate (C18:1), glyceryl monooleate, glycerol monooleate/linoleate, glycerol monolinoleate (Maisine), glyceryl ricinoleate, glyceryl monolaurate, glycerol monopalmitate, glycerol monostearate (CAMPUL®GMS), glyceryl mono-, dioleate, glyceryl palmitic/stearic, glyceryl acetate, glyceryl laurate (IMWITOR®312), glyceryl citrate/lactate/oleate/linoleate (IMWITOR®375), glyceryl caprylate (IMWITOR®308), glyceryl caprylate/caprate, caprylic acid mono diglycerides, caprylic/capric glycerides, mono- and diacetylated monoglycerides (MYVACET®9-45), glyceryl monostearate (ARLACEL® 129), lactic acid esters of mono diglycerides, dicaproin (C6), dicaprin (C10), dioctanoin (C8), dimyristin (C14), dipalmitin (C16), distearin, glyceryl diluarate (C12), glyceryl dioleate, glycerol esters of fatty acids (GELUCIRE® 39/01), dipalmitolein (C16:1), 1,2 and 1,3-diolein (C18:1), dielaidin (C18:1) and dilinolein (C18:2).

Suitable examples of sterol and sterol derivatives include, but are not limited to, cholesterol, sitosterol, lanosterol, phytosterol, PEG-5 soya sterol, PEG-10 soya sterol and PEG-20 soya sterol.

Suitable examples of polyethylene glycol sorbitan fatty acid esters include, but are not limited to, PEG-4 sorbitan mono-stearate (Tween-61), PEG-6 sorbitan tetrastearate (Nikkol GS-6), PEG-6 sorbitan tetraoleate, PEG-6 sorbitol hexastearate.

Suitable examples of polyethylene glycol alkyl ethers include, but are not limited to, PEG-2 oleyl ether oleth-2 (Brij 92/93), PEG-3 oleyl ether oleth-3 (Volpo 3), PEG-5 oleyl ether oleth-5 (Volpo 5), PEG-4 lauryl ether laureth-4 (Brij 30), PEG-2 cetyl ether and PEG-2 stearyl ether.

Examples of sugar ethers include, but are not limited to, sucrose distearate, sucrose dipalmitate.

Suitable examples of polyoxyethylene-polyoxypropylene block copolymers include, but are not limited to, Poloxamer 105, Poloxamer 122, Poloxamer 123, Poloxamer 124, Poloxamer 181, Poloxamer 182, Poloxamer 183, Poloxamer 184, Poloxamer 185 and Poloxamer 331.

Suitable examples of sorbitan fatty acid esters include, but are not limited to, sorbitan monolaurate (Span-20 or Arlacel 20), sorbitan monopalmitate (Span-40), sorbitan monooleate (Span-80), sorbitan monostearate (Span-60), sorbitan trioleate (Span-85), sorbitan sesquioleate (Arlacel-C), sorbitan tristearate (Span-65), sorbitan monoisostearate and sorbitan sesquistearate.

Suitable examples of lower alcohol fatty acid esters surfactants include, but are not limited to, ethyl oleate (CRODAMOL™ EO), isopropyl myristate, isopropyl palmitate, ethyl linoleate and isopropyl linoleate.

The compositions of the present application optionally include one or more pharmaceutically acceptable carrier materials. The term “carrier” refers to a compound that transports the medicament across the biological membrane or within a biological fluid. Carrier material can also facilitate solubilization of CETP inhibitors and helps in self-emulsification. Further it can increase the fraction of CETP inhibitors transported via the intestinal lymphatic system, thereby increasing the absorption from the GI tract.

Carrier material may be of triglycerides or digestible oil. The term “triglyceride” as used herein means glycerol triesters. The term “digestible oil” refers to any oil which is capable of undergoing deesterification in the presence of pancreatic lipase in vivo under normal physiological conditions. The triglyceride/digestible oil not only function as providing a base carrier for the drug, but also serve as an in vivo source of lipolytic products whereby the in vivo absorption of the CETP inhibitors is enhanced.

Suitable examples of triglycerides/digestible oils include, but are not limited to, aceituno oil, almond oil, araehis oil, babassu oil, blackcurrant seed oil, borage oil, buffalo ground oil, candlenut oil, canola oil, castor oil, chinese vegetable tallow oil, cocoa butter coconut oil, coffee seed oil, corn oil, cottonseed oil, crambe oil, cuphea species oil, evening primrose oil, grapeseed oil, groundnut oil, hemp seed oil, illipe butter, kapok seed oil, linseed oil, menhaden oil, mowrah butter mustard seed oil, oiticica oil, olive oil, palm oil, palm kernel oil, peanut oil, poppy seed oil, rapeseed oil, rice bran oil, safflower oil, sal fat sesame oil, shark liver oil, shea nut oil soybean oil, stillingia oil, sunflower oil, tall oil, tea sead oil, tobacco seed oil, tung oil (china wood oil), ucuhuba vernonia oil, wheat germ oil, hydrogenated castor oil (castor wax), hydrogenated coconut oil, hydrogenated cottonseed oil, hydrogenated palm oil, hydrogenated soybean oil, hydrogenated vegetable oil, hydrogenated cottonseed and castor oil, partially hydrogenated soybean oil, partially soy and cottonseed oil, glyceryl tributyrate, glyceryl tricaproate, glyceryl tricaprylate, glyceryl tricaprate (CAPTEX® 1000), glyceryl trundecanoate (CAPTEX® 8227), glyceryl trilaurate, glyceryl trimyristate, glyceryl tripalmitate, glyceryl tristearate, glyceryl triarcidate, glyceryl trimyristoleate, glyceryl tripalmitoleate, glyceryl trioleate, glyceryl trilinoleate, glyceryl trilinolenate, glyceryl tricaprylate/caprate (CAPTEX® 300, CAPTEX® 355, MIGLYOL® 810, MIGLYOL® 812), glyceryl tricaprylate/caprate/laurate (CAPTEX® 350), glyceryl tricaprylate/caprate/linoleate (CAPTEX® 810, MIGLYOL® 818), glyceryl tricaprylate/caprate/stearate, glyceryl tricaprylate/laurate/stearate, glyceryl 1,2-caprylate-3-linoleate, glyceryl 1,2-caprate-3-stearate, glyceryl 1,2-laurate-3 myristate, glyceryl 1,2-myristate-3-laurate, glyceryl 1,3-palmitate-2-butyrate, glyceryl 1,3-stearate-2-caprate, glyceryl 1,2-linoleate-3-caprylate, polyglycolized glycerides (GELUCIRE® 44/14, GELUCIRE® 50/13 and GELUCIRE® 53/10), linoleic glycerides (MAISINE™ 35-1), and caprylic/capric glycerides (IMWITOR® 742), fractionated triglycerides, modified triglycerides, synthetic triglycerides, and mixtures comprising one or more of the foregoing material.

The amount of carrier material that can be included in compositions is not particularly limited. Of course, when such compositions are ultimately administered to a patient, the amount of a given carrier material is limited to a bioacceptable amount, which is readily determined by one of skill in the art. Thus, if present, the carrier material can be present in an amount of from about 1% to about 100%, from about 5% to about 75%, from about 10% to about 50% of weight, based on the amount of surfactant.

The compositions of the present application may optionally include one or more additional compounds to enhance the solubility of the CETP inhibitors or the triglycerides in the composition. Such compounds are referred to “solubilizers” or “solvents”.

Suitable examples of solubilizers/solvents may be of those known and employed in the art, that including, but are not limited to, alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (GLYCOFUROL®) or methoxy PEG; amides, such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide, and polyvinylpyrrolidone; esters, such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, F-caprolactone and isomers thereof, δ-valerolactone and isomers thereof, β-butyrolactone and isomers thereof; and other solubilizers known in the art, such as dimethyl acetamide, dimethyl isosorbide (ARLASOLVE® DMI), N-methyl pyrrolidones (PHARMNASOLVE®), monooctanoin, diethylene glycol monoethyl ether (TRANSCUTOL®), and water. Mixtures of solubilizers are also within the scope of the present application.

The amount of solubilizer that can be included in compositions is not particularly limited. Of course, when such compositions are ultimately administered to a patient, the amount of a given solubilizer is limited to a bioacceptable amount, which is readily determined by one of skill in the art. Thus, if present, the solubilizer can be present in an amount of from about 1% to about 100%, from about 5% to about 75%, from about 10% to about 50% of weight, based on the amount of surfactant.

The compositions of the present application may optionally contain suitable amounts of pharmaceutical acceptable excipients that are conventionally used in the compositions and these excipients are well known in the art.

Such excipients may include antioxidants such as tocopherol, tocopherol acetate, ascorbyl palmitate, ascorbic acid, butylhydroxytoluene, butylhydroxyanisole and propyl gallate; pH stabilisers such as citric acid, tartaric acid, fumaric acid, acetic acid, glycine, arginine, lysine and potassium hydrogen phosphate; thickeners/suspending agents such as hydrogenated vegetable oils, beeswax, colloidal silicon dioxide, gums, celluloses, silicates and bentonite; fillers such as corn starch, lactose, white sugar, sucrose, sugar compressible, sugar confectioners, glucose, sorbitol, calcium carbonate, calcium dihydrogen phosphate dihydrate, calcium phosphate-dibasic, calcium phosphate-tribasic, calcium sulfate, microcrystalline cellulose, silicified microcrystalline cellulose, cellulose powdered, dextrates, dextrins, dextrose, fructose, kaolin, lactitol, mannitol, starch and starch pregelatinized; binders such as povidones, various starches known in the art, including corn starch, pregelatinized starch, microcrystalline celluloses (MCC), silicified MCC (e.g., PROSOLV® HD 90), microfine celluloses, lactose, calcium carbonate, calcium sulfate, sugar, mannitol, sorbitol, dextrates, dextrin, maltodextrin, dextrose, dibasic calcium phosphate dihydrate, tribasic calcium phosphate, magnesium carbonate, magnesium oxide, stearic acid, gums and hydroxypropyl methylcellulose or hypromelloses (e.g., KLUCEL™EF, METHOCEL™ E5 premium); lubricants and glidants such as colloidal silicon dioxide, such as AEROSIL® 200, talc, stearic acid, magnesium stearate, calcium stearate, solid polyethylene glycols, sodium stearyl fumarate and silica gel; disintegrants such as cross-linked polyvinyl pyrrolidone, corn starch, potato starch, maize starch and modified starches, agar-agar, calcium carbonate, sodium carbonate, alginic acids, cross-carmellose sodium, sodium starch glycolate and microcrystalline cellulose; flavouring agents such as cherry, lemon and aniseed flavours; sweeteners such as aspartame, saccharin and cyclamates; etc.

The compositions of the present application may be prepared as oral dosage forms and in the form of liquid, solid or semi-solid at room temperatures.

The compositions may be prepared by uniformly and thoroughly mixing the CETP inhibitor, the surfactant, optionally the carrier material and solubilizer together at room temperature or at slightly elevated temperature, such as temperatures up to about 60° C. until a clear solution is obtained, and then cooled to room temperature. The other pharmaceutically acceptable excipients indicated hereinabove are then thoroughly admixed therewith. In this preparation, the CETP inhibitor remains in solution and does not crystallize or precipitate out.

In a typical procedure for the preparation, the carrier material is weighed out into a suitable stainless steel vessel and the surfactant is then weighed and added to the container. Mixing of the two liquids is effected by use of a homogenising mixer or other high shear device. If the material(s) is solid at room temperature, sufficient heat is applied to ensure fluidity without chemical decomposition. The solubilizer, if required is added last with mixing. The CETP inhibitor is then weighed and added to the combined liquids and mixing continued until either a homogenous solution or suspension is prepared. The composition is then normally de-aerated before encapsulation in either soft or hard capsules. In some instances the fill composition may be held at elevated temperature using a suitable jacketed vessel to aid processing.

In one aspect, the pharmaceutical composition of the present application forms a microemulsion when brought into contact with water or an aqueous medium. The microemulsion thus formed is thermodynamically stable when it comes into contact with the water or aqueous medium, as in the G.I. fluids of mammals. However, until the composition comes in contact with an aqueous medium, it is not a microemulsion; instead, when the various components are mixed, it forms what in the art is known as a preconcentrate of an emulsion i.e., microemulsion pre-concentrate, i.e., a system capable of forming microemulsion respectively, on contact with water or aqueous system.

In one embodiment, the pharmaceutical composition in the form of emulsion can be adsorbed/absorbed onto adsorbents/absorbents (these two terms are collectively referred to as “adsorbent” or “adsorbents”). Adsorbents should be nontoxic and should include fine particles. Suitable adsorbents include, but are not limited to, clays such as kaolin, bentonite, hectorite, colloidal magnesium aluminum silicate, silicon dioxide (CAB-O-SILO or AEROSIL®), magnesium trisilicate, microcrystalline cellulose, aluminum hydroxide, magnesium hydroxide, magnesium oxide or talc. This adsorbed emulsion provides free-flowing and compressible powder. The powder can be mixed with tableting excipients know in the art, to compress into tablets.

In another embodiment, the pharmaceutical composition of the present application is presented in a form appropriate or adapted for oral administration, in particular, in oral unit dosage form, e.g., in the form of tablets, capsules, drink solutions or dry powder for reconstitution; or a sohxlet form prepared by standard techniques known in the art, such as by spray coating on deposition. Especially suitable unit dosage forms for oral administration include encapsulated forms, e.g., soft or hard gelatin encapsulated forms.

When the composition of the present invention is prepared in the form of a soft or hard capsule, the composition may be encapsulated in a gelatin shell which contains any conventional plasticizer. The plasticizer that can be included in the gelatin capsule shell, include but not limited to, glycerine, sorbitol, hexanetriol propylene carbonate, hexane glycol, sorbitans, tetrahydrofuryl alcohol ether, diethylene glycol monoethyl ether, 1,3-trimethyl-2-imidazolidone, dimethylisosorbide and mixtures thereof.

In further aspect compositions of the present invention may be used to treat any condition which is subject to treatment by administering a CETP inhibitor.

One aspect of this invention is directed to a method for treating atherosclerosis, peripheral vascular disease, dyslipidemia, hyperbetalipoproteinemia, hypoalphalipoproteinemia, hypercholesterolemia, hypertriglyceridemia, familial hypercholesterolemia, cardiovascular disorders, angina, ischemia, cardiac ischemia, stroke, myocardial infarction, reperfusion injury, angioplastic restenosis, hypertension, vascular complications of diabetes, obesity or endotoxemia in a mammal (including a human being) by administering to a mammal in need of such treatment an atherosclerotic treating amount of a composition of the present invention.

In another aspect of this application, the pharmaceutical compositions as disclosed herein are used in the treatment of various aforementioned diseases.

The present invention is illustrated below by reference to the following examples. However, one skilled in the art will appreciate that the specific methods and results discussed are merely illustrative of the invention, and not to be construed as limiting the invention.

### EXAMPLES

In the following Examples 1-32, various compositions in accordance with the present application were prepared comprising 3-(((3,5-bis(trifluoromethyl)benzyl)(2-methyl-2H-tetrazol-5-yl)amino)methyl)-N,N-bis(cyclopropylmethyl)-8-methylquinolin-2-amine as the CETP inhibitor.

### Examples 1-5

| | Percent w/w | | | | |
|---|---|---|---|---|---|
| Ingredients | Example 1 | Example 2 | Example 3 | Example 4 | Example 5 |
| 3-(((3,5- | 38.46 | 38.46 | 22.99 | 38.46 | 27.03 |
| bis(trifluoromethyl)benzyl)(2- | | | | | |
| methyl-2H-tetrazol-5- | | | | | |
| yl)amino)methyl)-N,N- | | | | | |
| bis(cyclopropylmethyl)-8- | | | | | |
| methylquinolin-2-amine | | | | | |
| Glyceryl tricaprylate/caprate | 15.38 | 16.92 | 22.99 | 7.69 | 11.89 |
| (MIGYOL ® 812) | | | | | |
| Triacetin | 1.85 | 2.31 | 3.45 | 11.54 | 1.62 |
| PEG-35 castor oil | 33.23 | 31.54 | 37.70 | 31.54 | 44.32 |
| (CREMOPHOR ® EL) | | | | | |
| PEG-8 caprylic/capric | 11.08 | 10.77 | 12.87 | 10.77 | 15.14 |
| glycerides | | | | | |
| (LABRASOL ®) | | | | | |

### Process:

1. Specified quantities of MIGLYOL® 812, triacetin, CREMOPHOR® EL and LABRASOL® were weighed and transferred into a suitable glass vessel and heated to about 50-60° C. to form clear solution,
2. To the solution of step 1, specified quantity of 3-(((3,5-bis(trifluoromethyl)benzyl)(2-methyl-2H-tetrazol-5-yl)amino)methyl)-N,N-bis(cyclopropylmethyl)-8-methylquinolin-2-amine is added and mixed gently until the drug material dissolved,
3. The mixture of step 2 is vortexed to get clear solution and then cooled to room temperature, and
4. The composition of step 3 is filled into hard gelatin capsule.

### Examples 6-9

| | Percent w/w | | | |
|---|---|---|---|---|
| Ingredients | Example 6 | Example 7 | Example 8 | Example 9 |
| 3-(((3,5-bis | 38.46 | 38.46 | 12.50 | 14.29 |
| (trifluoromethyl)benzyl) | | | | |
| (2-methyl-2H-tetrazol-5- | | | | |
| yl)amino)methyl)-N,N- | | | | |
| bis(cyclopropylmethyl)-8- | | | | |
| methylquinolin-2-amine | | | | |
| Glyceryl tricaprylate/ | 19.23 | 7.69 | 37.50 | — |
| caprate | | | | |
| (MIGLYOL ® 812) | | | | |
| PEG-35 castor oil | 31.54 | 43.08 | 50.00 | 42.86 |
| (CREMOPHOR ® EL) | | | | |
| PEG-8 caprylic/capric | 10.77 | 10.77 | — | — |
| glycerides | | | | |
| (LABRASOL ®) | | | | |
| Propylene glycol | — | — | — | 42.86 |
| monocaprylate | | | | |
| (CAPRYOL ® 90) | | | | |

Process:
1. Specified quantities of excipients (MIGLYOL® 812/CREMOPHOR® EL/LABRASOL®/CAPRYOL® 90) were weighed and transferred into a suitable glass vessel and heated to about 50-60° C. to form clear solution,
2. To the solution of step 1, specified quantity of 3-(((3,5-bis(trifluoromethyl)benzyl)(2-methyl-2H-tetrazol-5-yl)amino)methyl)-N,N-bis(cyclopropylmethyl)-8-methylquinolin-2-amine is added and mixed gently until the drug material dissolved,
3. The mixture of step 2 is vortexed to get clear solution and then cooled to room temperature, and
4. The composition of step 3 is filled into hard gelatin capsule.

### Examples 10-13

| | Percent w/w | | | |
|---|---|---|---|---|
| | Example | Example | Example | Example |
| Ingredients | 10 | 11 | 12 | 13 |
| 3-(((3 ,5-bis(trifluoromethyl) | 12.92 | 10.79 | 16.56 | 17.86 |
| benzyl)(2-methyl-2H-tetrazol- | | | | |
| 5-yl)amino)methyl)- | | | | |
| N,N-bis(cyclopropylmethyl)-8- | | | | |
| methylquinolin-2-amine | | | | |
| Glyceryl tricaprylate/caprate | 5.68 | 4.75 | 7.28 | 7.86 |
| (MIGLYOL ® 812) | | | | |
| Triacetin | 0.78 | 0.65 | 0.99 | 1.07 |
| PEG-35 castor oil | 10.59 | 8.85 | 10.26 | 7.32 |
| (CREMOPHOR ® EL) | | | | |
| PEG-8 caprylic/capricglycerides | 3.62 | 3.02 | 3.48 | 2.50 |
| (LABRASOL ®) | | | | |
| Microcrystalline cellulose | 51.68 | 53.97 | 49.67 | 53.57 |
| (MCC 112) | | | | |
| Colloidal silicon dioxide | 6.46 | 10.79 | 10.93 | 9.82 |
| (AEROSIL ® 200) | | | | |
| Lactose monohydrate | 7.75 | 6.48 | 0.00 | 0.00 |
| (PHARMATOSE ® 200M) | | | | |
| Magnesium stearate | 0.32 | 0.54 | 0.83 | 0.89 |
| Total weight | 100 | 100 | 100 | 100 |

Process:
1. Specified quantities of MIGLYOL® 812, triacetin, CREMOPHOR® EL and LABRASOL® were weighed and transferred into a suitable glass vessel and heated to about 50-60° C. to form clear solution,
2. To the solution of step 1 specified quantity of 3-(((3,5-bis(trifluoromethyl)benzyl)(2-methyl-2H-tetrazol-5-yl)amino)methyl)-N,N-bis(cyclopropylmethyl)-8-methylquinolin-2-amine is added and mixed gently until the drug material dissolved,
3. The mixture of step 2 is vortexed to get clear solution and then cooled to room temperature,
4. The solution of step 3 is adsorbed onto microcrystalline cellulose and dried,
5. The mixture of step 4 is mixed with colloidal silicon dioxide, lactose monohydrate and magnesium stearate, and
6. The mixture of step 5 is either compressed into tablets or filled in to capsules.

### Examples 14-19

| | Percent w/w | | | | | |
|---|---|---|---|---|---|---|
| | Example | Example | Example | Example | Example | Example |
| Ingredients | 14 | 15 | 16 | 17 | 18 | 19 |
| 3-(((3,5-bis(trifluoromethyl)benzyl)(2- | 33.90 | 31.25 | 28.99 | 33.90 | 31.25 | 28.99 |
| methyl-2H-tetrazol-5-yl)amino)methyl)- | | | | | | |
| N,N-bis(cyclopropylmethyl)-8- | | | | | | |
| methylquinolin-2-amine | | | | | | |
| Glyceryl tricaprylate/caprate | 40.68 | 37.50 | 34.78 | 40.68 | 37.50 | 34.78 |
| (MIGLYOL ®810N) | | | | | | |
| PEG-20 sorbitan monolaurate | 25.42 | 31.25 | 36.23 | — | — | — |
| (TWEEN ®80) | | | | | | |
| PEG-35 castor oil | — | — | — | 25.42 | 31.25 | 36.23 |
| (CREMOPHOR ® EL) | | | | | | |

Process:
1. Specified quantity of 3-(((3,5-bis(trifluoromethyl)benzyl)(2-methyl-2H-tetrazol-5-yl)amino)methyl)-N,N-bis(cyclopropylmethyl)-8-methylquinolin-2-amine is solubilized in specified quantity of MIGLY®C810N at 37° C.,
2. To the solution of step 1 specified quantity of TWEEN® 80/CREMOPHOR® EL is added and mixed gently,
3. The mixture of step 2 is vortexed to get clear solution and then cooled to room temperature, and
4. The composition of step 3 is filled into hard gelatin capsule.

### Examples 20-27

| | Percent w/w | | | |
|---|---|---|---|---|
| | Example | Example | Example | Example |
| Ingredients | 20 | 21 | 22 | 23 |
| 3-(((3,5- | 10.00 | 10.00 | 10.00 | 10.00 |
| bis(trifluoromethyl)benzyl)(2- | | | | |
| methyl-2H-tetrazol-5- | | | | |
| yl)amino)methyl)-N,N- | | | | |
| bis(cyclopropylmethyl)-8- | | | | |
| methylquinolin-2-amine | | | | |
| Glyceryl tricaprylate/caprate | 40.00 | — | — | — |
| (MIGLYOL ® 810) | | | | |
| Glyceryl tricaprylate/caprate | — | 40.00 | — | — |
| (MIGLYOL ® 812) | | | | |
| Propylene glycol dicaprylate/ | — | — | 40.00 | — |
| dicaprate | | | | |
| (LABRAFAC ® 1349) | | | | |
| Propylene glycol monocaprylate | — | — | — | 40.00 |
| (CAPRYOL ® 90) | | | | |
| PEG-35 castor oil | 40.00 | 40.00 | 40.00 | 40.00 |
| (CREMOPHOR ® EL) | | | | |
| PEG-8 caprylic/capricglycerides | 10.00 | 10.00 | 10.00 | 10.00 |
| (LABRASOL ®) | | | | |

Process:
1. Specified quantities of excipients (MIGLYOL® 810/MIGLYOL® 812/LABRAFAC® 1349/CAPRYOL® 90, CREMOPHOR® EL and LABRASOL®) were weighed and transferred into a suitable glass vessel and heated to about 50-60° C. to form clear solution,
2. To the solution of step 1, specified quantity of 3-(((3,5-bis(trifluoromethyl)benzyl)(2-methyl-2H-tetrazol-5-yl)amino)methyl)-N,N-bis(cyclopropylmethyl)-8-methylquinolin-2-amine is added and mixed gently until the drug material dissolved,
3. The mixture of step 2 is vortexed to get clear solution and then cooled to room temperature, and
4. The composition of step 3 is filled into hard gelatin capsule.

### Examples 20-27

| | Percent w/w | | | |
|---|---|---|---|---|
| | Example | Example | Example | Example |
| Ingredients | 24 | 25 | 26 | 27 |
| 3-(((3,5- | 10.00 | 10.00 | 10.00 | 10.00 |
| bis(trifluoromethyl)benzyl)(2- | | | | |
| methyl-2H-tetrazol-5 - | | | | |
| yl)amino)methyl)-N,N- | | | | |
| bis(cyclopropylmethyl)-8- | | | | |
| methylquinolin-2-amine | | | | |
| Glyceryl tricaprylate/caprate | 30.00 | — | — | — |
| (MIGLYOL ® 810) | | | | |
| Glyceryl tricaprylate/caprate | — | 30.00 | — | — |
| (MIGLYOL ® 812) | | | | |
| Propylene | — | — | 30.00 | — |
| glycoldicaprylate/dicaprate | | | | |
| (LABRAFAC ® 1349) | | | | |
| Propylene glycol monocaprylate | — | — | — | 30.00 |
| (CAPRYOL ®90) | | | | |
| PEG-35 castor oil | 60.00 | 60.00 | 60.00 | 60.00 |
| (CREMOPHOR ® EL) | | | | |

Process:
1. Specified quantities of excipients (MIGLYOL® 810/MIGLYOL® 812/LABRAFAC® 1349/CAPRYOL® 90 and CREMOPHOR® EL) were weighed and transferred into a suitable glass vessel and heated to about 50-60° C. to form clear solution,
2. To the solution of step 1, specified quantity of 3-(((3,5-bis(trifluoromethyl)benzyl)(2-methyl-2H-tetrazol-5-yl)amino)methyl)-N,N-bis(cyclopropylmethyl)-8-methylquinolin-2-amine added and mixed gently until the drug material dissolved,
3. The mixture of step 2 is vortexed to get clear solution and then cooled to room temperature, and
4. The composition of step 3 is filled into hard gelatin capsule.

### Examples 28-29

| | Percent w/w | |
|---|---|---|
| Ingredients | Examples 28 | Examples 29 |
| 3-(((3,5-bis(trifluoromethyl)benzyl)(2- | 14.29 | 12.50 |
| methyl-2H-tetrazol-5-y1)amino)methyl)- | | |
| N,N-bis(cyclopropylmethyl)-8- | | |
| methylquinolin-2-amine | | |
| Glyceryl tricaprylate/caprate (MIGLYOL ® | 42.86 | 37.50 |
| 812) | | |
| PEG-35 castor oil | 42.86 | 50.00 |
| (CREMOPHOR ®EL) | | |

Process:
1. Specified quantities of MIGLYOL® 812 and CREMOPHOR® EL were weighed and transferred into a suitable glass vessel and heated to about 50-60° C. to form clear solution,
2. To the solution of step 1, specified quantity of 3-(((3,5-bis(trifluoromethyl)benzyl)(2-methyl-2H-tetrazol-5-yl)amino)methyl)-N,N-bis(cyclopropylmethyl)-8-methylquinolin-2-amine is added and mixed gently until the drug material dissolved,
3. The mixture of step 2 is vortexed to get clear solution and then cooled to room temperature, and
4. The composition of step 3 is filled into hard gelatin capsule.

### Examples 30-31

| | Percent w/w | |
|---|---|---|
| | Examples | Examples |
| Ingredients | 30 | 31 |
| 3-(((3,5-bis(trifluoromethyl)benzyl)(2-methyl-2H- | 14.29 | 12.50 |
| tetrazol-5-yl)amino)methyl)-N,N- | | |
| bis(cyclopropylmethyl)-8-methylquinolin-2-amine | | |
| Propylene glycol monocaprylate | 42.86 | 37.50 |
| (CAPRYOL ® 90) | | |
| PEG-35 castor oil | 42.86 | 50.00 |
| (CREMOPHOR ® EL) | | |
| Total weight | 100.00 | 100.00 |

Process:
1. Specified quantities of CAPRYOL® 90 and CREMOPHOR® EL were weighed and transferred into a suitable glass vessel and heated to about 50-60° C. to form clear solution,
2. To the solution of step 1, specified quantity of 3-(((3,5-bis(trifluoromethyl)benzyl)(2-methyl-2H-tetrazol-5-yl)amino)methyl)-N,N-bis(cyclopropylmethyl)-8-methylquinolin-2-amine is added and mixed gently until the drug material dissolved,
3. The mixture of step 2 is vortexed to get clear solution and then cooled to room temperature, and
4. The composition of step 3 is filled into hard gelatin capsule.

### Example 32

Examples 29-30 were subjected to dissolution test in 900 mL of 0.01N HCl with 0.25% sodium lauryl sulfate (SLS) at 37° C. and 50 RPM with sinkers. Samples were withdrawn at designated time points and analyzed for drug release by UV absorption. The amount of drug released is shown in Table 1 below.

TABLE 1

| Time | Example 29 | Example 30 |
|---|---|---|
| 5 min | 9 | 74 |
| 10 min | 19 | 88 |
| 15 min | 28 | 93 |
| 30 min | 53 | 98 |
| 45 min | 74 | 99 |
| 60 min | 90 | 101 |

In one embodiment, various compositions in accordance with the present application can be prepared by substituting 1.3-(((3,5-bis(trifluoromethyl)benzyl)(2-methyl-2H-tetrazol-5-yl)amino)methyl)-N,N-bis(cyclopropylmethyl)-8-methylquinolin-2-amine, as described in Examples 1-31, with any one or more of the following compounds:

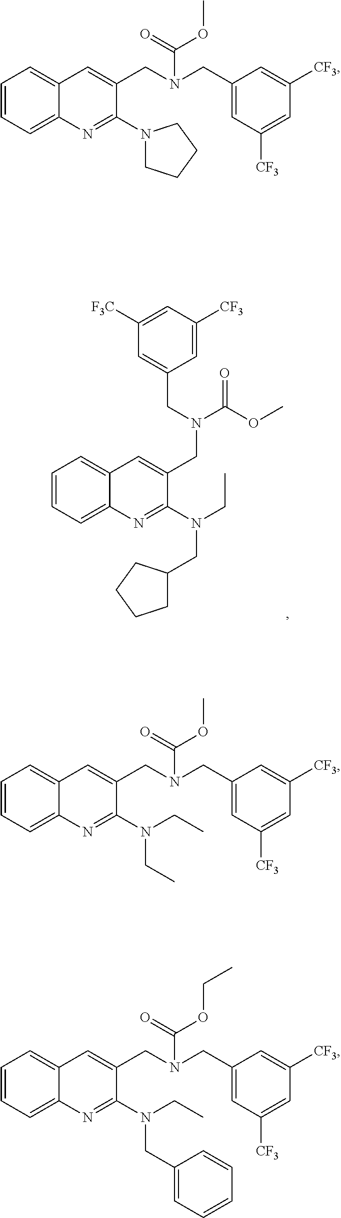 [SMILES: COC(=O)N(Cc1cc(C(F)(F)F)cc(C(F)(F)F)c1)Cc1cc2ccccc2nc1N1CCCC1 ; CCN(CC1CCCC1)c1nc2ccccc2cc1CN(Cc1cc(C(F)(F)F)cc(C(F)(F)F)c1)C(=O)OC ; CCN(CC)c1nc2ccccc2cc1CN(Cc1cc(C(F)(F)F)cc(C(F)(F)F)c1)C(=O)OC ; CCOC(=O)N(Cc1cc(C(F)(F)F)cc(C(F)(F)F)c1)Cc1cc2ccccc2nc1N(CC)Cc1ccccc1]

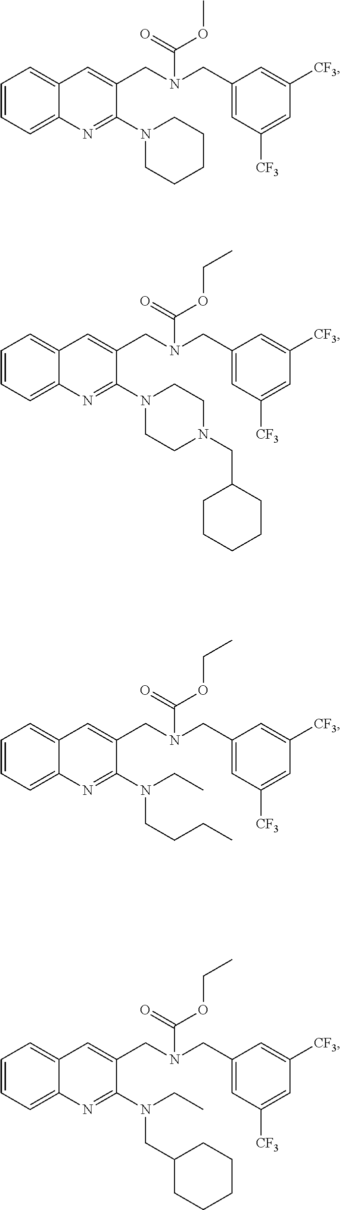 [SMILES: COC(=O)N(Cc1cc(C(F)(F)F)cc(C(F)(F)F)c1)Cc1cc2ccccc2nc1N1CCCCC1 ; CCOC(=O)N(Cc1cc(C(F)(F)F)cc(C(F)(F)F)c1)Cc1cc2ccccc2nc1N1CCN(CC2CCCCC2)CC1 ; CCCCN(CC)c1nc2ccccc2cc1CN(Cc1cc(C(F)(F)F)cc(C(F)(F)F)c1)C(=O)OCC ; CCOC(=O)N(Cc1cc(C(F)(F)F)cc(C(F)(F)F)c1)Cc1cc2ccccc2nc1N(CC)CC1CCCCC1]

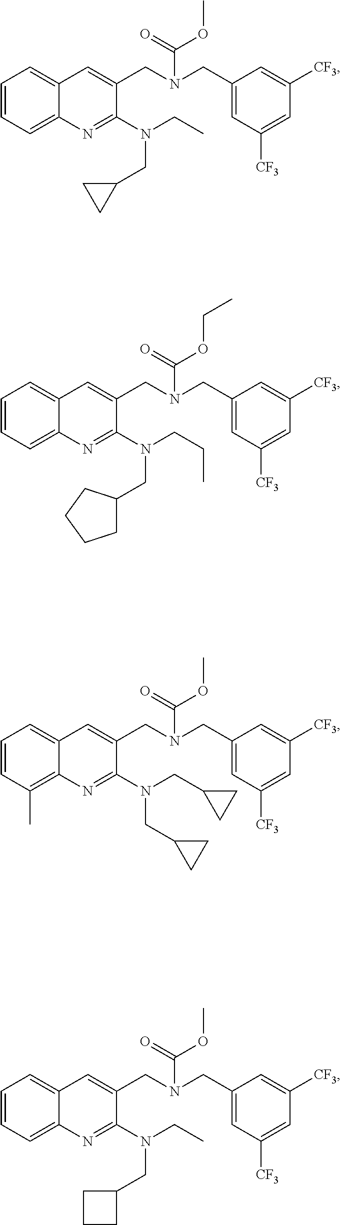 [SMILES: CCN(CC1CC1)c1nc2ccccc2cc1CN(Cc1cc(C(F)(F)F)cc(C(F)(F)F)c1)C(=O)OC ; CCCN(CC1CCCC1)c1nc2ccccc2cc1CN(Cc1cc(C(F)(F)F)cc(C(F)(F)F)c1)C(=O)OCC ; COC(=O)N(Cc1cc(C(F)(F)F)cc(C(F)(F)F)c1)Cc1cc2cccc(C)c2nc1N(CC1CC1)CC1CC1 ; CCN(CC1CCC1)c1nc2ccccc2cc1CN(Cc1cc(C(F)(F)F)cc(C(F)(F)F)c1)C(=O)OC]

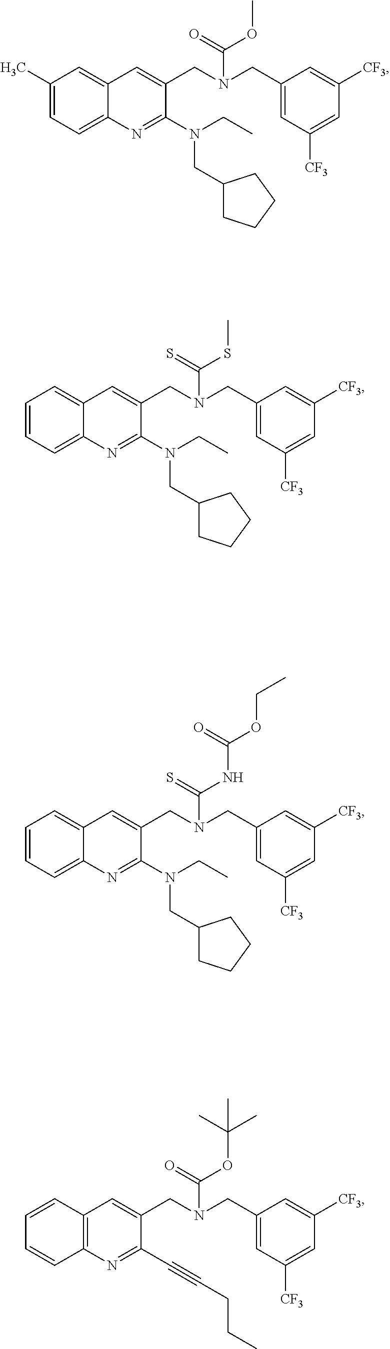 [SMILES: CCN(CC1CCCC1)c1nc2ccc(C)cc2cc1CN(Cc1cc(C(F)(F)F)cc(C(F)(F)F)c1)C(=O)OC ; CCN(CC1CCCC1)c1nc2ccccc2cc1CN(Cc1cc(C(F)(F)F)cc(C(F)(F)F)c1)C(=S)SC ; CCOC(=O)NC(=S)N(Cc1cc(C(F)(F)F)cc(C(F)(F)F)c1)Cc1cc2ccccc2nc1N(CC)CC1CCCC1 ; CCCC#Cc1nc2ccccc2cc1CN(Cc1cc(C(F)(F)F)cc(C(F)(F)F)c1)C(=O)OC(C)(C)C]

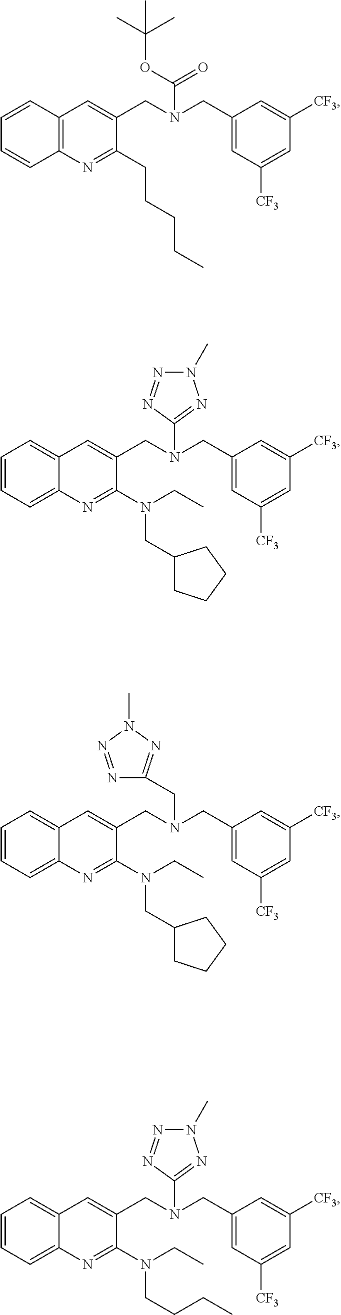 [SMILES: CCCCCc1nc2ccccc2cc1CN(Cc1cc(C(F)(F)F)cc(C(F)(F)F)c1)C(=O)OC(C)(C)C ; CCN(CC1CCCC1)c1nc2ccccc2cc1CN(Cc1cc(C(F)(F)F)cc(C(F)(F)F)c1)c1nnn(C)n1 ; CCN(CC1CCCC1)c1nc2ccccc2cc1CN(Cc1cc(C(F)(F)F)cc(C(F)(F)F)c1)Cc1nnn(C)n1 ; CCCCN(CC)c1nc2ccccc2cc1CN(Cc1cc(C(F)(F)F)cc(C(F)(F)F)c1)c1nnn(C)n1]

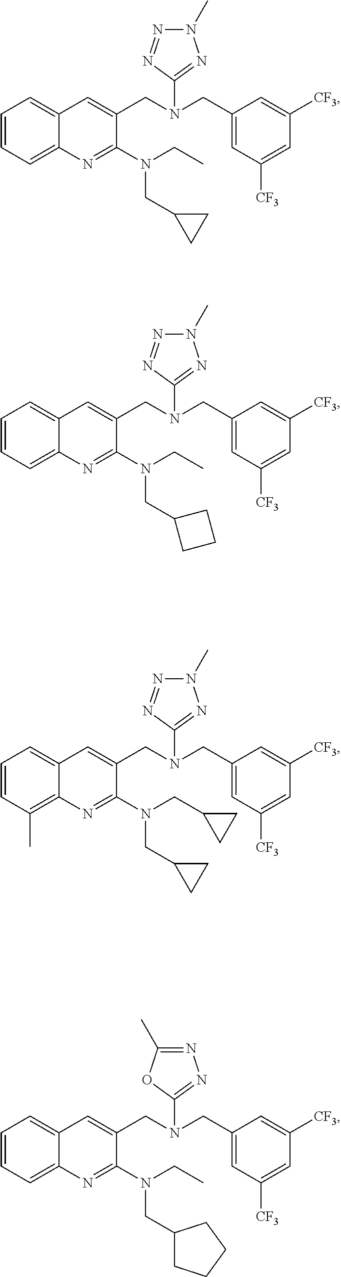 [SMILES: CCN(CC1CC1)c1nc2ccccc2cc1CN(Cc1cc(C(F)(F)F)cc(C(F)(F)F)c1)c1nnn(C)n1 ; CCN(CC1CCC1)c1nc2ccccc2cc1CN(Cc1cc(C(F)(F)F)cc(C(F)(F)F)c1)c1nnn(C)n1 ; Cc1cccc2cc(CN(Cc3cc(C(F)(F)F)cc(C(F)(F)F)c3)c3nnn(C)n3)c(N(CC3CC3)CC3CC3)nc12 ; CCN(CC1CCCC1)c1nc2ccccc2cc1CN(Cc1cc(C(F)(F)F)cc(C(F)(F)F)c1)c1nnc(C)o1]

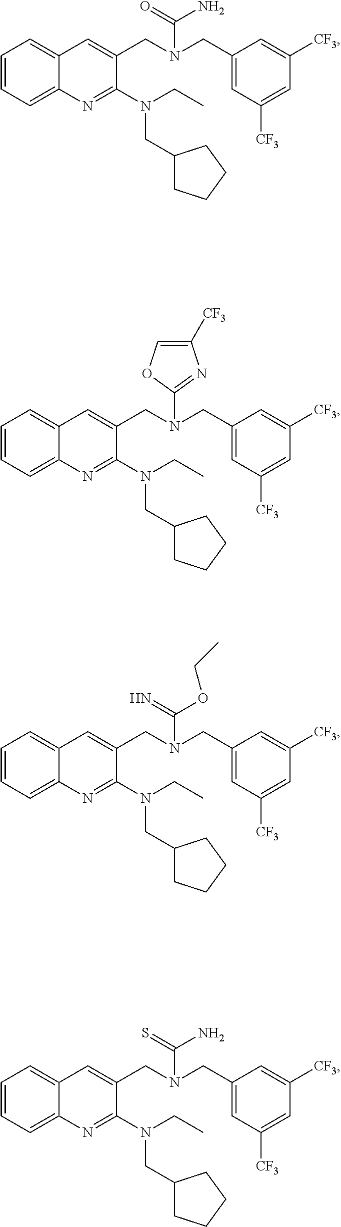 [SMILES: CCN(CC1CCCC1)c1nc2ccccc2cc1CN(Cc1cc(C(F)(F)F)cc(C(F)(F)F)c1)C(N)=O ; CCN(CC1CCCC1)c1nc2ccccc2cc1CN(Cc1cc(C(F)(F)F)cc(C(F)(F)F)c1)c1nc(C(F)(F)F)co1 ; CCOC(=N)N(Cc1cc(C(F)(F)F)cc(C(F)(F)F)c1)Cc1cc2ccccc2nc1N(CC)CC1CCCC1 ; CCN(CC1CCCC1)c1nc2ccccc2cc1CN(Cc1cc(C(F)(F)F)cc(C(F)(F)F)c1)C(N)=S]

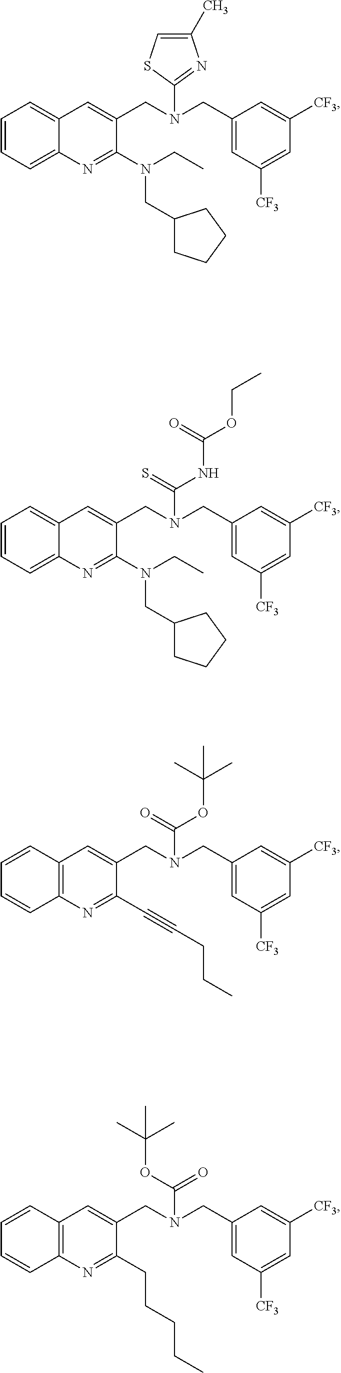 [SMILES: CCN(CC1CCCC1)c1nc2ccccc2cc1CN(Cc1cc(C(F)(F)F)cc(C(F)(F)F)c1)c1nc(C)cs1 ; CCOC(=O)NC(=S)N(Cc1cc(C(F)(F)F)cc(C(F)(F)F)c1)Cc1cc2ccccc2nc1N(CC)CC1CCCC1 ; CCCC#Cc1nc2ccccc2cc1CN(Cc1cc(C(F)(F)F)cc(C(F)(F)F)c1)C(=O)OC(C)(C)C ; CCCCCc1nc2ccccc2cc1CN(Cc1cc(C(F)(F)F)cc(C(F)(F)F)c1)C(=O)OC(C)(C)C]

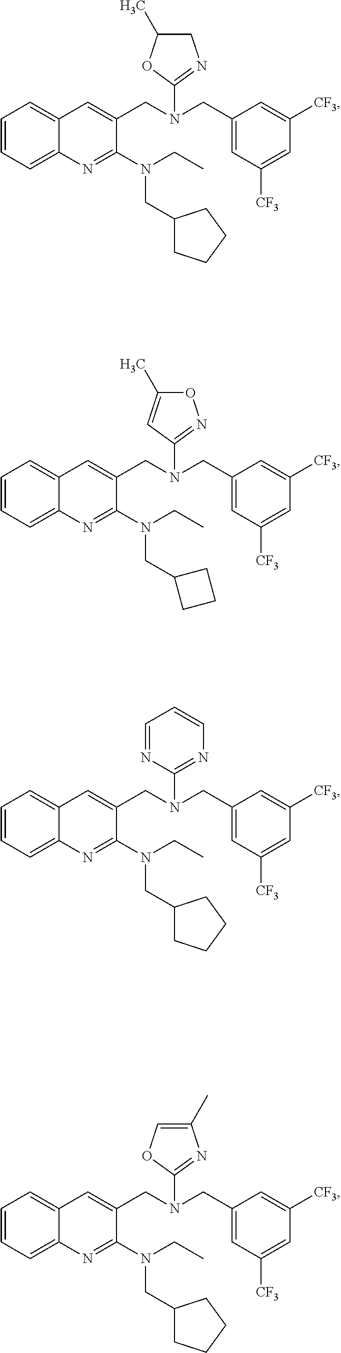 [SMILES: CCN(CC1CCCC1)c1nc2ccccc2cc1CN(Cc1cc(C(F)(F)F)cc(C(F)(F)F)c1)C1=NCC(C)O1 ; CCN(CC1CCC1)c1nc2ccccc2cc1CN(Cc1cc(C(F)(F)F)cc(C(F)(F)F)c1)c1cc(C)on1 ; CCN(CC1CCCC1)c1nc2ccccc2cc1CN(Cc1cc(C(F)(F)F)cc(C(F)(F)F)c1)c1ncccn1 ; CCN(CC1CCCC1)c1nc2ccccc2cc1CN(Cc1cc(C(F)(F)F)cc(C(F)(F)F)c1)c1nc(C)co1]

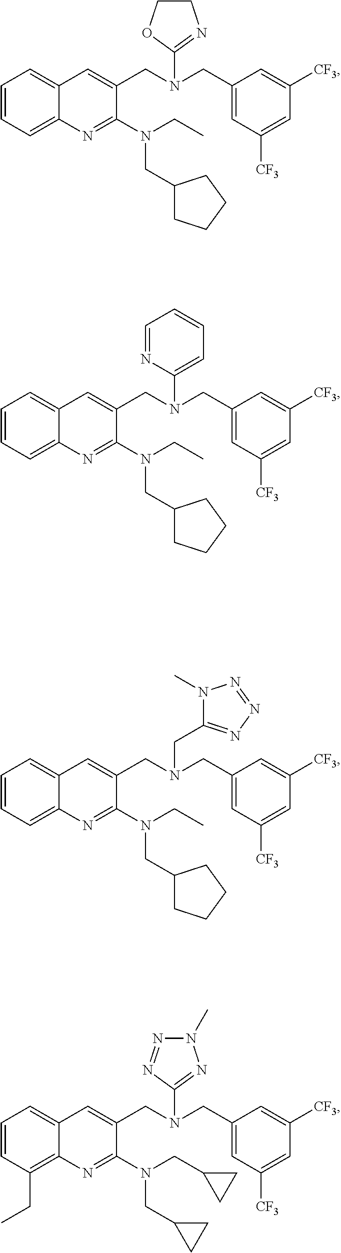 [SMILES: CCN(CC1CCCC1)c1nc2ccccc2cc1CN(Cc1cc(C(F)(F)F)cc(C(F)(F)F)c1)C1=NCCO1 ; CCN(CC1CCCC1)c1nc2ccccc2cc1CN(Cc1cc(C(F)(F)F)cc(C(F)(F)F)c1)c1ccccn1 ; CCN(CC1CCCC1)c1nc2ccccc2cc1CN(Cc1cc(C(F)(F)F)cc(C(F)(F)F)c1)Cc1nnnn1C ; CCc1cccc2cc(CN(Cc3cc(C(F)(F)F)cc(C(F)(F)F)c3)c3nnn(C)n3)c(N(CC3CC3)CC3CC3)nc12]

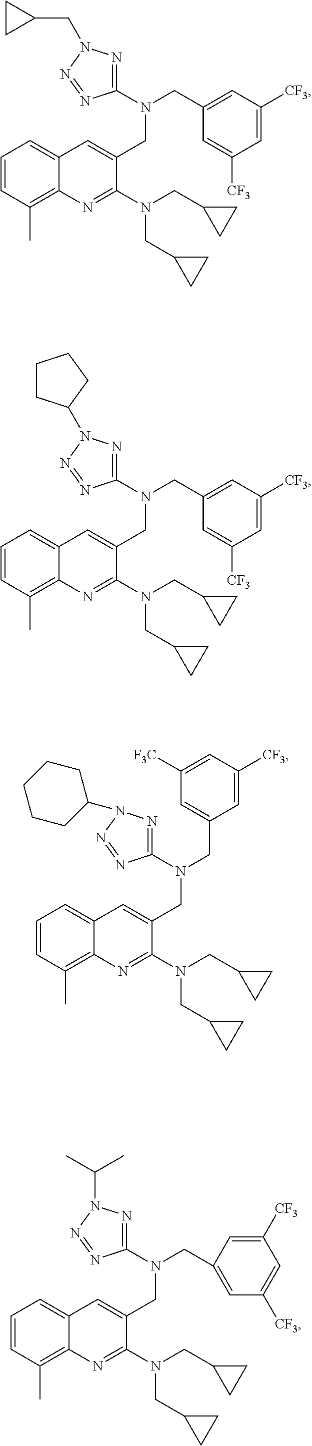 [SMILES: Cc1cccc2cc(CN(Cc3cc(C(F)(F)F)cc(C(F)(F)F)c3)c3nnn(CC4CC4)n3)c(N(CC3CC3)CC3CC3)nc12 ; Cc1cccc2cc(CN(Cc3cc(C(F)(F)F)cc(C(F)(F)F)c3)c3nnn(C4CCCC4)n3)c(N(CC3CC3)CC3CC3)nc12 ; Cc1cccc2cc(CN(Cc3cc(C(F)(F)F)cc(C(F)(F)F)c3)c3nnn(C4CCCCC4)n3)c(N(CC3CC3)CC3CC3)nc12 ; Cc1cccc2cc(CN(Cc3cc(C(F)(F)F)cc(C(F)(F)F)c3)c3nnn(C(C)C)n3)c(N(CC3CC3)CC3CC3)nc12]

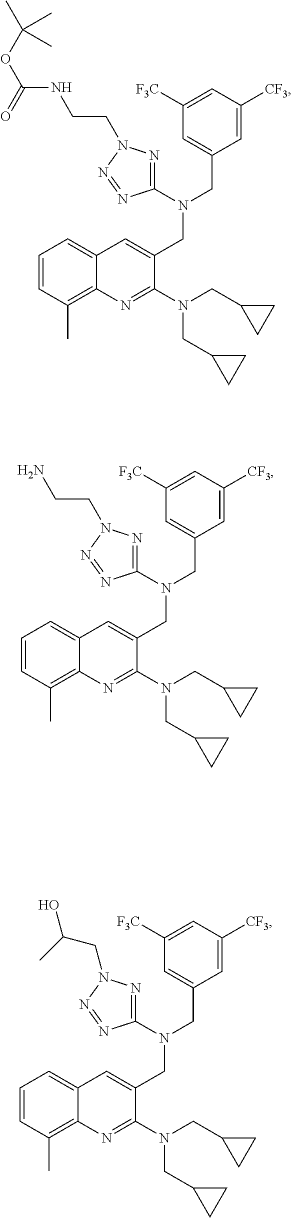 [SMILES: Cc1cccc2cc(CN(Cc3cc(C(F)(F)F)cc(C(F)(F)F)c3)c3nnn(CCNC(=O)OC(C)(C)C)n3)c(N(CC3CC3)CC3CC3)nc12 ; Cc1cccc2cc(CN(Cc3cc(C(F)(F)F)cc(C(F)(F)F)c3)c3nnn(CCN)n3)c(N(CC3CC3)CC3CC3)nc12 ; Cc1cccc2cc(CN(Cc3cc(C(F)(F)F)cc(C(F)(F)F)c3)c3nnn(CC(C)O)n3)c(N(CC3CC3)CC3CC3)nc12]

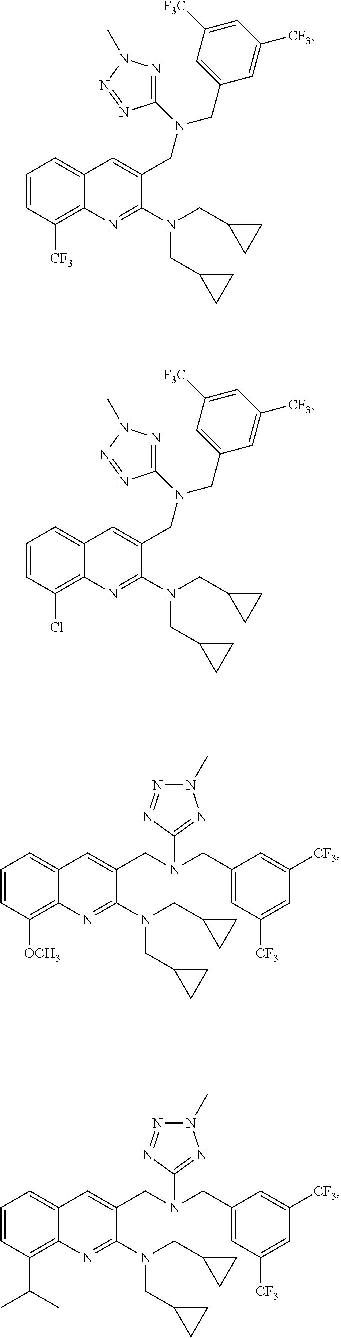 [SMILES: Cn1nnc(N(Cc2cc(C(F)(F)F)cc(C(F)(F)F)c2)Cc2cc3cccc(C(F)(F)F)c3nc2N(CC2CC2)CC2CC2)n1 ; Cn1nnc(N(Cc2cc(C(F)(F)F)cc(C(F)(F)F)c2)Cc2cc3cccc(Cl)c3nc2N(CC2CC2)CC2CC2)n1 ; COc1cccc2cc(CN(Cc3cc(C(F)(F)F)cc(C(F)(F)F)c3)c3nnn(C)n3)c(N(CC3CC3)CC3CC3)nc12 ; CC(C)c1cccc2cc(CN(Cc3cc(C(F)(F)F)cc(C(F)(F)F)c3)c3nnn(C)n3)c(N(CC3CC3)CC3CC3)nc12]

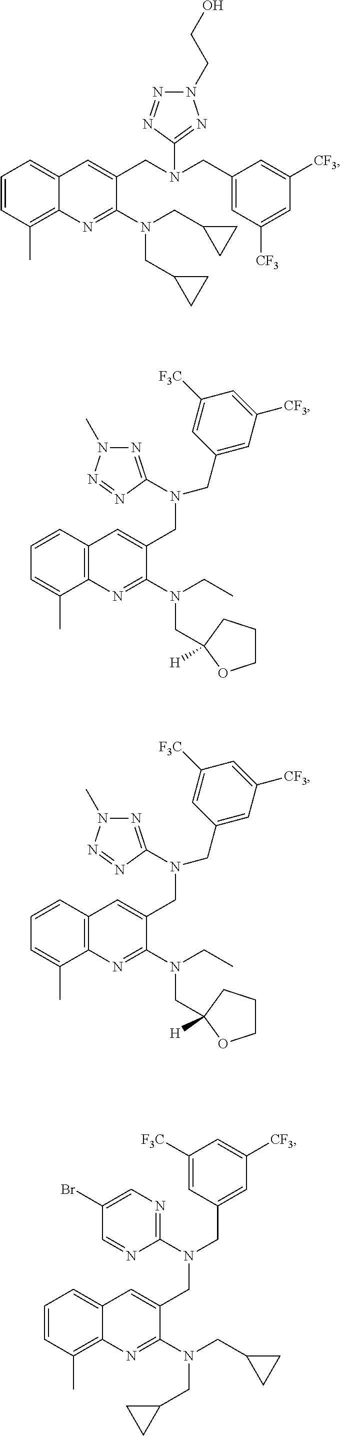 [SMILES: Cc1cccc2cc(CN(Cc3cc(C(F)(F)F)cc(C(F)(F)F)c3)c3nnn(CCO)n3)c(N(CC3CC3)CC3CC3)nc12 ; CCN(C[C@H]1CCCO1)c1nc2c(C)cccc2cc1CN(Cc1cc(C(F)(F)F)cc(C(F)(F)F)c1)c1nnn(C)n1 ; CCN(C[C@@H]1CCCO1)c1nc2c(C)cccc2cc1CN(Cc1cc(C(F)(F)F)cc(C(F)(F)F)c1)c1nnn(C)n1 ; Cc1cccc2cc(CN(Cc3cc(C(F)(F)F)cc(C(F)(F)F)c3)c3ncc(Br)cn3)c(N(CC3CC3)CC3CC3)nc12]

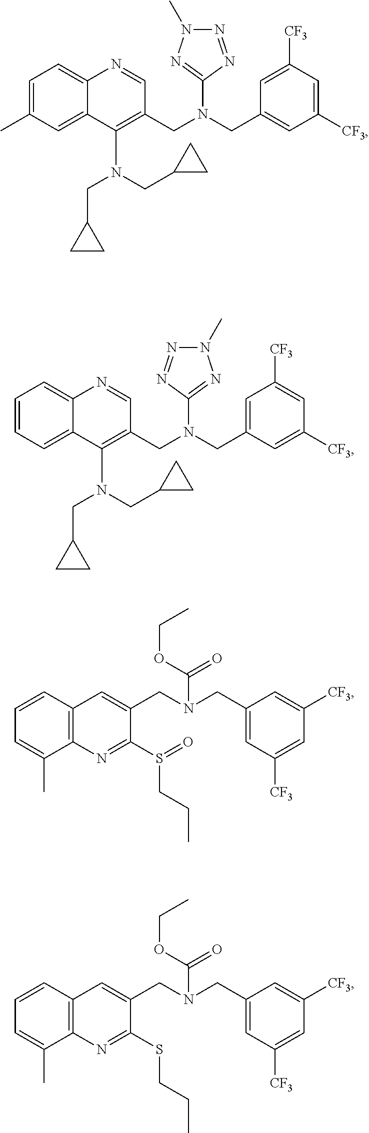 [SMILES: Cc1ccc2ncc(CN(Cc3cc(C(F)(F)F)cc(C(F)(F)F)c3)c3nnn(C)n3)c(N(CC3CC3)CC3CC3)c2c1 ; Cn1nnc(N(Cc2cc(C(F)(F)F)cc(C(F)(F)F)c2)Cc2cnc3ccccc3c2N(CC2CC2)CC2CC2)n1 ; CCCS(=O)c1nc2c(C)cccc2cc1CN(Cc1cc(C(F)(F)F)cc(C(F)(F)F)c1)C(=O)OCC ; CCCSc1nc2c(C)cccc2cc1CN(Cc1cc(C(F)(F)F)cc(C(F)(F)F)c1)C(=O)OCC]

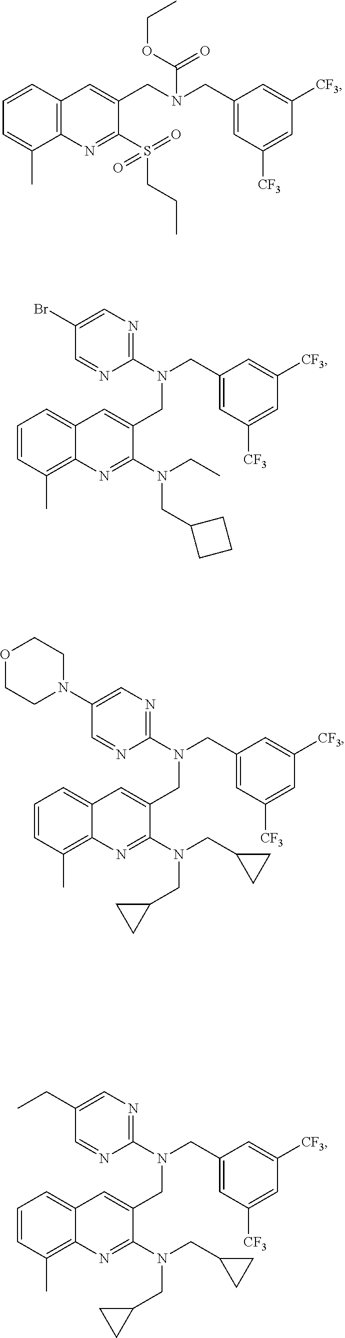 [SMILES: CCCS(=O)(=O)c1nc2c(C)cccc2cc1CN(Cc1cc(C(F)(F)F)cc(C(F)(F)F)c1)C(=O)OCC ; CCN(CC1CCC1)c1nc2c(C)cccc2cc1CN(Cc1cc(C(F)(F)F)cc(C(F)(F)F)c1)c1ncc(Br)cn1 ; Cc1cccc2cc(CN(Cc3cc(C(F)(F)F)cc(C(F)(F)F)c3)c3ncc(N4CCOCC4)cn3)c(N(CC3CC3)CC3CC3)nc12 ; CCc1cnc(N(Cc2cc(C(F)(F)F)cc(C(F)(F)F)c2)Cc2cc3cccc(C)c3nc2N(CC2CC2)CC2CC2)nc1]

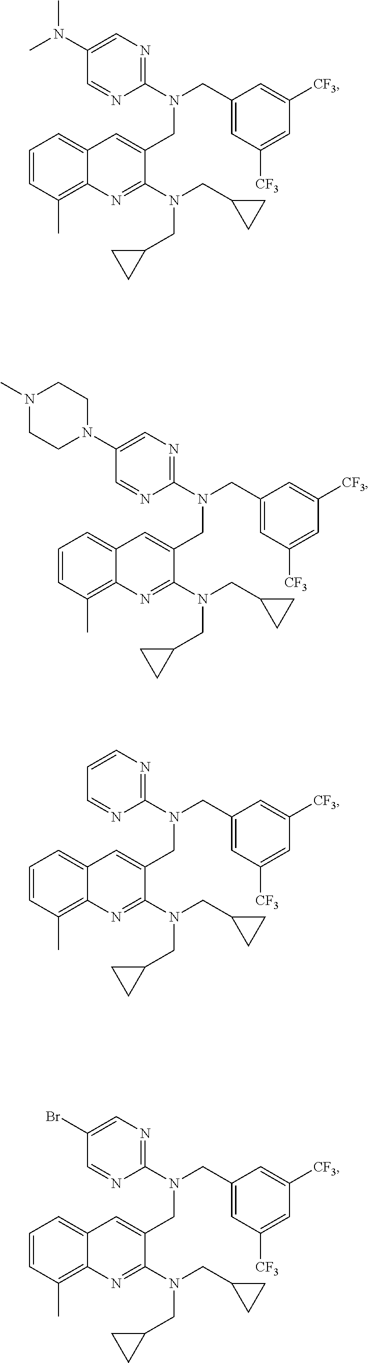 [SMILES: Cc1cccc2cc(CN(Cc3cc(C(F)(F)F)cc(C(F)(F)F)c3)c3ncc(N(C)C)cn3)c(N(CC3CC3)CC3CC3)nc12 ; Cc1cccc2cc(CN(Cc3cc(C(F)(F)F)cc(C(F)(F)F)c3)c3ncc(N4CCN(C)CC4)cn3)c(N(CC3CC3)CC3CC3)nc12 ; Cc1cccc2cc(CN(Cc3cc(C(F)(F)F)cc(C(F)(F)F)c3)c3ncccn3)c(N(CC3CC3)CC3CC3)nc12 ; Cc1cccc2cc(CN(Cc3cc(C(F)(F)F)cc(C(F)(F)F)c3)c3ncc(Br)cn3)c(N(CC3CC3)CC3CC3)nc12]

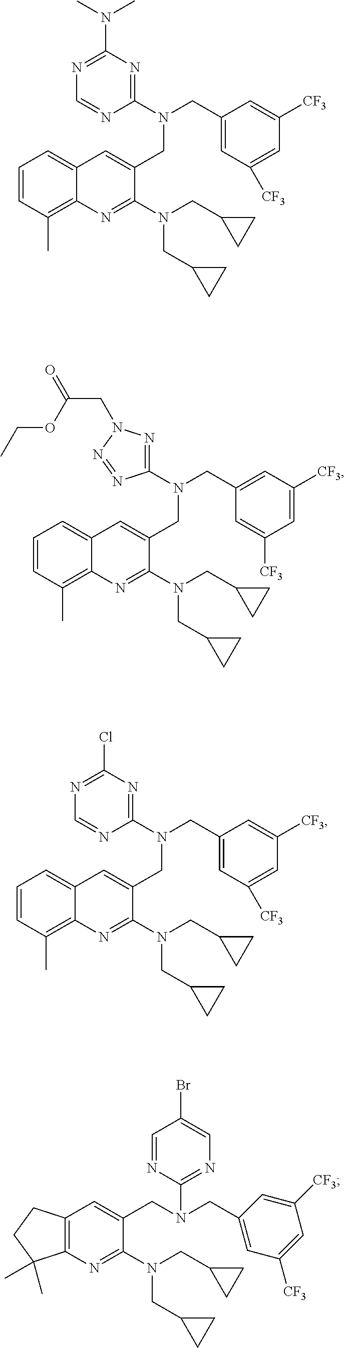 [SMILES: Cc1cccc2cc(CN(Cc3cc(C(F)(F)F)cc(C(F)(F)F)c3)c3ncnc(N(C)C)n3)c(N(CC3CC3)CC3CC3)nc12 ; CCOC(=O)Cn1nnc(N(Cc2cc(C(F)(F)F)cc(C(F)(F)F)c2)Cc2cc3cccc(C)c3nc2N(CC2CC2)CC2CC2)n1 ; Cc1cccc2cc(CN(Cc3cc(C(F)(F)F)cc(C(F)(F)F)c3)c3ncnc(Cl)n3)c(N(CC3CC3)CC3CC3)nc12 ; CC1(C)CCc2cc(CN(Cc3cc(C(F)(F)F)cc(C(F)(F)F)c3)c3ncc(Br)cn3)c(N(CC3CC3)CC3CC3)nc21]

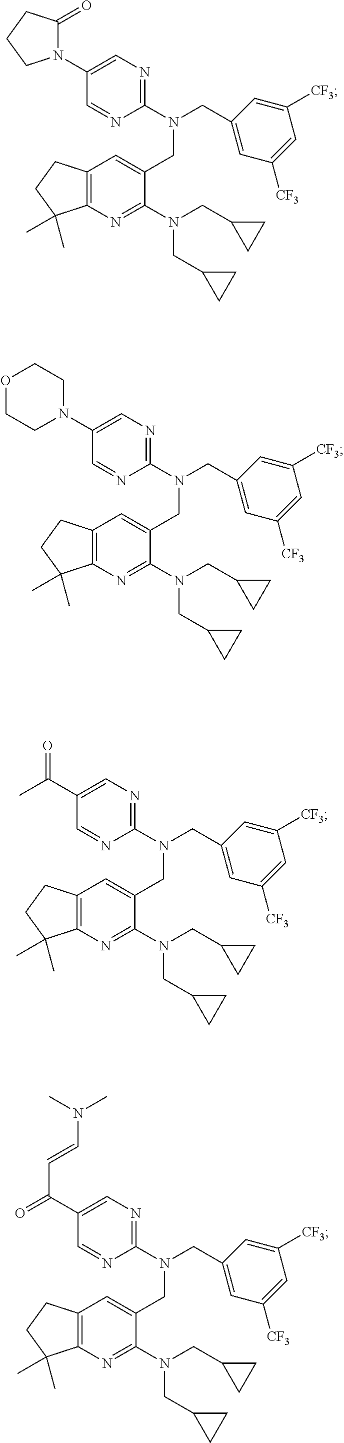 [SMILES: CC1(C)CCc2cc(CN(Cc3cc(C(F)(F)F)cc(C(F)(F)F)c3)c3ncc(N4CCCC4=O)cn3)c(N(CC3CC3)CC3CC3)nc21 ; CC1(C)CCc2cc(CN(Cc3cc(C(F)(F)F)cc(C(F)(F)F)c3)c3ncc(N4CCOCC4)cn3)c(N(CC3CC3)CC3CC3)nc21 ; CC(=O)c1cnc(N(Cc2cc(C(F)(F)F)cc(C(F)(F)F)c2)Cc2cc3c(nc2N(CC2CC2)CC2CC2)C(C)(C)CC3)nc1 ; CN(C)/C=C/C(=O)c1cnc(N(Cc2cc(C(F)(F)F)cc(C(F)(F)F)c2)Cc2cc3c(nc2N(CC2CC2)CC2CC2)C(C)(C)CC3)nc1]

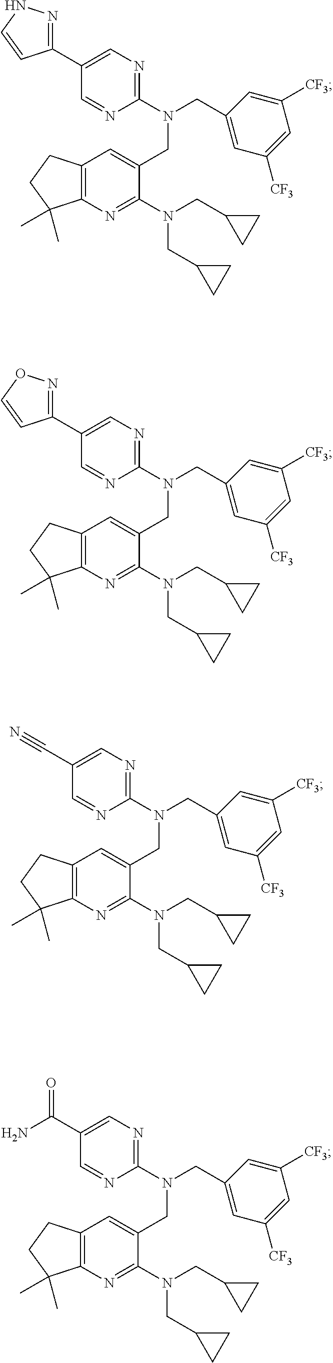 [SMILES: CC1(C)CCc2cc(CN(Cc3cc(C(F)(F)F)cc(C(F)(F)F)c3)c3ncc(-c4cc[nH]n4)cn3)c(N(CC3CC3)CC3CC3)nc21 ; CC1(C)CCc2cc(CN(Cc3cc(C(F)(F)F)cc(C(F)(F)F)c3)c3ncc(-c4ccon4)cn3)c(N(CC3CC3)CC3CC3)nc21 ; CC1(C)CCc2cc(CN(Cc3cc(C(F)(F)F)cc(C(F)(F)F)c3)c3ncc(C#N)cn3)c(N(CC3CC3)CC3CC3)nc21 ; CC1(C)CCc2cc(CN(Cc3cc(C(F)(F)F)cc(C(F)(F)F)c3)c3ncc(C(N)=O)cn3)c(N(CC3CC3)CC3CC3)nc21]

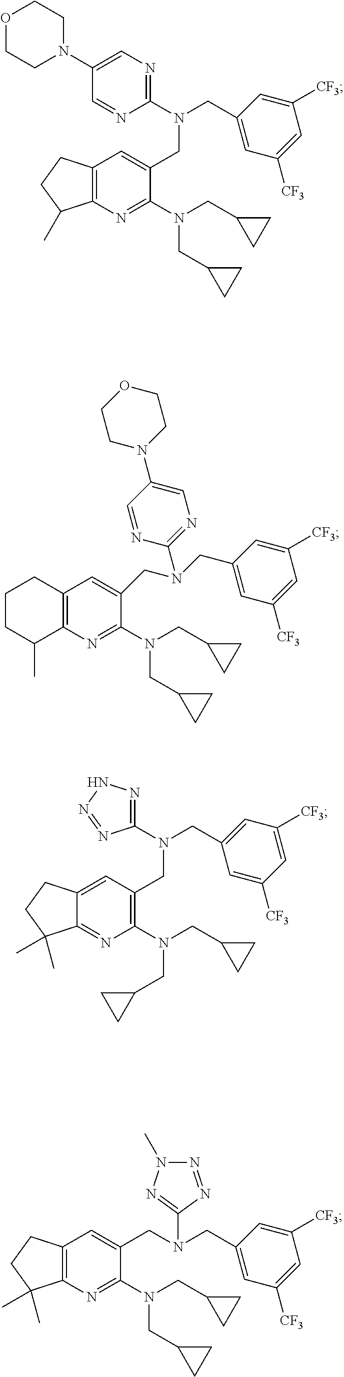 [SMILES: CC1CCc2cc(CN(Cc3cc(C(F)(F)F)cc(C(F)(F)F)c3)c3ncc(N4CCOCC4)cn3)c(N(CC3CC3)CC3CC3)nc21 ; CC1CCCc2cc(CN(Cc3cc(C(F)(F)F)cc(C(F)(F)F)c3)c3ncc(N4CCOCC4)cn3)c(N(CC3CC3)CC3CC3)nc21 ; CC1(C)CCc2cc(CN(Cc3cc(C(F)(F)F)cc(C(F)(F)F)c3)c3nn[nH]n3)c(N(CC3CC3)CC3CC3)nc21 ; Cn1nnc(N(Cc2cc(C(F)(F)F)cc(C(F)(F)F)c2)Cc2cc3c(nc2N(CC2CC2)CC2CC2)C(C)(C)CC3)n1]

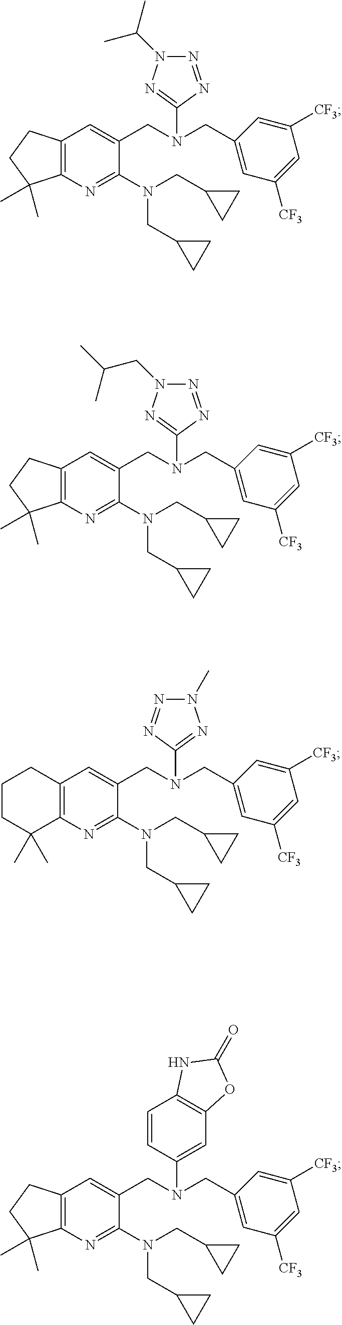 [SMILES: CC(C)n1nnc(N(Cc2cc(C(F)(F)F)cc(C(F)(F)F)c2)Cc2cc3c(nc2N(CC2CC2)CC2CC2)C(C)(C)CC3)n1 ; CC(C)Cn1nnc(N(Cc2cc(C(F)(F)F)cc(C(F)(F)F)c2)Cc2cc3c(nc2N(CC2CC2)CC2CC2)C(C)(C)CC3)n1 ; Cn1nnc(N(Cc2cc(C(F)(F)F)cc(C(F)(F)F)c2)Cc2cc3c(nc2N(CC2CC2)CC2CC2)C(C)(C)CCC3)n1 ; CC1(C)CCc2cc(CN(Cc3cc(C(F)(F)F)cc(C(F)(F)F)c3)c3ccc4[nH]c(=O)oc4c3)c(N(CC3CC3)CC3CC3)nc21]

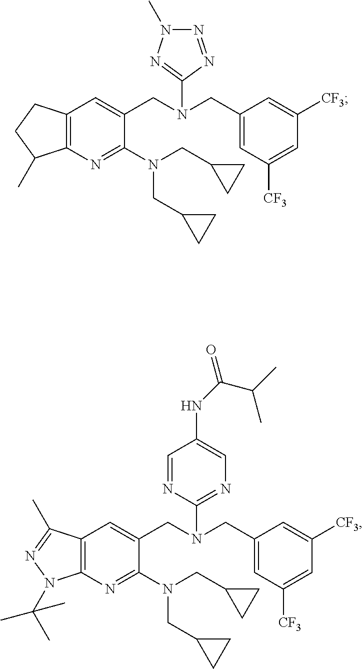 [SMILES: CC1CCc2cc(CN(Cc3cc(C(F)(F)F)cc(C(F)(F)F)c3)c3nnn(C)n3)c(N(CC3CC3)CC3CC3)nc21 ; Cc1nn(C(C)(C)C)c2nc(N(CC3CC3)CC3CC3)c(CN(Cc3cc(C(F)(F)F)cc(C(F)(F)F)c3)c3ncc(NC(=O)C(C)C)cn3)cc12]

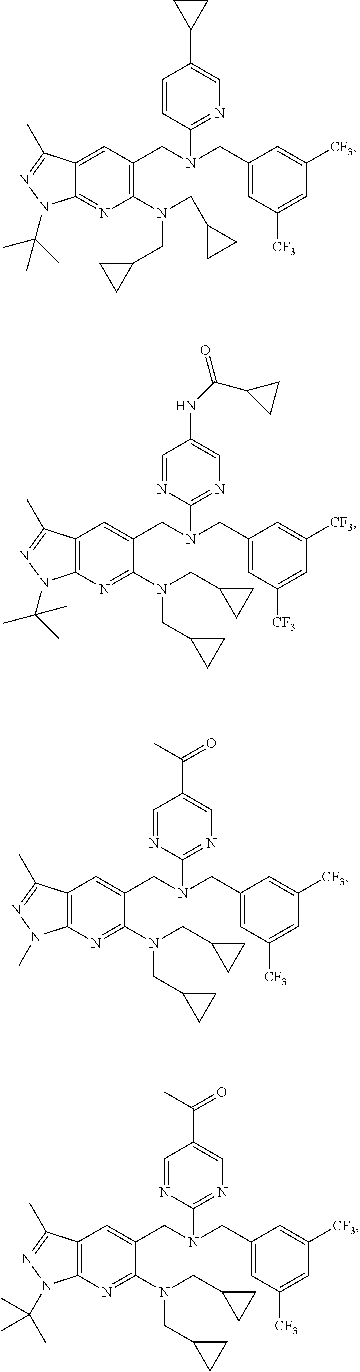 [SMILES: Cc1nn(C(C)(C)C)c2nc(N(CC3CC3)CC3CC3)c(CN(Cc3cc(C(F)(F)F)cc(C(F)(F)F)c3)c3ccc(C4CC4)cn3)cc12 ; Cc1nn(C(C)(C)C)c2nc(N(CC3CC3)CC3CC3)c(CN(Cc3cc(C(F)(F)F)cc(C(F)(F)F)c3)c3ncc(NC(=O)C4CC4)cn3)cc12 ; CC(=O)c1cnc(N(Cc2cc(C(F)(F)F)cc(C(F)(F)F)c2)Cc2cc3c(C)nn(C)c3nc2N(CC2CC2)CC2CC2)nc1 ; CC(=O)c1cnc(N(Cc2cc(C(F)(F)F)cc(C(F)(F)F)c2)Cc2cc3c(C)nn(C(C)(C)C)c3nc2N(CC2CC2)CC2CC2)nc1]

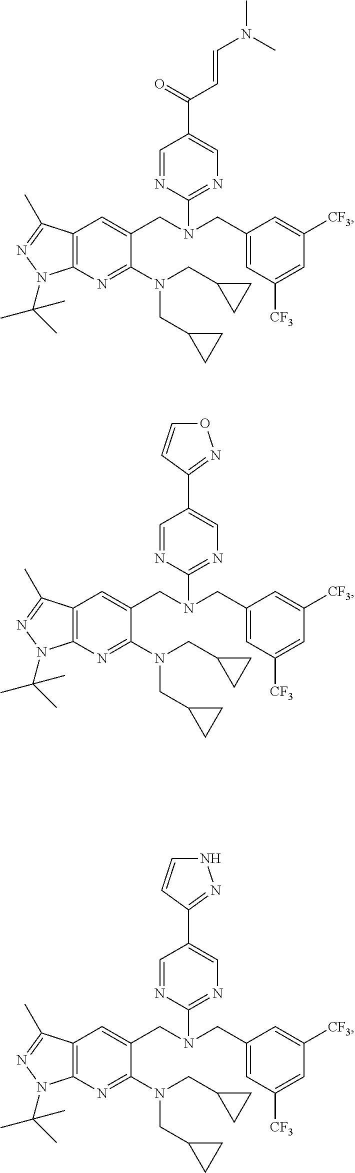 [SMILES: Cc1nn(C(C)(C)C)c2nc(N(CC3CC3)CC3CC3)c(CN(Cc3cc(C(F)(F)F)cc(C(F)(F)F)c3)c3ncc(C(=O)/C=C/N(C)C)cn3)cc12 ; Cc1nn(C(C)(C)C)c2nc(N(CC3CC3)CC3CC3)c(CN(Cc3cc(C(F)(F)F)cc(C(F)(F)F)c3)c3ncc(-c4ccon4)cn3)cc12 ; Cc1nn(C(C)(C)C)c2nc(N(CC3CC3)CC3CC3)c(CN(Cc3cc(C(F)(F)F)cc(C(F)(F)F)c3)c3ncc(-c4cc[nH]n4)cn3)cc12]

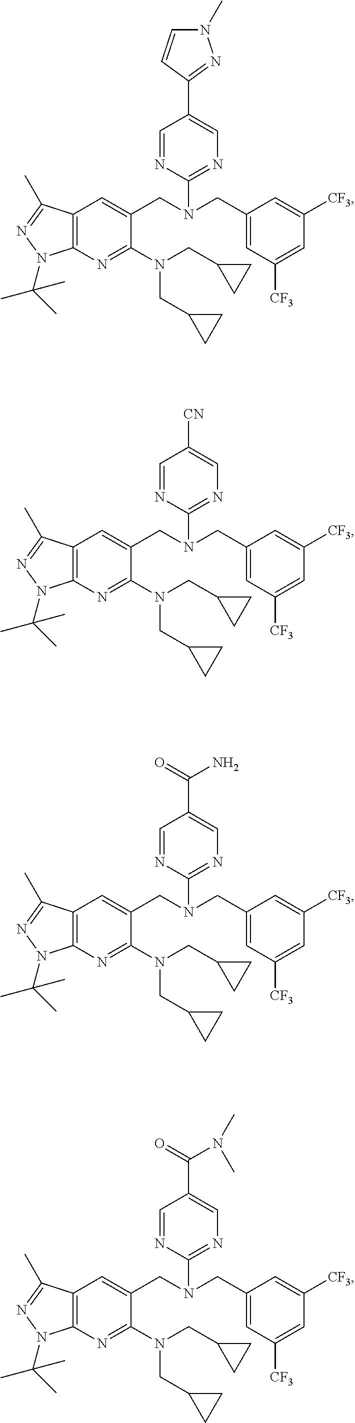 [SMILES: Cc1nn(C(C)(C)C)c2nc(N(CC3CC3)CC3CC3)c(CN(Cc3cc(C(F)(F)F)cc(C(F)(F)F)c3)c3ncc(-c4ccn(C)n4)cn3)cc12 ; Cc1nn(C(C)(C)C)c2nc(N(CC3CC3)CC3CC3)c(CN(Cc3cc(C(F)(F)F)cc(C(F)(F)F)c3)c3ncc(C#N)cn3)cc12 ; Cc1nn(C(C)(C)C)c2nc(N(CC3CC3)CC3CC3)c(CN(Cc3cc(C(F)(F)F)cc(C(F)(F)F)c3)c3ncc(C(N)=O)cn3)cc12 ; Cc1nn(C(C)(C)C)c2nc(N(CC3CC3)CC3CC3)c(CN(Cc3cc(C(F)(F)F)cc(C(F)(F)F)c3)c3ncc(C(=O)N(C)C)cn3)cc12]

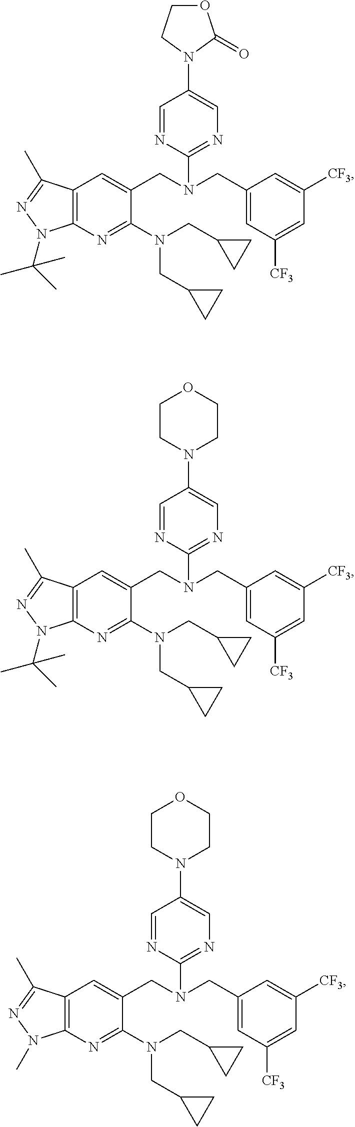 [SMILES: Cc1nn(C(C)(C)C)c2nc(N(CC3CC3)CC3CC3)c(CN(Cc3cc(C(F)(F)F)cc(C(F)(F)F)c3)c3ncc(N4CCOC4=O)cn3)cc12 ; Cc1nn(C(C)(C)C)c2nc(N(CC3CC3)CC3CC3)c(CN(Cc3cc(C(F)(F)F)cc(C(F)(F)F)c3)c3ncc(N4CCOCC4)cn3)cc12 ; Cc1nn(C)c2nc(N(CC3CC3)CC3CC3)c(CN(Cc3cc(C(F)(F)F)cc(C(F)(F)F)c3)c3ncc(N4CCOCC4)cn3)cc12]

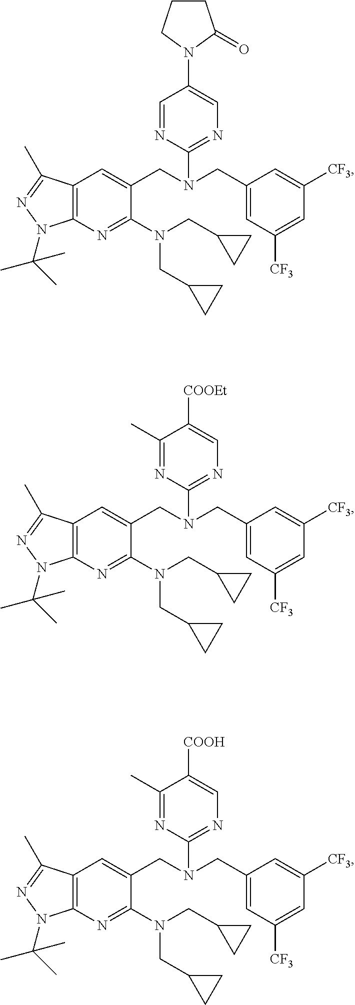 [SMILES: Cc1nn(C(C)(C)C)c2nc(N(CC3CC3)CC3CC3)c(CN(Cc3cc(C(F)(F)F)cc(C(F)(F)F)c3)c3ncc(N4CCCC4=O)cn3)cc12 ; CCOC(=O)c1cnc(N(Cc2cc(C(F)(F)F)cc(C(F)(F)F)c2)Cc2cc3c(C)nn(C(C)(C)C)c3nc2N(CC2CC2)CC2CC2)nc1C ; Cc1nc(N(Cc2cc(C(F)(F)F)cc(C(F)(F)F)c2)Cc2cc3c(C)nn(C(C)(C)C)c3nc2N(CC2CC2)CC2CC2)ncc1C(=O)O]

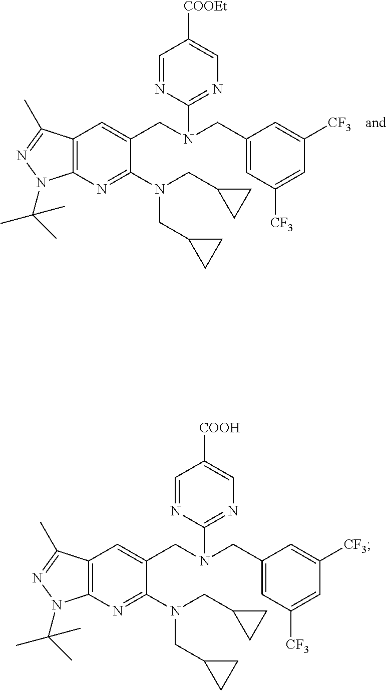 [SMILES: CCOC(=O)c1cnc(N(Cc2cc(C(F)(F)F)cc(C(F)(F)F)c2)Cc2cc3c(C)nn(C(C)(C)C)c3nc2N(CC2CC2)CC2CC2)nc1 ; Cc1nn(C(C)(C)C)c2nc(N(CC3CC3)CC3CC3)c(CN(Cc3cc(C(F)(F)F)cc(C(F)(F)F)c3)c3ncc(C(=O)O)cn3)cc12]

or a stereoisomer thereof or a pharmaceutically acceptable salt thereof

In another embodiment, various compositions in accordance with the present application can be prepared by substituting 1.3-(((3,5-bis(trifluoromethyl)benzyl)(2-methyl-2H-tetrazol-5-yl)amino)methyl)-N,N-bis(cyclopropylmethyl)-8-methylquinolin-2-amine, as described in Examples 1-31, with any one of the following compounds:

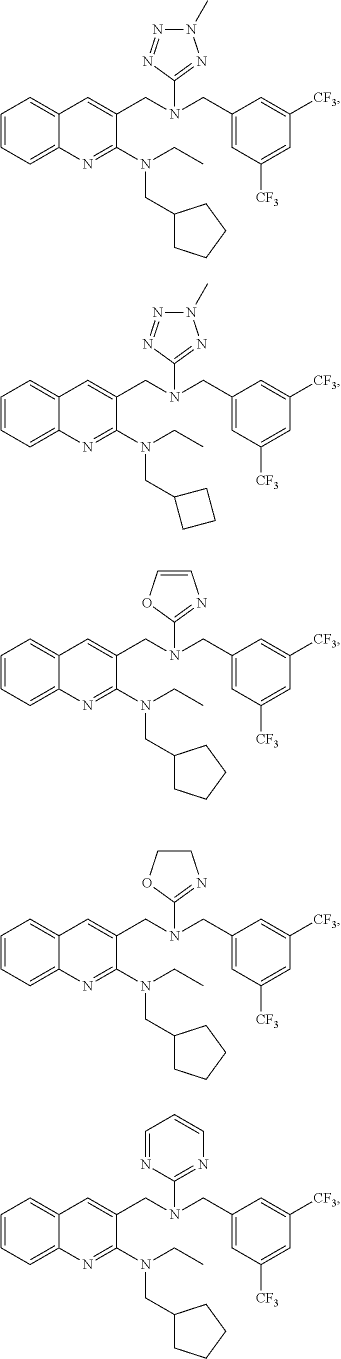

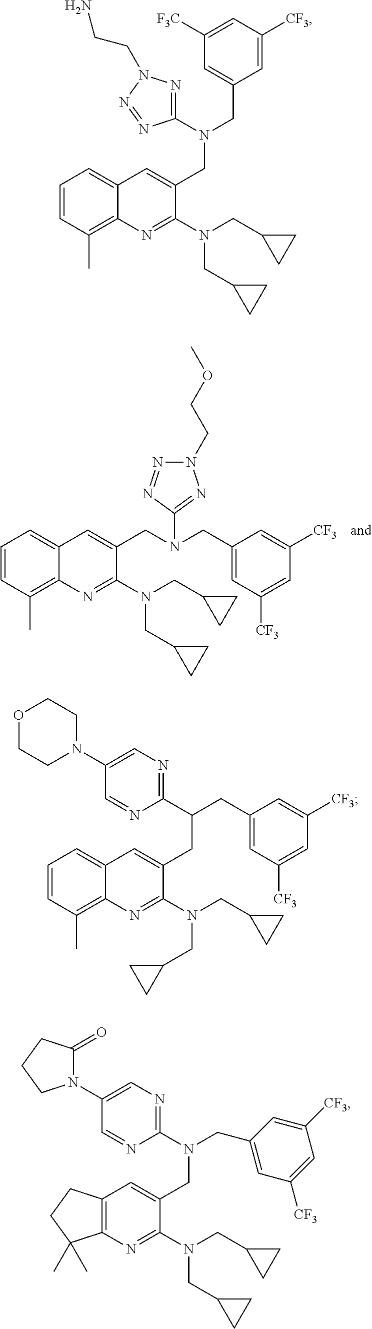 [SMILES: Cc1cccc2cc(CN(Cc3cc(C(F)(F)F)cc(C(F)(F)F)c3)c3nnn(CCN)n3)c(N(CC3CC3)CC3CC3)nc12 ; COCCn1nnc(N(Cc2cc(C(F)(F)F)cc(C(F)(F)F)c2)Cc2cc3cccc(C)c3nc2N(CC2CC2)CC2CC2)n1 ; Cc1cccc2cc(CC(Cc3cc(C(F)(F)F)cc(C(F)(F)F)c3)c3ncc(N4CCOCC4)cn3)c(N(CC3CC3)CC3CC3)nc12 ; CC1(C)CCc2cc(CN(Cc3cc(C(F)(F)F)cc(C(F)(F)F)c3)c3ncc(N4CCCC4=O)cn3)c(N(CC3CC3)CC3CC3)nc21]

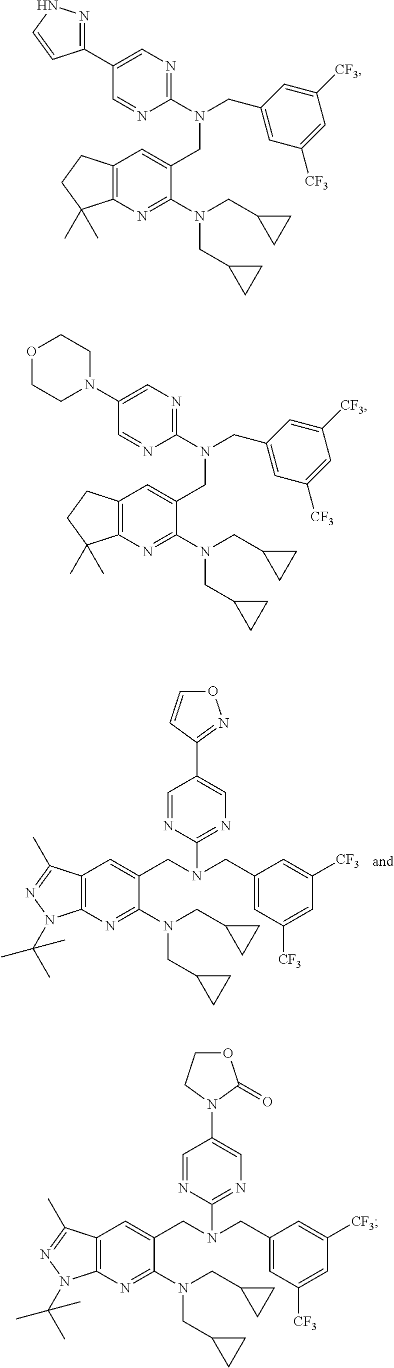 [SMILES: CC1(C)CCc2cc(CN(Cc3cc(C(F)(F)F)cc(C(F)(F)F)c3)c3ncc(-c4cc[nH]n4)cn3)c(N(CC3CC3)CC3CC3)nc21 ; CC1(C)CCc2cc(CN(Cc3cc(C(F)(F)F)cc(C(F)(F)F)c3)c3ncc(N4CCOCC4)cn3)c(N(CC3CC3)CC3CC3)nc21 ; Cc1nn(C(C)(C)C)c2nc(N(CC3CC3)CC3CC3)c(CN(Cc3cc(C(F)(F)F)cc(C(F)(F)F)c3)c3ncc(-c4ccon4)cn3)cc12 ; Cc1nn(C(C)(C)C)c2nc(N(CC3CC3)CC3CC3)c(CN(Cc3cc(C(F)(F)F)cc(C(F)(F)F)c3)c3ncc(N4CCOC4=O)cn3)cc12]

or a stereoisomer thereof or a pharmaceutically acceptable salt thereof

Although the invention has been illustrated by certain of the preceding examples, it is not to be construed as being limited thereby; but rather, the invention encompasses the generic area as hereinbefore disclosed. Various modifications and embodiments can be made without departing from the spirit and scope thereof.

What is claimed is:

1. A pharmaceutical composition comprising:
a) a cholesteryl ester transfer protein (CETP) inhibitor having formula (I) or (Ia′) or (II) or (III),
b) at least one surfactant,
c) optionally a carrier material, and
d) optionally one or more pharmaceutically acceptable excipients, wherein
(i) the CETP inhibitor having formula (I) is: 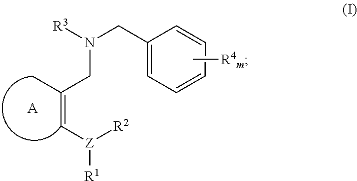
or a stereoisomer thereof or a pharmaceutically acceptable salt thereof,
wherein:
A is a substituted or an unsubstituted quinoline moiety having the formula: 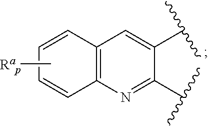
wherein R^{a}, in each occurrence, is selected independently from: 1) a halogen; a hydroxyl, or a cyano; 2) an alkyl or an alkoxy, any of which having up to 12 carbon atoms; or 3) CO₂R⁶; and p is an integer from 0 to 3, inclusive;
R¹ and R² are selected independently from: 1) hydrogen; 2) a substituted or an unsubstituted alkyl, cycloalkyl, haloalkyl, aryl, heterocyclyl, heteroaryl, any of which having up to 12 carbon atoms, wherein any heterocyclyl or heteroaryl comprises at least one heteroatom or heterogroup selected independently from O, N, S, NR¹⁰, SO₂, or CO; 3) CO₂R⁶, COR⁸, SO₂R⁸, SO₂NR⁶R⁷, or CONR⁶R⁷; or 4) (CHR^{x})ₙR⁵ or (CH₂)ₙR^{d}CO₂R^{e}, wherein n, in each occurrence, is 1, 2, or 3; R^{x}, in each occurrence, is selected independently from an alkyl or an alkoxy, either of which having up to 12 carbon atoms, or hydrogen; R^{d}, in each occurrence, is selected independently from an alkyl, a cycloalkyl, an aryl, a heterocyclyl, or a heteroaryl, any of which having up to 12 carbon atoms, wherein any heterocyclyl or heteroaryl comprises at least one heteroatom or heterogroup selected independently from O, N, S, NR¹⁰, SO₂, or CO; and R^{e}, in each occurrence, is selected independently from an alkyl or a cycloalkyl, either of which having up to 12 carbon atoms, or hydrogen;
or R¹ and R² together with the diradical Z to which they are attached form a substituted or an unsubstituted monocyclic or bicyclic moiety comprising up to 12 carbon atoms, and optionally comprising 1, 2, or 3 heteroatoms or heterogroups in addition to Z, selected independently from O, N, S, NR¹⁰, SO₂, or CO;
R³ is selected from: 1) hydrogen or cyano; 2) a substituted alkyl having up to 12 carbon atoms; 3) a substituted or an unsubstituted aryl, or a substituted or an unsubstituted 5-, 6-, or 7-membered heterocyclyl or heteroaryl, any of which having up to 12 carbon atoms, comprising 1, 2, or 3 heteroatoms or heterogroups selected independently from O, N, S, NR¹⁰, SO₂, or CO; CO₂R⁶, COR⁸, SO₂R⁸, SO₂NR⁶R⁷, CONR⁶R⁷, C(S)NR⁶R⁷, C(S)NC(O)OR⁸, or C(S)SR⁸; or 5) a substituted or an unsubstituted group selected from 4,5-dihydro-oxazolyl, tetrazolyl, isoxazolyl, pyridyl, pyrimidinyl, oxadiazolyl, thiazolyl, or oxazolyl; wherein any optional substituent is selected independently from: a) an alkyl or haloalkyl, any of which having up to 12 carbon atoms; or b) CO₂R⁹, wherein R⁹ is an alkyl having up to 12 carbon atoms;
wherein when R³ is an aryl, a heterocyclyl, or a heteroaryl, R³ is optionally substituted with up to three substituents selected independently from a halogen, a hydroxyl, a cyano, an alkoxy having up to 12 carbon atoms, or R¹¹;
R⁴, in each occurrence, is selected independently from: 1) halogen, cyano, or hydroxy; 2) an alkyl, a cycloalkyl, a cycloalkoxy, an alkoxy, a haloalkyl, or a haloalkoxy, any of which having up to 12 carbon atoms; 3) a substituted or an unsubstituted aryl, aralkyl, aryloxy, heteroaryl, or heteroaryloxy, any of which having up to 12 carbon atoms, wherein any heteroaryl or heteroaryloxy comprises at least one heteroatom or heterogroup selected independently from O, N, S, or NR¹⁰; or 4) CO₂R⁶, COR⁸, SO₂R⁸, SO₂NR⁶R⁷, CONR⁶R⁷, or (CH₂)NR⁶R⁷, wherein q is an integer from 0 to 5, inclusive;
m is an integer from 0 to 3, inclusive;
or R⁴ₘ is a fused cyclic moiety comprising from 3 to 5 additional ring carbon atoms, inclusive, and optionally comprising at least one heteroatom or heterogroup selected independently from O, N, S, NR¹⁰, SO₂, or CO;
R⁵, in each occurrence, is selected independently from: 1) an alkoxy, a haloalkoxy, or a cycloalkyl, any of which having up to 12 carbon atoms; 2) a substituted or an unsubstituted aryl, heterocyclyl, or heteroaryl, any of which having up to 12 carbon atoms, wherein any heterocyclyl or heteroaryl comprises at least one heteroatom or heterogroup selected independently from O, N, S, NR¹⁰, SO₂, or CO; 3) hydroxyl, NR⁶R⁷, CO₂R⁶, COR⁸, or SO₂R⁸; or 4) a substituted or an unsubstituted heterocycloalkyl comprising from 3 to 7 ring carbon atoms, and from 1 to 3 heteroatoms or heterogroups, inclusive, selected independently from O, N, S, NR¹⁰, SO₂, or CO;
R⁶ and R⁷, in each occurrence, are selected independently from: 1) hydrogen; 2) an alkyl, a cycloalkyl, or a haloalkyl, any of which having up to 12 carbon atoms; or 3) a substituted or an unsubstituted aryl, aralkyl, heterocyclyl, or heteroaryl, any of which having up to 12 carbon atoms, wherein any heterocyclyl or heteroaryl comprises at least one heteroatom or heterogroup selected independently from O, N, S, NR¹⁰, SO₂, or CO;
or R⁶ and R⁷ together with the nitrogen atom to which they are attached form a substituted or an unsubstituted cyclic moiety having from 3 to 7 ring carbon atoms, and optionally comprising 1, 2, or 3 heteroatoms in addition to the nitrogen atom to which R⁶ and R⁷ are bonded, selected independently from O, N, S, or NR¹⁰;
R⁸, in each occurrence, is selected independently from: 1) an alkyl, a cycloalkyl, or a haloalkyl, any of which having up to 12 carbon atoms; or 2) a substituted or an unsubstituted aryl, heterocyclyl, or heteroaryl, any of which having up to 12 carbon atoms, wherein any heterocyclyl or heteroaryl comprises at least one heteroatom or heterogroup selected independently from O, N, S, NR¹⁰, SO₂, or CO;
R¹⁰, in each occurrence, is selected independently from: 1) hydrogen; or 2) an alkyl, a cycloalkyl, a haloalkyl, an aryl, or an aralkyl, any of which having up to 12 carbon atoms;
Z is N or CH; or the ZR¹ moiety is S, CO, or SO₂; or the ZR¹R² moiety is —C≡CR²;
R¹¹ is selected independently from:
1) an alkyl, a haloalkyl, a cycloalkyl, or an alkoxycarbonyl, any of which having up to 12 carbon atoms;
2) a substituted or an unsubstituted heteroaryl or heterocyclyl, any of which having up to 12 carbon atoms, comprises at least one heteroatom or heterogroup selected independently from O, N, S, NR¹⁰, SO₂, or CO, wherein any substituted heteroaryl or heterocyclyl is substituted with up to three substituents selected independently from an alkyl having up to 12 carbon atoms or a hydroxyl; or
3) —CO—Z²—R¹³, —CO—R¹², —CO—Z²—(CH₂)ᵣ—CO—Z²—R¹³, —NR¹⁵R¹⁶, —Z²—CO—(CH₂)ᵣ—Z²—R¹³, —Z²—CO—(CH₂)ᵣ—CO—Z²—R¹³, —O—(CH₂)ᵣ—CO—Z²—R¹³, —O—(CH₂)ᵣ—R¹⁴, —O—R¹²—(CH₂)ᵣ—R¹³, —O—R¹⁴—CO—O—R¹³, —O—(CH₂)ᵣ—R¹², —O—(CH₂)ᵣ—NR′R″, —O—(CH₂)ᵣ—CO₂—(CH₂)ᵣ—R¹³, —O—(CH₂)ᵣ—SR⁸, —O—(CH₂)ᵣ—CO₂—R¹³, —O—(CH₂)ᵣ—CONR′R″, —O—(CH₂)ᵣ—CONH—(CH₂)ᵣ—OR¹³, —O—(CH₂)ᵣ—SO₂R⁸, —O—(CH₂)ᵣ—R¹³, —O—(CH₂)ᵣ—OR¹³, —O—(CH₂)ᵣ—O—(CH₂)ᵣ—OR¹³, —S—(CH₂)ᵣ—CONR′R″, —SO₂—(CH₂)ᵣ—OR¹³, —SO₂—(CH₂)ᵣ—CONR′R″, —(CH₂)ᵣ—O—CO—R⁸, —(CH₂)ᵣ—R¹², —(CH₂)ᵣ—R¹³, —(CH₂)ᵣ—CO—Z²—R¹³, —(CH₂)ᵣ—Z²—R¹³, or -alkenylene-CO₂—(CH₂)ᵣ—R¹³;
r, in each occurrence, is independently 1, 2, or 3;
R¹², in each occurrence, is independently selected from a substituted or an unsubstituted heterocyclyl having up to 12 carbon atoms, comprising at least one heteroatom or heterogroup selected independently from O, N, S, NR¹⁰, SO₂, or CO, wherein any substituted heterocyclyl is substituted with up to three substituents selected independently from an acyl, an alkyl, or an alkoxycarbonyl, any of which having up to 12 carbon atoms, or —COOH;
R¹³, in each occurrence, is independently selected from: 1) hydrogen; or 2) a cycloalkyl, an aryl, a haloalkyl, a heterocyclyl, or an alkyl group optionally substituted with at least one hydroxyl, any of which having up to 12 carbon atoms, wherein any heterocyclyl comprises at least one heteroatom or heterogroup selected independently from O, N, S, NR¹⁰, SO₂, or CO;
R¹⁴, in each occurrence, is independently selected from a heterocyclyl, a cycloalkyl, or an aryl, any of which having up to 12 carbon atoms, wherein any heterocyclyl comprises at least one heteroatom or heterogroup selected independently from O, N, S, NR¹⁰, SO₂, or CO;
Z², in each occurrence, is selected independently from NR¹⁰ or O;
R′ and R″, in each occurrence, are independently selected from hydrogen or an alkyl having up to 12 carbon atoms; and
R¹⁵ and R¹⁶, in each occurrence, are independently selected from: 1) hydrogen; 2) an alkyl having up to 12 carbon atoms; or 3) —(CH₂)ᵣ—O—R¹³, —(CH₂)ᵣ—R¹⁴, —COR¹³, —(CH₂)ᵣ—CO—Z²—R¹³, —CO₂R¹³, —CO₂—(CH₂)ᵣ—R¹³, —CO₂—(CH₂)ᵣ—R¹², —CO₂—(CH₂)ᵣ—CO—Z²—R¹³, —CO₂—(CH₂)ᵣ—OR¹³, —CO—(CH₂)ᵣ—O—(CH₂)ᵣ—O—(CH₂)ᵣ—R¹³, —CO—(CH₂)ᵣ—O(CH₂)ᵣ—OR¹³, or —CO—NH—(CH₂)ᵣ—OR¹³;
or R¹⁵ and R¹⁶ together with the nitrogen atom to which they are attached form a substituted or an unsubstituted cyclic moiety comprising up to 12 carbon atoms, optionally comprising at least one additional heteroatom or heterogroup selected independently from O, N, S, NR¹⁰, SO₂, or CO; wherein any substituted cyclic moiety is substituted with up to three substituents selected independently from: 1) hydroxyl; 2) an alkyl or a heteroaryl, any of which having up to 12 carbon atoms, wherein any heteroaryl comprises at least one heteroatom or heterogroup selected independently from O, N, S, or NR¹⁰; or 3) COOR¹³, —Z²—(CH₂)ᵣ—R¹³, —COR¹³, —CO₂—(CH₂)ᵣ—R¹³, —CO(CH₂)ᵣ—O—R¹³, —(CH₂)ᵣ—CO₂—R¹³, —SO₂R⁸, —SO₂NR′R″, or —NR′R″;
wherein the —(CH₂)ᵣ— linking moiety, in any occurrence, is optionally substituted with at least one group selected independently from hydroxyl, amino, or an alkyl having up to 3 carbon atoms;
when R¹ and R² do not form a monocyclic or bicyclic moiety, R¹ and R² are optionally substituted with 1 or 2 substituents, and when substituted, the substituents are selected independently from: 1) an alkyl, a cycloalkyl, a haloalkyl, an alkoxy, an aryl, a heteroaryl, or a heterocyclyl, any of which having up to 12 carbon atoms, wherein any heteroaryl or heterocyclyl comprises at least one heteroatom or heterogroup selected independently from O, N, S, NR¹⁰, SO₂, or CO; or 2) halogen, cyano, or hydroxyl;
when R¹ and R² together with the diradical Z to which they are attached form a monocyclic or a bicyclic moiety, the cyclic moiety is optionally substituted with at least one substituent selected independently from: 1) halogen, cyano, or hydroxyl; 2) an alkyl, a haloalkyl, a cycloalkyl, an alkoxy, a cycloalkyl-substituted alkyl, an alkoxyalkyl, a cycloalkoxy, a haloalkoxy, an aryl, an aryloxy, an aralkyl, a heteroaryl or a heteroaryloxy, any of which having up to 12 carbon atoms, wherein any heteroaryl or heteroaryloxy comprises at least one heteroatom or heterogroup selected independently from O, N, S, or NR¹⁰; or 3) CO₂R⁶, COR⁸, SO₂R⁸, SO₂NR⁶R⁷, or CONR⁶R⁷;
R⁴, R⁶, R⁷, and R⁸ are optionally substituted with at least one substituent, and when substituted, the substituents are selected independently from: 1) halogen, hydroxy, cyano, or NR⁶R⁷; or 2) an alkyl or an alkoxy, any of which having up to 12 carbon atoms;
and R⁵ is optionally substituted with at least one substituent, and when substituted, the substituents are selected independently from: 1) halogen, hydroxy, cyano, or NR⁶R⁷; or 2) an alkyl having up to 12 carbon atoms;
(ii) the CETP inhibitor having formula (Ia′), 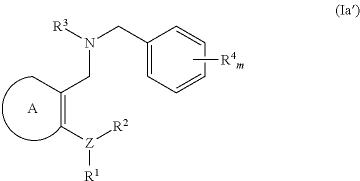
or a stereoisomer thereof or a pharmaceutically acceptable salt thereof, wherein A-ZR¹R² is: 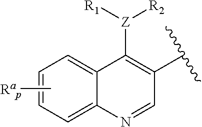
wherein R^{a}, in each occurrence, is selected independently from: 1) a hydrogen, a halogen, a cyano, or a hydroxyl; 2) an alkyl, a haloalkyl, a cycloalkyl, a (cycloalkyl)alkyl, an alkoxy, a cycloalkoxy, a haloalkoxy, an aryl, an aralkyl, a heteroaryl or a heterocyclyl, any of which having up to 12 carbon atoms, wherein any heteroaryl or heterocyclyl, comprises at least one heteroatom or heterogoup selected independently from O, N, S, NR¹⁰, SO₂, or CO; 3) CO₂R⁶, COR⁸, NR⁶R⁷ or SO₂R⁸;
p is an integer from 0 to 3, inclusive;
Z is N or CH; or the ZR¹ moiety is S, SO, CO, or SO₂; or the ZR¹R² moiety is C≡CR² or —C(O)Z³R^{f}, wherein R^{f} is an alkyl, a cycloalkyl, or a (cycloalkyl)alkyl, any of which having up to 12 carbon atoms, or hydrogen; and Z³ is O or —NR^{k}, wherein R^{k} is an alkyl, a cycloalkyl, or a (cycloalkyl)alkyl, any of which having up to 12 carbon atoms, or hydrogen;
R¹ and R² are selected independently from: 1) hydrogen; 2) an alkyl having up to 6 carbon atoms; 3) a cycloalkyl having up to 6 carbon atoms; 4) COR⁸; or 5) (CH₂)ₙR⁵ or (CH₂)ₙR^{d}CO₂R^{e}; wherein n, in each occurrence, is 1 or 2; R^{d}, in each occurrence, is selected independently from an alkyl, a cycloalkyl, an aryl, a heterocyclyl, or a heteroaryl, any of which having up to 12 carbon atoms, wherein any heterocyclyl or heteroaryl comprises at least one heteroatom or heterogroup selected independently from O, N, S, NR¹⁰, SO₂, or CO; and R^{e}, in each occurrence, is selected independently from an alkyl or a cycloalkyl, either of which having up to 12 carbon atoms, or hydrogen;
or R¹ and R² together form a substituted or an unsubstituted monocyclic or bicyclic moiety comprising up to 12 carbon atoms, and optionally comprising 1 or 2 heteroatoms or heterogroups selected independently from O, N, or NR¹⁰; wherein any optional substituent on the cyclic moiety selected from: 1) a cycloalkyl having up to 6 carbon atoms; or 2) an alkyl having up to 2 carbon atoms;
R³ is selected from: 1) cyano: 2) a substituted or an unsubstituted alkyl having up to 12 carbon atoms; 3) a substituted or an unsubstituted aryl, or a substituted or an unsubstituted 5-, 6-, or 7-membered heterocyclyl or heteroaryl, comprising 1, 2, or 3 heteroatoms or heterogroups selected independently from O, N, S, NR¹⁰, SO₂, or CO; any of which having up to 12 carbon atoms; or 4) CO₂R⁶, COR⁸, SO₂R⁸, SO₂NR⁶R⁷, CONR⁶R⁷, C(S)NR⁶R⁷, C(═NH)OR⁸, C(S)NHC(O)OR⁸, or C(S)SR⁸; wherein when R³ is an alkyl, an aryl, a heterocyclyl, or a heteroaryl, R³ is optionally substituted with up to three substituents selected independently from R¹¹;
R⁴, in each occurrence, is selected independently from: 1) halogen, hydroxy or cyano; or 2) an alkyl, an alkoxy, a haloalkyl, or a haloalkoxy any of which having up to 4 carbon atoms; and m is an integer from 1-3, inclusive;
R⁵, in each occurrence, is selected independently from: 1) a substituted or an unsubstituted cycloalkyl, heterocyclyl, or heteroaryl, any of which having up to 12 carbon atoms wherein any heterocyclyl or heteroaryl comprises at least one heteroatom or heterogroup selected independently from O, N, S, NR¹⁰, SO₂, or CO;
R⁶ and R⁷, in each occurrence, are selected independently from: 1) hydrogen; 2) an alkyl, a cycloalkyl, or a haloalkyl, any of which having up to 12 carbon atoms; or 3) a substituted or an unsubstituted aryl, aralkyl, heterocyclyl, or heteroaryl, any of which having up to 12 carbon atoms, wherein any heterocyclyl or heteroaryl comprises at least one heteroatom or heterogroup selected independently from O, N, S, NR¹⁰, SO₂, or CO;
R⁸, in each occurrence, is selected independently from: 1) an alkyl, a cycloalkyl, or a haloalkyl, any of which having up to 12 carbon atoms; or 2) a substituted or an unsubstituted aryl heterocyclyl, or heteroaryl, any of which having up to 12 carbon atoms, wherein any heterocyclyl or heteroaryl comprises at least one heteroatom or heterogroup selected independently from O, N, S, NR¹⁰, SO₂, or CO;
R¹⁰, in each occurrence, is selected independently from: 1) hydrogen; or 2) an alkyl, a cycloalkyl, a haloalkyl, an aryl, or an aralkyl, any of which having up to 12 carbon atoms;
R¹¹ is selected independently from:
1) a halogen, a hydroxyl or a cyano,
2) an alkyl, a haloalkyl, an alkoxy, a cycloalkyl, or an alkoxycarbonyl, any of which having up to 12 carbon atoms;
3) a substituted or an unsubstituted heteroaryl or heterocyclyl, any of which having up to 12 carbon atoms, comprises at least one heteroatom or heterogroup selected independently from O, N, S, NR¹⁰, SO₂, or CO, wherein any substituted heteroaryl or heterocycyl is substituted with up to three substituents selected independently from an alkyl having up to 12 carbon atoms or a hydroxyl; or
4) —CO—Z²—R¹³, —CO—R¹², —CO—Z²—(CH₂)ᵣ—CO—Z²—R¹³, —NR¹⁵R¹⁶,
—Z²—CO—(CH₂)ᵣ—Z²—R¹³, —Z²—CO—(CH₂)ᵣ—CO—Z²—R¹³, —O—(CH₂)ᵣ—CO—Z²—R¹³,
—O—(CH₂)ᵣ—R¹⁴, —O—R¹²—(CH₂)ᵣ—R¹³, —O—R¹⁴—CO—O—R¹³, —O—(CH₂)ᵣ—R¹²,
—O—(CH₂)ᵣ—NR′R″, —O—(CH₂)ᵣ—CO₂—(CH₂)ᵣ—R¹³, —O—(CH₂)ᵣ—SR⁸, —O—(CH₂)ᵣ—CO₂—R¹³,
—O—(CH₂)ᵣ—O—(CH₂)ᵣ—OR¹³,
—O—(CH₂)ᵣ—CONR′R″, —O—(CH₂)ᵣ—CONH—(CH₂)ᵣ—OR¹³, —O—(CH₂)ᵣ—SO₂R⁸,
—O—(CH₂)ᵣ—R¹³, —O—(CH₂)ᵣ—OR¹³, —S—(CH₂)ᵣ—CONR′R″, —SO₂—(CH₂)ᵣ—OR¹³, —SO₂—(CH₂)ᵣ—CONR′R″,
—(CH₂)ᵣ—O—CO—R⁸, —(CH₂)ᵣ—R¹², —(CH₂)ᵣ—R¹³, —(CH₂)ᵣ—NH—(CH₂)ᵣ—OR¹³,
—(CH₂)ᵣ—CO—Z²—R¹³, —(CH₂)ᵣ—Z²—R¹³, —(CH₂)ᵣ—NH—CO—Z²—R¹³, or -alkenylene-CO₂(CH₂)ᵣ—R¹³;
r, in each occurrence, is independently 1, 2, or 3,
R¹², in each occurrence, is independently selected from a substituted or an unsubstituted heterocyclyl having up to 12 carbon atoms, comprising at least one heteroatom or heterogroup selected independently from O, N, S, NR¹⁰, SO₂, or CO, wherein any substituted heterocyclyl is substituted with up to three substituents selected independently from an acyl, an alkyl, or an alkoxycarbonyl, any of which having up to 12 carbon atoms, or —COOH;
R¹³, in each occurrence, is independently selected from: 1) hydrogen; or 2) a cycloalkyl, an aryl, a haloalkyl, a heterocyclyl, or an alkyl group optionally substituted with at least one hydroxyl, any of which having up to 12 carbon atoms, wherein any heterocyclyl comprises at least one heteroatom or heterogroup selected independently from O, N, S, NR¹⁰, SO₂, or CO;
R¹⁴, in each occurrence, is independently selected from a heterocyclyl, a cycloalkyl, or an aryl, any of which having up to 12 carbon atoms, wherein any heterocyclyl comprises at least one heteroatom or heterogroup selected independently from O, N, S, NR¹⁰, SO₂, or CO;
Z², in each occurrence, is selected independently from NR¹⁰ or O;
R′ and R″, in each occurrence, are independently selected from hydrogen or an alkyl having up to 12 carbon atoms; and
R¹⁵ and R¹⁶, in each occurrence, are independently selected from: 1) hydrogen; 2) an alkyl having up to 12 carbon atoms; or 3) —(CH₂)ᵣ—O—R¹³, —(CH₂)ᵣ—R¹⁴, —COR¹³, —(CH₂)ᵣ—CO—Z²—R¹³, —CO₂R¹³, —CO₂—(CH₂)ᵣ—R¹³, —CO₂—(CH₂)ᵣ—R¹², —CO₂—(CH₂)ᵣ—CO—Z²—R¹³, —CO₂—(CH₂)ᵣ—OR¹³, —CO—(CH₂)ᵣ—O—(CH₂)ᵣ—O—(CH₂)ᵣ—R¹³, —CO—(CH₂)ᵣ—O(CH₂)ᵣ—OR¹³, or —CO—NH—(CH₁)ᵣ—OR¹³;
or R¹⁵ and R¹⁶ together form a substituted or an unsubstituted cyclic moiety comprising up to 12 carbon atoms, optionally comprising at least one additional heteroatom or heterogroup selected independently from O, N, S, NR¹⁰, SO₂, or CO; wherein any substituted cyclic moiety is substituted with up to three substituents selected independently from: 1) hydroxyl; 2) an alkyl or a heteroaryl, any of which having up to 12 carbon atoms, wherein any heteroaryl comprises at least one heteroatom or heterogroup selected independently from O, N, S, or NR¹⁰, or 3) COOR¹³, —Z²—(CH₂)ᵣ—R¹³, —COR¹³, —CO₂—(CH₂)ᵣ—R¹³, —CO(CH₂)ᵣ—O—R¹³, —(CH₂)ᵣ—CO₂—R¹³, —SO₂R⁸, —SO₂NR′R″, or —NR′R″; and
wherein the —(CH₂)ᵣ— linking moiety, in any occurrence, is optionally substituted with at least one group selected independently from hydroxyl, amino, or an alkyl having up to 3 carbon atoms;
wherein when R¹ and R² do not form a monocyclic or bicyclic moiety, R¹ and R² are optionally substituted with 1 or 2 substituents, and when substituted, the substituents are selected independently from: 1) an alkyl, a cycloalkyl, a haloalkyl, an alkoxy, an aryl, a heteroaryl, or a heterocyclyl, any of which having up to 12 carbon atoms, wherein any heteroaryl or heterocyclyl comprises at least one heteroatom or heterogroup selected independently from O, N, S, NR¹⁰, SO₂, or CO; or 2) halogen, cyano, or hydroxyl;
wherein when R¹ and R² together form a monocyclic or a bicyclic moiety, the monocyclic or bicyclic moiety is optionally substituted with at least one substituent selected independently from: 1) halogen, cyano, or hydroxyl; 2) an alkyl, a haloalkyl, a cycloalkyl, an alkoxy, a cycloalkyl-substituted alkyl, an alkoxyalkyl, a cycloalkoxy, a haloalkoxy, an aryl, an aryloxy, an aralkyl, a heteroaryl, or a heteroaryloxy, any of which having up to 12 carbon atoms, wherein any heteroaryl or heteroaryloxy comprises at least one heteroatom or heterogroup selected independently from O, N, S, or NR¹⁰; or 3) CO₂R⁶, (CH₂)_{q}COR⁸, SO₂R⁸, SO₂NR⁶R⁷, or CONR⁶R⁷; or 4) (CH₂)_{q}CO₂(CH₂)_{q}CH₃, wherein q is selected independently from an integer from 0 to 3, inclusive; and
R⁴, R⁶, R⁷, and R⁸ are optionally substituted with at least one substituent, and when substituted, the substituents are selected independently from: 1) halogen, hydroxy, cyano or NR⁶R⁷; or 2) an alkyl or an alkoxy, any of which having up to 12 carbon atoms; and
R⁵ is optionally substituted with at least one substituent selected independently from: 1) halogen, hydroxy, cyano, or NR⁶R⁷; or 2) an alkyl or an alkoxy, any of which having up to 12 carbon atoms; or 3) (CH₂)ₜOR^{j} or (CH₂)ₜCOOR^{j} wherein t is an integer from 1 to 3, inclusive, and R^{j} is hydrogen or alkyl having up to 12 carbon atoms;
(iii) the CETP inhibitor having formula (II) is: 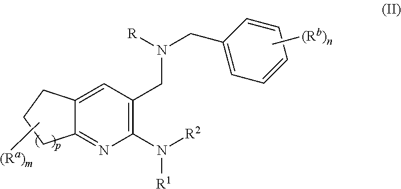
or a stereoisomer thereof or a pharmaceutically acceptable salt thereof; wherein,
R represents 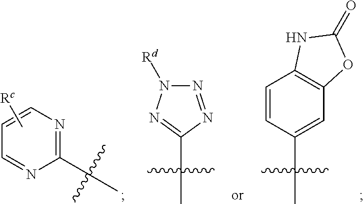
R¹ and R² are independently selected from hydrogen, acyl, haloalkyl, —(CHR^{e})_{q}R³, an optionally substituted group selected from alkyl or cycloalkyl, wherein optional substituent, in each occurrence, is independently selected from halogen, cyano, hydroxyl, an alkyl, a haloalkyl or an alkoxy;
R³ is a group selected from alkoxy, haloalkoxy, cycloalkyl, aryl, heterocyclyl or heteroaryl, wherein R³ is optionally substituted with a group selected from halogen, cyano, hydroxyl, alkyl, haloalkyl or alkoxy;
R^{a}, in each occurrence, is independently selected from halogen, cyano, hydroxy, alkyl, haloalkyl or alkoxy;
R^{b}, in each occurrence, is independently selected from halogen, alkyl, haloalkyl, hydroxy, alkoxy or haloalkoxy;
R^{c} is independently selected from hydrogen, cyano, halogen, —C(═O)—R^{f}, —CONR^{g}R^{h}, —C(═O)—CH═CH—NRⁱR^{j}, an optionally substituted group selected from cycloalkyl, aryl, heteroaryl or heterocyclyl ring, wherein the optional substituent, in each occurrence, is selected independently from hydrogen, halogen, cyano, hydroxyl, alkyl, haloalkyl, alkoxy, alkoxyalkyl or haloalkoxy;
R^{d} is hydrogen or alkyl;
R^{e}, in each occurrence, is independently selected from hydrogen, alkyl or alkoxy;
R^{f} is hydrogen or alkyl;
R^{g} and R^{h} independently represent hydrogen or alkyl;
Rⁱ and Rⱼ independently represent hydrogen or alkyl;
m is 0, 1 or 2;
n is 0, 1, 2 or 3;
p is 1 or 2; and
q is 0, 1, 2, 3, 4 or 5; and
(iv) the CETP inhibitor having formula (III) is: 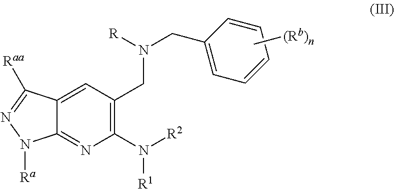
or a stereoisomer thereof or a pharmaceutically acceptable salt thereof;
wherein,
R represents hydrogen or 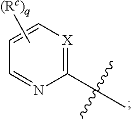
X represents —CH or —N;
R¹ and R² are independently of each other selected from hydrogen, acyl, alkyl or —(CH₂)ₚ-cycloalkyl;
R^{a} and R^{aa} are independently of each other selected from hydrogen or alkyl;
R^{b}, in each occurrence, is independently selected from halogen, alkyl, haloalkyl, hydroxy, alkoxy or haloalkoxy;
R^{c}, in each occurrence, is independently selected from hydrogen, cyano, halogen, alkyl, alkoxy, haloalkoxy, —COOR^{d}, —C(═O)—R^{e}, —CONR^{g}R^{h}, —C(═O)—CH═CH—NRⁱR^{j}, —NHCOR^{t}, an optionally substituted group selected from cycloalkyl, aryl, heteroaryl or heterocycle ring, wherein the optional substituent, in each occurrence, is selected independently from hydrogen, halogen, cyano, hydroxyl, alkyl, haloalkyl, alkoxy, alkoxyalkyl or haloalkoxy;
R^{d}, R^{e}, R^{g}, R^{h}, Rⁱ and R^{j}, in each occurrence, independently of each other represents hydrogen or alkyl;
R^{t} is selected from hydrogen, alkyl or cycloalkyl;
n is 0, 1, 2 or 3;
p is 0, 1, or 2; and
q is 1 or 2.

2. (canceled)

3. The composition according to claim 1, wherein formula (Ia′) is defined as follows 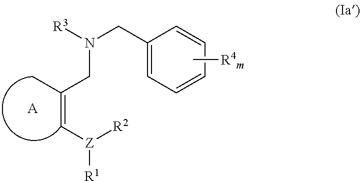
or a stereoisomer thereof or a pharmaceutically acceptable salt thereof,
wherein A-ZR¹R² is: 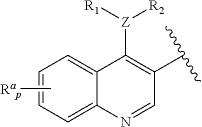
wherein R^{a}, in each occurrence, is selected independently from: 1) a hydrogen, a halogen, a cyano, or a hydroxyl; 2) an alkyl, a haloalkyl, a cycloalkyl, a (cycloalkyl)alkyl, an alkoxy, a cycloalkoxy, a haloalkoxy, an aryl, an aralkyl, a heteroaryl or a heterocyclyl, any of which having up to 12 carbon atoms, wherein any heteroaryl or heterocyclyl, comprises at least one heteroatom or heterogroup selected independently from O, N, S, NR¹⁰, SO₂, or CO; or 3) CO₂R⁶, COR⁸, NR⁶R⁷ or SO₂R⁸;
p is an integer from 0 to 3, inclusive;
Z is N or CH; or the ZR¹ moiety is S, SO, CO, or SO₂; or the ZR¹R² moiety is C≡CR² or —C(O)Z³R^{f}, wherein R^{f} is an alkyl, a cycloalkyl, or a (cycloalkyl)alkyl, any of which having up to 12 carbon atoms, or hydrogen; and Z³ is O or NR^{k}, wherein R^{k} is an alkyl, a cycloalkyl, or a (cycloalkyl)alkyl, any of which having up to 12 carbon atoms, or hydrogen;
R¹ and R² are selected independently from: 1) hydrogen; 2) an alkyl having up to 6 carbon atoms; 3) a cycloalkyl having up to 6 carbon atoms; 4) COR⁸; or 5) (CH₂)₁R⁵ or (CH₂)R^{d}CO₂R^{e}; wherein n, in each occurrence, is 1 or 2; R^{d}, in each occurrence, is selected independently from an alkyl, a cycloalkyl, an aryl, a heterocyclyl, or a heteroaryl, any of which having up to 12 carbon atoms, wherein any heterocyclyl or heteroaryl comprises at least one heteroatom or heterogroup selected independently from O, N, S, NR¹⁰, SO₂, or CO; and R^{e}, in each occurrence, is selected independently from an alkyl or a cycloalkyl, either of which having up to 12 carbon atoms, or hydrogen;
or R¹ and R² together form a substituted or an unsubstituted monocyclic or bicyclic moiety comprising up to 12 carbon atoms, and optionally comprising 1 or 2 heteroatoms or heterogroups selected independently from O, N, or NR¹⁰; wherein any optional substituent on the cyclic moiety selected from: 1) a cycloalkyl having up to 6 carbon atoms; or 2) an alkyl having up to 2 carbon atoms;
R³ is selected from: 1) cyano; 2) a substituted or an unsubstituted alkyl having up to 12 carbon atoms; 3) a substituted or an unsubstituted aryl, or a substituted or an unsubstituted 5-, 6-, or 7-membered heterocyclyl or heteroaryl, comprising 1, 2, or 3 heteroatoms or heterogroups selected independently from O, N, S, NR¹⁰, SO₂, or CO; any of which having up to 12 carbon atoms; or 4) CO₂R⁶, COR⁸, SO₂R⁸, SO₂NR⁶R⁷, CONR⁶R⁷, C(S)NR⁶R⁷, C(═NH)OR⁸, C(S)NHC(O)OR⁸, or C(S)SR⁸; wherein when R³ is an alkyl, an aryl, a heterocyclyl, or a heteroaryl, R³ is optionally substituted with up to three substituents selected independently from R¹¹;
R⁴, in each occurrence, is selected independently from: 1) halogen, hydroxy or cyano; or 2) an alkyl, an alkoxy, a haloalkyl, or a haloalkoxy any of which having up to 4 carbon atoms; and m is an integer from 1-3, inclusive;
R⁵, in each occurrence, is selected independently from: 1) a substituted or an unsubstituted cycloalkyl, heterocyclyl, or heteroaryl, any of which having up to 12 carbon atoms, wherein any heterocyclyl or heteroaryl comprises at least one heteroatom or heterogroup selected independently from O, N, S, NR¹⁰, SO₂, or CO;
R⁶ and R⁷, in each occurrence, are selected independently from: 1) hydrogen; 2) an alkyl, a cycloalkyl, or a haloalkyl, any of which having up to 12 carbon atoms; or 3) a substituted or an unsubstituted aryl, aralkyl, heterocyclyl, or heteroaryl, any of which having up to 12 carbon atoms, wherein any heterocyclyl or heteroaryl comprises at least one heteroatom or heterogroup selected independently from O, N, S, NR¹⁰, SO₂, or CO;
R⁸, in each occurrence, is selected independently from: 1) an alkyl, a cycloalkyl, or a haloalkyl, any of which having up to 12 carbon atoms; or 2) a substituted or an unsubstituted aryl, heterocyclyl, or heteroaryl, any of which having up to 12 carbon atoms, wherein any heterocyclyl or heteroaryl comprises at least one heteroatom or heterogroup selected independently from O, N, S, NR¹⁰, SO₂, or CO;
R¹⁰, in each occurrence, is selected independently from: 1) hydrogen; or 2) an alkyl, a cycloalkyl, a haloalkyl, an aryl, or an aralkyl, any of which having up to 12 carbon atoms;
R¹¹ is selected independently from:
1) a halogen, a hydroxyl or a cyano;
2) an alkyl, a haloalkyl, an alkoxy, a cycloalkyl, or an alkoxycarbonyl, any of which having up to 12 carbon atoms;
3) a substituted or an unsubstituted heteroaryl or heterocyclyl, any of which having up to 12 carbon atoms, comprises at least one heteroatom or heterogroup selected independently from O, N, S, NR¹⁰, SO₂, or CO, wherein any substituted heteroaryl or heterocyclyl is substituted with up to three substituents selected independently from an alkyl having up to 12 carbon atoms or a hydroxyl; or
4) —CO—Z²—R¹³, —CO—R¹², —CO—Z²—(CH₂)ᵣ—CO—Z²—R¹³, —NR¹⁵R¹⁶,
—Z²—CO—(CH₂)ᵣ—Z²—R¹³, —Z²—CO—(CH₂)ᵣ—CO—Z²—R¹³, —O—(CH₂)ᵣ—CO—Z²—R¹³,
—O—(CH₂)ᵣ—R¹⁴, —O—R¹²—(CH₂)ᵣ—R¹³, —O—R¹⁴—CO—O—R¹³, —O—(CH₂)ᵣ—R¹²,
—O—(CH₂)ᵣ—NR′R″, —O—(CH₂)ᵣ—CO₂—(CH₂)ᵣ—R¹³, —O—(CH₂)ᵣ—SR⁸, —O—(CH₂)ᵣ—CO₂—R¹³,
—O—(CH₂), —O—(CH₂)ᵣ—OR¹³,
—O—(CH₂)ᵣ—CONR′R″, —O—(CH₂)ᵣ—CONH—(CH₂)ᵣ—OR¹³, —O—(CH₂)ᵣ—SO₂R⁸,
—O—(CH₂)ᵣ—R¹³, —O—(CH₂)ᵣ—OR¹³, —S—(CH₂)ᵣ—CONR′R″, —SO₂—(CH₂)ᵣ—OR¹³, —SO₂—(CH₂)ᵣ—CONR′R″,
—(CH₂)ᵣ—O—CO—R⁸, —(CH₂)ᵣ—R¹², —(CH₂)ᵣ—R¹³, —(CH₂)ᵣ—NH—(CH₂)ᵣ—OR¹³,
—(CH₂)ᵣ—CO—Z²—R¹³, —(CH₂)ᵣ—Z²—R¹³, —(CH₂)ᵣ—NH—CO—Z²—R¹³, or -alkenylene-CO₂—(CH₂)ᵣ—R¹³;
r, in each occurrence, is independently 1, 2, or 3;
R¹², in each occurrence, is independently selected from a substituted or an unsubstituted heterocyclyl having up to 12 carbon atoms, comprising at least one heteroatom or heterogroup selected independently from O, N, S, NR¹⁰, SO₂, or CO, wherein any substituted heterocyclyl is substituted with up to three substituents selected independently from an acyl, an alkyl, or an alkoxycarbonyl, any of which having up to 12 carbon atoms, or —COOH;
R¹³, in each occurrence, is independently selected from: 1) hydrogen; or 2) a cycloalkyl, an aryl, a haloalkyl, a heterocyclyl, or an alkyl group optionally substituted with at least one hydroxyl, any of which having up to 12 carbon atoms, wherein any heterocyclyl comprises at least one heteroatom or heterogroup selected independently from O, N, S, NR¹⁰, SO₂, or CO;
R¹⁴, in each occurrence, is independently selected from a heterocyclyl, a cycloalkyl, or an aryl, any of which having up to 12 carbon atoms, wherein any heterocyclyl comprises at least one heteroatom or heterogroup selected independently from O, N, S, NR¹⁰, SO₂, or CO;
Z², in each occurrence, is selected independently from NR¹⁰ or O;
R′ and R″, in each occurrence, are independently selected from hydrogen or an alkyl having up to 12 carbon atoms; and
R¹⁵ and R¹⁶, in each occurrence, are independently selected from: 1) hydrogen; 2) an alkyl having up to 12 carbon atoms; or 3) —(CH₂)ᵣ—O—R¹³, —(CH₂)ᵣ—R¹⁴, —COR¹³, —(CH₂)ᵣ—CO—Z²—R¹³, —CO₂R¹³, —CO₂—(CH₂)ᵣ—R¹³, —CO₂—(CH₂)ᵣ—R¹², —CO₂—(CH₂)ᵣ—CO—Z²—R¹³, —CO₂—(CH₂)ᵣ—OR¹³, —CO—(CH₂)ᵣ—O—(CH₂)ᵣ—O—(CH₂)ᵣ—R¹³, —CO—(CH₂)ᵣ—O(CH₂)ᵣ—OR¹³, or —CO—NH—(CH₂)ᵣ—OR¹³;
or R¹⁵ and R¹⁶ together form a substituted or an unsubstituted cyclic moiety comprising up to 12 carbon atoms, optionally comprising at least one additional heteroatom or heterogroup selected independently from O, N, S, NR¹⁰, SO₂, or CO; wherein any substituted cyclic moiety is substituted with up to three substituents selected independently from: 1) hydroxyl; 2) an alkyl or a heteroaryl, any of which having up to 12 carbon atoms, wherein any heteroaryl comprises at least one heteroatom or heterogroup selected independently from O, N, S, or NR¹⁰; or 3) COOR¹³, —Z²—(CH₂)ᵣ—R¹³, —COR¹³, —CO₂—(CH₂)ᵣ—R¹³, —CO(CH₂)ᵣ—O—R¹³, —(CH₂)ᵣ—CO₂—R¹³, —SO₂R⁸, —SO₂NR′R″, or —NR′R″; and
wherein the —(CH₂)ᵣ— linking moiety, in any occurrence, is optionally substituted with at least one group selected independently from hydroxyl, amino, or an alkyl having up to 3 carbon atoms;
wherein when R¹ and R² do not form a monocyclic or bicyclic moiety, R¹ and R² are optionally substituted with 1 or 2 substituents, and when substituted, the substituents are selected independently from: 1) an alkyl, a cycloalkyl, a haloalkyl, an alkoxy, an aryl, a heteroaryl, or a heterocyclyl, any of which having up to 12 carbon atoms, wherein any heteroaryl or heterocyclyl comprises at least one heteroatom or heterogroup selected independently from O, N, S, NR¹⁰, SO₂, or CO; or 2) halogen, cyano, or hydroxyl;
wherein when R¹ and R² together form a monocyclic or a bicyclic moiety, the monocyclic or bicyclic moiety is optionally substituted with at least one substituent selected independently from: 1) halogen, cyano, or hydroxyl; 2) an alkyl, a haloalkyl, a cycloalkyl, an alkoxy, a cycloalkyl-substituted alkyl, an alkoxyalkyl, a cycloalkoxy, a haloalkoxy, an aryl, an aryloxy, an aralkyl, a heteroaryl or a heteroaryloxy, any of which having up to 12 carbon atoms, wherein any heteroaryl or heteroaryloxy comprises at least one heteroatom or heterogroup selected independently from O, N, S, or NR¹⁰; CO₂R⁶, (CH₂)_{q}COR⁸, SO₂R⁸, SO₂NR⁶R⁷, or CONR⁶R⁷; or 4) (CH₂)CO₂(CH₂)CH₃, wherein q is selected independently from an integer from 0 to 3, inclusive; and
R⁴, R⁶, R⁷, and R⁸ are optionally substituted with at least one substituent, and when substituted, the substituents are selected independently from: 1) halogen, hydroxy, cyano, or NR⁶R⁷; or 2) an alkyl or an alkoxy, any of which having up to 12 carbon atoms; and
R⁵ is optionally substituted with at least one substituent selected independently from: 1) halogen, hydroxy, cyano, or NR⁶R⁷; or 2) an alkyl or an alkoxy, any of which having up to 12 carbon atoms; or 3) (CH₂)ₜOR^{j} or (CH₂)ₜCOOR^{j} wherein t is an integer from 1 to 3, inclusive, and R^{j} is hydrogen or alkyl having up to 12 carbon atoms.

4. The composition according to claim 1, wherein formula (II) is defined as follows 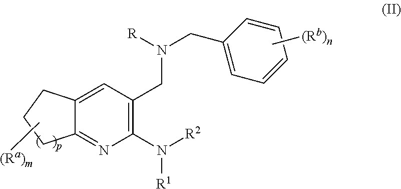
or a stereoisomer thereof or a pharmaceutically acceptable salt thereof;
wherein,
R represents 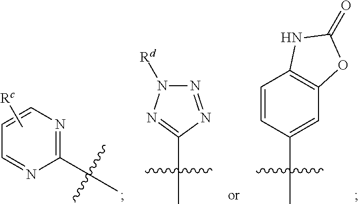
R¹ and R² are independently selected from hydrogen, acyl, haloalkyl, —(CHR^{e})_{q}R³, an optionally substituted group selected from alkyl or cycloalkyl, wherein optional substituent, in each occurrence, is independently selected from halogen, cyano, hydroxyl, an alkyl, a haloalkyl or an alkoxy;
R³ is a group selected from alkoxy, haloalkoxy, cycloalkyl, aryl, heterocyclyl or heteroaryl, wherein R³ is optionally substituted with a group selected from halogen, cyano, hydroxyl, alkyl, haloalkyl or alkoxy;
R^{a}, in each occurrence, is independently selected from halogen, cyano, hydroxy, alkyl, haloalkyl or alkoxy;
R^{b}, in each occurrence, is independently selected from halogen, alkyl, haloalkyl, hydroxy, alkoxy or haloalkoxy;
R^{c} is independently selected from hydrogen, cyano, halogen, —C(═O)—R^{f}, —CONR^{g}R^{h}, —C(═O)—CH═CH—NRⁱR^{j}, an optionally substituted group selected from cycloalkyl, aryl, heteroaryl or heterocyclyl ring, wherein the optional substituent, in each occurrence, is selected independently from hydrogen, halogen, cyano, hydroxyl, alkyl, haloalkyl, alkoxy, alkoxyalkyl or haloalkoxy;
R^{d} is hydrogen or alkyl;
R^{e}, in each occurrence, is independently selected from hydrogen, alkyl or alkoxy;
R^{f} is hydrogen or alkyl;
R^{g} and R^{h} independently represent hydrogen or alkyl;
Rⁱ and R^{j} independently represent hydrogen or alkyl;
m is 0, 1 or 2;
n is 0, 1, 2 or 3;
p is 1 or 2; and
q is 0, 1, 2, 3, 4 or 5.

5. The composition according to claim 1, wherein formula (III) is defined as follows 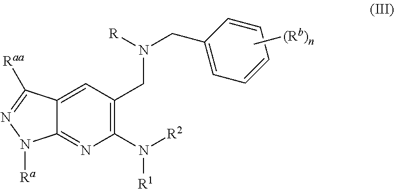
or a stereoisomer thereof or a pharmaceutically acceptable salt thereof,
wherein,
R represents hydrogen or 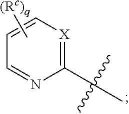
X represents —CH or —N;
R¹ and R² are independently of each other selected from hydrogen, acyl, alkyl or —(CH₂)ₚ-cycloalkyl;
R^{a} and R^{aa} are independently of each other selected from hydrogen or alkyl;
R^{b}, in each occurrence, is independently selected from halogen, alkyl, haloalkyl, hydroxy, alkoxy or haloalkoxy;
R^{c}, in each occurrence, is independently selected from hydrogen, cyano, halogen, alkyl, alkoxy, haloalkoxy, —COOR^{d}, —C(═O)—R^{e}, —CONR^{g}R^{h}, —C(═O)—CH═CH—NRⁱR^{j}, —NHCOR^{t}, an optionally substituted group selected from cycloalkyl, aryl, heteroaryl or heterocycle ring, wherein the optional substituent, in each occurrence, is selected independently from hydrogen, halogen, cyano, hydroxyl, alkyl, haloalkyl, alkoxy, alkoxyalkyl or haloalkoxy;
R^{d}, R^{e}, R^{g}, R^{h}, Rⁱ and R^{j}, in each occurrence, independently of each other represents hydrogen or alkyl;
R^{t} is selected from hydrogen, alkyl or cycloalkyl;
n is 0, 1, 2 or 3;
p is 0, 1, or 2; and
q is 1 or 2.

6. The composition according to claim 1, wherein the surfactant is hydrophilic, hydrophobic or mixtures thereof.

7. The composition according to claim 1, wherein the surfactant comprises from about 1% to about 90% weight of the composition.

8. The composition according to claim 6, wherein the hydrophilic surfactant is anionic, cationic, zwitterionic or non-ionic.

9. The composition according to claim 8, wherein the anionic surfactant is selected from group consisting of fatty acid salts, bile salts, phospholipids, phosphoric acid esters, carboxylates, acyl lactylates, alginate salts, sulfates and sulfonates, cationic surfactants and combinations thereof.

10-17. (canceled)

18. The composition according to claim 8, wherein the cationic surfactant is selected from group comprising lauroyl carnitine, palmitoyl carnitine, myristoyl carnitine, hexadecyl triammonium bromide, decyl trimethyl ammonium bromide, cetyl trimethyl ammonium bromide, dodecyl ammonium chloride, alkyl benzyldimethylammonium salts, diisobutyl phenoxyethoxydimethyl benzylammonium salts, alkylpyridinium salts, betaines (trialkylglycine): lauryl betaine (N-lauryl, N,N-dimethylglycine), ethoxylated amines: polyoxyethylene-15 coconut amine and combinations thereof.

19. The composition according to claim 8, wherein the non-ionic surfactant is selected from group comprising polyethoxylated fatty Acids, PEG fatty acid diesters, PEG-fatty acid mono- and di-ester mixtures, polyethylene glycol glycerol fatty acid esters, alcohol-oil transesterification products, polyglycerized fatty acids, sterol and sterol derivatives, polyethylene glycol sorbitan fatty acid esters, polyethylene glycol alkyl ethers, sugar ethers, polyethylene glycol alkyl phenols, polyoxyethylene polyoxypropylene block copolymers and combinations thereof.

20. The composition according to claim 6, wherein the hydrophobic surfactants comprise reaction mixtures of polyols and fatty acids, glycerides, vegetable oils, hydrogenated vegetable oils and sterols; polyethoxylated fatty acids, PEG-fatty acid diesters, alcohol-oil transesterification products, polyglycerized fatty acids, propylene glycol fatty acid esters, mixtures of propylene glycol esters-glycerol esters, mono- and diglycerides, sterol and sterol derivatives; polyethylene glycol sorbitan fatty acid esters, polyethylene glycol alkyl ethers, sugar ethers, polyoxyethylene-polyoxypropylene block copolymers, sorbitan fatty acid esters, lower alcohol fatty acid esters surfactants and combinations thereof.

21. The composition according to claim 1, wherein the carrier material comprises from about 1% to about 90% weight of the composition.

22. The composition according to claim 1, wherein the carrier material is a triglyceride or digestible oil comprising aceituno oil, almond oil, araehis oil, babassu oil, blackcurrant seed oil, borage oil, buffalo ground oil, candlenut oil, canola oil, castor oil, chinese vegetable tallow oil, cocoa butter coconut oil, coffee seed oil, corn oil, cottonseed oil, crambe oil, cuphea species oil, evening primrose oil, grapeseed oil, groundnut oil, hemp seed oil, illipe butter, kapok seed oil, linseed oil, menhaden oil, mowrah butter mustard seed oil, oiticica oil, olive oil, palm oil, palm kernel oil, peanut oil, poppy seed oil, rapeseed oil, rice bran oil, safflower oil, sal fat sesame oil, shark liver oil, shea nut oil soybean oil, stillingia oil, sunflower oil, tall oil, tea sead oil, tobacco seed oil, tung oil (china wood oil), ucuhuba vernonia oil, wheat germ oil, hydrogenated castor oil (castor wax), hydrogenated coconut oil, hydrogenated cottonseed oil, hydrogenated palm oil, hydrogenated soybean oil, hydrogenated vegetable oil, hydrogenated cottonseed and castor oil, partially hydrogenated soybean oil, partially soy and cottonseed oil, glyceryl tributyrate, glyceryl tricaproate, glyceryl tricaprylate, glyceryl tricaprate (CAPTEX® 1000), glyceryl trundecanoate (CAPTEX® 8227), glyceryl trilaurate, glyceryl trimyristate, glyceryl tripalmitate, glyceryl tristearate, glyceryl triarcidate, glyceryl trimyristoleate, glyceryl tripalmitoleate, glyceryl trioleate, glyceryl trilinoleate, glyceryl trilinolenate, glyceryl tricaprylate/caprate (CAPTEX® 300, CAPTEX® 355, MIGLYOL® 810, MIGLYOL® 812), glyceryl tricaprylate/caprate/laurate (CAPTEX® 350), glyceryl tricaprylate/caprate/linoleate (CAPTEX® 810, MIGLYOL® 818), glyceryl tricaprylate/caprate/stearate, glyceryl tricaprylate/laurate/stearate, glyceryl 1,2-caprylate-3-linoleate, glyceryl 1,2-caprate-3-stearate, glyceryl 1,2-laurate-3 myristate, glyceryl 1,2-myristate-3-laurate, glyceryl 1,3-palmitate-2-butyrate, glyceryl 1,3-stearate-2-caprate, glyceryl 1,2-linoleate-3-caprylate, polyglycolized glycerides (GELUCIRE® 44/14, GELUCIRE® 50/13 and GELUCIRE® 53/10), linoleic glycerides (MAISINE™ 35-1), and caprylic/capric glycerides (IMWITOR® 742), fractionated triglycerides, modified triglycerides, synthetic triglycerides and combinations thereof.

23-24. (canceled)

25. The composition according to claim 24, wherein the composition is in the form of a solution, suspension, emulsion or a pre-concentrate.

26. (canceled)

27. A composition comprising a CETP inhibitor of formula (I), (Ia′), (II) or (III) and at least one surfactant, wherein said composition releases not less than 20% at a period of 15 minutes in 900 ml of 0.01N HCl, when tested in a USP Type 2 apparatus at 50 rpm and 37° C.

28. (canceled)

29. The composition according to claim 27, wherein the composition releases not less than 75% at a period of 60 minutes.

30-31. (canceled)

32. The composition according to claim 1, wherein the CETP inhibitor is selected from a group consisting of: 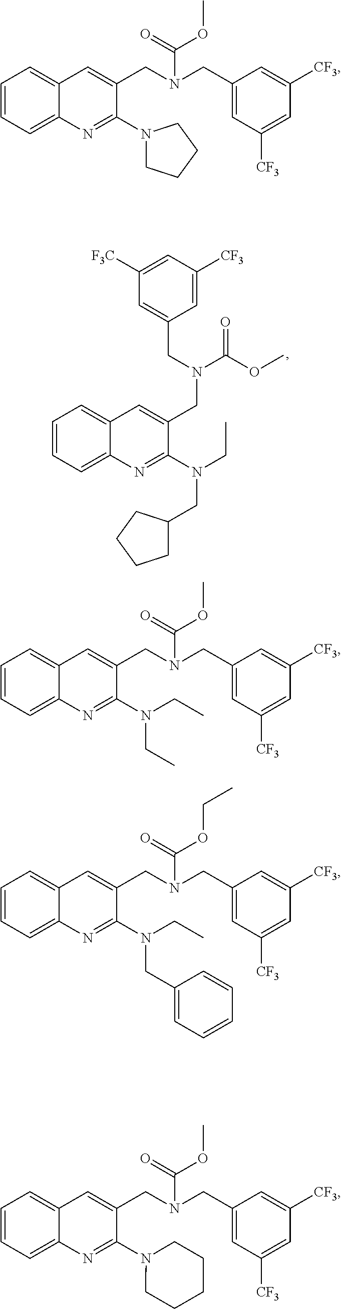 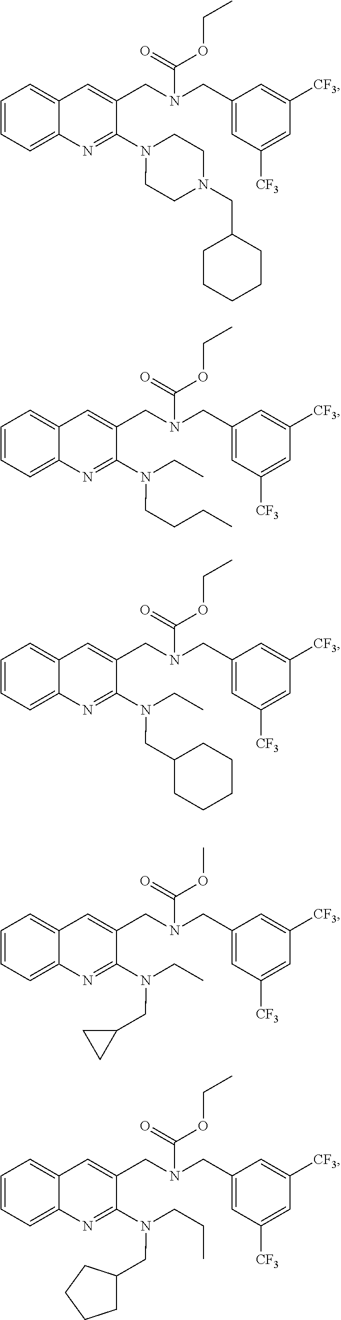 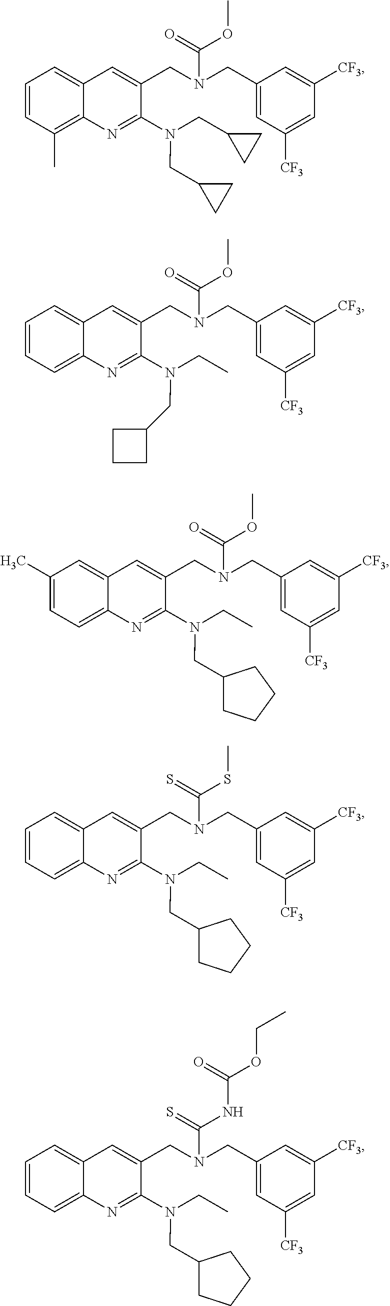 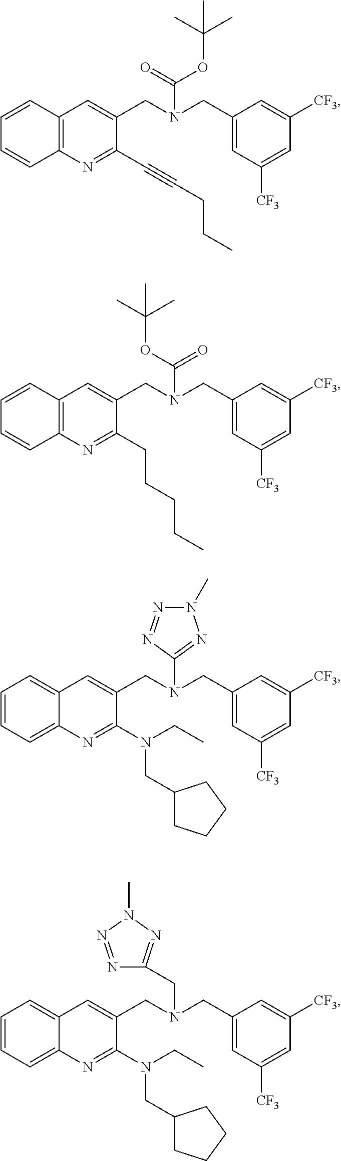 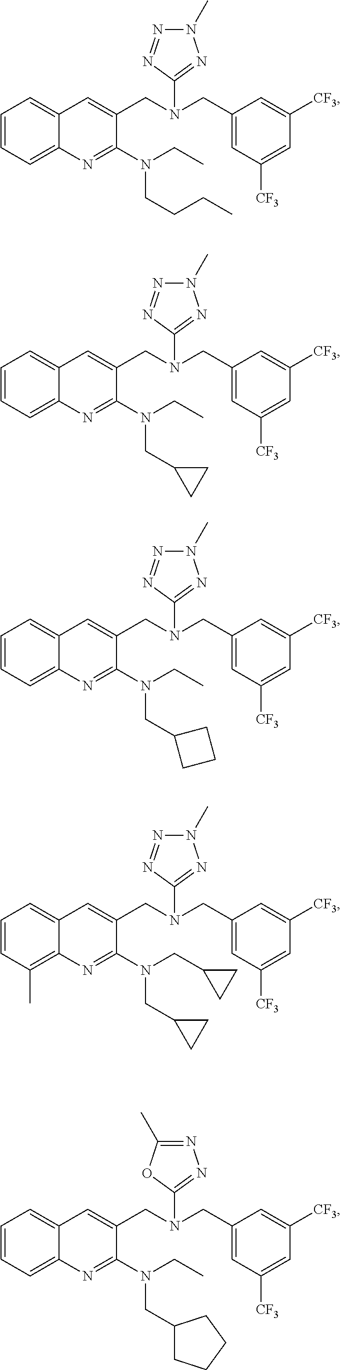 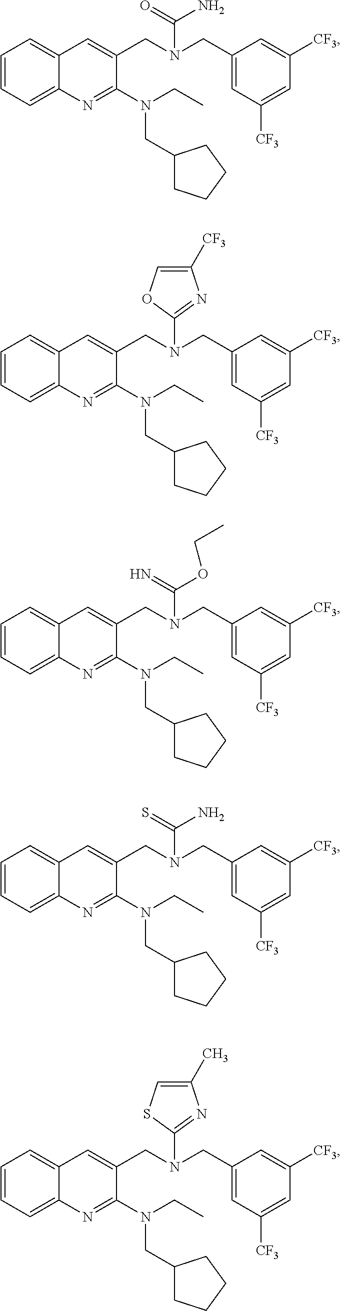 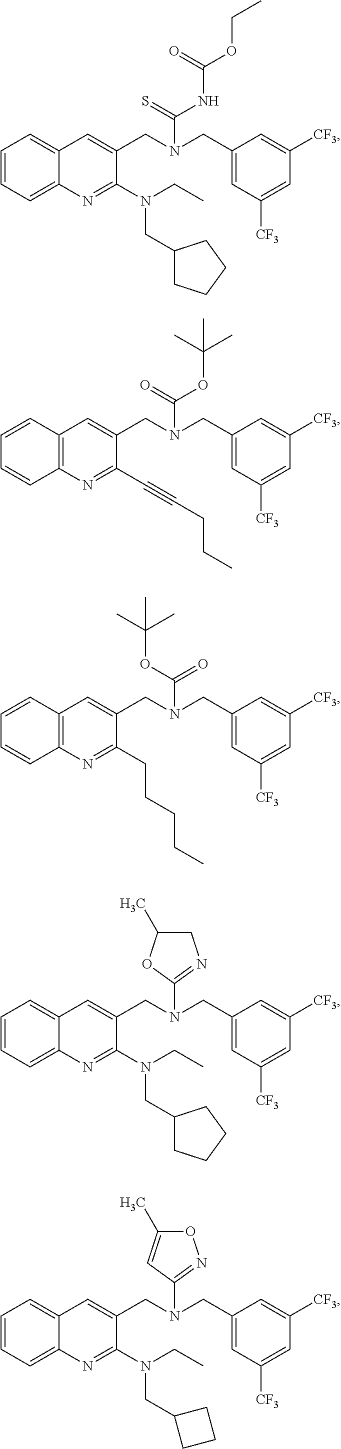 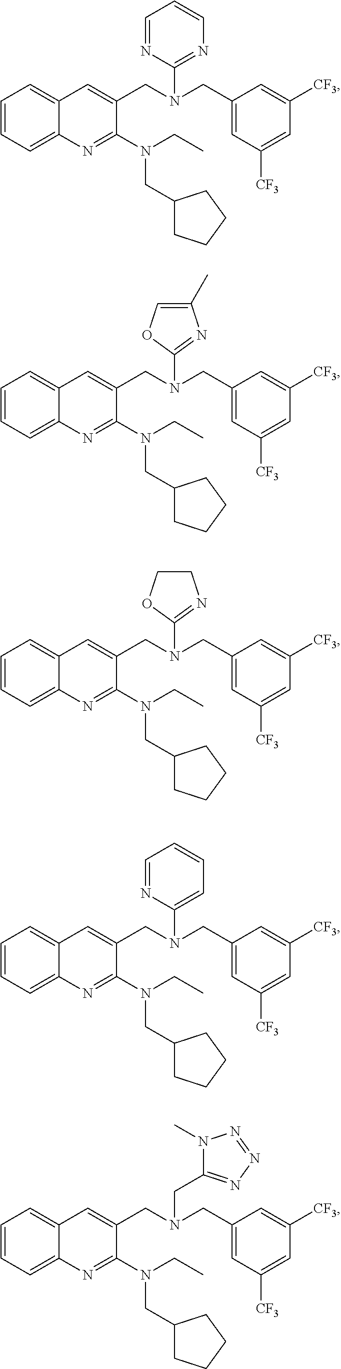 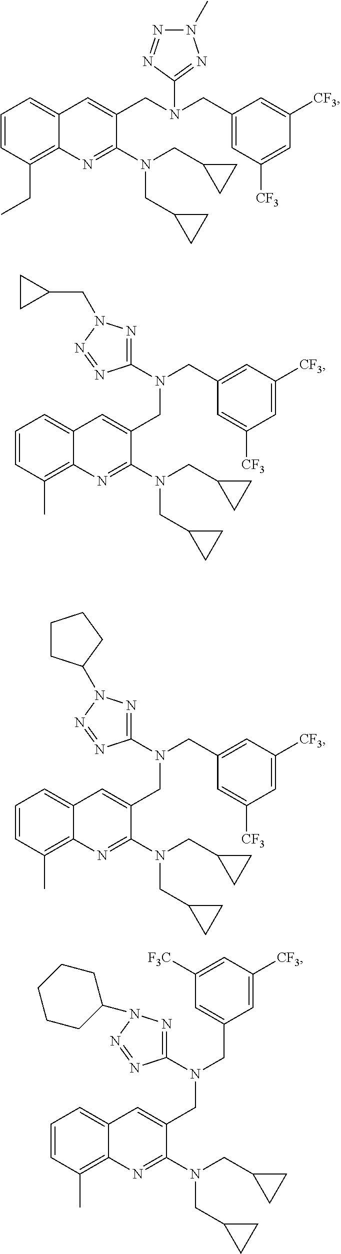 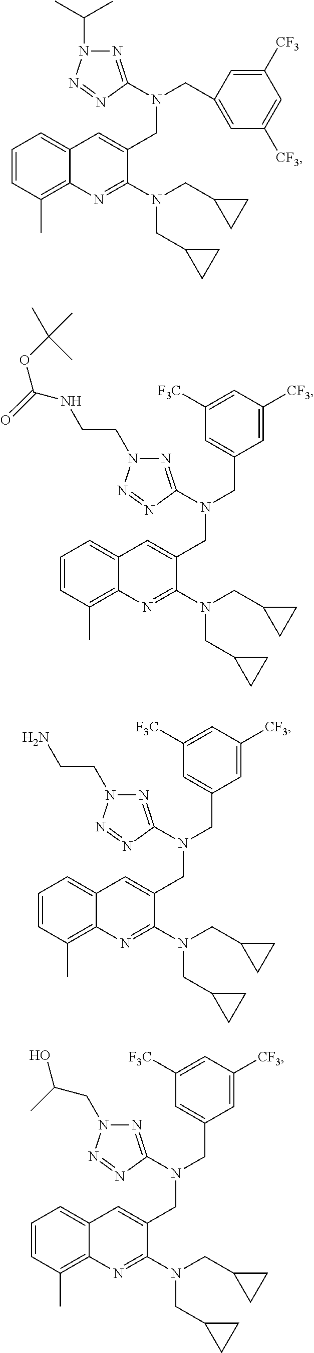 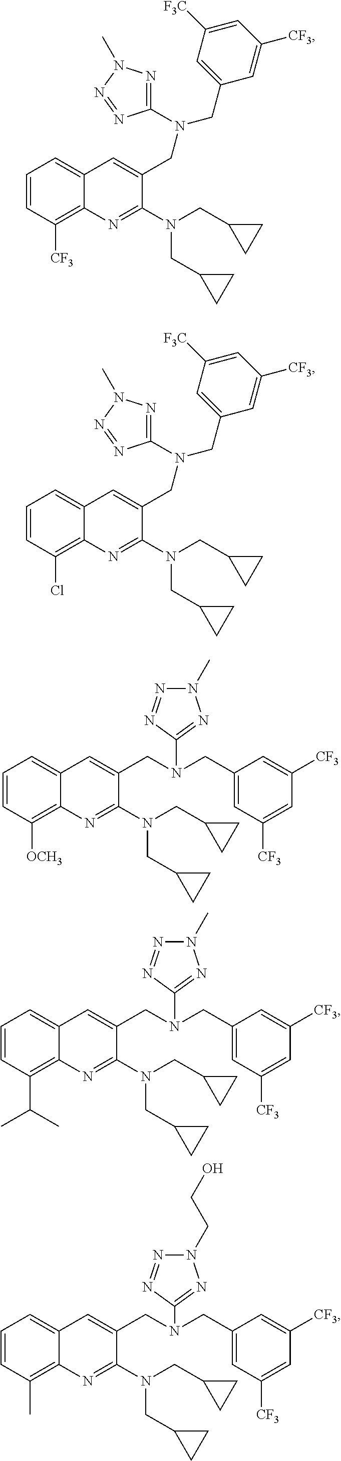 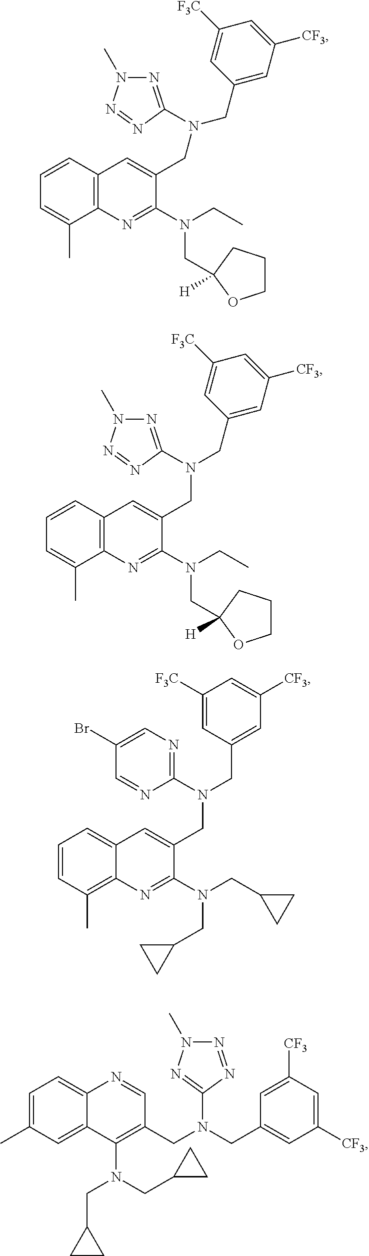 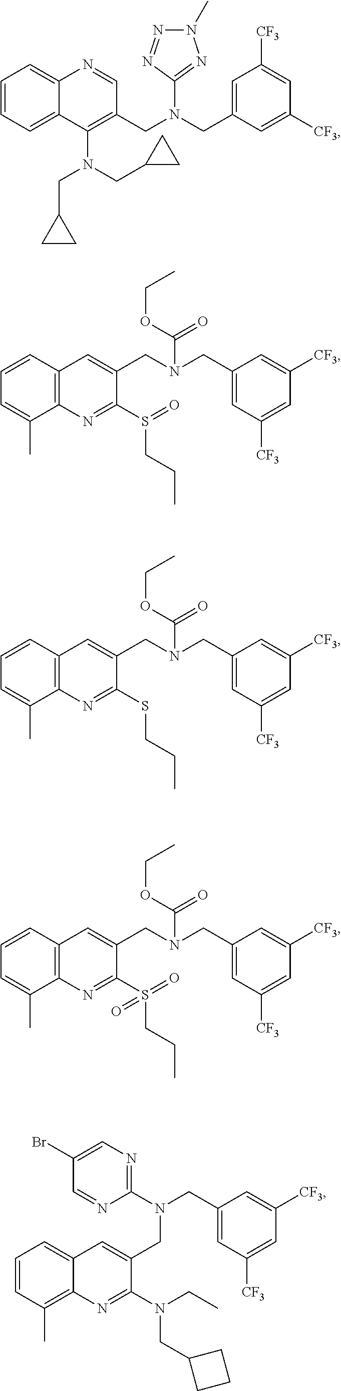 [SMILES: Cn1nnc(N(Cc2cc(C(F)(F)F)cc(C(F)(F)F)c2)Cc2cnc3ccccc3c2N(CC2CC2)CC2CC2)n1 ; CCCS(=O)c1nc2c(C)cccc2cc1CN(Cc1cc(C(F)(F)F)cc(C(F)(F)F)c1)C(=O)OCC ; CCCSc1nc2c(C)cccc2cc1CN(Cc1cc(C(F)(F)F)cc(C(F)(F)F)c1)C(=O)OCC ; CCCS(=O)(=O)c1nc2c(C)cccc2cc1CN(Cc1cc(C(F)(F)F)cc(C(F)(F)F)c1)C(=O)OCC ; CCN(CC1CCC1)c1nc2c(C)cccc2cc1CN(Cc1cc(C(F)(F)F)cc(C(F)(F)F)c1)c1ncc(Br)cn1] 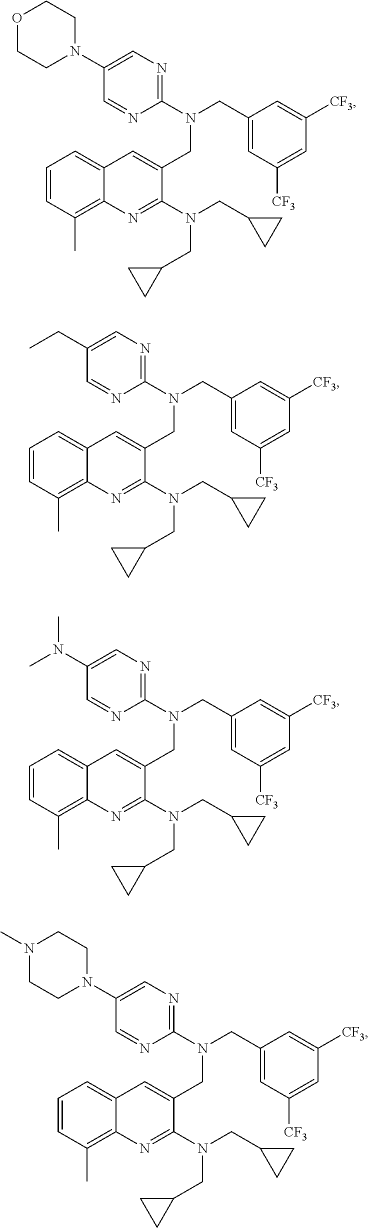 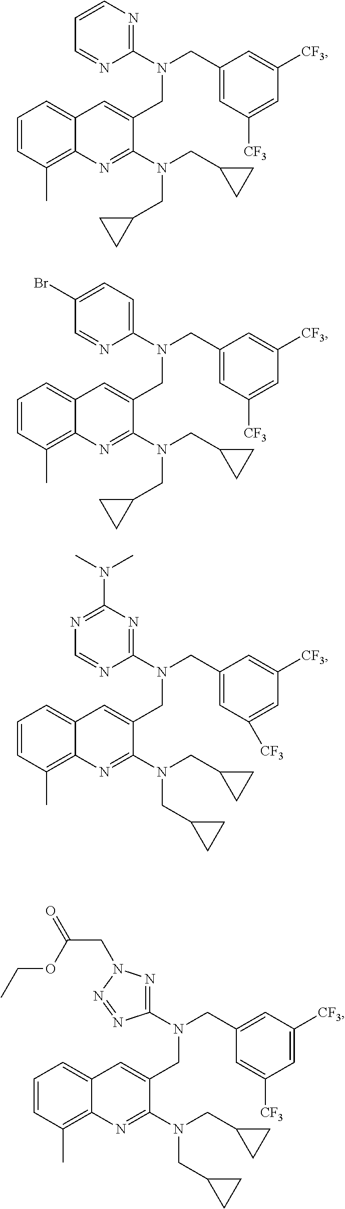 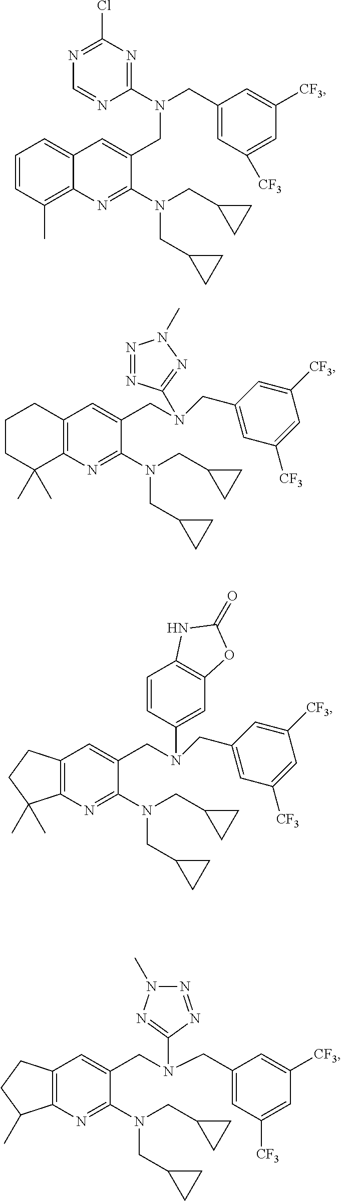 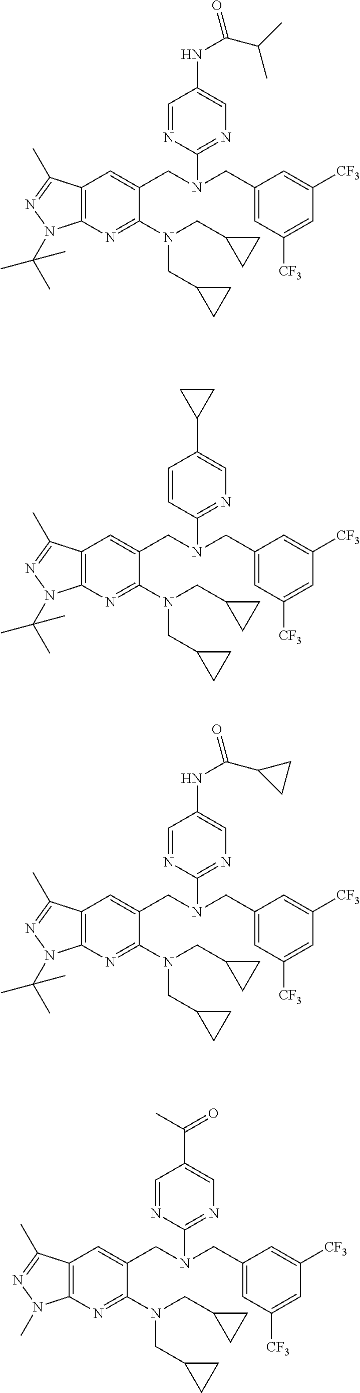 [SMILES: Cc1nn(C(C)(C)C)c2nc(N(CC3CC3)CC3CC3)c(CN(Cc3cc(C(F)(F)F)cc(C(F)(F)F)c3)c3ncc(NC(=O)C(C)C)cn3)cc12 ; Cc1nn(C(C)(C)C)c2nc(N(CC3CC3)CC3CC3)c(CN(Cc3cc(C(F)(F)F)cc(C(F)(F)F)c3)c3ccc(C4CC4)cn3)cc12 ; Cc1nn(C(C)(C)C)c2nc(N(CC3CC3)CC3CC3)c(CN(Cc3cc(C(F)(F)F)cc(C(F)(F)F)c3)c3ncc(NC(=O)C4CC4)cn3)cc12 ; CC(=O)c1cnc(N(Cc2cc(C(F)(F)F)cc(C(F)(F)F)c2)Cc2cc3c(C)nn(C)c3nc2N(CC2CC2)CC2CC2)nc1] 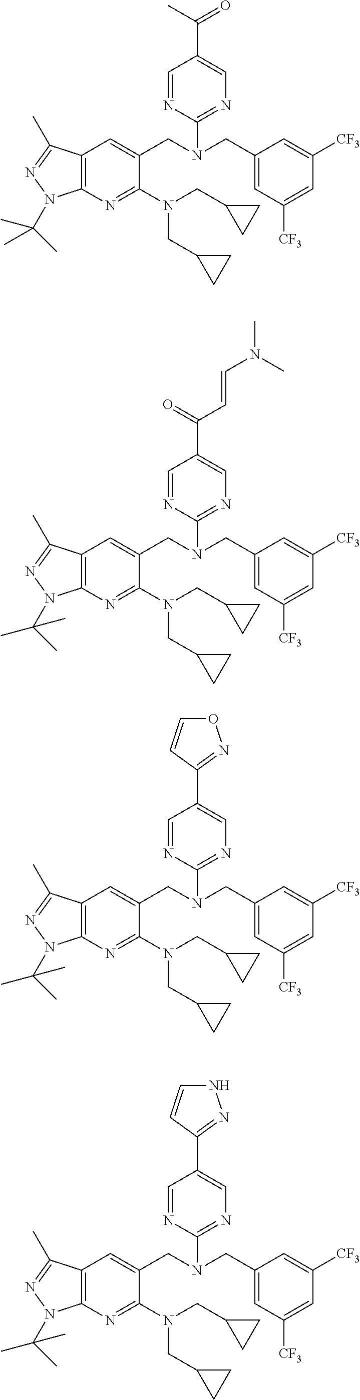 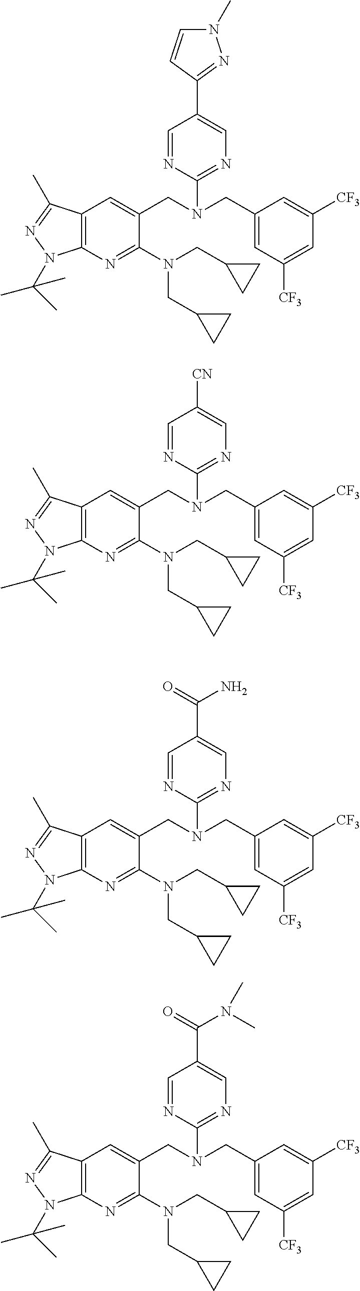 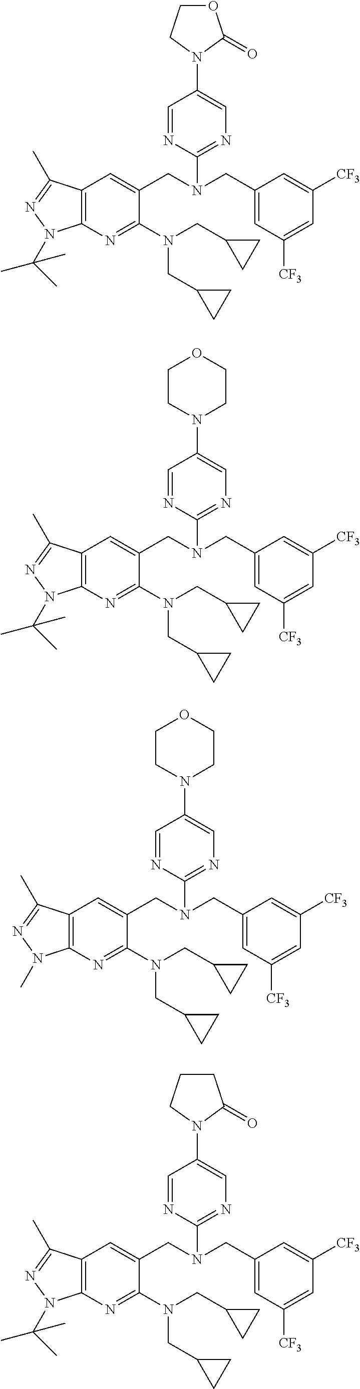 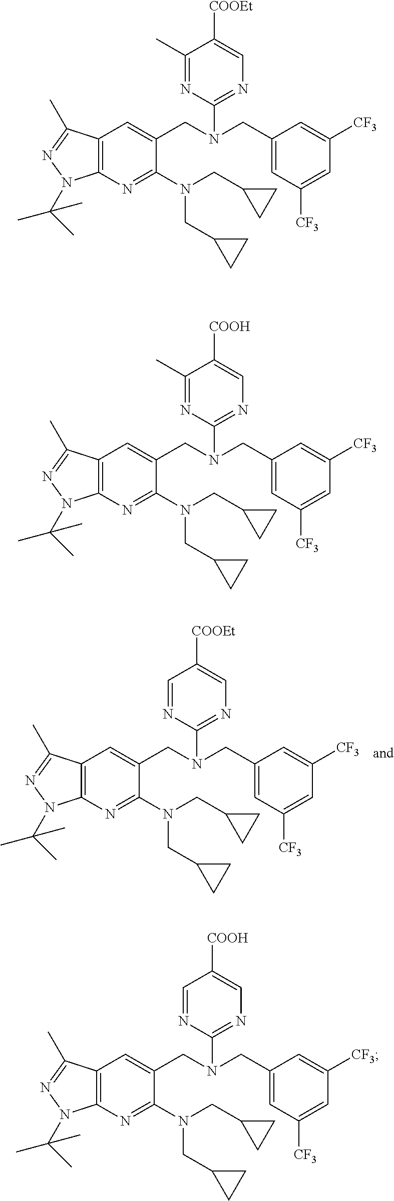
or a stereoisomer thereof or a pharmaceutically acceptable salt thereof.

33. The composition according to claim 1, wherein the CETP inhibitor is selected from a group consisting of: 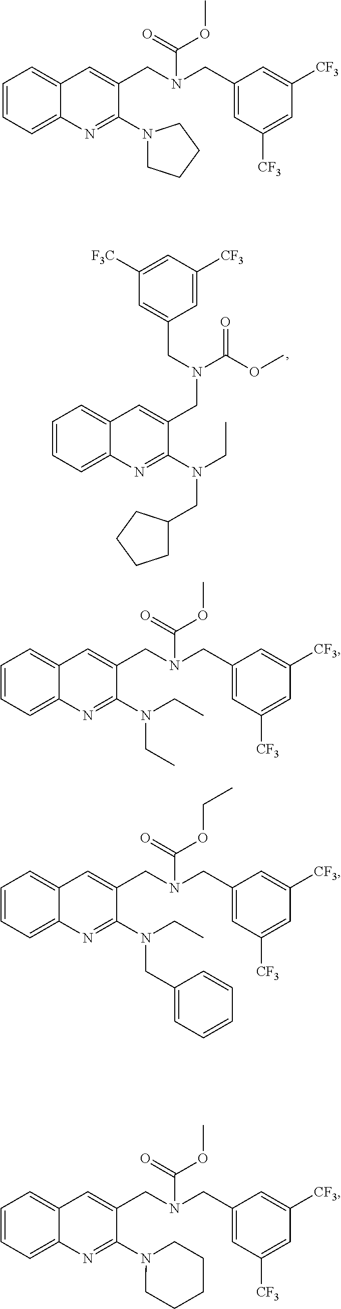 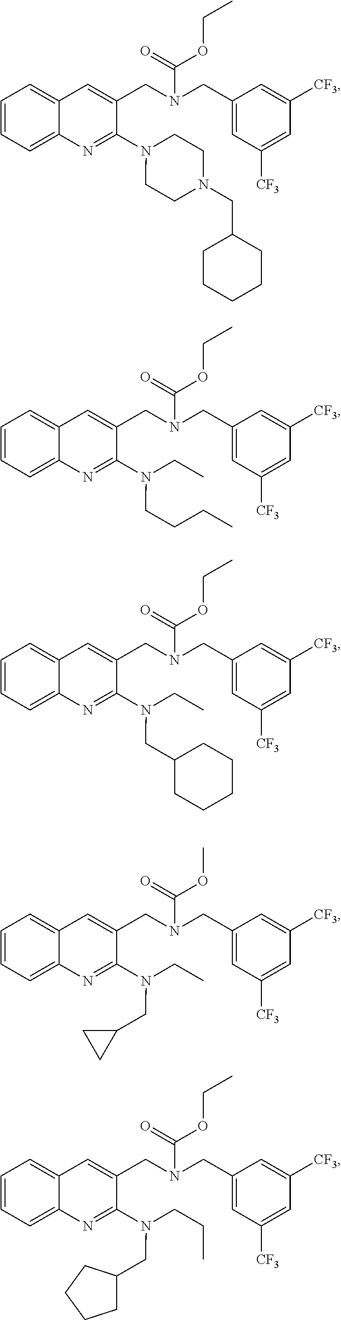 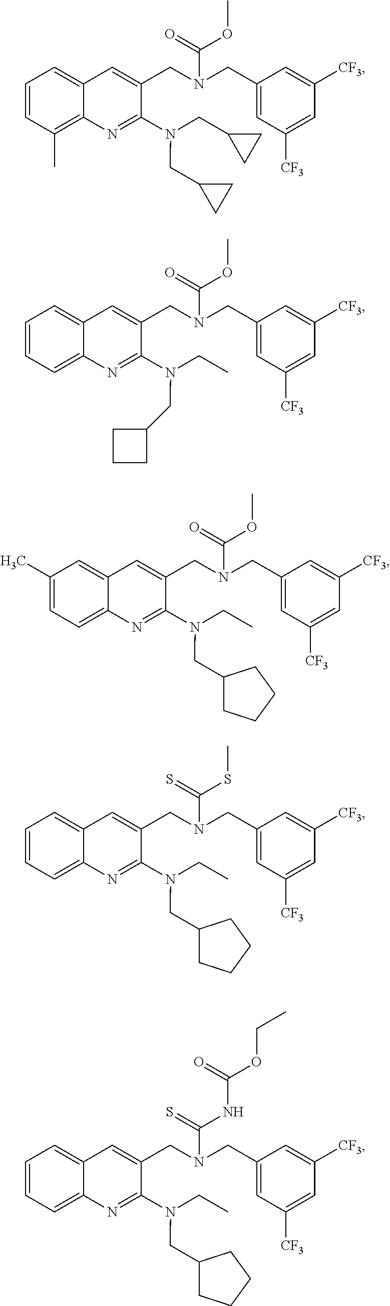 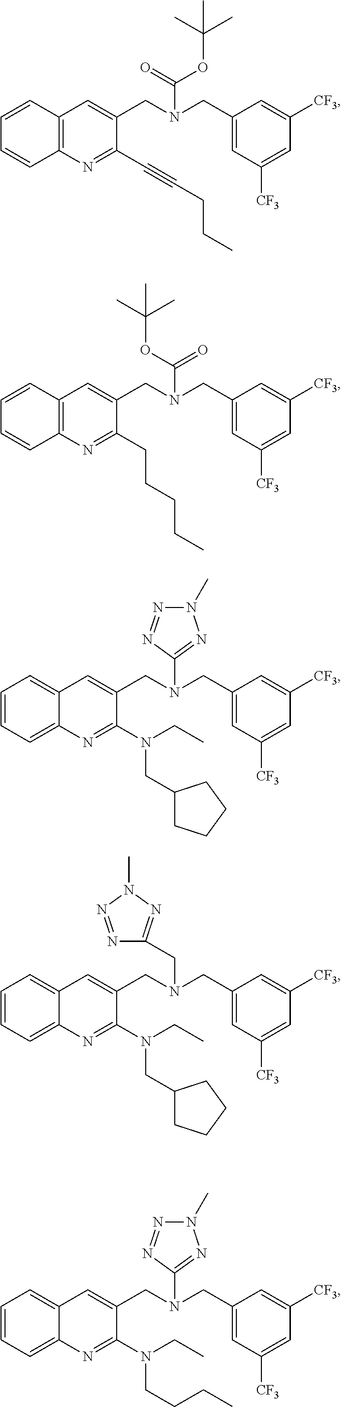 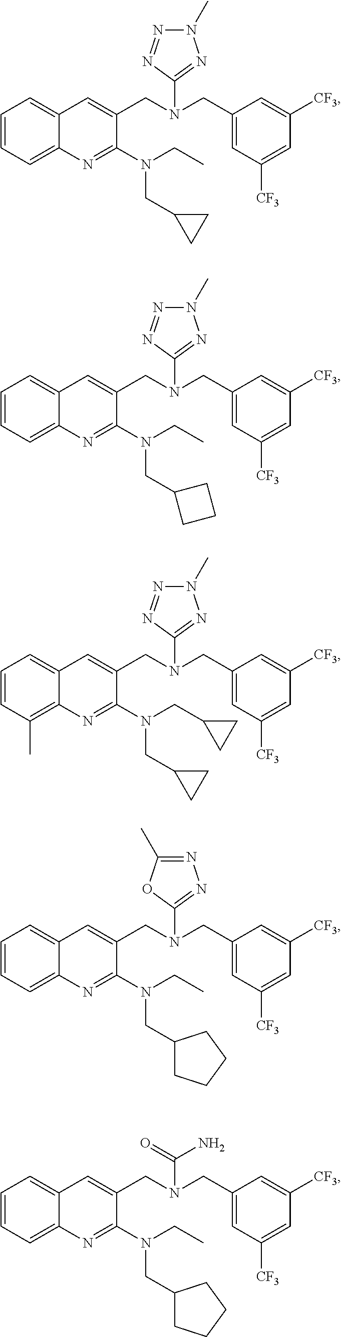 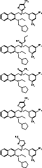 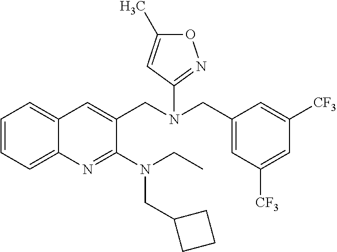 [SMILES: CCN(CC1CCC1)c1nc2ccccc2cc1CN(Cc1cc(C(F)(F)F)cc(C(F)(F)F)c1)c1cc(C)on1] 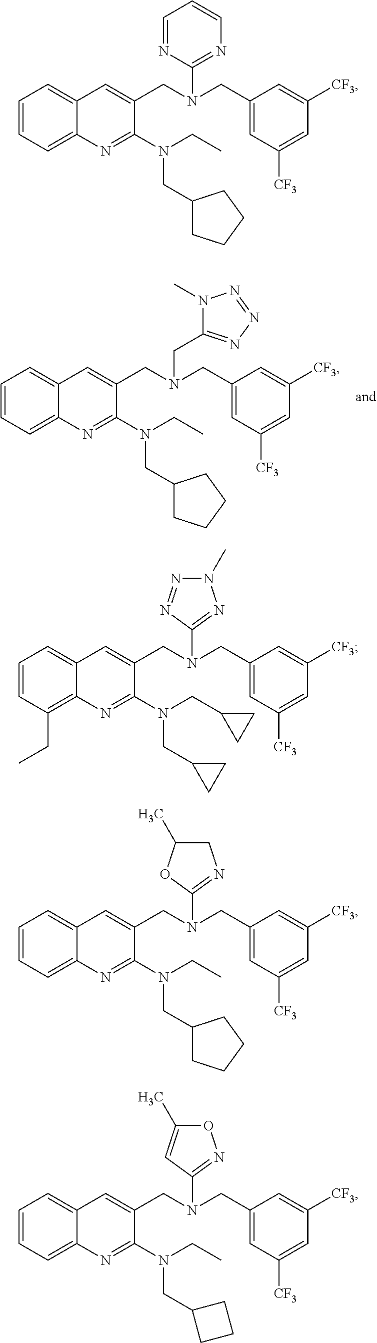 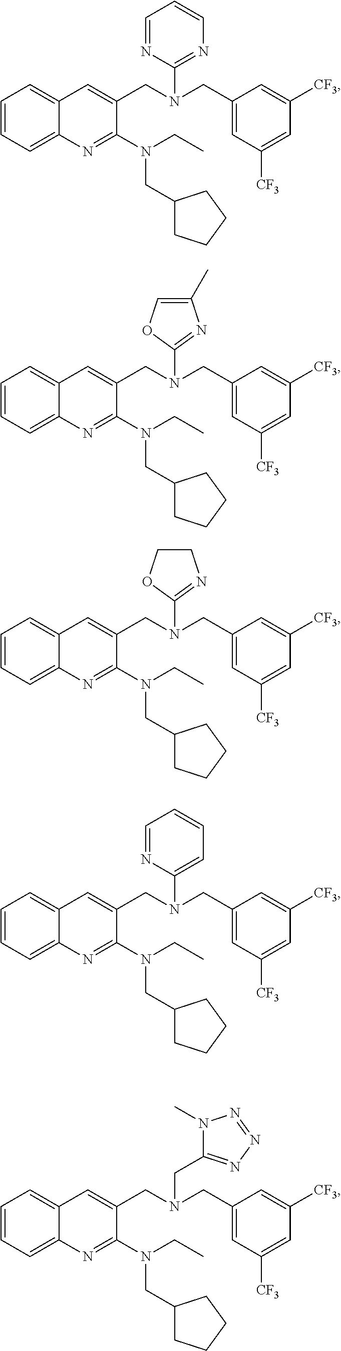 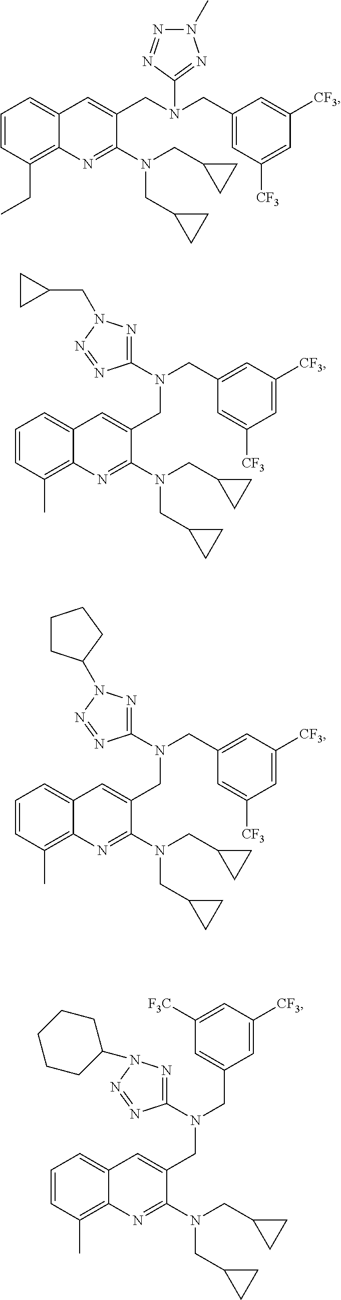 [SMILES: CCc1cccc2cc(CN(Cc3cc(C(F)(F)F)cc(C(F)(F)F)c3)c3nnn(C)n3)c(N(CC3CC3)CC3CC3)nc12 ; Cc1cccc2cc(CN(Cc3cc(C(F)(F)F)cc(C(F)(F)F)c3)c3nnn(CC4CC4)n3)c(N(CC3CC3)CC3CC3)nc12 ; Cc1cccc2cc(CN(Cc3cc(C(F)(F)F)cc(C(F)(F)F)c3)c3nnn(C4CCCC4)n3)c(N(CC3CC3)CC3CC3)nc12 ; Cc1cccc2cc(CN(Cc3cc(C(F)(F)F)cc(C(F)(F)F)c3)c3nnn(C4CCCCC4)n3)c(N(CC3CC3)CC3CC3)nc12] 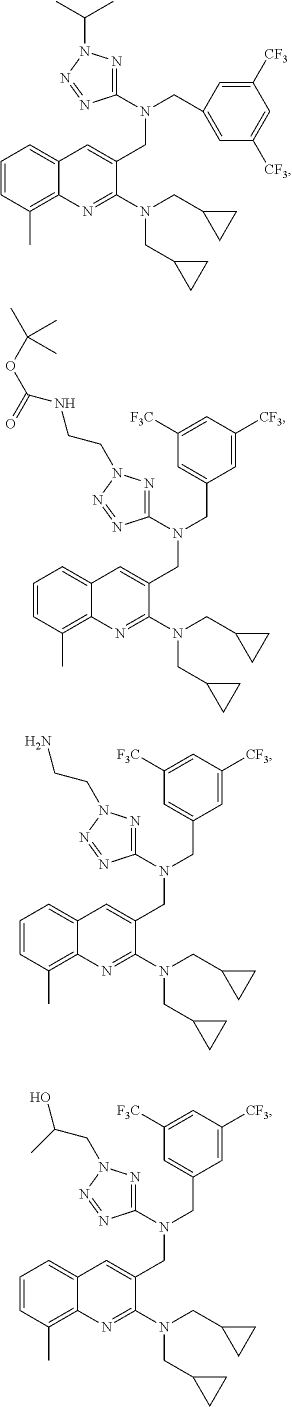 [SMILES: Cc1cccc2cc(CN(Cc3cc(C(F)(F)F)cc(C(F)(F)F)c3)c3nnn(C(C)C)n3)c(N(CC3CC3)CC3CC3)nc12 ; Cc1cccc2cc(CN(Cc3cc(C(F)(F)F)cc(C(F)(F)F)c3)c3nnn(CCNC(=O)OC(C)(C)C)n3)c(N(CC3CC3)CC3CC3)nc12 ; Cc1cccc2cc(CN(Cc3cc(C(F)(F)F)cc(C(F)(F)F)c3)c3nnn(CCN)n3)c(N(CC3CC3)CC3CC3)nc12 ; Cc1cccc2cc(CN(Cc3cc(C(F)(F)F)cc(C(F)(F)F)c3)c3nnn(CC(C)O)n3)c(N(CC3CC3)CC3CC3)nc12] 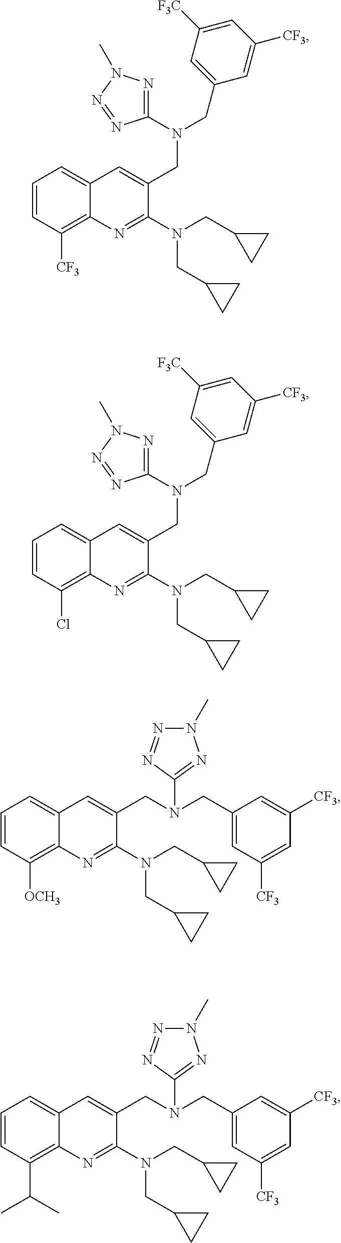 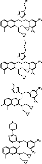 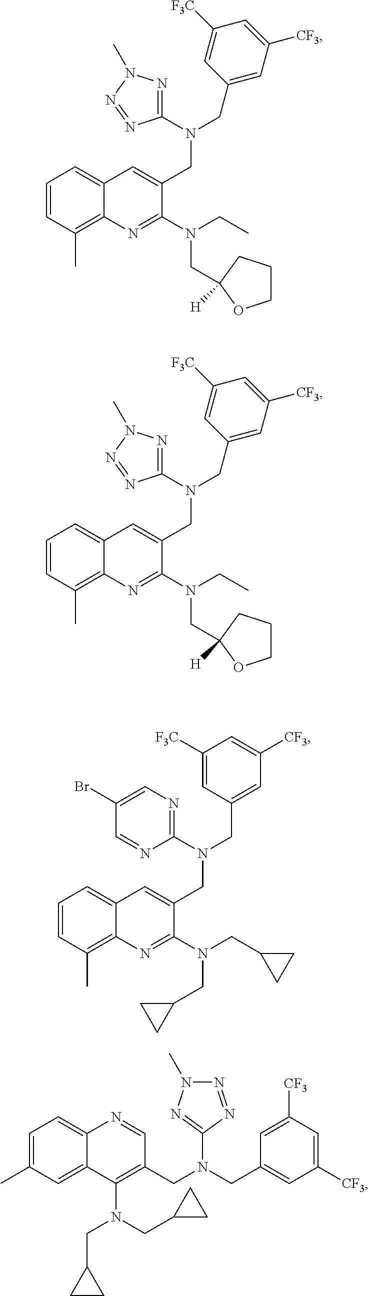 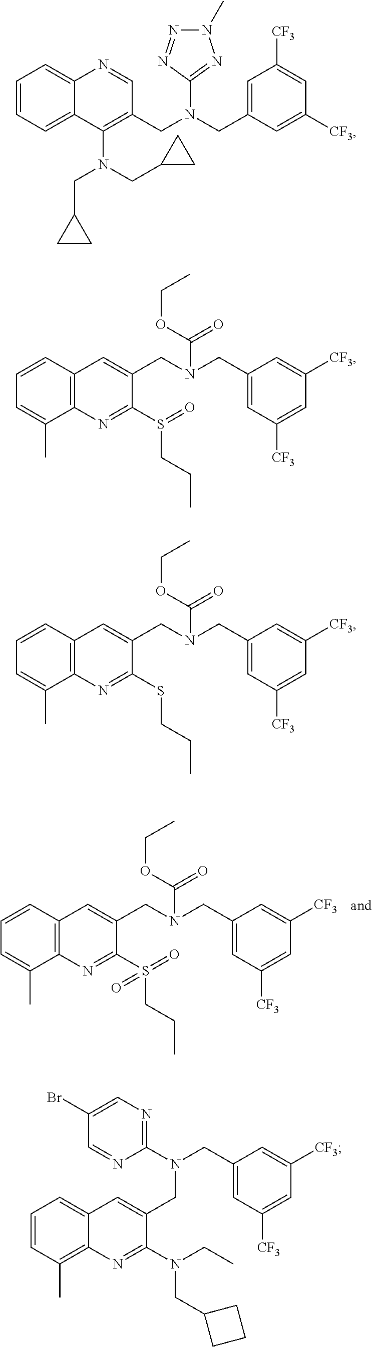
or a stereoisomer thereof or a pharmaceutically acceptable salt thereof.

34. The composition according to claim 1, wherein the CETP inhibitor is selected from a group consisting of: 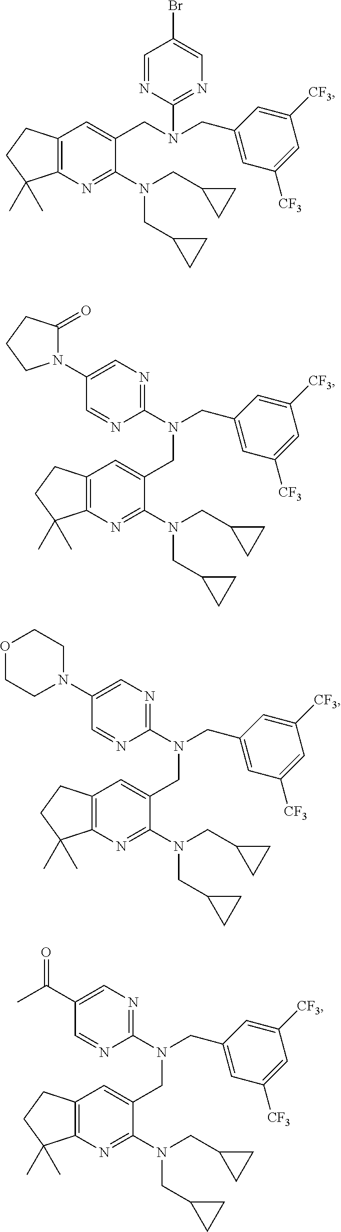 [SMILES: CC1(C)CCc2cc(CN(Cc3cc(C(F)(F)F)cc(C(F)(F)F)c3)c3ncc(Br)cn3)c(N(CC3CC3)CC3CC3)nc21 ; CC1(C)CCc2cc(CN(Cc3cc(C(F)(F)F)cc(C(F)(F)F)c3)c3ncc(N4CCCC4=O)cn3)c(N(CC3CC3)CC3CC3)nc21 ; CC1(C)CCc2cc(CN(Cc3cc(C(F)(F)F)cc(C(F)(F)F)c3)c3ncc(N4CCOCC4)cn3)c(N(CC3CC3)CC3CC3)nc21 ; CC(=O)c1cnc(N(Cc2cc(C(F)(F)F)cc(C(F)(F)F)c2)Cc2cc3c(nc2N(CC2CC2)CC2CC2)C(C)(C)CC3)nc1] 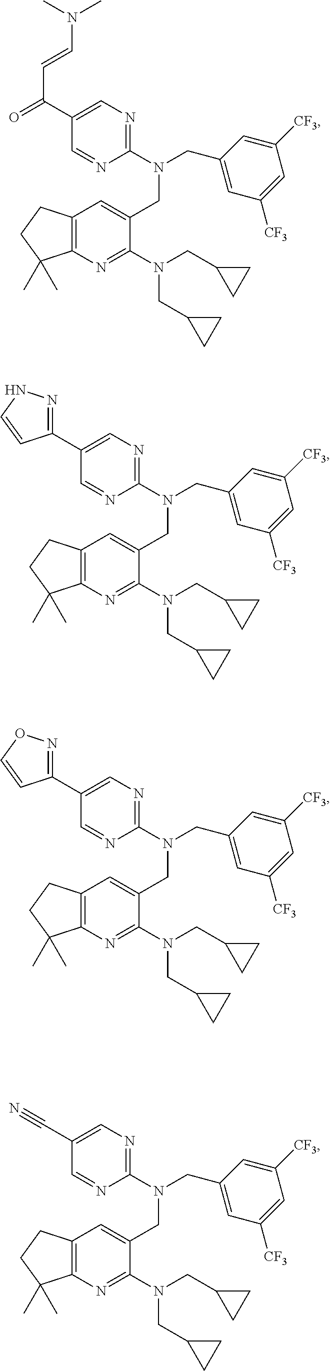 [SMILES: CN(C)/C=C/C(=O)c1cnc(N(Cc2cc(C(F)(F)F)cc(C(F)(F)F)c2)Cc2cc3c(nc2N(CC2CC2)CC2CC2)C(C)(C)CC3)nc1 ; CC1(C)CCc2cc(CN(Cc3cc(C(F)(F)F)cc(C(F)(F)F)c3)c3ncc(-c4cc[nH]n4)cn3)c(N(CC3CC3)CC3CC3)nc21 ; CC1(C)CCc2cc(CN(Cc3cc(C(F)(F)F)cc(C(F)(F)F)c3)c3ncc(-c4ccon4)cn3)c(N(CC3CC3)CC3CC3)nc21 ; CC1(C)CCc2cc(CN(Cc3cc(C(F)(F)F)cc(C(F)(F)F)c3)c3ncc(C#N)cn3)c(N(CC3CC3)CC3CC3)nc21] 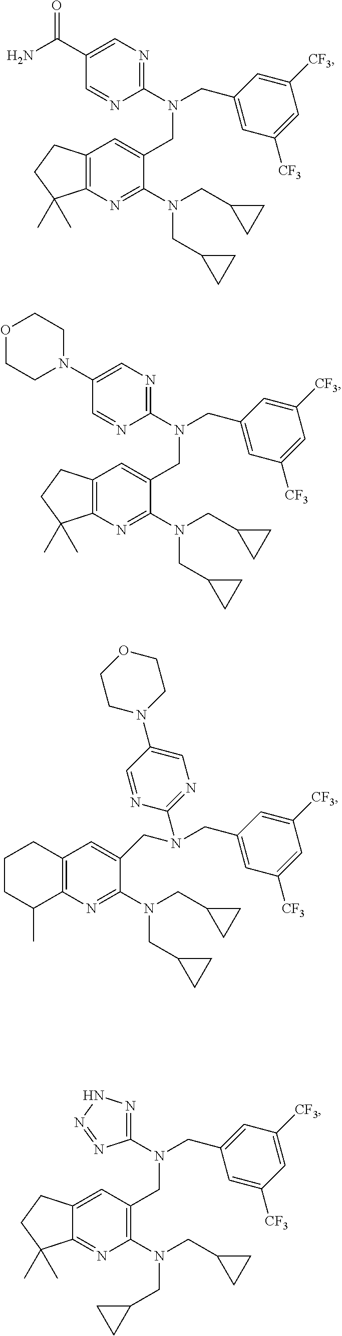 [SMILES: CC1(C)CCc2cc(CN(Cc3cc(C(F)(F)F)cc(C(F)(F)F)c3)c3ncc(C(N)=O)cn3)c(N(CC3CC3)CC3CC3)nc21 ; CC1(C)CCc2cc(CN(Cc3cc(C(F)(F)F)cc(C(F)(F)F)c3)c3ncc(N4CCOCC4)cn3)c(N(CC3CC3)CC3CC3)nc21 ; CC1CCCc2cc(CN(Cc3cc(C(F)(F)F)cc(C(F)(F)F)c3)c3ncc(N4CCOCC4)cn3)c(N(CC3CC3)CC3CC3)nc21 ; CC1(C)CCc2cc(CN(Cc3cc(C(F)(F)F)cc(C(F)(F)F)c3)c3nn[nH]n3)c(N(CC3CC3)CC3CC3)nc21] 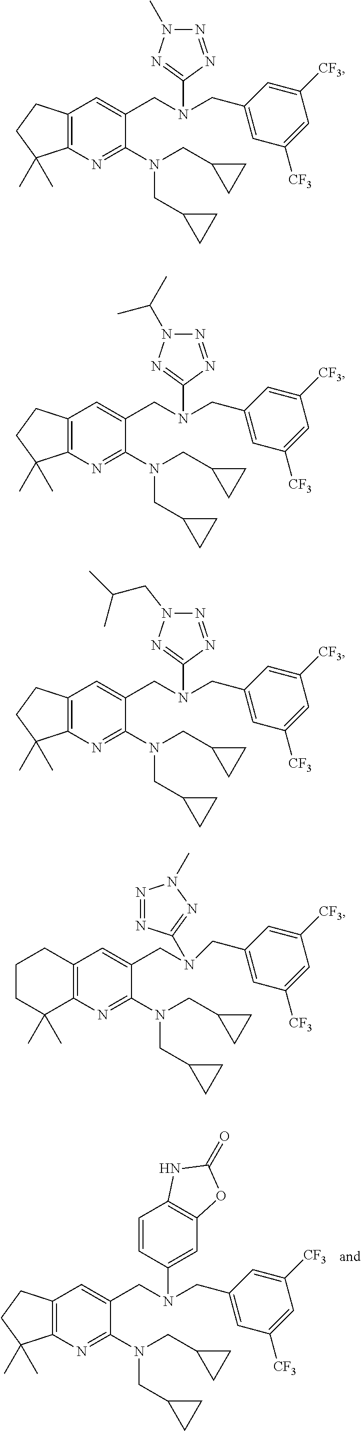 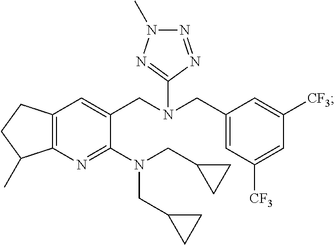 [SMILES: CC1CCc2cc(CN(Cc3cc(C(F)(F)F)cc(C(F)(F)F)c3)c3nnn(C)n3)c(N(CC3CC3)CC3CC3)nc21]
or a stereoisomer thereof or a pharmaceutically acceptable salt thereof.

35. The composition according to claim 1, wherein the CETP inhibitor is selected from a group consisting of: 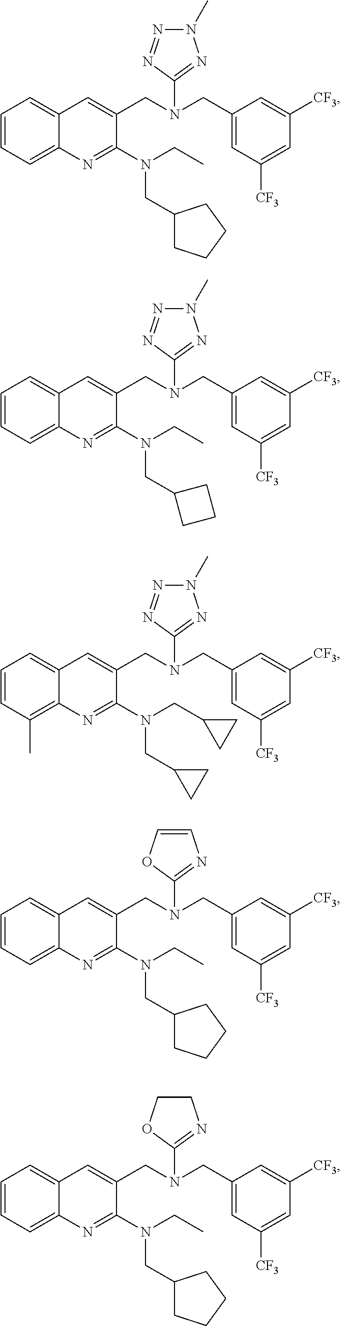 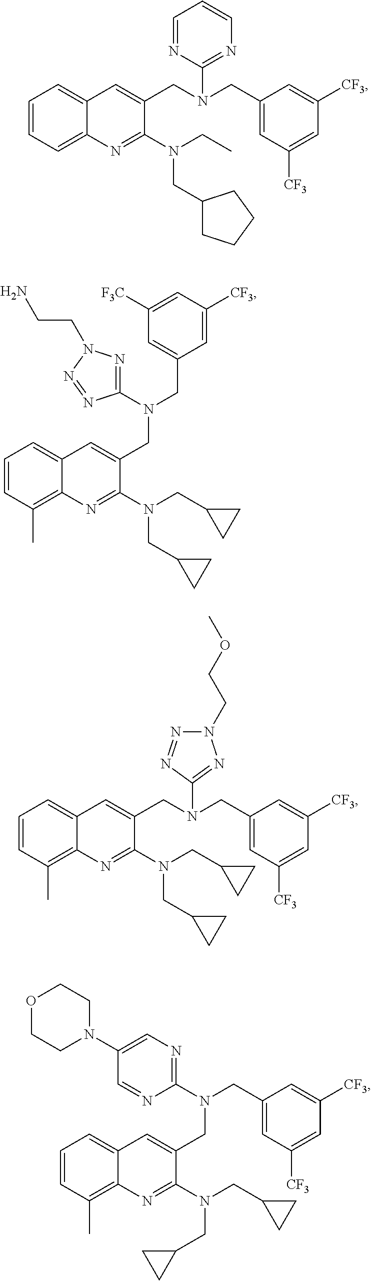 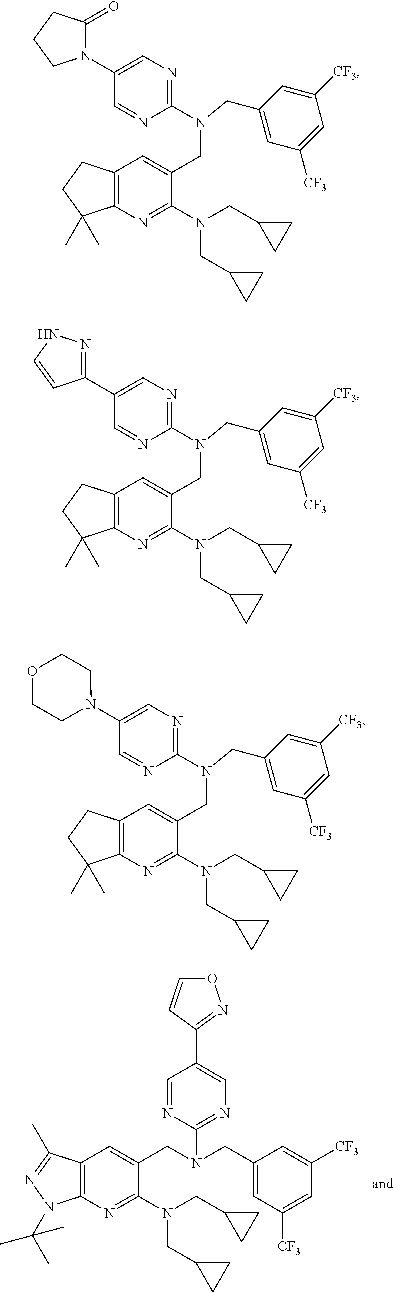 [SMILES: CC1(C)CCc2cc(CN(Cc3cc(C(F)(F)F)cc(C(F)(F)F)c3)c3ncc(N4CCCC4=O)cn3)c(N(CC3CC3)CC3CC3)nc21 ; CC1(C)CCc2cc(CN(Cc3cc(C(F)(F)F)cc(C(F)(F)F)c3)c3ncc(-c4cc[nH]n4)cn3)c(N(CC3CC3)CC3CC3)nc21 ; CC1(C)CCc2cc(CN(Cc3cc(C(F)(F)F)cc(C(F)(F)F)c3)c3ncc(N4CCOCC4)cn3)c(N(CC3CC3)CC3CC3)nc21 ; Cc1nn(C(C)(C)C)c2nc(N(CC3CC3)CC3CC3)c(CN(Cc3cc(C(F)(F)F)cc(C(F)(F)F)c3)c3ncc(-c4ccon4)cn3)cc12] 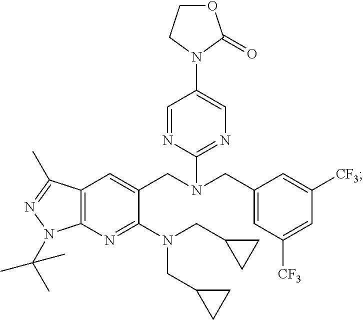 [SMILES: Cc1nn(C(C)(C)C)c2nc(N(CC3CC3)CC3CC3)c(CN(Cc3cc(C(F)(F)F)cc(C(F)(F)F)c3)c3ncc(N4CCOC4=O)cn3)cc12]
or a stereoisomer thereof or a pharmaceutically acceptable salt thereof.
